(19) United States
(12) Patent Application Publication
(10) Pub. No.: US 2018/0305719 A1
(43) Pub. Date: Oct. 25, 2018

(54) Vectors For Integration Of DNA Into Genomes And Methods For Altering Gene Expression And Interrogating Gene Function

(71) Applicant: The Board of Trustees of the University of Illinois, Urbana, IL (US)

(72) Inventors: Pablo Perez-Pinera, Urbana, IL (US); Alexander Brown, Champaign, IL (US); Wendy S. Woods, Champaign, IL (US)

(21) Appl. No.: 15/957,861

(22) Filed: Apr. 19, 2018

Related U.S. Application Data
(60) Provisional application No. 62/487,001, filed on Apr. 19, 2017.

(51) Int. Cl. C12N 15/90; C12N 15/11; C12N 9/22

(52) U.S. Cl. CPC C12N 15/907; C12N 15/11; A61K 48/00; C12N 2310/20; C12N 9/22

(57) ABSTRACT

The present disclosure provides vectors and methods for rapid and efficient integration of DNA at target sites in genomes with high efficiency. The present disclosure also provides methods for creating cell lines to model human diseases, for activating gene expression to correct genetic diseases or even for performing genetic screenings.

### CROSS-REFERENCE TO RELATED APPLICATIONS

This application claims priority to U.S. Provisional Patent Application No. 62/487,001, filed Apr. 19, 2017, the disclosure of which is hereby incorporated by cross-reference in its entirety.

### BACKGROUND

Gene editing technologies rely on the use of engineered nucleases to introduce targeted modifications in the genomes of living cells. In particular, the clustered regularly interspaced short palindromic repeats (CRISPR)/Cas9 RNA-guided nuclease (RGN) system, has revolutionized this field, providing a simple and efficient means of inducing DNA double-strand breaks (DSBs) at targeted genomic loci. In Streptococcus pyogenes, the CRISPR RNAs (crRNAs) and the trans-activating-crRNA (tracrRNA) form a complex that guides the Cas9 nuclease to the target DNA. The only constraint for target sequences is that they must immediately precede a suitable protospacer adjacent motif (PAM) of the form NGG⁵ or NGA⁶. This bacterial CRISPR system has been further simplified to utilize a single-guide RNA (sgRNA) molecule, which is a chimeric RNA that replaces both the crRNA and tracrRNA elements.

The CRISPR system has been adapted for use in mammalian cells, where gene knock out can be accomplished by introducing DSBs at the target locus that, when repaired by error-prone DNA repair pathways such as non-homologous end joining (NHEJ), cause inactivating mutations. Despite the high rates of allele modification that can be achieved with RGNs, the laborious and costly screening needed for identification and isolation of isogenic cell lines remains challenging in genetic engineering.

Alternatively, strain development can be streamlined by co-delivering engineered nucleases with donor vectors containing expression cassettes that confer antibiotic resistance for rapid clonal screening. These donor vectors often share a common architecture that consists of two DNA sequences homologous to the region of DNA upstream and downstream of the intended DSB, flanking the DNA that will be incorporated into the genome following repair of the DSB. Donor vectors stimulate DNA repair through homologous recombination (HR), a pathway that can be hijacked for targeted integration of DNA sequences into genomes. This method has been used successfully for multiple applications, including gene knock-out, delivery of therapeutic genes, or for tagging endogenous proteins. Gene editing via donor vectors is precise, however, it is inefficient and it relies on construction of lengthy homology arms using complex cloning strategies, costly synthesis of DNA fragments, or both.

Furthermore, an important drawback for genome engineering applications, which often requires integration of constructs in excess of 5 kb, is that the efficiency of HR decreases as the size of the DNA insert between the homology arms increases. More importantly, since homology between the donor vector and the target site is critical, each donor vector is necessarily associated with a specific sgRNA. Consequently, the time frame necessary for design, testing and validation of new strains generated using HR is excessively long. Platforms for rapid and low cost multiplexed genomic integration are needed.

Additionally, genome-scale gain-of-function screening is a powerful tool to systematically identify genes that regulate biological processes. The activation of endogenous genes with artificial transcription factors (ATFs) is an enticing technology, not only for developing gene therapies or disease models, but also for interrogating gene function through genome-wide screenings. ATFs consist of a programmable DNA binding domain that can be customized to target a transcriptional activation domain to the appropriate locus for upregulation of gene expression. While zinc finger proteins and Transcriptional Activator-Like Effectors (TALE) have been used for gene activation, the RNA guided nuclease (RGN) platform is arguably the most popular since the DNA binding specificity can be engineered rapidly and at low cost. RGN-based gene activation, also known as CRISPR (Clustered Regularly Interspaced Short Palindromic Repeats) activation or CRISPRa, requires a single-guide RNA (sgRNA) and catalytically dead Cas9 (dCas9) coupled with a transcriptional activator. First generation transcriptional activators, which typically used VP64 or VP16 activation domains, required multiple ATFs acting in synergy near the transcriptional start site (TSS) of the gene of interest for optimal gene activation. This important limitation is lessened when using second-generation transcriptional activators, including VP160, SAM, VPR, suntag, VP64-dCas9-BFP-VP64, Scaffold, and P300, which are capable of activating expression of some target genes when used individually.

A key application of second generation transcriptional activators has been the interrogation of gene function by introducing genetic perturbations at genome-scale using libraries of sgRNAs. However, the success of gain-of-function screenings fundamentally relies on the effective activation of target genes by the ATFs in order to overcome the applied selection pressure. Unfortunately, it is becoming evident that even second generation CRISPRa technologies are often limited by their need for multiple sgRNA to achieve adequate activation of many genes and the lack of established parameters to best position ATFs within endogenous promoters for effective upregulation of gene expression. These constraints in gain-of-function screenings by ATFs may lead to results that are skewed in favor of select subgroups of sgRNAs for which activation is readily achieved with a single sgRNA.

To address shortcomings in loss-of-function genome-scale screenings, hits from CRISPR knock out screenings can be refined by simultaneously considering hits from short hairpin RNA (shRNA) screenings. Unfortunately, there are no such alternatives to CRISPRa that function by a different mechanism and that, by having different advantages and limitations, can be used in parallel with CRISPRa screenings to comprehensively identify targets. While ideal outcomes from screenings require robust activation of target gene expression, current CRISPRa technologies often exhibit relatively weak, variable, or unpredictable activation across targets.

To address these limitations, a novel universal vector integration platform system for gene activation is described herein, which bypasses native promoters to achieve unprecedented levels of endogenous gene activation. Since genomic context at the promoter greatly impacts output expression when using ATFs, it is possible to circumvent this problem through insertion of a synthetic promoter near the transcriptional start site (TSS) of target genes. This system not only overrides negative regulatory elements, but is also highly customizable, given the existing assortment of well-characterized synthetic promoters capable of both constitutive and inducible gene expression.

This platform enables rapid, robust and inducible activation of both individual and multiplexed gene transcripts. This gene activation system is multiplexable and easily tuned for precise control of expression levels. Importantly, since promoter vector integration requires just one variable sgRNA to target each gene of interest, this procedure can be adapted for gain-of-function screenings. Collectively, these results demonstrate a novel system for gene modulation with wide adaptability in cell line engineering and genome-scale functional screenings.

### BRIEF SUMMARY OF THE INVENTION

The present disclosure relates to a system for targeted genome engineering and methods for altering the expression of genes and interrogating the function of genes.

One aspect of the present invention provides a system for targeted genome engineering, the system comprising one or more vectors comprising: (i) nucleic acids for integration in genomic DNA with no significant homology to the target sequence in genomic DNA; (ii) a single guide RNA (sgRNA) that binds one or more vectors; (iii) a sgRNA that binds a double-stranded nucleic sequence in genomic DNA where the vectors can be integrated; and (iv) a nuclease that causes a double-stranded nucleic acid break of the targeted nucleic acid molecules.

In some embodiments of the invention disclosed herein, the nucleic acids for integration in genomic DNA with no significant homology to the target sequence in genomic DNA; the single guide RNA (sgRNA) that binds one or more vectors; the sgRNA that binds a double-stranded nucleic sequence in genomic DNA where the vectors can be integrated; and the nuclease that causes a double-stranded nucleic acid break of the targeted nucleic acid molecules are located on the same or different vectors of the system.

In some embodiments of the invention disclosed herein, the sgRNA that binds one or more vectors and the sgRNA that binds a double-stranded nucleic sequence in genomic DNA where the vectors can be integrated are the same sgRNA. In other embodiments of the above aspect of the invention, the sgRNA that binds one or more vectors and the sgRNA that binds a double-stranded nucleic sequence in genomic DNA where the vectors can be integrated are different sgRNAs.

In some embodiments of the invention disclosed herein, the sgRNA that binds one or more vectors is a universal sgRNA.

In some embodiments of the invention disclosed herein, the nuclease is expressed from an expression cassette.

In some embodiments of the invention disclosed herein, the one or more vectors further comprises a polynucleotide encoding for a marker protein. In other embodiments of the invention disclosed herein, a sgRNA target site is cloned upstream of the marker protein. In other embodiments of the invention disclosed herein, the marker protein is an antibiotic resistance protein or a florescent protein.

In some embodiments of the invention disclosed herein, the polynucleotide encoding for a marker protein is expressed on a vector separate from the one or more vectors comprising the nucleic acids for integration in genomic DNA with no significant homology to the target sequence in genomic DNA; the single guide RNA (sgRNA) that binds one or more vectors; the sgRNA that binds a double-stranded nucleic sequence in genomic DNA where the vectors can be integrated; and the nuclease that causes a double-stranded nucleic acid break of the targeted nucleic acid molecules.

In some embodiments of the invention disclosed herein, the sgRNA that binds a double-stranded nucleic sequence in genomic DNA where the vectors can be integrated is complementary to a portion of the nucleic acid sequence of a target DNA.

In some embodiments of invention disclosed herein, the nucleic acids with no significant homology to the target nucleic acid molecule are about 0.1 kilobase to about 50 kilobases in size.

In some embodiments of the invention disclosed herein, the nuclease is a Zinc finger nuclease (ZFN), RNA guided nucleases (RGN), or transcription activator-like effector nucleases (TALEN). In other embodiments of the invention disclosed herein, the RGN is Caspase 9 (Cas9).

In some embodiments of the invention disclosed herein, the one or more vectors are plasmids or viral vectors. In other embodiments of the invention disclosed herein, the viral vector is a lentivirus vector, an adenovirus vector, or an adeno-associated vector (AAV).

In some embodiments of the invention disclosed herein, the system for targeted genome engineering further comprises one or more additional sgRNA molecules that causes a double-stranded nucleic acid break of one or more additional target nucleic acid molecules.

In some embodiments of the invention disclosed herein, the system does not require the entire vector that can be integrated to have any homology with the target site.

Another aspect of the present invention provides a method of altering the expression of at least one gene product, the method comprising: (i) introducing into a cell a system for targeted genome engineering as disclosed herein; and (ii) selecting for successfully transfected cells by applying selective pressure; wherein the expression of at least one gene product is reduced or eliminated relative to a cell that has not been transfected with the system for targeted genome engineering.

In some embodiments of the invention disclosed herein, the method occurs in vivo or in vitro. In other embodiments of the invention disclosed herein, the cell is a eukaryotic cell.

Another aspect of the present invention provides a system for targeted genome engineering, the system comprising one or more vectors comprising: (i) at least one nucleic acid with no significant homology to the target genomic DNA site and that contains a promoter for controlling gene expression; (ii) a primary sgRNA that binds the target nucleic acid molecule at or near the transcription start site of a gene in the target nucleic acid molecule; (iii) a universal secondary sgRNA that binds one or more vectors; and (iv) a nuclease that causes a double-stranded nucleic acid break of the targeted nucleic acid molecules.

In some embodiments of the invention disclosed herein, the at least one nucleic acid with no significant homology to the target genomic DNA site and that contains a promoter for controlling gene expression comprises: (1) a nucleic acid promoter followed by a universal secondary sgRNA; (2) two opposing, constitutive promoters separated by a universal secondary sgRNA; or (3) two inducible promoters in opposite orientations separated by an universal secondary sgRNA.

In some embodiments of the invention disclosed herein, the at least one nucleic acid with no significant homology to the target genomic DNA site and that contains a promoter for controlling gene expression; the primary sgRNA that binds the target nucleic acid molecule at or near the transcription start site of a gene in the target nucleic acid molecule; the universal secondary sgRNA that binds one or more vectors; and the nuclease that causes a double-stranded nucleic acid break of the targeted nucleic acid molecules are located on the same or different vectors of the system.

In some embodiments of the invention disclosed herein, each inducible promoter of the two inducible promoters in opposite orientations separated by a universal secondary sgRNA contains multiple TetO repeats and a transferase gene operatively linked to a reverse tetracycline transactivator (rtTA) via a T2A peptide.

In some embodiments of the invention disclosed herein, the one or more vectors further comprise a polynucleotide encoding for a marker protein. In other embodiments of the invention disclosed herein, the marker protein is an antibiotic resistance protein or a florescent protein.

In some embodiments of the invention disclosed herein, the nucleic acid promotor is heterologous to the promoter of the target nucleic acid molecule.

In some embodiments of the invention disclosed herein, the nuclease is a Zinc finger nuclease (ZFN), RNA guided nucleases (RGN), or transcription activator-like effector nucleases (TALEN). In other embodiments of the invention disclosed herein, the RGN is Caspase 9 (Cas9).

In some embodiments of the invention disclosed herein, the one or more vectors are plasmid or viral vectors. In other embodiments of the invention disclosed herein, the viral vector is a lentivirus vector, an adenovirus vector, or an adeno-associated vector (AAV).

Another aspect of the present invention provides a method of altering the expression of at least one gene product, the method comprising: (i) introducing into a cell a system for targeted genome engineering as disclosed herein; and (ii) selecting for successfully transfected cells by applying selective pressure, wherein the expression of at least one gene product is activated relative to a cell that is not transfected with the system of targeted genome engineering.

In some embodiments of the invention disclosed herein, the method occurs in vivo or in vitro. In other embodiments of the invention disclosed herein, the cell is a eukaryotic cell.

Another aspect of the present invention provides a method of identifying the genetic basis of one or more medical symptoms exhibited by a subject, the method comprising: (i) obtaining a biological sample from the subject and isolating a population of cells having a first phenotype from the biological sample; (ii) transfecting a library of sgRNA into the cells; (iii) introducing into the cells a system of targeted genome engineering as disclosed herein; (iv) selecting for successfully transfected cells by applying the selective pressure; (v) selecting the cells that survive under the selective pressure, (vi) determining the genomic loci of the DNA molecule that interacts with the first phenotype and identifying the genetic basis of the one or more medical symptoms exhibited by the subject.

In some embodiments of the invention disclosed herein, selective pressure is applied by contacting the cells with an antibiotic and selecting the cells that survive. In some embodiments of the method disclosed herein, the antibiotic is puromycin or hygromycin.

Additional features and advantages are described herein, and will be apparent from the following Detailed Description, Drawings and the claims.

### BRIEF DESCRIPTION OF THE DRAWINGS

The features, objects and advantages other than those set forth above will become more readily apparent when consideration is given to the detailed description below. Such detailed description makes reference to the following drawings, wherein:

FIG. 1 shows a schematic representation of the traditional approach to integrate heterologous DNA at target genomic loci using homologous recombination of donor vectors. The donor vector contains a homology region consisting of genomic DNA up to position −4 on the left and from position −3 onward (length ranges from 300 to 2,000 bp). Separation of the target sequence in 2 fragments is needed to prevent Cas9 from recognizing and degrading the donor.

FIG. 2A-2C shows a schematic representation of the major systems for targeted genome modification. FIG. 2A shows that in the absence of a template, mammalian cells prefer to use NHEJ to repair DSBs introduced with RGN at the target site. NHEJ is a mutagenic pathway that, by introducing insertions and deletions, can be used for gene inactivation. FIG. 2B shows homologous recombination is used in mammalian cells when a repair template is present. A repair template can be a donor vector with two arms that are homologous to the genomic DNA flanking the DSB. Heterologous DNA positioned between the homology arms can be integrated in the genome at the target site. FIG. 2C shows introduction of a DSB simultaneously in genomic DNA and a vector results in efficient integration of the entire vector at the target site by an unknown mechanism.

FIG. 3 shows a schematic representation of a proposed system for using Cas9 as RGN for Integration of DNA at Target Loci. The entire target CRISPR target sequence, including the PAM, is cloned into a preexisting vector where the DNA encoding the elements that need to be integrated is located.

FIG. 4 shows a gel of insertions and deletions with co-transfection of Cas9 and sgRNA in the ACTB, GAPDH, TUBB, NR0B2, CTTN-EX9, CTTN-EX8 target sites relative to control samples with GFP.

FIG. 5A shows a schematic of the transfer vectors. FIG. 5B is a gel image showing proof-of-principle studies with the genes ACTB (β-actin), GAPDH, and TUBB (β-tubulin), and NR0B2 (SHP1). Four gene specific transfer vectors containing the sequence targeted by the sgRNA in genomic DNA were prepared. When Cas9 and locus specific sgRNA were co-transfected with a donor vector that contains the same target sequence, the plasmids were integrated at the target site in the genome.

FIG. 6A-6B shows that NAVI is multiplexable but integration is not strand specific. FIG. 6A shows a schematic and gel image of the analysis of genomic integration of two different transfer vectors that target GFP to the GAPDH locus or RFP to the ACTB locus by co-transfection with Cas9 and sgRNAs targeting GAPDH or ACTB. PCR detecting integration of GFP at the GAPDH locus demonstrates that Cas9, GAPDH sgRNA as well that the GAPDH-GFP transfer vector are required, however, when ACTB sgRNA is also expressed, integration of GFP can also occur at the ACTB locus. Similarly, analysis of RFP integration the ACTB locus demonstrates that Cas9, ACTB sgRNA and the ACTB-RFP transfer vector are required, but a simultaneous DSB at GAPDH results in integration of ACTB-RFP at the ACTB locus. FIG. 6B shows a schematic and gel image of the target sequence of two ACTB sgRNAs that target the plus or minus strand of the ACTB gene were inserted in a transfer vector in orientations plus or minus. Each of these transfer vectors was transfected in combination with Cas9 and each of the ACTB sgRNAs. Introduction of a DSB in genomic DNA led to integration of each transfer vector in both orientations regardless of the strand targeted by the sgRNA.

FIG. 7A shows a schematic of the generation of clonal cell lines with integration of a transfer vector at the NR0B2 locus by co-transfection of Cas9, NR0B2 sgRNA, and a NR0B2 transfer vector. FIG. 7B shows a gel image visualizing out-in and in-out PCRs with various primer combinations to detect integration of different fragments of the NR0B2 transfer vector in genomic DNA. The length of the different fragments detected shows that the entire vector was integrated.

FIG. 8A shows a schematic of the generation of TALENs targeting the ACTB locus and included their target sequence into a transfer vector. FIG. 8B shows a gel image showing that when the TALENs were transfected together with the transfer vector, specific integration of the vector at the target locus was readily detected. While GAPDH RGNs were not sufficient to integrate the circular transfer vector containing the TALEN ACTB site, when the vector was linearized with ACTB specific TALENs, it was incorporated successfully at the GAPDH locus upon induction of a DSB with RGNs.

FIG. 9A-9B shows that NAVI can efficiently introduce large vectors, including BACs and phage genomes, into genomic DNA of mammalian cells using universal RGNs. FIG. 9A shows a schematic and gel image of GAPDH RGNs that were transfected with T7 sgRNA and 4 different transfer vectors with sizes ranging from 6.3 kb to 12.1 kb. Each of these plasmids contained a T7 priming site compatible with the T7 sgRNA. The transfer vectors were transfected both individually and in combination. PCR with primer pairs that bind genomic DNA and each of the vectors successfully detected integration at the GAPDH locus for each of the vectors. When the four vectors were transfected simultaneously, each of them was detected at the target site in a pooled cell population. FIG. 9B shows a schematic and gel images of either the bacterial artificial chromosome (˜25 kb) or the lambda phage genome (˜50 kb) that were transfected in combination with Cas9, a TUBB sgRNA and a vector-specific RGN. PCRs in pooled cells with primers that amplify the expected junction of genomic DNA with each of the vectors demonstrated successful integration of both DNAs at the target site.

FIG. 10A-10D shows rapid biallelic modification introduced by NAVI can be used to generate gene knock outs or orthogonal gene knock out and gene activation. FIG. 10A shows a schematic and gel images of HCT116 cells that were transfected with CTTN sgRNA, transfer vectors encoding PuroR and/or HygroR genes and vector specific RGNs. Only when Cas9 introduced a DSB simultaneously in the transfer vector and in the target loci in genomic DNA was the transfer vector integrated and CTTN disrupted. When both transfer vectors were transfected in conjunction with Cas9 and both CTTN and sgRNAs, integration of both vectors was detected at the same locus indicating biallelic modification in this diploid cell line. FIG. 10B shows gel images of cell lines transfected with CTTN, sgRNAs, Cas9 and both PuroR and HygroR transfer vectors underwent selection with puromycin and hygromycin before 5 clones and a control cell line (C) were isolated and analyzed for integration of the transfer vectors at the CTTN locus. Four of the five clones were homozygous for the mutation, whereas one clone was heterozygous. FIG. 10C shows a Western blot of CTTN expression in the four homozygous clones, which confirmed that CTTN was effectively knocked out. FIG. 10D shows schematics and gel images of HCT116 cells that were transfected with two RGNs targeting the CTTN and HLA-DRA loci as well as 4 plasmids encoding genes that provide resistance to puromycin, hygromycin, blasticidin or neomycin. Simultaneous treatment with the four antibiotics selected cell lines that incorporated one plasmid in each allele of the 2 genes targeted with RGNs. One of the ten cell lines analyzed had four alleles modified, 5 cell lines had 3 alleles modified, 2 cell lines had 2 alleles modified, one cell line had one allele modified and one was wt.

FIG. 11 shows a gel image visualizing potential off-site target sites of the RGN.

FIG. 12 shows a schematic of the identification of mutations at the junctions of genomic DNA (plus vector integration GAPDH—left set of sequence top to bottom are SEQ ID NO:177, 178, 179 and 180 respectively; plus vector integration GAPDH—right set of sequence top to bottom are SEQ ID NO:181, 182, 183 and 184 respectively; minus vector integration GAPDH—left set of sequence top to bottom are SEQ ID NO:185, 186, 187 and 188 respectively; minus vector integration GAPDH—right set of sequence top to bottom are SEQ ID NO:189, 190, 191 and 192 respectively; and plus vector integration ACTB—left set of sequence top to bottom are SEQ ID NO:193, 194 and 195 respectively; plus vector integration ACTB—right set of sequence top to bottom are SEQ ID NO:196, 197, and 198 respectively; minus vector integration ACTB—left set of sequence top to bottom are SEQ ID NO:199, 200, and 201 respectively; minus vector integration ACTB—right set of sequence top to bottom are SEQ ID NO:202, 203 and 204 respectively).

FIG. 13 shows a schematic representation of a procedure for gene activation using RGNs. This method consists of three stages: (1) sgRNA expression vectors are designed and generated using a single-step digestion, phosphorylation, and ligation reaction, (2) native gene expression is activated by co-delivery of sgRNA and dCas9-transcriptional activator expression plasmids into the target cells, and (3) RNA is isolated and analyzed using qPCR to quantify relative changes in gene expression.

FIG. 14A-14B shows that the NAVIa activation of native gene expression is tunable and surpasses CRISPRa. FIG. 14A shows a schematic of the architecture of the NAVIa system includes a plasmid containing a human codon-optimized expression cassette for active Cas9, which is co-transfected with two separate sgRNA plasmids and a targeting vector (idpTV, cdpTV or cspTV). The primary sgRNA is designed to bind and target Cas9 to the 5′ region of the gene of interest, while the secondary sgRNA target site is at the 3′ end of the cspTV promoter, or between the diametric promoters of the cdpTV and idpTV. After Cas9 cuts the TV, the resulting linearized vector is integrated at the target site in genomic DNA, presumably via NHEJ repair of the double-stranded breaks. FIG. 14B is a graph showing the ability of NAVIa to upregulate the expression of target transcript within pooled, selected 293T cells across a panel of three genes: ASCL1, NEUROD1, and POUF51. Each sgRNA employed within NAVIa was also used for CRISPRa (dCas9-VPR) either alone or in conjunction with three additional sgRNAs, previously reported to activate expression of the target mRNA measured by qPCR. Data shown as the mean±s.e.m. (n=3 independent experiments). P-values were determined by t-test: idpTV versus 4 sgRNAs: p≤0.05 for all targets, cdpTV versus 4 sgRNA: p≤0.05 for ASCL1, idpTV, cspTV or cdpTV versus 1 sgRNA: p≤0.05 for all targets.

FIG. 15 is a graph showing expression of a single-guide RNA targeted to the NeuroD1 locus in the cell lines HCT116, MRCS and Neuro2a, which was was co-transfected with plasmids encoding active Cas9, the secondary sgRNA and the cdpTV. Expression of NeuroD1 was evaluated using qPCR (n=1).

FIG. 16 is a graph showing a representation of levels of activation relative to distance between sgRNA targeting and the canonical TSS.

FIG. 17 shows a schematic of sequencing the PCR amplicon of the TV-NEUROD1 juncture from eight NAVIa clones, which revealed limited indel formation in only two clones, while six of the eight clones contained flawless ligation of each DSB end (Exp(top), C2, and C3 are SEQ ID NO:205; C6 is SEQ ID NO:206; C8 is SEQ ID NO:207; C1, C4, C5, C7 and Exp(bottom) are SEQ ID NO:208).

FIG. 18. is a graph showing expression levels of NEUROD1 that was induced using NAVIa for a period of 4 days at concentrations of doxycycline ranging from 2 ng/mL to 2 μg/mL and measured using qPCR.

FIG. 19 is a graph showing expression of NeuroD1 that was measured by qPCR upon induction with 200 ng/mL doxycycline for 12, 24, 48 and 96 hours in 293T cells in which NeuroD1 was edited using NAVIa. Data in b, d and e are shown as the mean±s.e.m. (n=3 independent experiments).

FIG. 20 is a graph showing that the idpTV was integrated at the TERT locus in SF7996 primary glioblastoma cells and expression of TERT was increased in a dose-dependent manner by addition of doxycycline compared with untreated control cells (n=4, p<0.005). N.D.: not detected.

FIG. 21 is a graph showing the relative proliferation rate over 120 days, which was calculated as the ratio of number of cells cultured in doxycycline-free medium and number of cells in cultures treated with doxycycline (n=2).

FIG. 22 is a graph showing 293T cells transfected with CRISPRa or NAVIa targeting simultaneously the genes ASCL1, NEUROD1, POUF51, IL1B, IL1R2, LIN28A and ZFP42. Expression of the target genes without selection was measured at day 3 without using qPCR (n=2 independent experiments). Data is shown as mean±s.e.m. P-values were determined by t-test (NAVIa versus VPR, p≤0.001 ASCL1, p≤0.02 IL1B (Ct value of control sample was not detected and assumed to be 40), p≤0.004 IL1R2, p≤0.001 LIN28A, p≤0.001 NEUROD1, p≤0.007 POUF51, p≤0.001 ZFP42).

FIG. 23 is a graph showing the average background gene expression levels achieved for each gene target, which were represented in relation with the distance between the target of the sgRNA and the ATG codon. Linear regression modeling indicates lack of a relationship.

FIG. 24 is a graph showing linear regression modeling between basal gene expression and average background activation levels after idpTV integration without induction. No corollary relationship was revealed. This finding denotes another important difference between NAVIa and CRISPRa, which achieves highest levels of activation from genes that are not expressed at steady state.

FIG. 25 is a graph showing mRNA expression levels from a single sgRNA that was designed to target four additional promoters, prior to their inclusion within multiplexed transfections. Induction of expression was achieved by treatment of the cells with 200 ng/mL doxycycline for four days and evaluated by qPCR. Data represents mean±s.e.m.

FIG. 26 is a graph showing a comparison of background and induced expression of NEUROD1 targeted using NAVIa between pooled HCT116 cells (diploid) and clones that were positive for idpTV integration at either one or both alleles (n=3 independent experiments). Untreated pooled cells versus heterozygous, p≤0.003. Untreated heterozygous versus homozygous, p≤0.07. Untreated pooled cells versus homozygous, p≤0.0005. Doxycycline treated heterozygous versus homozygous, p≤0.001. Doxycycline treated pooled cells versus homozygous, p≤0.001. Data in a, b and c are shown as the mean±s.e.m.

FIG. 27A-27G shows that NAVIa is compatible with genome-scale gain-of-function screens. FIG. 27A shows a schematic of the workflow of a NAVIa genome-scale gain-of-function screen, which involves sgRNA library production and incorporation into a lentiviral delivery system, followed by lentiviral transduction into the cell line of interest. Then, the pre-transduced cells are transfected with active Cas9, the NAVIa transfer vector of choice, and the universal secondary sgRNA. After puromycin selection, the cell pool is ready for gain-of-function screens, followed by NGS to analyze results. FIG. 27B is a graph showing P-values of the top ranked gene hits from each screening method, CRISPRa and NAVIa, illustrating that each technique yields similar statistical significance across top candidate genes FIG. 27C is a graph showing MAGeCK assigned p-values for positive selection obtained from NAVIa and CRISPRa screening ordered by chromosomal position, illustrating that similar levels of enrichment were achieved by CRISPRa and NAVIa. FIG. 27D is a graph showing the top hits of CRISPRa (X-axis) and NAVIa (Y-axis) screenings were ranked by p-value of the positively-selected sgRNAs. Each screen yielded significant hits but only one gene within the top 25 hits, IPO9, was identified by both methods. FIG. 27E are graphs showing the p-values of the top 25 hits from NAVIa screening, which are represented in conjunction with the p-values for the same hits in the CRISPRa screening and the top 25 hits from CRISPRa screening are represented in conjunction with the p-values for the same hits in the NAVIa. FIG. 27F is a graph showing that the activation of CHSY1, GDF9, MFSD2B, HMGCL, and IPO9 expression was accomplished in MCF7 cells using NAVIa. The cells were treated with 5 μM 4-hydroxytamoxifen for 10 days and the number of surviving cells was estimated by manual counting. Results are represented as ratio of 4-hydroxytamoxifen-treated/untreated cells. *, p<0.1. **, p<0.05 (n=4 independent experiments). FIG. 27G is a graph showing TCGA expression data for the top ten genome-wide 4-hydroxytamoxifen resistance screen hits from both the CRISPRa and NAVIa in ER+ (left bar) and ER− (right bar) breast cancers.

FIG. 28 is a schematic showing a template with the NGS primers (U6 F2 is SEQ ID NO:209; EF1a rev is SEQ ID NO:210; SAM lib FWD1 is SEQ ID NO:211; SAM lib FWD3 is SEQ ID NO:212; SAM lib FWD5 is SEQ ID NO:213; SAM lib FWD7 is SEQ ID NO:214; SAM lib FWD9 is SEQ ID NO:215; SAM lib REV1 is SEQ ID NO:216; SAM lib REV2 is SEQ ID NO:217; Amplicon is SEQ ID NO:218).

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the embodiments above and the claims below. Reference should therefore be made to the embodiments above and claims below for interpreting the scope of the invention.

### DETAILED DESCRIPTION OF THE INVENTION

The system and methods now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements.

Likewise, many modifications and other embodiments of the system and methods described herein will come to mind to one of skill in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

Articles “a” and “an” are used herein to refer to one or to more than one (i.e. at least one) of the grammatical object of the article. By way of example, “an element” means at least one element and can include more than one element.

As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The term “about” in association with a numerical value means that the numerical value can vary plus or minus by 5% or less of the numerical value.

Overview

The present disclosure provides a multiplexable and universal nuclease-assisted vector integration system for rapid generation of gene knockouts using selection that does not require customized targeting vectors, thereby minimizing the cost and time needed for gene editing. Importantly, this system is capable of remodeling native genomes (e.g. mammalian) through integration of large DNA, (e.g., about 50 kb), enabling rapid generation and screening of multigene knockouts from a single transfection. These results support that nuclease assisted vector integration is a robust tool for genome-scale gene editing that will facilitate diverse applications in synthetic biology and gene therapy.

Also described herein are vectors and methods for rapid and efficient integration of heterologous DNA at target sites in genomes with high efficiency. These methods can be adapted to precisely manipulate and activate native gene expression. Furthermore, these techniques can be used for creating cell lines to model human diseases, for activating gene expression to correct genetic diseases or even for performing genetic screenings.

In one aspect, a system for targeted genome engineering, the system comprising one or more vectors comprising: (i) nucleic acids for integration in genomic DNA with no significant homology to the target sequence in genomic DNA; (ii) a single guide RNA (sgRNA) that binds one or more vectors; (iii) a sgRNA that binds a double-stranded nucleic sequence in genomic DNA where the vectors will be integrated; and (iv) a nuclease that causes a double-stranded nucleic acid break of the targeted nucleic acid molecules.

As used herein, the term “targeted genome engineering” refers to a type of genetic engineering in which DNA is inserted, deleted, modified, or replaced in the genome of a living organism or cell. Targeted genome engineering can involve integrating nucleic acids into genomic DNA at a target site of interest in order to manipulate (e.g., increase, decrease, knockout, activate) the expression of one or more genes.

As used herein, the term “knockout” refers to a genetic technique in which one of an organism's genes is made inoperative. Knocking out two genes simultaneously in an organism is known as a double knockout. Similarly, triple knockout (TKO) and quadruple knockouts (QKO) are used to describe three or four knocked out genes, respectively. Heterozygous knockouts refer to when only one of the two gene copies (alleles) is knocked out, and homozygous knockouts refer to when both gene copies are knocked out.

As used herein the term “activate” refers to activation of native gene expression, which can include, but is not limited to, increasing the levels of gene products or initiating gene expression of a previously inactive gene. Robust and controllable systems for activation of native gene expression have been pursued for multiple applications in gene therapy, regenerative medicine and synthetic biology. These systems, rather than introducing heterologous genes that are expressed from constitutive or tunable promoters, use proteins that regulate transcription of genes in their natural chromosomal context. There are several advantages to activating native gene expression compared with overexpressing exogenous genes including ease of cloning, simple delivery, tunability and potential for simultaneous regulation of multiple gene splicing isoforms.

As used herein, “single guide RNA” (the terms “single guide RNA” and “sgRNA” may be used interchangeably herein) refers to a single RNA species capable of directing RNA-guided nuclease (RGN) mediated cleavage of target DNA. In some embodiments, a single guide RNA may contain the sequences necessary for RGN nuclease activity and a target sequence complementary to a target DNA of interest.

As used herein, the terms “universal sgRNA,” “secondary sgRNA,” or “universal secondary sgRNA” are used interchangeably to refer to sgRNA that binds to and directs RGN-mediated cleavage of one or more vectors.

As used herein, the term “primary sgRNA” is used to refer to the sgRNA that binds to and directs RGN-mediated cleavage genomic DNA. The primary sgRNA can be customized to integrate nucleic acids (e.g., vectors) at any target site in the genome.

As used herein, the term “no significant homology to the target sequence in genomic DNA” means that the nucleic acids to be inserted into the genomic DNA have less than about 20%, 15%, 10%, 5%, or 1% homology to the genomic DNA. As used herein, the term “homology” refers to the similarity between two nucleic acid sequences. Homology among DNA, RNA, or proteins is typically inferred from their nucleotide or amino acid sequence similarity. Significant similarity is strong evidence that two sequences are related by evolutionary changes from a common ancestral sequence. Alignments of multiple sequences are used to indicate which regions of each sequence are homologous. The term “percent homology” is used herein to mean “sequence similarity.” The percentage of identical nucleic acids or residues (percent identity) or the percentage of nucleic acids residues conserved with similar physicochemical properties (percent similarity), e.g. leucine and isoleucine, is used to quantify the homology.

As described herein, sequence identity is related to sequence homology. Homology comparisons may be conducted by eye or using sequence comparison programs. These commercially available computer programs may calculate percent (%) homology between two or more sequences and may also calculate the sequence identity shared by two or more amino acid or nucleic acid sequences. Sequence homologies may be generated by any of a number of computer programs known in the art, for example BLAST or FASTA.

Percentage (%) sequence homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Ungapped alignments are performed only over a relatively short number of residues. Although this is a very simple and consistent method, it fails to take into consideration that, for example, in an otherwise identical pair of sequences, one insertion or deletion may cause the following amino acid residues to be put out of alignment, thus potentially resulting in a large reduction in percent homology when a global alignment is performed. Therefore, most sequence comparison methods are designed to produce optimal alignments that take into consideration possible insertions and deletions without unduly penalizing the overall homology or identity score. This is achieved by inserting “gaps” in the sequence alignment to try to maximize local homology or identity.

In some embodiments, the nucleic acids for integration in genomic DNA with no significant homology to the target sequence in genomic DNA; the single guide RNA (sgRNA) that binds one or more vectors; the sgRNA that binds a double-stranded nucleic sequence in genomic DNA where the vectors can be integrated; and the nuclease that causes a double-stranded nucleic acid break of the targeted nucleic acid molecules are located on the same or different vectors of the system. In other embodiments, the sgRNA that binds one or more vectors and the sgRNA that binds a double-stranded nucleic sequence in genomic DNA where the vectors can be integrated are the same sgRNA. In yet other embodiments, the sgRNA that binds one or more vectors and the sgRNA that binds a double-stranded nucleic sequence in genomic DNA where the vectors can be integrated are diffrent sgRNAs. In yet other embodiments, the sgRNA that binds one or more vectors is a universal sgRNA.

In some embodiments, multiple vectors can be integrated into one genomic site, where the multiple vectors are linearized by being cut by a single sgRNA, the vectors all having the target nucleic acid sequence for one sgRNA, so a single sgRNA can target the RGN to cut and linearize the vectors at a particular sequence located in all the vectors. All the vectors can be integrated into a target DNA of interest that has been cut by the RGN and inserted into a target DNA of interest that has been cut by an RGN targeted by a sgRNA complementary to a nucleic acid sequence located in the target DNA of interest.

In other embodiments, the nuclease is expressed from an expression cassette. The term “expression cassette” as used herein refers to a distinct component of vector DNA consisting of a gene and regulatory sequence to be expressed by a transfected cell, whereby the expression cassette directs the cell to make RNA and protein. Different expression cassettes can be transfected into different organisms including bacteria, yeast, plants, and mammalian cells as long as the correct regulatory sequences are used.

In other embodiments, the one or more vectors further comprises a polynucleotide encoding for a marker protein. In yet other embodiments, a sgRNA target site is cloned upstream of the marker protein. In yet other embodiments, the marker protein is an antibiotic resistance protein or a florescence protein. In some embodiments, the polynucleotide encoding for a marker protein is expressed on a separate vector.

As used herein, the terms “marker protein” or “selectable marker” are used interchangeably herein to refer to proteins encoded by a gene that when introduced into a cell (prokaryotic or eukaryotic) confers a trait suitable for artificial selection. Marker proteins or selectable markers are used in laboratory, molecular biology, and genetic engineering applications to indicate the success of a transfection or other procedure meant to introduce foreign DNA into a cell. Selectable markers include, but are not limited to, resistance to antibiotics, herbicides or other compounds, which would be lethal to cells, organelles or tissues not expressing the resistance gene or allele. Selection of transformants is accomplished by growing the cells or tissues under selective pressure, i.e., on media containing the antibiotic, herbicide or other compound. If the selectable marker is a “lethal” selectable marker, cells which express the selectable marker will live, while cells lacking the selectable marker will die. If the selectable marker is “non-lethal,” transformants (i.e., cells expressing the selectable marker) will be identifiable by some means from non-transformants, but both transformants and non-transformants will live in the presence of the selection pressure.

Antibiotic resistance genes for use as selectable markers include, but are not limited to, genes encoding for proteins resistant to puromycin, hygromycin, blasticidin, and neomycin. The genes encoding resistance to antibiotics such as ampicillin, chloroamphenicol, tetracycline or kanamycin, are examples of selectable markers for E. coli.

Examples of marker proteins include, but are not limited to an antibiotic resistance protein. In particular, beta-lactamase confers ampicillin resistance to bacterial host, neo gene from Tn5 confers resistance to kanamycin in bacteria and geneticin in eukaryotic cells. Other examples of marker proteins include, but are not limited to, florescence proteins, such as green fluorescent protein (GFP), red fluorescent protein (RFP), bilirubin-inducible fluorescent protein UnaG, dsRed, eqFP611, Dronpa, TagRFPs, KFP, EosFP, Dendra, and IrisFP.

In other embodiments, the sgRNA that binds a double-stranded nucleic sequence in genomic DNA where the vectors will be integrated is complementary to a portion of the nucleic acid sequence of a target DNA.

In other embodiments, the nucleic acids with no significant homology to the target nucleic acid molecule are about 0.001 kilobases to 100 kilobases in size, such as about 0.001, 0.002, 0.003, 0.005, 0.010, 0.020, 0.030, 0.040, 0.050, 0.060, 0.070, 0.080, 0.090, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, or about 100 kilobases in size. In other embodiments, the nucleic acids with no significant homology to the target nucleic acid molecule are about 0.1 kilobase to about 50 kilobases in size.

As used herein, the term “nuclease” refers to an enzyme capable of cleaving the phosphodiester bonds between monomers of nucleic acids. Nucleases variously effect single and double stranded breaks in their target molecules. In living organisms, they are essential machinery for many aspects of DNA repair. Nucleases are used in genetic engineering. There are two primary classifications based on the locus of activity. Exonucleases digest nucleic acids from the ends. Endonucleases act on regions in the middle of target molecules. They are further subcategorized as deoxyribonucleases and ribonucleases. The former acts on DNA, the latter on RNA. Examples of nucleases include, but are not limited to artificial restriction enzymes and artificial transcription factors (ATFs).

There are multiple approaches to controlling native gene expression, however recent advances in genetic engineering have made it possible to rapidly design and assemble artificial transcription factors (ATFs) that are both efficient and highly specific. One key feature of ATFs is that they typically have a modular structure, with two distinct and independent domains: (1) a DNA-binding domain, and (2) a transcriptional activation domain. Through customization of the DNA binding and transcriptional activation domains, it is possible to select a genomic target and activate gene expression exclusively at that locus.

First generation transcriptional activation domains are relatively weak and require binding of multiple ATFs in close proximity, within the promoter, in order to function synergistically and efficiently initiate transcription. However, second-generation transcriptional activation domains can facilitate high levels of gene activation, even when using a single ATF.

TABLE 1
Summary of Transcriptional Activators Used in Artificial Transcription
Factors to Stimulate Gene Expression

| Transcriptional | |
|---|---|
| Activating System | Notes |
| NFkB/p65 | Transcriptional activator |
| VP16 | Transcriptional activator |
| VP64 | Four Tandem repeats of the minimal activation |
| | domain of VP16 |
| CIB1-Cry2 | Light inducible system. ATF-CIB1 is used with |
| | CRY2-VP64 |
| GI-LOV | Light inducible system. ATF-GI is used with |
| | LOV-VP16 |
| GCN4 peptide | SunTag System |
| (10× or 24×) | |
| p300 HAT core | Epigenetic modifier |
| VPR | Tripartite VP64, p65, and Rta |
| SAM | Modified sgRNA used to recruit multiple effector |
| | domains |

Artificial transcription factors are classified according to the nature of the DNA-binding domain in three main groups: Zinc Finger Proteins (ZFP), Transcriptional Activator-Like Effectors (TALEs), and RNA-guided nucleases (RGNs). Each of these ATFs is effective at activating native gene expression.

As used herein, the terms “genomic DNA” or “genomic target DNA” or “target DNA” refer to chromosomal DNA. Most organisms have the same genomic DNA in every cell, but only certain genes are active in each cell to allow for cell function and differentiation within the body. The genome of an organism (encoded by the genomic DNA) is the (biological) information of heredity which is passed from one generation of organism to the next.

As used herein, “RNA-guided nuclease” or “RGN” means a nuclease capable of DNA or RNA cleavage directed by RNA base paring. Examples of RGNs include, but are not limited to, Caspase 9 (Cas9), Zinc Finger nuclease (ZFN), and TALENs.

CrSPR-CAS9-sgRNA

The Clustered Regularly Interspersed Short Palindromic Repeats/CRISPR-associated (CRISPR/Cas) system includes a recently identified type of SSN. CRISPR/Cas molecules are components of a prokaryotic adaptive immune system that is functionally analogous to eukaryotic RNA interference, using RNA base pairing to direct DNA or RNA cleavage. Directing DNA DSBs requires two components: the Cas9 protein, which functions as an endonuclease, and CRISPR RNA (crRNA) and tracer RNA (tracrRNA) sequences that aid in directing the Cas9/RNA complex to target DNA sequence (Makarova et al., Nat Rev Microbiol, 9(6):467-477, 2011). The modification of a single targeting RNA can be sufficient to alter the nucleotide target of a Cas protein. In some cases, crRNA and tracrRNA can be engineered as a single cr/tracrRNA hybrid to direct Cas9 cleavage activity (Jinek et al., Science, 337(6096):816-821, 2012). The CRISPR/Cas system can be used in bacteria, yeast, humans, and zebrafish, as described elsewhere (see, e.g., Jiang et al., Nat Biotechnol, 31(3):233-239, 2013; Dicarlo et al., Nucleic Acids Res, doi:10.1093/nar/gkt135, 2013; Cong et al., Science, 339(6121):819-823, 2013; Mali et al., Science, 339(6121):823-826, 2013; Cho et al., Nat Biotechnol, 31(3):230-232, 2013; and Hwang et al., Nat Biotechnol, 31(3):227-229, 2013).

TALENS

Transcription Activator-Like Effector Nucleases (TALENs) are artificial restriction enzymes generated by fusing the TAL effector DNA binding domain to a

DNA cleavage domain. These reagents enable efficient, programmable, and specific DNA cleavage and represent powerful tools for genome editing in situ. Transcription activator-like effectors (TALEs) can be quickly engineered to bind practically any DNA sequence. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety.

TAL effectors are proteins secreted by Xanthomonas bacteria. The DNA binding domain contains a highly conserved 33-34 amino acid sequence with the exception of the 12th and 13th amino acids. These two locations are highly variable (Repeat Variable Diresidue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The non-specific DNA cleavage domain from the end of the Fokl endonuclease can be used to construct hybrid nucleases that are active in a yeast assay. These reagents are also active in plant cells and in animal cells. Initial TALEN studies used the wild-type Fokl cleavage domain, but some subsequent TALEN studies also used Fokl cleavage domain variants with mutations designed to improve cleavage specificity and cleavage activity. The Fokl domain functions as a dimer, requiring two constructs with unique DNA binding domains for sites in the target genome with proper orientation and spacing. Both the number of amino acid residues between the TALEN DNA binding domain and the Fokl cleavage domain and the number of bases between the two individual TALEN binding sites are parameters for achieving high levels of activity. The number of amino acid residues between the TALEN DNA binding domain and the Fokl cleavage domain may be modified by introduction of a spacer (distinct from the spacer sequence) between the plurality of TAL effector repeat sequences and the Fokl endonuclease domain. The spacer sequence may be 12 to 30 nucleotides.

The relationship between amino acid sequence and DNA recognition of the TALEN binding domain allows for designable proteins. In this case artificial gene synthesis is problematic because of improper annealing of the repetitive sequence found in the TALE binding domain. One solution to this is to use a publicly available software program (DNAWorks) to calculate oligonucleotides suitable for assembly in a two-step PCR; oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms. In this manner, they can be used to correct mutations in the genome which, for example, cause disease.

Zinc Finger Nuclease (ZFNs)

Zinc finger nucleases (ZFNs) are enzymes having a DNA cleavage domain and a DNA binding zinc finger domain. ZFNs may be made by fusing the nonspecific DNA cleavage domain of an endonuclease with site-specific DNA binding zinc finger domains. Such nucleases are powerful tools for gene editing and can be assembled to induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

In some embodiments, the nuclease is Zinc finger nuclease (ZFN), RNA guided nucleases (RGN), or transcription activator-like effector nucleases (TALEN). In yet other embodiments, RGN is Caspase 9 (Cas9).

In some embodiments, the one or more vectors are plasmids or viral vectors. In other embodiments, the viral vector is a lentivirus vector, an adenovirus vector, or an adeno-associated vector (AAV).

In some embodiments, the system further comprises one or more additional sgRNA molecules that causes a double-stranded nucleic acid break of one or more additional target nucleic acid molecules. In this aspect, the genome can be cut is at several different sites (e.g., 1, 2, 3, 4, 5, 6, 7, 8, 9, or 10 sites) at or near the same time, and vector DNA is being inserted into those one or more sites.

In other embodiments, the system does not require the entire vector that can be integrated to have any homology with the target site.

Yet another aspect of the present invention provides a system for targeted genome engineering, the system comprising one or more vectors comprising: (i) at least one nucleic acid with no significant homology to the target genomic DNA site and that contains a promoter for controlling gene expression; (ii) a primary sgRNA that binds the target nucleic acid molecule at or near the transcription start site of a gene in the target nucleic acid molecule; (iii) a universal secondary sgRNA that binds one or more vectors; and (iv) a nuclease that causes a double-stranded nucleic acid break of the targeted nucleic acid molecules.

In some embodiments, the at least one nucleic acid with no significant homology to the target genomic DNA site and that contains a promoter for controlling gene expression comprises: (i) a nucleic acid promoter followed by a universal secondary sgRNA; (ii) two opposing constitutive promoters separated by a universal secondary sgRNA; or (iii) two inducible promoters in opposite orientations separated by an universal secondary sgRNA.

In some embodiments, the at least one nucleic acid with no significant homology to the target genomic DNA site and that contains a promoter for controlling gene expression; the primary sgRNA that binds the target nucleic acid molecule at or near the transcription start site of a gene in the target nucleic acid molecule; the universal secondary sgRNA that binds one or more vectors; and the a nuclease that causes a double-stranded nucleic acid break of the targeted nucleic acid molecules are located on the same or different vectors of the system.

The term “constitutive promoter” as used herein refers to an unregulated promoter that allows for continual transcription of its associated gene. These promoters direct expression in virtually all tissues and are independent of environmental and developmental factors. As their expression is normally not conditioned by endogenous factors, constitutive promoters are usually active across species and even across kingdoms. Examples of constitutive promoters include, but are not limited to, CMV, EF1A, and SV40 promoters.

In some embodiments, the two opposing constitutive promoters have similar activity or are identical to one another. In other embodiments, the two opposing constitutive promoters are non-identical to one another.

The term “inducible promoter” as used herein refers to a regulated promoter that allows for controlled transcription of its associated gene. The performance of inducible promoters is not conditioned to endogenous factors but to environmental conditions and external stimuli that can be artificially controlled. Inducible promoters can be modulated by factors such as light, oxygen levels, heat, cold and wounding, as well as chemicals, steroids, and alcohol. Since some of these factors are difficult to control outside an experimental setting, promoters that respond to chemical compounds, not found naturally in the organism of interest, are useful for genetic engineering. Examples of inducible promoters include, but are not limited to, the tetracycline ON (Tet-On) system, the negative inducible pLac promoter, the negative inducible promoter pBad, heat shock-inducible Hsp70 or Hsp90-derived promoters, and heat shock-inducible Cre and Cas9.

The terms “opposing” or “opposite” as it is used herein in connection with the terms “opposing constitutive promoters” or “inducible promoters in opposite orientations” means that the promoters are arranged to direct the expression in both directions on the vector and ensures that there is always a promoter correctly positioned regardless of integration orientation of the vector nucleic acids into the target nucleic acids.

In yet other embodiments, each inducible promotor of the two inducible promoters in opposite orientations separated by a universal secondary sgRNA contains multiple TetO repeats and a transferase gene operatively linked to a reverse tetracycline transactivator (rtTA) via a T2A peptide. In some embodiments, the number of TetO repeats of the inducible promoters can be 2, 3, 4, 5, 6, 7, 8, 9, or 10.

In some embodiments, the one or more vectors further comprise a polynucleotide encoding for a marker protein. In other embodiments, the marker protein is an antibiotic resistance protein or a florescence protein.

In some embodiments, the nucleic acid promotor is heterologous to the promoter of the target nucleic acid molecule.

In some embodiments, the nuclease is Zinc finger nuclease (ZFN), RNA guided nucleases (RGN), or transcription activator-like effector nucleases (TALEN). In other embodiments, the RGN is Caspase 9 (Cas9).

In some embodiments, the one or more vectors are plasmid or viral vectors. In other embodiments, the viral vector is a lentivirus vector, an adenovirus vector, or an adeno-associated vector (AV).

Another aspect of the present disclosure provides a method of altering the expression of at least one gene product, the method comprising: (i) introducing into a cell a system of targeted genome engineering as described herein; and (ii) selecting for successfully transfected cells by applying selective pressure; wherein the expression of at least one gene product is reduced or eliminated relative to a cell that has not been transfected with the system of targeted genome engineering.

As used herein, the term “altering expression of at least one gene product” refers to increasing, decreasing, knocking out, or activating the expression of a gene product of a cell using the targeted genome engineering systems described herein, relative to an unaltered cell.

As used herein, the term “gene product” refers to the biochemical material, either RNA or protein, resulting from expression of a gene.

In some embodiments, the method occurs in vivo or in vitro. In other embodiments, the cell is a eukaryotic cell.

The terms “cell,” “cell line,” and “cell culture” include progeny thereof. It is also understood that all progeny may not be precisely identical, such as in DNA content, due to deliberate or inadvertent mutation. Variant progeny that have the same function or biological property of interest, as screened for in the original cell, are included.

Yet another aspect of the present invention provides a method of altering the expression of at least one gene product, the method comprising: (i) introducing into a cell a system for targeted engineering as described herein; and (ii) selecting for successfully transfected cells by applying selective pressure, wherein the expression of at least one gene product is activated relative to a cell that is not transfected with the system for targeted engineering. In some embodiments, the method occurs in vivo or in vitro. In other embodiments, the cell is a eukaryotic cell.

Yet another aspect of the present invention provides a method of identifying the genetic basis of one or more medical symptoms exhibited by a subject, the method comprising: (i) obtaining a biological sample from the subject and isolating a population of cells having a first phenotype from the biological sample; (ii) transfecting a library of sgRNA into the cells; (iii) introducing into the cells a system for targeting genome engineering; (iv) selecting for successfully transfected cells by applying the selective pressure; (v) selecting the cells that survive under the selective pressure; and (vi) determining the genomic loci of the DNA molecule that interacts with the first phenotype and identifying the genetic basis of the one or more medical symptoms exhibited by the subject.

As used herein, the term “selective pressure” refers to the influence exerted by some factor (such as an antibiotic, heat, light, pressure, or a marker protein) on natural selection to promote one group of organisms or cells over another. In the case of antibiotic resistance, applying antibiotics cause a selective pressure by killing susceptible cells, allowing antibiotic-resistant cells to survive and multiply.

In some embodiments, selective pressure is applied by contacting the cells with an antibiotic and selecting the cells that survive. In other embodiments, the antibiotic is puromycin.

In another embodiment, the polynucleotide can encode for a fluorescent protein for easier monitoring of genome integration and expression, and to label or track particular cells.

As used herein, the term “phenotype” refers to any observable characteristic or functional effect that can be measured in an assay such as changes in cell growth, proliferation, morphology, enzyme function, signal transduction, expression patterns, downstream expression patterns, reporter gene activation, hormone release, growth factor release, neurotransmitter release, ligand binding, apoptosis, and product formation. Such assays include, but are not limited to, transformation assays, changes in proliferation, anchorage dependence, growth factor dependence, foci formation, growth in soft agar, tumor proliferation in nude mice, and tumor vascularization in nude mice; apoptosis assays, e.g, DNA laddering and cell death, expression of genes involved in apoptosis; signal transduction assays, e.g., changes in intracellular calcium, cAMP, cGMP, IP3, changes in hormone and neurotransmittor release; receptor assays, e.g., estrogen receptor and cell growth; growth factor assays, e.g., EPO, hypoxia and erythrocyte colony forming units assays; enzyme product assays, e.g., FAD-2 induced oil desaturation; transcription assays, e.g., reporter gene assays; and protein production assays, e.g., VEGF ELISAs. A candidate gene is “associated with” a selected phenotype if modulation of gene expression of the candidate gene causes a change in the selected phenotype.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), single guide RNA (sgRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “complementary” or “substantially complementary” as used herein refers the hybridization or Watson-Crick base pairing between nucleotides or nucleic acids, such as, for instance, between the two strands of a double stranded DNA molecule or between an oligonucleotide primer and a primer binding site on a single stranded nucleic acid to be sequenced or amplified or between a sgRNA and a target nucleic acid molecule. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 60%, 70%, 80%, 85%, 86%, 87%, 88%, 89%, 90%, 91%, 92%, 93%, 94%, 95%, 96%, 97%, 98%, or 100% of the nucleotides of the other strand. Alternatively, substantial complementarity exists when an RNA or DNA strand will hybridize under selective hybridization conditions to its complement. Typically, selective hybridization occurs when there is at least about 65%, 70%, 80%, 85%, 86%, 87%, 88%, 89%, 90%, 91%, 92%, 93%, 94%, 95%, 96%, 97%, 98%, or 99% complementarity over a stretch of at least 14 to 25 nucleotides.

As used herein, “expression” refers to the process by which a polynucleotide is transcribed from a DNA template (such as into and sgRNA or mRNA) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. The term “capable of expression” means the vector has all the components necessary to express the sgRNA or the heterologous gene product, as described below and known to one of ordinary skill in the art. The polynucleotide of the first vector can encode for a protein to tag the cells it is integrated into, to knock out a gene located within the DNA target of interest, to introduce a mutant version of the gene located within the target DNA of interest, to express inhibitory RNAs, or any polynucleotide of interest.

As used herein, the term “subject” refers to any animal classified as a mammal, including humans, mice, rats, domestic and farm animals, non-human primates, and zoo, sport or pet animals, such as dogs, horses, cats, and cows.

As used herein, the terms “library” or “library of sgRNA” refers to a plurality of sgRNAs that are capable of targeting a plurality of genomic loci in a population of cells.

Several aspects of the disclosure relate to vector systems comprising one or more vectors, or vectors as such. Vectors can be designed for expression of RGNs and polynucleotides (e.g. nucleic acid transcripts, proteins, or enzymes) in prokaryotic or eukaryotic cells. For example, RGN or polynucleotides can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

A “vector” is a replicon, such as a plasmid, phage, or cosmid, to which another nucleic acid segment may be attached so as to bring about the replication of the attached segment. A vector is capable of transferring polynucleotides (e.g. gene sequences) to target cells (e.g., bacterial plasmid vectors, particulate carriers and liposomes).

Typically, the terms “vector construct,” “expression vector,” “gene expression vector,” “gene delivery vector,” “gene transfer vector,” “transfer vector,” and “expression cassette” all refer to an assembly which is capable of directing the expression of a sequence or gene of interest. Thus, the terms include cloning and expression vehicles.

As used herein, a “promoter” may refer to any nucleic acid sequence that regulates the initiation of transcription for a particular polypeptide-encoding nucleic acid under its control. A promoter minimally includes the genetic elements necessary for the initiation of transcription (e.g., RNA polymerase Ill-mediated transcription), and may further include one or more genetic regulatory elements that serve to specify the prerequisite conditions for transcriptional initiation.

The term “regulatory element” as used herein includes promoters, enhancers, internal ribosomal entry sites (IRES), and other expression control elements (e.g. transcription termination signals, such as polyadenylation signals and poly-U sequences). Such regulatory elements are described, for example, in Goeddel, (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter, one or more pol II promoters, one or more pol I promoters, or combinations thereof. Examples of pol III promoters include, but are not limited to, U6 and H1 promoters.

Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

A promoter may be encoded by the endogenous genome of a host cell, or it may be introduced as part of a recombinantly engineered polynucleotide. A promoter sequence may be taken from one host species and used to drive expression of a gene in a host cell of a different species. A promoter sequence may also be artificially designed for a particular mode of expression in a particular species, through random mutation or rational design. In recombinant engineering applications, specific promoters are used to express a recombinant gene under a desired set of physiological or temporal conditions or to modulate the amount of expression of a recombinant nucleic acid.

Methods for transforming a host cell with an expression vector may differ depending upon the species of the desired host cell. For example, yeast cells may be transformed by lithium acetate treatment (which may further include carrier DNA and PEG treatment) or electroporation. These methods are included for illustrative purposes and are in no way intended to be limiting or comprehensive. Routine experimentation through means well known in the art may be used to determine whether a particular expression vector or transformation method is suited for a given host cell. Furthermore, reagents and vectors suitable for many different host microorganisms are commercially available and/or well known in the art.

Many suitable expression vectors and features thereof are known in the art; for example, various vectors and techniques are illustrated in Current Protocols in Molecular Expression vectors may contain, without limitation, a centromeric (CEN) sequence, an autonomous replication sequence (ARS), a promoter, an origin of replication, and a marker gene (e.g., auxotrophic, antibiotic, or other selectable markers). Examples of expression vectors may include plasmids, yeast artificial chromosomes, 2μπι plasmids, yeast integrative plasmids, yeast replicative plasmids, shuttle vectors, and episomal plasmids.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A. respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include, but are not limited to, pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Conventional and standard techniques may be used for recombinant DNA molecule, protein, and antibody production, as well as for tissue culture and cell transformation. Enzymatic reactions and purification techniques are typically performed according to the manufacturer's specifications or as commonly accomplished in the art using conventional procedures known in the art, or as described herein. Unless specific definitions are provided, the nomenclature utilized in connection with, and the laboratory procedures and techniques of analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation, and delivery, and treatment of patients.

Further, the terminology used herein is for the purpose of exemplifying particular embodiments only and is not intended to limit the scope of the invention as disclosed herein. Any method and material similar or equivalent to those described herein can be used in the practice of the invention as disclosed herein and only exemplary methods, devices, and materials are described herein.

The invention now will be exemplified for the benefit of the artisan by the following non-limiting examples that depict some of the embodiments by and in which the invention can be practiced.

### Example 1: Demonstration of the Nuclease Assisted Vector Integration (NAVI) System

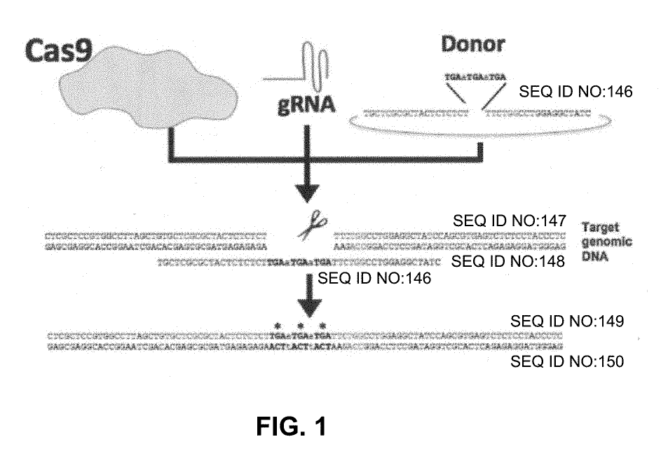

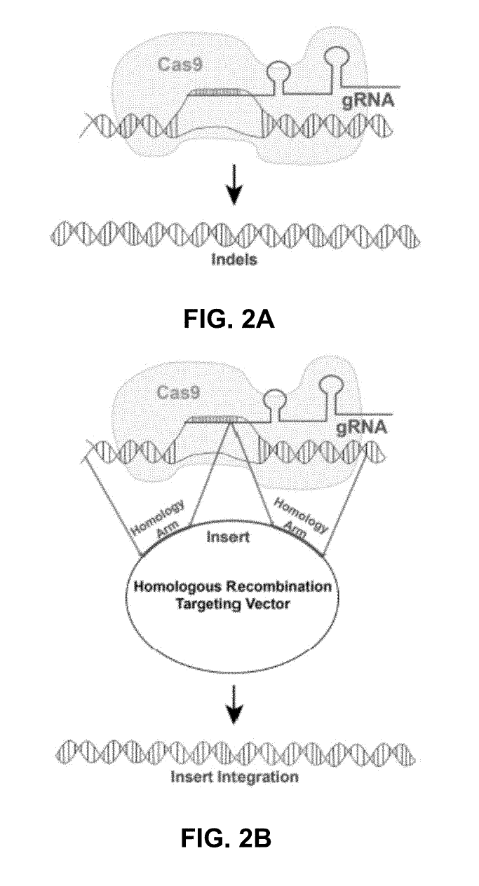

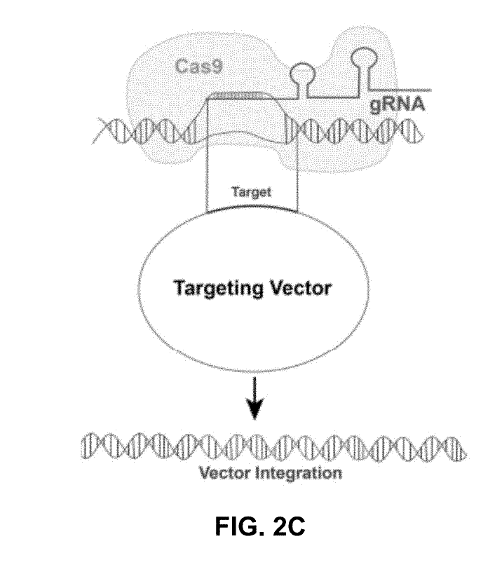

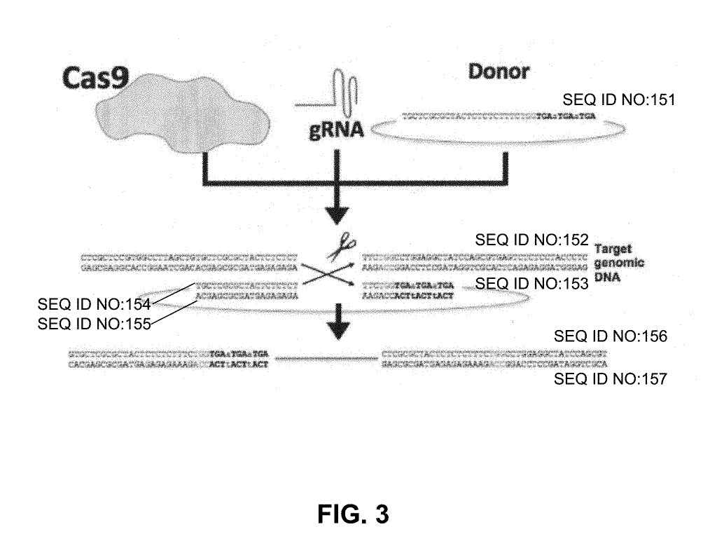

The traditional approach to integrate heterologous DNA at target genomic loci using homologous recombination of donor vectors is shown in the schematic of FIG. 1 and FIG. 2A. The integration efficiencies that can be achieved with this traditional system are very low and decrease as the size of the insert increases, non-specific integration occurs often, and it requires time-consuming cloning of homology arms. FIG. 2B is a schematic of DNA integration utilizing homologous recombination. The NAVI system for targeted genome modification are shown in the schematics of FIG. 2C and FIG. 3. The DNA repair mechanisms stimulated by this method facilitate integration of the entire vector in genomic DNA at the target site. This method is as efficient as homologous recombination and integration occurs regardless of the size of the plasmid. Since cloning of homology arms is not needed, the effort and cost needed to implement this system is low.

Cell Culture and Transfection

HEK293T and HCT116 cells were obtained from the American Tissue Collection Center (ATCC) and were maintained in DMEM supplemented with 10% fetal bovine serum and 1% penicillin/streptomycin at 37° C. with 5% CO₂. HEK293T and HCT116 cells were transfected with Lipofectamine 2000 (Invitrogen) according to manufacturer's instructions. Transfection efficiency in 293T cells was routinely higher than 80% whereas transfection efficiency in HCT116 cells was ˜55% as determined by FACS following delivery of a control GFP expression plasmid. The antibiotics used for selection of clonal populations of HCT116 cells were Puromycin 0.5 μg/ml, Hygromycin 100 μg/ml, Blasticidin 10 μg/ml and Neomycin 1 mg/ml.

Plasmids and Oligonucleotides

The plasmids encoding spCas9 and sgRNA were obtained from Addgene (Plasmids #41815 and #47108). The backbone for the transfer vectors was synthesized by IDT Technologies as gene blocks and cloned into a pCDNA3.1 backbone. Oligonucleotides for construction of sgRNAs were obtained from IDT Technologies, hybridized, phosphorylated and cloned in the sgRNA and transfer vectors using BbsI sites as previously described in Perez-Pinera et. al, Nat Methods 10, 973-976, 2013. The target sequences of the gRNAs are provided in Table 2.

TABLE 2
Target sequence of the different sgRNAs
used in this these studies

| | | SEQ | | |
|---|---|---|---|---|
| | | ID | | |
| Target | Protospacer | NO. | PAM | Strand |
| ACTB Plus | AGCAGGAGTATGACGAGTC | 1 | CGG | + |
| Strand | | | | |
| ACTB Minus | CGGTGGACGATGGAGGGGC | 2 | CGG | |
| Strand | | | | |
| GAPDH | ATGGCCCACATGGCCTCCA | 3 | AGG | + |
| TUBB | GGTGAGGAGGCCGAAGAGG | 4 | AGG | + |
| TUBBN20 | CGGTGAGGAGGCCGAAGAGG | 5 | AGG | + |
| NROB2 | CAGGGGCCTGCCCATGCCA | 6 | GGG | + |
| CITNEX9 | AAGTGGATAAGAGCGCCGT | 7 | TGG | − |
| CTTN EX8 | GCGCTCTTGTCTACTCGGT | 8 | CGG | − |
| HLA-DRA | GCTGTGCTGATGAGCGCTC | 9 | AGG | + |
| IL 1R1 | AAGCAGAAACTACCCGTTGC | 10 | AGG | + |
| IL1RN | TGTACTCTCTGAGGTGCTC | 11 | TGG | + |
| ETV sgRNA | ACCGGGTCTTCGAGAAGACC | 12 | TGG | +/− |
| CMV sgRNA | TCGATAAGCCAGTAAGCAGT | 13 | GGG | +/− |
| T7 sgRNA | CGTAATACGACTCACTATA | 14 | GGG | +/− |
| BAC sgRNA | TGAGGGCCAAGTTTTCCGCG | 15 | AGG | − |
| 1 | | | | |
| Lambda | TTACGGGGCGGCGACCTCGC | 16 | GGG | |
| sgRNA 1 | | | | |

PCR

Seventy-two hours after transfection genomic DNA was isolated using DNeasy Blood & Tissue Kit (Qiagen). PCRs were performed using KAPA2G Robust PCR kits. A typical 25 μL reaction used 20-100 ng of genomic DNA, Buffer A (5 μL), Enhancer (5 μL), dNTPs (0.5 μL), 10 μM forward primer (1.25 μL), 10 μM reverse primer (1.25 μL), KAPA2G Robust DNA Polymerase (0.5 U) and water (up to 25 μL). The DNA sequences of the primers for each target are provided in Table 4. The PCR products were visualized in 2% agarose gels and images were captured using a ChemiDoc-It² (UVP).

Surveyor Assay

Seventy-two hours after transfection genomic DNA was isolated using DNeasy Blood & Tissue Kit (Qiagen). The region surrounding the RGN target site was amplified by PCR with the AccuPrime PCR kit (Invitrogen) and 50-200 ng of genomic DNA as template with primers provided in Table 3. The PCR products were melted and reannealed using the temperature program: 95° C. for 180 s, 85° C. for 20 s, 75° C. for 20 s, 65° C. for 20 s, 55° C. for 20 s, 45° C. for 20 s, 35° C. for 20 s and 25° C. for 20 s with a 0.1° C./s decrease rate in between steps. Eighteen microliters of the reannealed duplex was combined with 1 μl of the Surveyor nuclease and 1 μl of enhancer solution (Integrated DNA Technologies), incubated at 42° C. for 60 min and then separated on a 10% TBE polyacrylamide gel. The gels were stained with ethidium bromide and visualized using a ChemiDoc-It² (UVP). Quantification was performed using methods previously described in Guschin et. al. Methods Mol Biol 649, 247-256, 2010.

TABLE 3
Sequence of the different primers used
in these studies.

| | | SEQ |
|---|---|---|
| | | ID |
| Primer | Sequence | NO: |
| ACTB FW | GTCACATCCAGGGTCCTCAC | 17 |
| ACTB REV | TCTGCGCAAGTTAGGTTTTG | 18 |
| GAPDH FW | AGGGCCCTGACAACTCTTTT | 19 |
| GAPDH REV | AGGGGTCTACATGGCAACTG | 20 |
| TUBB FW | CATGGACGAGATGGAGTTCA | 21 |
| TUBB REV | GAATGGGCACCAGAAAGAAA | 22 |
| NR0B2 FW | GATAAGGGGCAGCTGAGTGA | 23 |
| NR0B2 REV | GTGCGATGAGGTGCACATAG | 24 |
| GFP REV | TGCCCTTGTCTTGTAGTTTCC | 25 |
| RFP REV | ATATCTGCGGGGTGTTTCAC | 26 |
| PUROR REV | GCCTGACTGTGGGCTTGTAT | 27 |
| HYGROR REV | GCGGTGAGTTCAGGCTTTTT | 28 |
| CTTN EX9FW | CTCCCTTCTCAGCCTCCTG | 29 |
| CTTN EX9REV | GTTTTTCCTTTTCCGGTGTG | 30 |
| CTTN EX8FW | GCGCTTGATGTGTTTGTGAG | 31 |
| CTTN EX8REV | CCTCATACGATGGGGAACTG | 32 |
| ACTB TALEN FW | CCTCCATCGTCCACCGCAA | 33 |
| ACTB TALEN REV | GTGGATCAGCAAGCAGGAGT | 34 |
| HLA-DRA FW | TCCCGAGCTCTACTGACTCC | 35 |
| HLA-DRA REV | TTGGCTTGTAGCAGGACCTT | 36 |
| IL1R1 FW | TGCAAAATTTGTGGAGAATGA | 37 |
| 1L1R1 REV | ATGCTTTTCAGCCACATTCA | 38 |
| GAPDH QPCR FW | CAATGACCCCTTCATTGACC | 39 |
| GAPDH QPCR REV | TTGATTTTGGAGGGATCTCG | 40 |
| IL1RN QPCR FW | GGAATCCATGGAGGGAAGAT | 41 |
| IL1RN QPCR REV | TGTTCTCGCTCAGGTCAGTG | 42 |
| BACFW1 | TTACAGCCAGTAGTGCTCGC | 43 |
| BACREV1 | CCCAGGCTTGTCCACATCAT | 44 |
| BACREV2 | GCACTTATCCCCAGGCTTGT | 45 |
| LAMBDAFW | GGTTGTTGTTCTGCGGGTTC | 46 |
| LAMBDAREV | CCATTTTATGACGGCGGCAG | 47 |
| ww331 | GTGCGATGAGGTGCACATAG | 48 |
| ww330 | GATAAGGGGCAGCTGAGTGA | 49 |
| ww442 | GAGAAACACTGGACCCCGTA | 50 |
| M13F (−21) | TGTAAAACGACGGCCAGT | 51 |
| M13REV | CAGGAAACAGCTATGAC | 52 |
| ww499 | GATAACACTGCGGCCAACTT | 53 |
| ww293 | GGCACCTATCTCAGCGATCT | 54 |
| ww286 | CCTTCTAGTTGCCAGCCATC | 55 |

Western Blot

Cells were lysed with loading buffer, boiled for 5 min, loaded in NuPAGE® Novex 4-12% Bis-Tris Gel polyacrylamide gels and transferred to nitrocellulose membranes. Non-specific antibody binding was blocked with 50 mM Tris/150 mM NaCl/0.1% Tween-20 (TBS-T) with 5% nonfat milk for 30 min. The membranes were incubated with primary antibodies anti-GAPDH (Cell Signaling Technology) or anti-CTTN (Cell Signaling Technology) in 5% BSA or 5% nonfat milk in TBS-T diluted 1:1,000 for 60 min and the membranes were washed with TBS-T for 30 min. Membranes labeled with primary antibodies were incubated with anti-rabbit HRP-conjugated antibody (Sigma-Aldrich) diluted 1:10,000 for 30 min, and washed with TBS-T for 30 minutes. Membranes were visualized using the Clarity™ ECL Western Blotting Substrate (Bio-Rad) and images were captured using a ChemiDoc-It² (UVP).

Quantification of Integration Efficiency

HCT116 cells were transfected with individual RGNs targeting either CTTN exon 8 or HLA-DRA, as well as Cas9, one universal RGN, and either one or two transfer vectors with expression cassettes conferring resistance to puromycin or puromycin and hygromycin. A total of 450,000 cells were transfected using 100 ng of each plasmid. The transfection efficiency was ˜55% as determined by FACS following delivery of a control GFP expression plasmid. Three days post transfection, 90% of cells from each well were harvested and replated into 10 cm dishes for selection with the appropriate antibiotics. Cells with monoallelic modifications were selected with puromycin whereas cells with biallelic modifications were selected with puromycin and hygromycin. Media and antibiotics were replenished every three days. Visible colonies appeared after approximately after one week. The number of clones for each transfection was counted and integration efficiency was determined as the ratio of the number of clonal cells derived from each transfection relative to the number of alleles modified by each specific sgRNA, as measured in experimental control samples using the surveyor assay.

Results

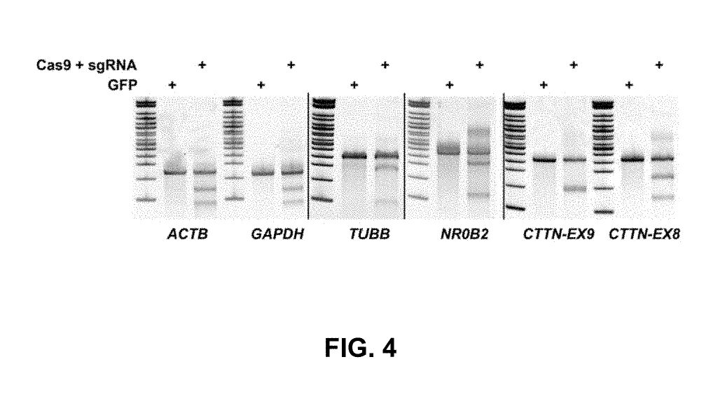

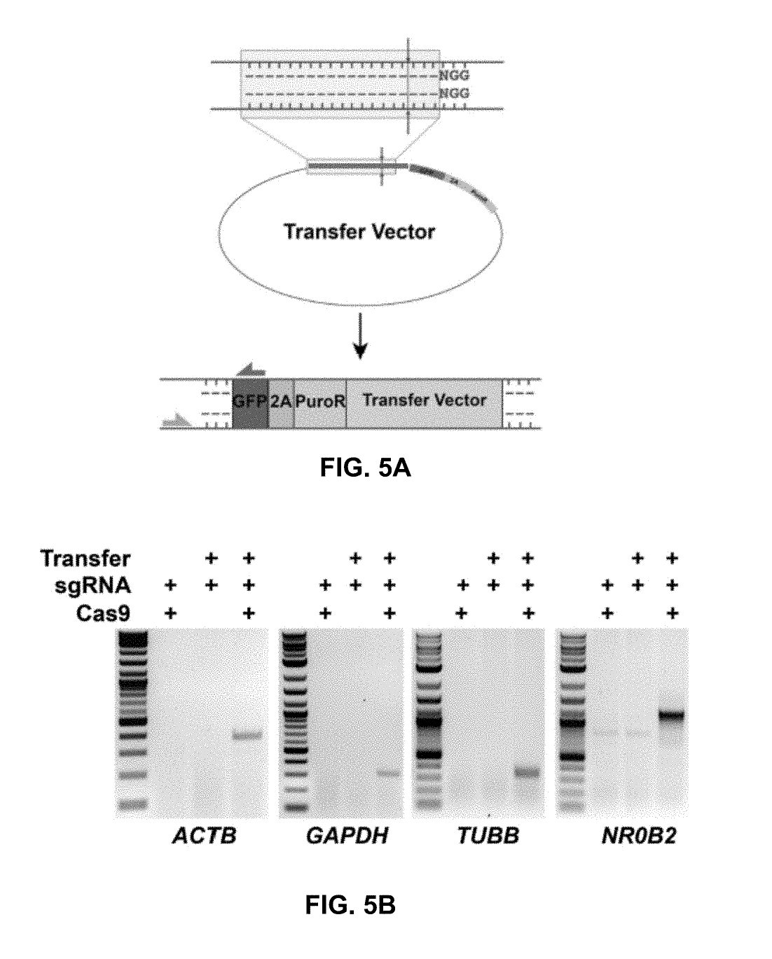

The first version of a genomic DNA integration system relied upon a sgRNA capable of introducing DSBs at genetic loci of interest and a vector where the sgRNA target site was cloned upstream of a GFP transgene. Single guide RNAs were validated using the Surveyor Assay three days after transfection. No gene modification was detected in control samples, however, co-transfection of Cas9 and sgRNA effectively introduced insertions and deletions in all the target sites analyzed in these studies (FIG. 4). These vectors are referred to as “transfer vectors”, FIG. 5A. For proof-of-principle studies with the genes ACTB (β-actin), GAPDH, and TUBB (β-tubulin), and NR0B2 (SHP1) were conducted. Four gene specific transfer vectors containing the sequence targeted by the sgRNA in genomic DNA were prepared. Cotransfection of Cas9 with the sgRNA and the transfer vector stimulates integration of each transfer vector at the specific target site (FIG. 5B). These results suggest that this integration system is sequence specific and that it can be used to multiplex integration of various vectors at different loci.

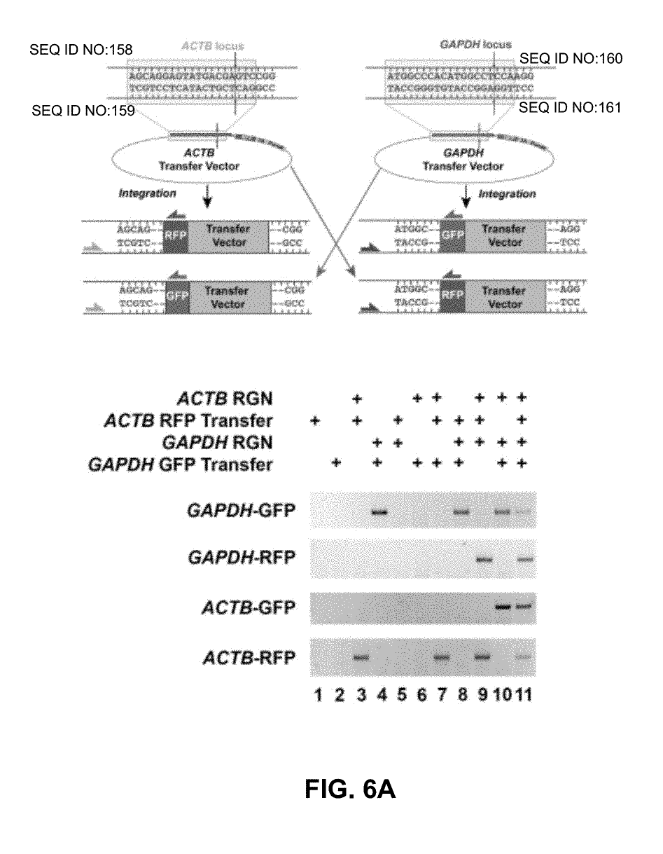

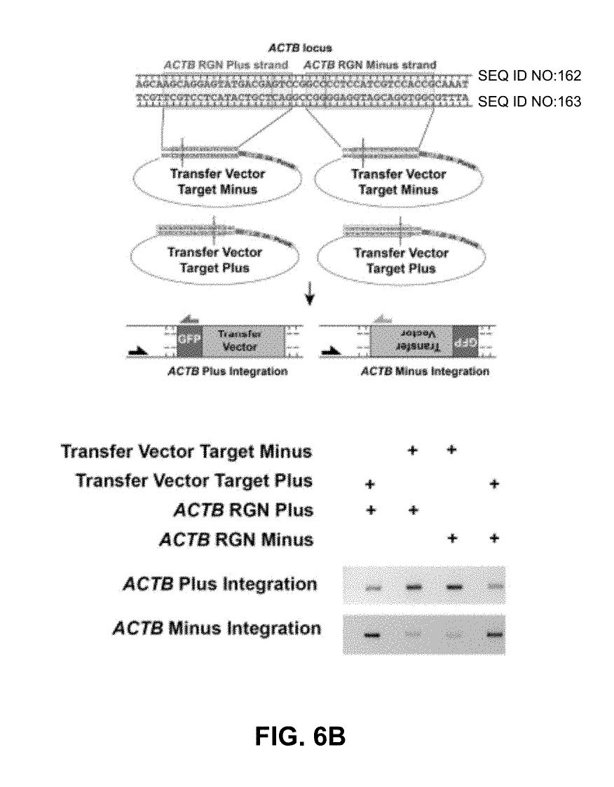

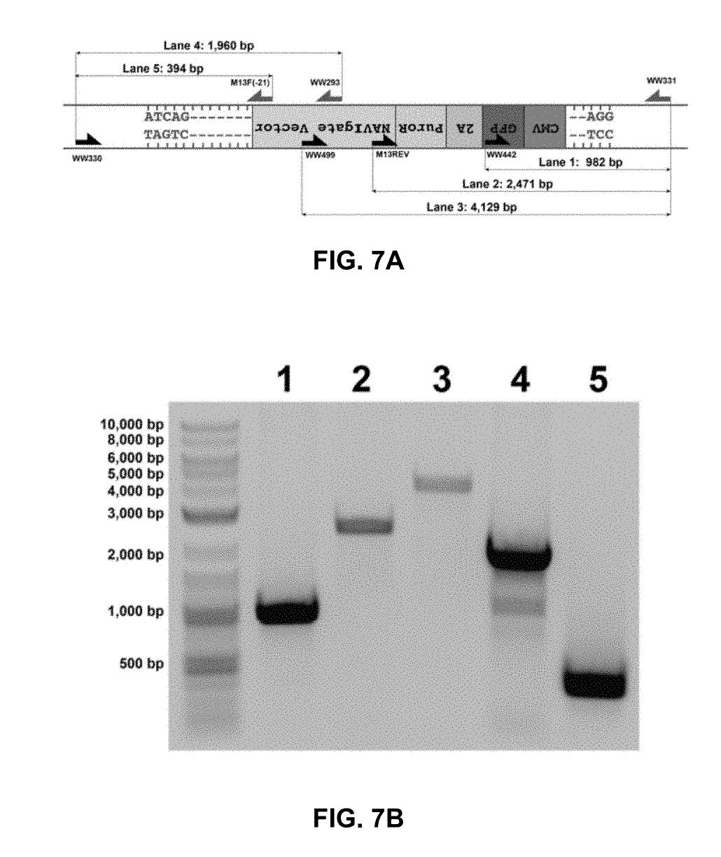

Multiplex integration was evaluated by comprehensively characterizing genomic incorporation of two transfer vectors intended for two distinct loci: one that expresses GFP and contains a GAPDH RGN target sequence, and another that expresses RFP but contains an ACTB RGN target sequence (FIG. 6A). As expected, integration of GFP at GAPDH required Cas9, GAPDH sgRNA and GAPDH transfer vectors (lanes 4, 8, 10 and 11). Similarly, integration of RFP at the ACTB locus required Cas9, ACTB sgRNA and ACTB transfer vectors (lanes 3, 7, 9 and 11). Strikingly, when both ACTB and GAPDH RGNs were used but only one transfer vector was present, integration occurred at both loci (lanes 9, 10 and 11). Furthermore, when ACTB and GAPDH RGNs and the corresponding transfer vectors were transfected simultaneously, each transfer vector was integrated at both loci (lane 11). Specific recombination were ruled out between both target sites in the vector and in the genome by testing the directionality of the integration. Two sgRNAs were designed that target the plus or minus strand of the ACTB locus and we introduced the target sequence of each sgRNAs in the plus or minus orientations in two separate transfer vectors. PCR analysis demonstrated that integration occurs in the sense and antisense orientations whether the plus or the minus strands are targeted (FIG. 6B). Furthermore, PCRs from selected clonal cell lines demonstrated that the entire vector is integrated (FIG. 7A-7B).

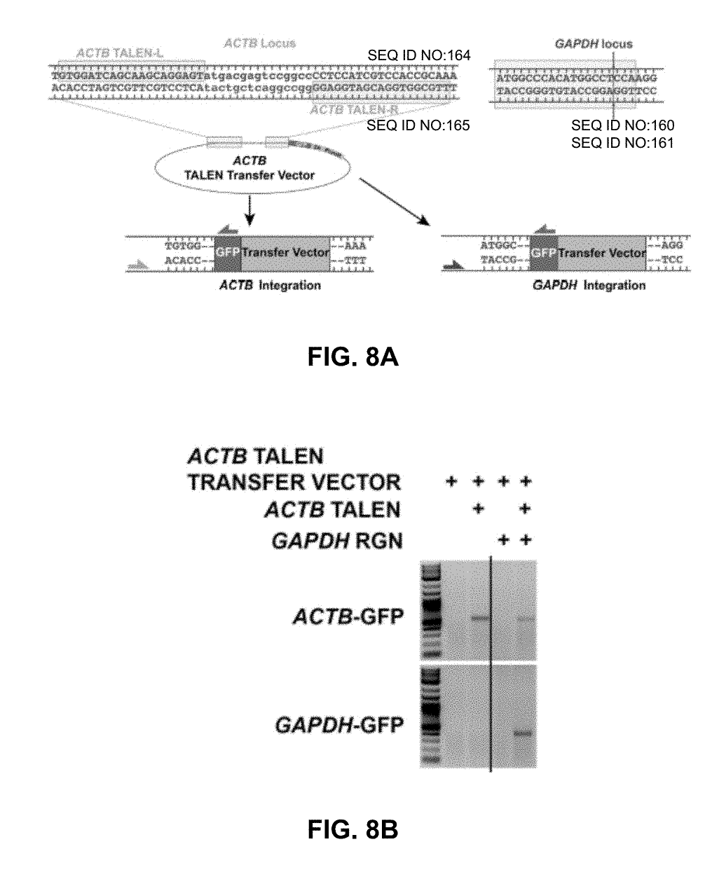

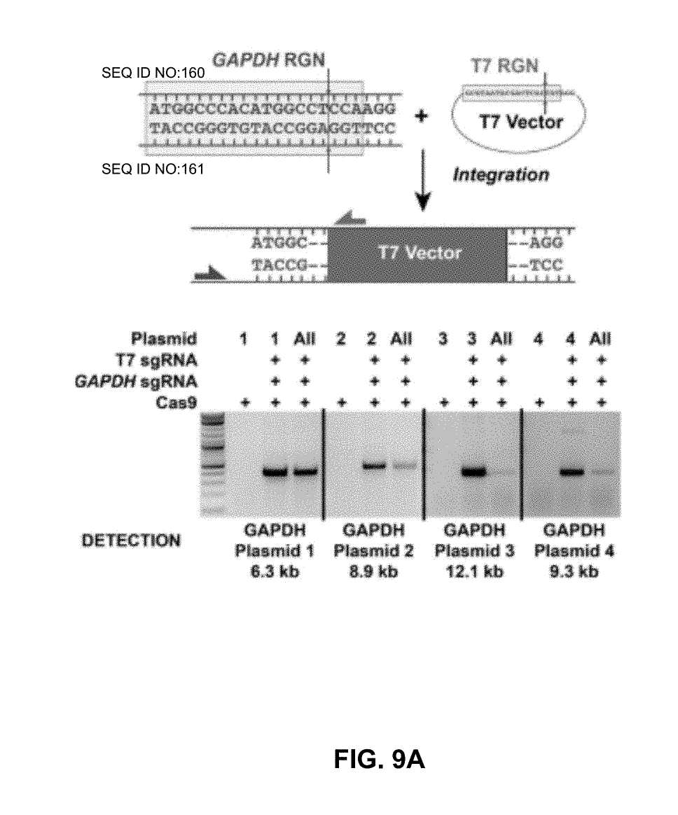

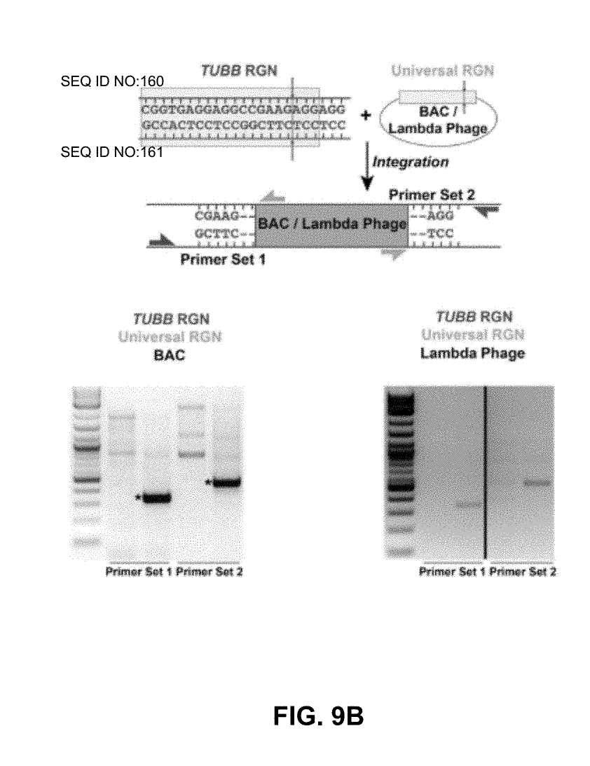

These findings show that DSBs in genomes can avidly capture linear DNA present in the nucleus regardless of homology whereas circular vectors are not efficiently integrated at DSBs. Since transfer vectors linearized with TALENs are also effectively integrated at DSBs generated with RGNs (FIGS. 8A-8B), introduction of a DSB in the donor vector should be sufficient to stimulate its integration without inclusion of the target site also found in genomic DNA (FIG. 9A). A panel of 4 vectors with sizes ranging from 6.3 to 12.1 kb, a sgRNA that targets the T7 promoter sequence found in all these vectors, Cas9, and a sgRNA that targets the GAPDH locus in genomic DNA were transfected. Although there is no homology between the GAPDH target site and any of the transfer vectors, every transfer vector was effectively integrated at the GAPDH locus when transfected individually and also when transfected simultaneously (FIG. 9A). These results demonstrate that this nuclease assisted vector integration (NAVI) system is multiplexable and that integration can be achieved using universal RGNs without modifying the transfer vectors.

### Example 2: Integration of Large Vectors into Genomic DNA

Unlike HR-based genomic integration systems, large size vectors can be fully integrated in genomic DNA very efficiently (FIG. 9B). To determine the size limit for plasmids to integrate in genomic DNA, NAVI was utilized by testing integration of a 25 kb bacterial artificial chromosome as well as a lambda phage circular genome, which contains 48.5 kb. sgRNAs were designed capable of linearizing each of these vectors and a sgRNA to introduce a DSB at the TUBB locus in genomic DNA. PCR reactions that amplify integration of both ends of the plasmids at the target locus in pooled cell populations confirmed successful integration (FIG. 9B).

### Example 3: Multiplexed Integration of a Vector at Multiple Loci

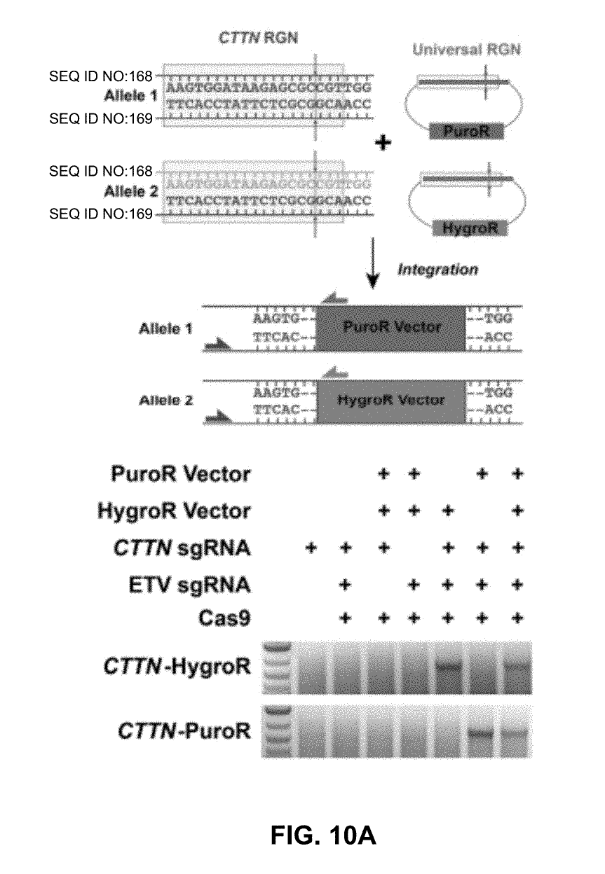

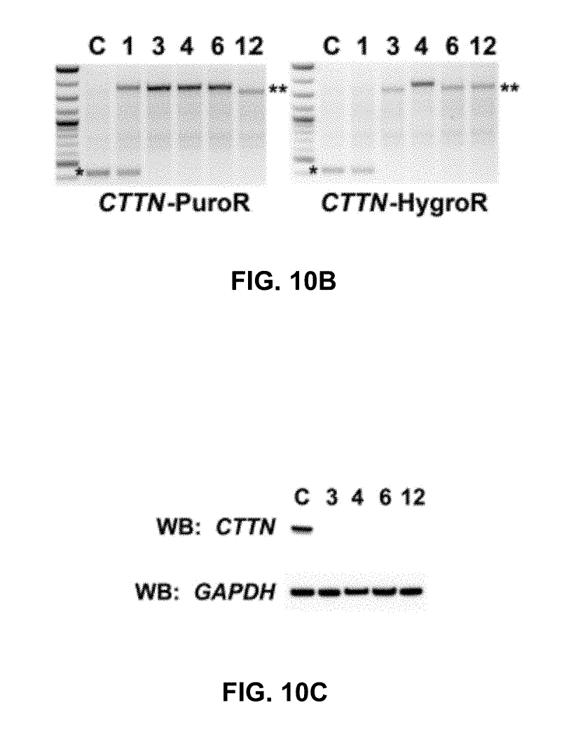

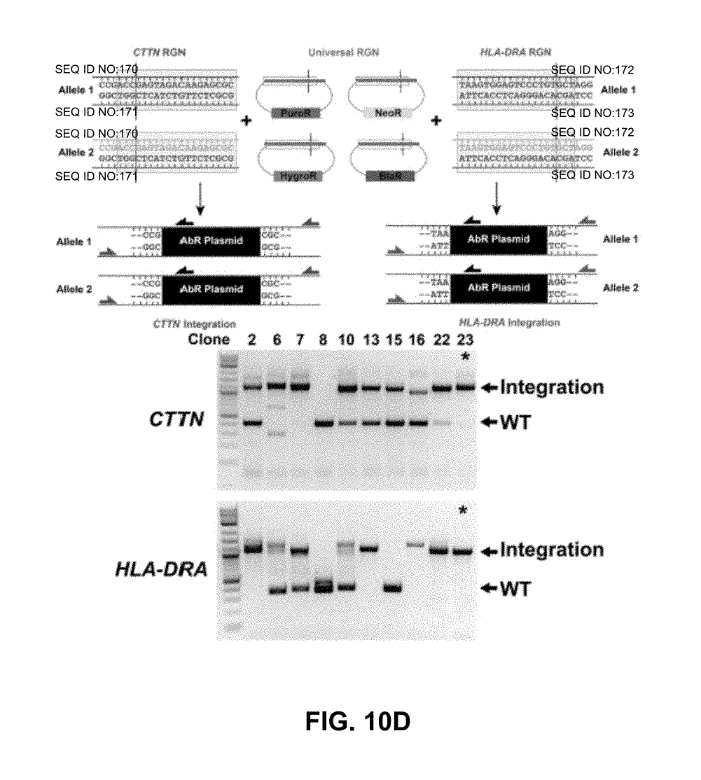

While multiplexed integration of a single vector at multiple loci has broad applications for synthetic biology, integration of multiple vectors at a single locus is particularly interesting for cell line engineering purposes, such as rapid gene knock out. By simply cotransfecting Cas9, a sgRNA targeting the CTTN locus and a universal sgRNA targeting two separate transfer vectors that encode puromycin or hygromycin resistance expression cassettes, one vector was successfully integrated into each allele of the CTTN gene (FIG. 10A). Simultaneous selection with hygromycin and puromycin ensured that most clonal populations generated contained biallelic modifications (FIG. 10B) that resulted in gene knock out as demonstrated by Western blot (FIG. 10C).

Overall, the timeframe from sgRNA design to HCT116 clonal cell verification and expansion was 2-3 weeks with minimal resources and screening effort required. Cell lines were generated with monoallelic or biallelic modifications at 4 loci tested, including CTTN exon 8 and HLA-DRA (FIG. 10D). The overall integration efficiency in one allele was ˜19% of the cells in which DSBs were introduced at the target site. Using dual selection, the apparent biallelic targeting efficiency was ˜5% of the cells with DSBs (Table 4).

TABLE 4
Bi-allelic target efficiency

| | | | % Efficiency | % |
|---|---|---|---|---|
| | | | (colonies/ | Efficiency |
| | | Avg. | transfected | (adjusted by |
| sgRNA | Selection | Colonies | cells) | indel %) |
| CTTN | Puromycin | 1726 | 0.38 | 12.00 |
| exon 8 | Puromycin + | 725 | 0.16 | 5.00 |
| | Hygromycin | | | |
| HLA-DRA | Puromycin | 2610 | 0.58 | 26.40 |
| | Puromycin + | 453 | 0.10 | 4.60 |
| | Hygromycin | | | |

The percent of total alleles modified by NAVI in diploid cells is 62.5% following selection with a single antibiotic, with 90% of clones containing at least a monoallelic modification. Under dual antibiotic selection, 75.4% of the clones contained biallelic modification and 98.2% of clones had at least one allele modified (Table 5).

Following selection in 10-cm plates with the appropriate antibiotic, total colonies were counted and divided by total cells transfected to obtain the overall editing efficiency of NAVI. This value was then adjusted to account for overall sgRNA editing efficiency, as measured by surveyor nuclease assay. This quantification was performed at 2 different loci using either a single or two antibiotics for selection.

Data collected from integration-specific PCR was used to determine allelic modification rates among clonal cell populations isolated selection. The total number of clones from each genotype (+/+, +/−, and −/−) was determined for each of four genomic targets analyzed. The frequency of allelic modification (total number of alleles modified divided by total number of alleles) was calculated for clones selected using one or two antibiotics.

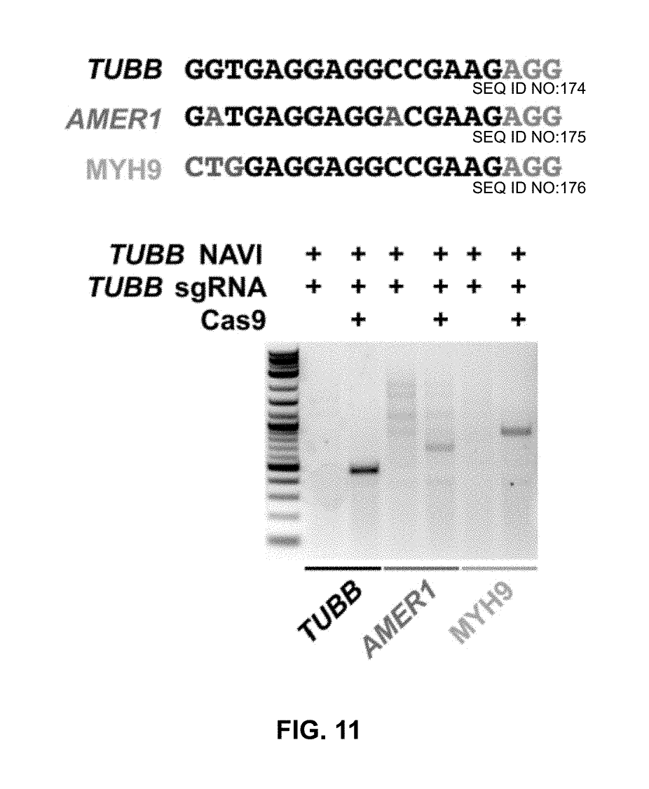

A limitation for multiplexing applications using NAVI is the potential for off-target integration. Since NAVI relies on linearized DNA integrating at DSBs, naturally occurring DSBs or DSBs derived from off-target binding of the sgRNAs become sites for potential unintended integration as demonstrated in FIG. 11. 293T cells were transfected with RGNs targeting the TUBB locus and a transfer vector that contains the TUBB target sequence. Analysis of potential off-target sites of the RGN, identified over 50 potential sites. Off-target integration at the coding sequences of the genes AMER1 and MYH9 using PCR primers bind in genomic DNA of the off-target site and in the vector backbone were analyzed. The transfer vector integrated efficiently at the off-target sites despite 2 or 3 mismatches between the on-target and off-target sequence.

In HCT116 cells, up to 4 antibiotics have been successfully used for rapid isolation of cell lines with dual gene knock-outs, however, only 10% of the clones contained the desired mutations simultaneously (FIG. 10D). This lower efficiency can be attributed to integration of the transfer vector at off-target sites or poor performance of the drugs used for screening under these conditions. These results suggest that, in addition to careful consideration of selection system, choosing sgRNAs with high off-target scores (see for example Hsu et. al., Nat Biotechnol 31, 827-832, 2013) or using RGN systems with higher specificity (see for example Bolukbasi et. al. Nat Methods 12, 1150-1156, 2015; Fu et. al. Nat Biotechnol 32, 279-284, 2014), are critical parameters for targeted integration.

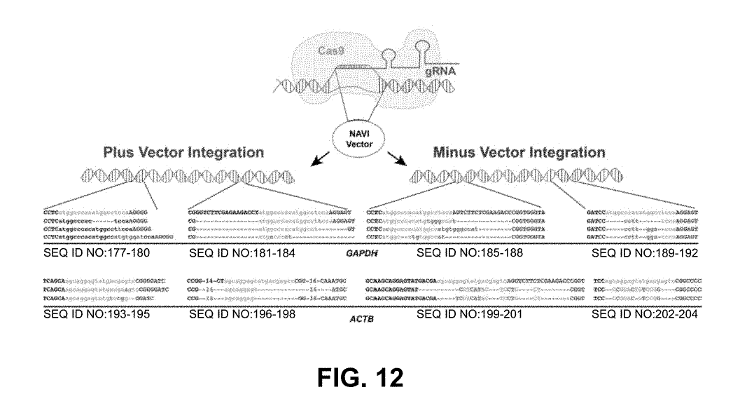

Mutations can often be found at the junction of genomic DNA with the integrated transfer vector suggesting that the integration mechanism involves an error-prone DNA repair pathway. Genomic DNA from pooled populations of 293 cells transfected and RGNs targeting GAPDH or ACTB and the corresponding transfer vectors was isolated and the regions flanking plasmid integration in genomic DNA were amplified by PCR. The PCR products corresponding to integration events in plus or minus orientation were cloned and sequenced. The sequencing results identified a wide range of mutations at the junction of genomic DNA with the vector suggesting that a mutagenic DNA repair pathway mediates integration of the vector into the target site (FIG. 12).

While mutagenesis generated via NHEJ remains a highly efficient and effective strategy for select applications, the insertion of large or complex sequences and the ability to easily select for modified cells often necessitates the use of homology directed repair (HDR) based strategies. The time-consuming construction of donor vectors for HDR gene editing is often technically challenging, costly, and leads to poor modification rates. By using customized single-stranded oligonucleotides (ssODN) the efficiency of gene editing increases, but the scale of possible genetic changes is greatly diminished. Additionally, as both donor vectors and ssODN require two discontiguous regions of homology, neither is well suited to multiplexing. Nuclease-Assisted Vector Integration (NAVI) is a unique strategy to bypass HDR and the need for customized donor vectors required for traditional genome editing technologies.

Multiplexed genome editing via nuclease assisted vector integration presents a unique opportunity for genome-scale engineering in mammalian cells. The results demonstrate that NAVI is capable of rapidly remodeling mammalian genomes by targeted insertion of large expression cassettes in one single step. NAVI eliminates the need for homologous sequence within donor vectors. While NAVI sacrifices single base pair resolution, it is capable of achieving predictable and robust patterns of integration into native genomes. Virtually any vector may be integrated at a target site in the genome without cloning, setting it apart from all prior integration systems. Importantly, facile integration of large constructs up to 50 kbp, including an entire phage genome were demonstrated, however no upper size limit was identified. Finally, through multiplexed NAVI, a novel system for targeted gene disruption was demonstrated, in which screening time is greatly reduced by via positive selection. In summary, this novel approach to gene editing extends the capacity of structural and functional mammalian genome engineering for applications in synthetic biology and creates new opportunities for developing more efficient gene therapies.

### Example 4. Targeted Gene Activation of ASCL1 Using RNA-Guided Nucleases

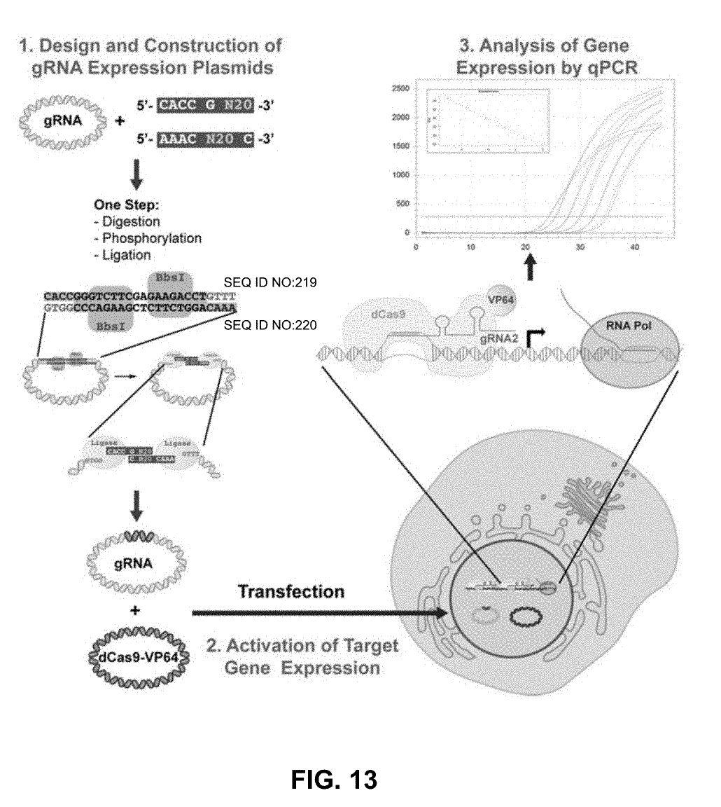

This Example describes a protocol for activation of ASCL1 expression using RGNs consisting of S. pyogenes Cas9 and single guide RNAs (FIG. 13). See also Brown, et al., Chapter 16: Targeted Gene Activation Using RNA-Guided Nucleases, Enhancer RNAs: Methods and Protocols (2017) 235-250 (incorporated herein by reference). In Streptococcus pyogenes, clustered regularly interspaced short palindromic repeats (CRISPR) RNAs (crRNAs) are expressed in conjunction with a scaffold RNA, known as the trans-activating-crRNA (tracrRNA), and guide Cas9 to the target DNA. The only constraint for target sequences is that they must immediately precede a suitable protospacer adjacent motif (PAM) of the form NGG. The bacterial CRISPR system has been further simplified to utilize a single-guide RNA molecule (sgRNA), which functions as a chimeric RNA to replace both the crRNA and tracrRNA elements. Furthermore, the native S. pyogenes Cas9 has been engineered to work within many eukaryotic systems, including mammalian cells, by delivering expression plasmids of codon-optimized Cas9 cDNA containing one, or more, nuclear localization signals (NLS). Point mutations in amino acids D10 and H840 of Cas9 render the enzyme catalytically inactive (dCas9), providing a programmable DNA binding protein without nuclease activity. Several groups have demonstrated that dCas9 can function as an effective ATF by fusion with transcription al activation domains.

The following protocol for designing, assembling and testing RGN transcription factors assumes that a dCas9-transcriptional activator has already been obtained. To aid the identification of a suitable activation system, Table 6 summarizes the different dCas9-transcriptional activators compatible with the gene activation systems described herein.

TABLE 6
Constructs Encoding dCas9-Transcriptional Activators for Stimulation
of Gene Expression in Mammalian Cells

| | Addgene | | Transcriptional |
|---|---|---|---|
| Plasmid name | # | Promoter | activation domain |
| SP-dCas9-VPR | 63798 | CMV | VPR (VP64-p65-Rta) |
| pcDNA-dCas9-p300 | 61357 | CMV | p300 Core (human, aa |
| Core | | | 1048-1664) |
| pcDNA-dCas9-VP64 | 47107 | CMV | VP64 |
| pAC93-pmax- | 48225 | CAGGS | VP160 |
| dCas9VP160 | | | |
| pAC91-pmax- | 48223 | CAGGS | VP64 |
| dCas9VP64 | | | |
| pAC92-pmax- | 48224 | CAGGS | VP96 |
| dCas9VP96 | | | |
| pSL690 | 47753 | CMV | VP64 |
| pCMV_dCas9_VP64 | 49015 | CMV | VP64 |
| CMVp-dCas9-3xNLS- | 55195 | UBC | VP64 |
| VP64 Construct 1 | | | |
| pMSCV-LTR-dCas9- | 46913 | MSCV | p65AD |
| p65AD-BFP | | LTR | |
| pMSCV-LTR-dCas9- | 46912 | MSCV | VP64 |
| VP64-BFP | | LTR | |
| EF_dCas9-VP64 | 68417 | EF1a | VP64 |
| pHAGE TRE dCas9- | 50916 | TRE | VP64 |
| VP64 | | | |
| pHAGE EF1α dCas9- | 50918 | EF1a | VP64 |
| VP64 | | | |
| dCAS9-VP64_GFP | 61422 | EF1a | VP64 |
| lenti dCAS-VP64_Blast | 61425 | EF1a | VP64 |
| pHRdSV40-NLS- | 60910 | SV40 | GCN4/SunTag system |
| dCas9-24xGCN4_ | | | |
| v4-NLS-P2A-BFP- | | | |
| dWPRE | | | |

Construction of sgRNA Expression Plasmids

1. An appropriate sgRNA vector should be chosen prior to guide design. Examples of sgRNA vectors for cloning and expression of custom sgRNAs using include, but are not limited to, those described in Table 7.

TABLE 7
Vectors for Cloning and Expression of Custom sgRNAs

| | Addgene | | Cloning |
|---|---|---|---|
| Plasmid name | # | Promoter | enzymes(s) |
| gRNA_Cloning Vector | 41824 | Human | AfIII |
| | | U6 | |
| pLKO5.sgRNA.EFS.GFP | 57822 | U6 | BsmBI |
| pLKO5.sgRNA.EFS.tRFP | 57823 | U6 | BsmBI |
| pLKO5.sgRNA.EFS.tRFP657 | 57824 | U6 | BsmBI |
| pLKO5.sgRNA.EFS.PAC | 57825 | U6 | BsmBI |
| pSPgRNA | 47108 | Human | BbsI |
| | | U6 | |
| phH1-gRNA | 53186 | Human | BbsI |
| | | H1 | |
| pmU6-gRNA | 53187 | Mouse | BbsI |
| | | U6 | |
| phU6-gRNA | 53188 | Human | BbsI |
| | | U6 | |
| ph7SK-gRNA | 53189 | Human | BbsI |
| | | 7SK | |
| pHL-H1-ccdB-mEF1a-RiH | 60601 | H1 | BamHI/EcoRI |
| pUC57-sgRNA expression vector | 51132 | T7 | BsaI |
| pGL3-U6-sgRNA-PGK- | 51133 | Human | BsaI |
| puromycin | | U6 | |
| pUC-H1-gRNA | 61089 | H1 | BsaI |
| pAC155-pCR8-sgExpression | 49045 | Human | BbsI |
| | | U6 | |
| pSQT1313 | 53370 | Human | BsmBI |
| | | U6 | |
| BPK1520 | 65777 | Human | BsmBI |
| | | U6 | |
| pU6_RNA_handle_U6t | 49016 | U6 | SacI |
| pGuide | 64711 | Human | BbsI |
| | | U6 | |
| pgRNA-humanized | 44248 | Mouse | BstXI + XhoI |
| | | U6 | |
| pLX-sgRNA | 50662 | Human | OE-PCR |
| | | U6 | |
| pLenti-sgRNA-Lib | 53121 | Human | BsmBI |
| | | U6 | |
| pU6-sgRNA EF1Alpha-puro- | 60955 | Mouse | BstXI + BlpI |
| T2A-BFP | | U6 | |
| pLKO.1-puro U6 sgRNA BfuAI | 50920 | Human | BfuAI |
| stuffer | | U6 | |
| +pKLV-U6gRNA(BbsI)- | 50946 | Human | BbsI |
| PGKpuro2ABFP | | U6 | |
| pH1v1 | 60244 | H1 | Gibson |
| lentiGuide-Puro | 52963 | Human | BsmBI |
| | | U6 | |
| AAV:ITR-U6-sgRNA(backbone)- | 60226 | U6 | SapI |
| pEFS-Rluc-2ACre- | | | |
| WPRE-hGHpA-ITR | | | |
| AAV:ITR-U6-sgRNA(backbone)- | 60229 | U6 | SapI |
| pCBh-Cre- | | | |
| WPRE-hGHpA-ITR | | | |
| AAV:ITR-U6-sgRNA(backbone)- | 60231 | U6 | SapI |
| hSyn-Cre-2AEGFP- | | | |
| KASH-WPRE-shortPA-ITR | | | |
| PX552 | 60958 | Human | SapI |
| | | U6 | |
| sgRNA(MS2) cloning backbone | 61424 | U6 | BbsI |
| lenti sgRNA(MS2)_zeo backbone | 61427 | U6 | BsmBI |
| pAC2-dual-dCas9VP48- | 48236 | Human | BbsI |
| sgExpression | | U6 | |
| pAC5-dual-dCas9VP48-sgTetO | 48237 | Human | BbsI |
| | | U6 | |
| pAC152-dual-dCas9VP64- | 48238 | Human | BbsI |
| sgExpression | | U6 | |
| pAC153-dual-dCas9VP96- | 48239 | Human | BbsI |
| sgExpression | | U6 | |
| pAC154-dual-dCas9VP160- | 48240 | Human | BbsI |
| sgExpression | | U6 | |

Dual expression of Cas9 and sgRNA from a single plasmid is an alternative to a two plasmid system. This protocol uses pSPgRNA (Addgene #47108), which includes two BbsI/BpiI sites interspaced between a human U6 promoter and the sgRNA loop for cloning of oligonucleotides (FIG. 13).

2. Oligonucleotides for sgRNA construction. Target selection: The identification of optimal target sites for activation of gene expression remains, essentially, an empirical process. It has been shown that the region comprising −400 to −50 bp at the 5′ end of the transcriptional start site (TSS) is optimal. Since the TSS is clearly annotated in most genome browsers, the sequence of the gene of interest is imported into DNA analysis software and used to identify potential target sites. Benchling, a freely available web-based DNA analysis platform that incorporates a “Genome Engineering” tool to identify all possible sgRNAs within any sequence specified by the user can be used. Benchling provides on-target and off-target scores associated with each target site. Off-target changes in gene expression are uncommon when using multiple sgRNAs to activate gene expression, since all target sites must be found simultaneously near the TSS of the off-target gene. However, since second-generation systems for gene activation require one single sgRNA, it is important to identify high quality sgRNAs with favorable off-target scores. For each sgRNA, Benchling provides a detailed list of potential off-target sites that can be used for biased detection of off-target gene activation.

The target sequences chosen to activate ASCL1 gene expression are: 5′-GCTGGGTGTCCCATTGAAA-3′ (SEQ ID NO: 56); 5′-CAGCCGCTCGCTGCAGCAG-3′ (SEQ ID NO: 57); 5′-TGGAGAGTTTGCAAGGAGC-3′ (SEQ ID NO: 58); 5′-GTTTATTCAGCCGGGAGTC-3′ (SEQ ID NO: 59). For each target sequence, a sense oligonucleotide is generated in the format: 5′-CACC G NNNNNNNNNNNNNNNNNNNN-3′ (SEQ ID NO: 60), where N 20 represents the 20 bases of the genomic DNA at the 5′ end of the PAM. The number of nucleotides in the sgRNA complementary with the target site can range between 17 and 20 bp. In fact, it has been demonstrated that sgRNAs with 17 or 18 complementary nucleotides efficiently guide S. pyogenes Cas9 to the target site where it introduces double strand breaks with improved specificity. The first four bases are complementary to the sgRNA vector overhangs, while the fifth base is G in order to initiate transcription of RNA from the upstream U6 promoter. A second oligonucleotide, representing the antisense target sequence, is generated in the format: 5′-AAACY20 C-3′ (SEQ ID NO: 61). Here, AAAC are vector complementing overhangs, Y20 represents the reverse complement of the target sequence, and the last C complements the leading G of the sense oligonucleotide (FIG. 13).

The sequences of the oligonucleotides for assembly of sgRNAs that can target the ASCL1 promoter are:

| (SEQ ID NO: 62) | |
|---|---|
| TARGET1S: | 5′- CACC G GCTGGGTGTCCCATTGAAA-3′. |
| (SEQ ID NO: 63) | |
| TARGET1AS: | 5′- AAAC TTTCAATGGGACACCCAGC C- 3′; |
| (SEQ ID NO: 64) | |
| TARGET2S: | 5′- CACC G CAGCCGCTCGCTGCAGCAG-3′; |
| (SEQ ID NO: 65) | |
| TARGET2AS: | 5′- AAAC CTGCTGCAGCGAGCGGCTG C- 3′; |
| (SEQ ID NO: 66) | |
| TARGET3S: | 5′- CACC G TGGAGAGTTTGCAAGGAGC-3′; |
| (SEQ ID NO: 67) | |
| TARGET3AS: | 5′- AAAC GCTCCTTGCAAACTCTCCA C- 3′; |
| (SEQ ID NO: 68) | |
| TARGET4S: | 5′- CACC G GTTTATTCAGCCGGGAGTC-3′; |
| (SEQ ID NO: 69) | |
| TARGET4AS: | 5′- AAAC GACTCCCGGCTGAATAAAC C- 3′. |

3. Nuclease-free Molecular biology grade (MBG) water.

4. Tris Buffered Saline (TBS), 50 mM Tris pH 7.4 and 150 mM NaCl.

5. Restriction endonuclease BbsI/BpiI. There are multiple commercial sources for BbsI/BpiI. Some formulations of BbsI/BpiI require storage at −80° C. and, repeated cycles of freeze-thaw that occur when used frequently, result in decreased enzymatic activity and undesired background during cloning. Formulations of BbsI/BpiI that can be stored at −20° C.

6. T4 Polynucleotide Kinase (PNK).

7. T4 DNA ligase and T4 DNA Ligase Buffer with ATP. T4 DNA ligase buffer typically contains 10 mM dithiothreitol, which is not stable through repeated freeze-thaw cycles. Single use aliquots of T4 buffer can be prepared.

8. Transformation-competent E. coli. Any chemically competent cells or electro-competent cells can be used, such asHIT Competent Cells-DH5α. These chemically competent cells can be transformed very efficiently without heat-shock by mixing 1.5 μL of the ligation reaction with 30 μL of competent cells followed by incubation at 4° C. for 1-10 min and plating. When using this short protocol, plates prewarmed at 37° C. ensures transformation efficiency. If the transformation efficiency is too low, addition of 100 μL of SOC broth and incubation at 37° C. with shaking for 10 min should yield hundreds to thousands of colonies.

9. LB-Agar plates containing 100 μg/mL carbenicillin for bacterial culture.

10. KAPA2G Robust PCR Kit (KAPA Biosystems) and 10 mM dNTP mix.

11. Sequencing and colony PCR primer, M13 Forward: 5′-TGTAAAACGACGGCCAGT-3′ (SEQ ID NO:70).

12. Ethidium bromide, 10 mg/mL.

13. Electrophoresis Buffer (TAE) 40 mM Tris pH 7.2, 20 mM Acetate, and 1 mM EDTA.

14. Agarose.

15. LB broth containing 100 μg/mL carbenicillin.

16. Qiagen Spin Miniprep Kit.

Activation of Target Gene Expression

1. Mammalian cell line, such as HEK293T.

2. Phosphate-buffered saline (PBS), 8 mM Na2HPO4, 2 mM KH2PO4 pH 7.4, 137 mM NaCl and 2.7 mM KCl.

3. 0.25% Trypsin-EDTA.

4. Complete mammalian cell culture medium appropriate for the chosen cell line, such as DMEM supplemented with 10% Fetal Bovine Serum (FBS) and 1% penicillin/streptomycin.

5. Lipofectamine 2000 (Thermo Fisher Scientific) or other suitable transfection reagent(s).

6. Opti-MEM (Thermo Fisher Scientific) reduced serum media.

7. Twenty-four well tissue culture-treated plates.

8. Transfection plasmids: pSPgRNA(s) with target sequence. pcDNA-dCas9-VP64 (Addgene#47107) or other suitable dCas9 transcriptional activator expression vector. pMAX-GFP (Amaxa) or other suitable reporter plasmid for measuring transfection efficiency.

Analysis of mRNA Expression

1. 0.25% Trypsin-EDTA.

2. PBS.

3. QIAshredder (Qiagen).

4. RNeasy Plus RNA isolation kit (Qiagen).

5. qScript cDNA SuperMix (Quanta Biosciences).

6. RNase/DNase-free water.

7. PerfeCTa® SYBR® Green FastMix (Quanta Biosciences).

8. Oligonucleotides for qPCR. Using high quality primers helps ensure reproducible qPCR results. Repeated freeze-thaw cycles can alter primer binding to the template. Upon receipt, the primers are resuspended in MBG water and prepare single use aliquots that are stored at −80° C. Multiple oligonucleotides are often designed and tested for finding a suitable primer combination that is specific and amplifies the target transcript with 90-110% efficiency. Many design tools, such as Primer3Plus, are freely available as stand-alone or web-based applications. qPCR is performed using fast cycling two-step protocols with amplicons between 100 and 150 bp long. One consideration for primer design is to use primers that bind different exons separated, if possible, by several kilobases. This will ensure that any residual genomic DNA that might be present in the RNA sample will not be amplified during the PCR reaction.

| (SEQ ID NO: 71) | |
|---|---|
| ASCL FW: | 5′ GGAGCTTCTCGACTTCACCA-3′. |
| (SEQ ID NO: 72) | |
| ASCL REV: | 5′-AACGCCACTGACAAGAAAGC-3′. |
| (SEQ ID NO: 39) | |
| GAPDH FW: | 5′-CAATGACCCCTTCATTGACC-3′. |
| (SEQ ID NO: 40) | |
| GAPDH REV: | 5′ TTGATTTTGGAGGGATCTCG-3′. |

9. CFX96 Real-Time PCR Detection System (Bio-Rad).

Design and construction of sgRNA Expression Plasmids

The procedure utilized for generating sgRNA vectors accomplishes plasmid digestion, oligonucleotide phosphorylation and ligation in a single reaction without DNA purification steps. This is a low cost and highly efficient procedure that can be completed in less than two hours from annealing to transformation.

1. Design and synthesize/order oligonucleotides to target the regions of the promoter proximal to the TSS of the target transcript. Stocks of each oligonucleotide prepared at 100 μM in nuclease-free molecular biology grade water, can be stored frozen for extended periods.

2. Combine 1 μL of each sense and antisense oligonucleotide with 98 μL of TBS in a PCR tube. Anneal the oligonucleotide mix by incubation at 95° C. for 5 min, followed by 25° C. for 3 min.

3. Mix 1 μL of annealed and diluted oligonucleotides with 170 ng sgRNA vector, 2 μL 10×T4 ligase buffer, 1 μL of T4 ligase, 1 μL BbsI/BpiI, 1 μL T4 polynucleotide kinase (PNK), and MBG water to a final reaction volume of 20 μL. The sgRNA vector backbone is simultaneously digested and ligated with the annealed, phosphorylated oligonucleotides in a single reaction with the following thermocycling program: 37° C., 5 min. 16° C., 10 min. Repeat a and b for a total of three cycles.

4. Transform ligated plasmid by mixing 1.5 μL of the reaction product with 30 μL of competent E. coli, spread onto prewarmed LB agar containing 100 μg/mL carbenicillin, and incubate overnight at 37° C.

5. Correct ligation is ensured by analyzing four transformants per plate using colony PCR with KAPA2G Robust PCR Kits. 25 μL reactions containing MBG water (11.9 μL), 5×KAPA2G Buffer (5.0 μL), 5× Enhancer (5.0 μL), 10 mM dNTP mix (0.50 μL), 10 μM M13 Forward primer (1.25 μL), 10 μM Reverse primer (antisense cloning oligonucleotide) (1.25 μL), and 5 U/μL KAPA2G Robust (0.10 μL) are used for sequencing. With a pipette tip, scrape one colony from the plate, transfer to the PCR reaction and, immediately, to a second PCR tube containing LB broth. The PCR reactions are performed in a thermocycler according to manufacturer's instructions and the PCR products analyzed in 2% agarose gels containing 0.1-0.2 μg/mL ethidium bromide. The expected size of the correct PCR product is ˜330 bp.

6. One colony, verified by PCR, is grown overnight in 5 mL of LB broth with 100 μg/mL carbenicillin.

7. The plasmid DNA from the bacterial culture is purified using a plasmid purification kit such as the Qiagen Spin Miniprep Kit and the construct is verified by DNA sequencing with M13 Forward primer.

Activation of Target Gene Expression in Mammalian Cells

1. A typical experimental setup includes reactions containing plasmid mixtures such as the following: GFP (1 μg). sgRNA 1 and dCas9 (0.5 μg each). sgRNA 2 and dCas9 (0.5 μg each). sgRNA 3 and dCas9 (0.5 μg each). sgRNA 4 and dCas9 (0.5 μg each). sgRNA 1+sgRNA 2+sgRNA 3+sgRNA 4 (0.125 μg of each) and dCas9 (0.5 μg).

Plasmid DNA purified using Qiagen Spin Miniprep Kit is suitable for transfection of a variety of cell lines, however, the resulting plasmid prep contains significant levels of endotoxins from E. coli that can result in decreased viability in some cell types. DNA precipitation with ethanol is usually sufficient to obtain transfection grade DNA suitable for use in most cell types. A control transfection reaction containing a GFP or similar expression plasmid should be used to ensure adequate transfection efficiency is achieved under identical experimental conditions and to serve as a negative control for qPCR.

2. For optimal transfection efficiency, low passage 293T cells in logarithmic growth are trypsinized, harvested, and resuspended at 10⁶ cells/mL in DMEM.

3. As per manufacturer's instructions, the DNA is mixed with 50 μL of Opti-MEM in a microfuge tube and, in a separate tube, 2 μL of Lipofectamine 2000 are mixed with 50 μL of Opti-MEM. After 5 min, the contents of both tubes are combined and incubated for an additional 20 min. The 100 μL DNA-lipofectamine reagent mixture is pipetted into one well of a 24-well treated tissue culture dish and promptly mixed with 400 μL of freshly harvested and properly diluted cells. Transfections are typically performed in antibiotic free medium. Decreased transfection efficiency or viability by using antibiotics in 293T cells has not been observed.

4. Incubate the cells for 48-72 h before analyzing gene expression.

Analysis of Gene Expression by qPCR

1. The cells are trypsinized and washed with PBS once. Gene expression is analyzed in three independent experiments that are performed on three different days using biological duplicates in each experiment. Since RNA is unstable and degrades rapidly over time, it can be advantageous to harvest the cells and freeze cell pellets until all three experiments have been completed. At that point RNA extraction is performed from all samples simultaneously to minimize variability due to sample handling.

2. Total RNA is isolated using the RNeasy Plus RNA isolation kit (Qiagen) or another standard enzymatic removal method of genomic DNA after RNA isolation. The cells are lysed by adding an appropriate volume of RLT Plus with 10 μL/mL of β-mercaptoethanol and homogenized with QIAshredder columns. All other steps are performed according to manufacturer's instructions. It is recommended to prepare 70% ethanol and RPE buffer fresh before use.

3. cDNA synthesis is performed using the qScript cDNA SuperMix (Quanta Biosciences) by incubation of 1 μg of RNA with 4 μL of qScript cDNA SuperMix and RNase/DNase-free water up to 20 μL. The thermocycling parameters are: (a) 5 min at 25° C. (b) 30 min at 42° C. (c) 5 min at 85° C. For the cDNA synthesis reaction to occur identically in all samples, it is important to use equal amounts of RNA from all samples. cDNA can be prepared from 1 μg of RNA.

4. Real-time PCR is performed using PerfeCTa® SYBR® Green FastMix (Quanta Biosciences) with the CFX96 Real-Time PCR Detection System (Bio-Rad). The primers are designed using Primer3Plus, purchased from IDT and validated by agarose gel electrophoresis and melting curve analysis. For each sample, quantification of a housekeeping gene (such as GAPDH) must be performed in addition to analysis of the target gene. The qPCR reactions contain 10 μL PerfeCTa® SYBR® Green FastMix (2×), 2 μL forward primer (5 μM), 2 μL reverse primer (5 μM), cDNA and RNase/DNase-free water up to 20 μL. The optimal cycling parameters for each gene must be determined experimentally to ensure efficient amplification over an appropriate dynamic range. Standard curves are generated using tenfold dilutions with cDNA obtained from the sample presumed to have the highest transcript concentration. The use of plasmid DNA or other synthetic templates can lead to errors in determining the linear range of the PCR.

5. Calculate fold-increase mRNA expression of the gene of interest normalized to GAPDH expression using the ddCt method.

### Example 5. Demonstration of a Universal System of NAVI-Based Gene Activation (NAVIa)

A nuclease-assisted vector integration (NAVI) for insertion of promoters at target sites was selected. NAVI can be rapidly adapted to integrate heterologous DNA at virtually any locus via two simultaneous DSBs: first in the genome, guided by a primary sgRNA, and second within the targeting vector (TV), guided by a universal secondary sgRNA. The TV is then integrated into the genomic locus through Non-Homologous End Joining (NHEJ). This platform is universal since vector integration at any target site can be simply accomplished by customizing the primary sgRNA.

To develop a universal system of NAVI-based gene activation (NAVIa), two vectors for constitutive expression and one vector for inducible expression were designed.

Cell Culture and Transfection

293T and HCT116 cells were obtained from the American Tissue Collection Center (ATCC) and were maintained in DMEM supplemented with 10% fetal bovine serum and 1% penicillin/streptomycin at 37° C. with 5% CO₂. 293T and HCT116 cells were transfected with Lipofectamine 2000 (Invitrogen) according to manufacturer's instructions. Transfection efficiencies were routinely higher than 80% for 293T cells and higher than 50% for HCT116 cells as determined by fluorescent microscopy following delivery of a control GFP expression plasmid. Induction of gene expression, unless otherwise noted, was carried out with 200 ng/mL doxycycline in DMEM prepared with 10% tetracycline-free FBS for 4 days.

Plasmids and Oligonucleotides

The plasmids encoding SpCas9 (Plasmid #41815), sgRNA (#47108), SpdCas9-VPR (#63798) and sgRNA library (#1000000078) were obtained from Addgene. The backbone for the targeting vectors was synthesized by IDT Technologies as gene blocks and cloned into a pCDNA3.1 plasmid. Guide sequences were obtained from IDT Technologies, hybridized, phosphorylated and cloned in the sgRNA vector using BbsI sites (see also Example 3). The target sequences are provided in Table 8.

TABLE 8
Target Sequences

| | | | SEQ | | On- | Off- | | BP | BP | |
|---|---|---|---|---|---|---|---|---|---|---|
| | | | ID. | | target | target | 5′ | from | from | Pro- |
| Designation | GOI | Sequence | NO. | PAM | score | score | mismatch | TSS | ATG | moter |
| ASCL1.1 | ASCL1 | CACCGCTCTGATTCC | 73 | TGG | 43.9 | 82.4 | — | 541 | −18 | hU6 |
| | | GCGACTCCT | | | | | | | | |
| ASCL1.1 | ASCL1 | AAACAGGAGTCGCGG | 74 | TGG | 43.9 | 82.4 | — | 541 | −18 | hU6 |
| | | AATCAGAGC | | | | | | | | |
| ASCL1.2 | ASCL1 | CACCGCCAGAAGTGA | 75 | GGG | 54.5 | 44.5 | — | −9 | −568 | hU6 |
| | | GAGAGTGCT | | | | | | | | |
| ASCL1.2 | ASCL1 | AAACAGCACTCTCTC | 76 | GGG | 54.5 | 44.5 | — | −9 | −568 | hU6 |
| | | ACTTCTGGC | | | | | | | | |
| ASCL1.3 | ASCL1 | CACCGCGGGAGAAAG | 77 | GGG | 30.9 | 42.7 | — | −196 | −755 | hU6 |
| | | GAACGGGAGG | | | | | | | | |
| ASCL1.3 | ASCL1 | AAACCCTCCCGTTCC | 78 | GGG | 30.9 | 42.7 | — | −196 | −755 | hU6 |
| | | TTTCTCCCGC | | | | | | | | |
| ASCL1.4 | ASCL1 | CACCGAAGAACTTGA | 79 | AGG | 50.5 | 68.6 | G | −451 | −1010 | hU6 |
| | | AGCAAAGCGC | | | | | | | | |
| ASCL1.4 | ASCL1 | AAACGCGCTTTGCTT | 80 | AGG | 50.5 | 68.6 | G | −451 | −1010 | hU6 |
| | | CAAGTTCTTC | | | | | | | | |
| h7SK | ASCL1 | CCTCGAAGAACTTGA | 81 | AGG | 50.5 | 68.6 | G | −451 | −1010 | h7SK |
| ASCL1 | | AGCAAAGCGC | | | | | | | | |
| h7SK | ASCL1 | CCTCGAGGCCAATAG | 82 | AGG | 50.5 | 68.6 | G | −451 | −1010 | h7SK |
| ASCL1 | | GAACACTGCG | | | | | | | | |
| ASCL1.5 | ASCL1 | AAACCGGTGACCCTA | 83 | AGG | 68.4 | 76.3 | G | −572 | −1131 | hU6 |
| | | GAAATTGGAC | | | | | | | | |
| ASCL1.5 | ASCL1 | CACCGTCCAATTTCT | 84 | AGG | 68.4 | 76.3 | G | −572 | −1131 | hU6 |
| | | AGGGTCACCG | | | | | | | | |
| ASCL1.6 | ASCL1 | CACCGTTGTGAGCCG | 85 | TGG | 57.1 | 71.4 | — | −886 | −1445 | hU6 |
| | | TCCTGTAGG | | | | | | | | |
| ASCL1.6 | ASCL1 | AAACCCTACAGGACG | 86 | TGG | 57.1 | 71.4 | — | −886 | −1445 | hU6 |
| | | GCTCACAAC | | | | | | | | |
| 1L1B | IL1B | TCCCAGTATTGGTGG | 87 | GGG | 41.4 | 51.8 | A | −9 | −683 | hH1 |
| | | AAGCTTCTTA | | | | | | | | |
| IL1B | IL1B | AAACTAAGAAGCTTC | 88 | GGG | 41.4 | 51.8 | A | −9 | −683 | hH1 |
| | | CACCAATACT | | | | | | | | |
| IL1R2 | IL1R2 | TTGTTTGAGAGAATC | 89 | GGG | 63.7 | 53.2 | — | −62 | −123 | mU6 |
| | | CCTTGAAGACG | | | | | | | | |
| IL1R2 | IL1R2 | AAACCGTCTTCAAGG | 90 | GGG | 63.7 | 53.2 | — | −62 | −123 | mU6 |
| | | GATTCTCTCAA | | | | | | | | |
| LIN28A | LIN28A | TTGTTTGCTTCCCCC | 91 | TGG | 56.2 | 91.2 | G | −5 | −119 | mU6 |
| | | GCACAATAGCGG | | | | | | | | |
| LIN28A | LIN28A | AAACCCGCTATTGTG | 92 | TGG | 56.2 | 91.2 | G | −5 | −119 | mU6 |
| | | CGGGGGAAGCAA | | | | | | | | |
| NEUROD1.1 | NEURO | CACCGCGATTTCCTA | 93 | GGG | 51.9 | 47.5 | G | 1995 | −21 | hU6 |
| | D1 | CATTCAACAA | | | | | | | | |
| NEUROD1.1 | NEURO | AAACTTGTTGAATGT | 94 | GGG | 51.9 | 47.5 | G | 1995 | −21 | hU6 |
| | D1 | AGGAAATCGC | | | | | | | | |
| NEUROD1.2 | NEURO | CACCGAGGGGAGCGG | 95 | AGG | 30.9 | 69.3 | — | 171 | −1841 | hU6 |
| | D1 | TTGTCGGAGG | | | | | | | | |
| NEUROD1.2 | NEURO | AAACCCTCCGACAAC | 96 | AGG | 30.9 | 69.3 | — | 171 | −1841 | hU6 |
| | D1 | CGCTCCCCTC | | | | | | | | |
| NEUROD1.3 | NEURO | CACCGACCTGCCCAT | 97 | CGG | 55.4 | 80.8 | — | 50 | −1966 | hU6 |
| | D1 | TTGTATGCCG | | | | | | | | |
| NEUROD1.3 | NEURO | AAACCGGCATACAAA | 98 | CGG | 55.4 | 80.8 | — | 50 | −1966 | hU6 |
| | D1 | TGGGCAGGTC | | | | | | | | |
| hH1 | NEURO | TCCCACCTGCCCATT | 99 | CGG | 55.4 | 80.8 | — | 50 | −1966 | hH1 |
| NEUROD1 | D1 | TGTATGCCG | | | | | | | | |
| hH1 | NEURO | AAACCGGCATACAAA | 100 | CGG | 55.4 | 80.8 | — | 50 | −1966 | hH1 |
| NEUROD1 | D1 | TGGGCAGGT | | | | | | | | |
| NEUROD1.4 | NEURO | CACCGAGGTCCGCGG | 101 | TGG | 42.1 | 85.5 | G | −13 | −2029 | hU6 |
| | D1 | AGTCTCTAAC | | | | | | | | |
| NEUROD1.4 | NEURO | AAACGTTAGAGACTC | 102 | TGG | 42.1 | 85.5 | G | −13 | −2029 | hU6 |
| | D1 | CGCGGACCTC | | | | | | | | |
| NEUROD1.5 | NEURO | CACCGTCGCCAGTTA | 103 | CGG | 70.6 | 86.4 | — | −20 | −2036 | hU6 |
| | D1 | GAGACTCCG | | | | | | | | |
| NEUROD1.5 | NEURO | AAACCGGAGTCTCTA | 104 | CGG | 70.6 | 86.4 | — | −20 | −2036 | hU6 |
| | D1 | ACTGGCGAC | | | | | | | | |
| NEUROD1.6 | NEURO | CACCGTAGAGGGGCC | 105 | AGG | 38.8 | 83.2 | G | −369 | −2385 | hU6 |
| | D1 | GACGGAGATT | | | | | | | | |
| NEUROD1.6 | NEURO | AAACAATCTCCGTCG | 106 | AGG | 38.8 | 83.2 | G | −369 | −2385 | hU6 |
| | D1 | GCCCCTCTAC | | | | | | | | |
| POU5F1.1 | P0U5F1 | CACCGGTGAAATGAG | 107 | GGG | 58.5 | 68.2 | — | 24 | −49 | hU6 |
| | | GGCTTGCGAA | | | | | | | | |
| POU5F1.1 | P0U5F1 | AAACTTCGCAAGCCC | 108 | GGG | 58.5 | 68.2 | — | 24 | −49 | hU6 |
| | | TCATTTCACC | | | | | | | | |
| mU6 | P0U5F1 | TTGTTTGTGAAATGA | 109 | GGG | 58.5 | 68.2 | TT | 24 | −49 | mU6 |
| POU5F1 | | GGGCTTGCGAA | | | | | | | | |
| mU6 | P0U5F1 | AAACTTCGCAAGCCC | 110 | GGG | 58.5 | 68.2 | TT | 24 | −49 | mU6 |
| POU5F1 | | TCATTTCACAA | | | | | | | | |
| POU5F1.2 | P0U5F1 | CACCGCTCTCCTCCA | 111 | GGG | 62.4 | 42 | G | −47 | −120 | hU6 |
| | | CCCATCCAGG | | | | | | | | |
| POU5F1.2 | P0U5F1 | AAACCCTGGATGGGT | 112 | GGG | 62.4 | 42 | G | −47 | −120 | hU6 |
| | | GGAGGAGAGc | | | | | | | | |
| POU5F1.3 | P0U5F1 | CACCGACCTGCACTG | 113 | GGG | 53.4 | 44.4 | — | −165 | −238 | hU6 |
| | | AGGTCCTGGA | | | | | | | | |
| POU5F1.3 | P0U5F1 | AAACTCCAGGACCTC | 114 | GGG | 53.4 | 44.4 | — | −165 | −238 | hU6 |
| | | AGTGCAGGTC | | | | | | | | |
| POU5F1.4 | POU5F1 | CACCGCCTTTAATCA | 115 | CGG | 72.7 | 40.9 | — | −459 | −532 | hU6 |
| | | TGACACTGGG | | | | | | | | |
| POU5F1.4 | POU5F1 | AAACCCCAGTGTCAT | 116 | CGG | 72.7 | 40.9 | — | −459 | −532 | hU6 |
| | | GATTAAAGGC | | | | | | | | |
| POU5F1.5 | POU5F1 | CACCGGGAATGCCTA | 117 | TGG | 62.5 | 55.8 | | −759 | −832 | hU6 |
| | | GGATTCTGGA | | | | | | | | |
| POU5F1.5 | POU5F1 | AAACTCCAGAATCCT | 118 | TGG | 62.5 | 55.8 | | −759 | −832 | hU6 |
| | | AGGCATTCCC | | | | | | | | |
| CMV gRNA | TV | CACCGTCGATAAGCC | 119 | GGG | 45.7 | 73.8 | — | — | — | hU6 |
| 1 | | AGTAAGCAGT | | | | | | | | |
| CMV gRNA | TV | AAACACTGCTTACTG | 120 | GGG | 45.7 | 73.8 | — | — | — | hU6 |
| 1 | | GCTTATCGAC | | | | | | | | |
| h7SK CMV | TV | CCTCGTCGATAAGCC | 121 | GGG | 45.7 | 73.8 | — | — | — | h7SK |
| | | AGTAAGCAGT | | | | | | | | |
| h7SK CMV | TV | AAACACTGCTTACTG | 122 | GGG | 45.7 | 73.8 | — | — | — | h7SK |
| | | GCTTATCGAC | | | | | | | | |
| ZFP42 | ZFP42 | TCCCATTAGACCGCG | 123 | AGG | 59.1 | 94 | — | −50 | −7087 | hH1 |
| | | TCAGTCCGG | | | | | | | | |
| ZFP42 | ZFP42 | AAACCCGGACTGACG | 124 | AGG | 59.1 | 94 | — | −50 | −7087 | hH1 |
| | | CGGTCTAAT | | | | | | | | |

PCR

Seventy-two hours after transfection, genomic DNA was isolated using DNeasy Blood & Tissue Kit (Qiagen). PCRs were performed using KAPA2G Robust PCR kits (KAPA Biosystems). A typical 25 μL reaction used 20-100 ng of genomic DNA, Buffer A (5 μL), Enhancer (5 μL), dNTPs (0.5 μL), 10 μM forward primer (1.25 μL), 10 μM reverse primer (1.25 μL), KAPA2G Robust DNA Polymerase (0.5 U) and water (up to 25 μL). The DNA sequence of the primers for each target and the cycling parameters for each reaction are provided in Table 9. The PCR products were visualized in 2% agarose gels and images were captured using a ChemiDoc-It² (UVP).

TABLE 9
Integration Detection PCR Primers

| | | SEQ |
|---|---|---|
| | | ID |
| Target | Sequence (5′->3′) | NO. |
| ASCL1 | TTCCTTCTTTCACTCGCCCTCC | 125 |
| IL1B | CCAGTTTCTCCCTCGCTGTT | 126 |
| IL1R2 | GGCCCACACTTTGCTTTCTG | 127 |
| LIN28A | CTTTGGGCAGCCTAGGACTC | 128 |
| NEUROD1 | TGAGGGGCTAGCAGGTCTATGC | 129 |
| OCT4 | GGAATCCCCCACACCTCAGAG | 130 |
| TV | TGCTAGCTACGATGCACATCCA | 131 |
| TV | GCCCCGAATTCGAGCTCGGTAC | 132 |
| ZFP42 | TTTCCAATGCCACCTCCTCC | 133 |

qPCR

Cells were harvested and flash-frozen in liquid nitrogen prior to RNA-extraction using the RNeasy Plus RNA isolation kit (Qiagen) according to manufacturer's instructions. cDNA synthesis was carried out using the qScript cDNA Synthesis Kit (Quanta Biosciences) from 1 μg of RNA and reactions were performed as directed by the supplier. For RT-qPCR, SsoFast EvaGreen Supermix (Bio-Rad) was added to cDNA and primers targeting the gene of interest and GAPDH (Table 10). Following 30 s at 95° C., qPCR (5 s at 95° C., 20 s at 55° C., 40 total cycles) preceded melt-curve analysis of the product by the CFX Connect Real-Time System (Bio-Rad). Ct values were used to calculate changes in expression level, relative to GAPDH and control samples by the 2^{-ΔΔCt} method.

TABLE 10
RT-qPCR primers

| | | SEQ |
|---|---|---|
| | | ID |
| Designation | Sequence (5′->3′) | NO. |
| ASCL1 qPCRFW | GGAGCTTCTCGACTTCACCA | 71 |
| ASCL1 qPCRREV | AACGCCACTGACAAGAAAGC | 72 |
| NEUROD1 qPCRFW | ATGACGATCAAAAGCCCAAG | 134 |
| NEUROD1 | GAATAGCAAGGCACCACCTT | 135 |
| qPCRREV | | |
| IL1B qPCR F | AGCTGATGGCCCTAAACAGA | 136 |
| IL1B qPCR R | AAGCCCTTGCTGTAGTGGTG | 137 |
| IL1R2 qPCR F | CAGGAGGACTCTGGCACCTA | 138 |
| IL1R2 qPCR R | CGGCAGGAAAGCATCTGTAT | 139 |
| ZFP42 qPCR F | CTGGAGCCTGTGTGAACAGA | 140 |
| ZFP42 qPCR R | CAACCACCTCCAGGCAGTAG | 141 |
| LIN28A qPCR F | TTCGGCTTCCTGTCCATGAC | 142 |
| LIN28A qPCR R | CTGCCTCACCCTCCTTCAAG | 143 |
| POU5F1 qPCRFW | GAAGGAGAAGCTGGAGCAAA | 144 |
| POU5F1 qPCRREV | ATCCCAGGGTGATCCTCTTC | 145 |
| hGAPDH qPCRFW | CAATGACCCCTTCATTGACC | 39 |
| hGAPDH qPCRREV | TTGATTTTGGAGGGATCTCG | 40 |

Results

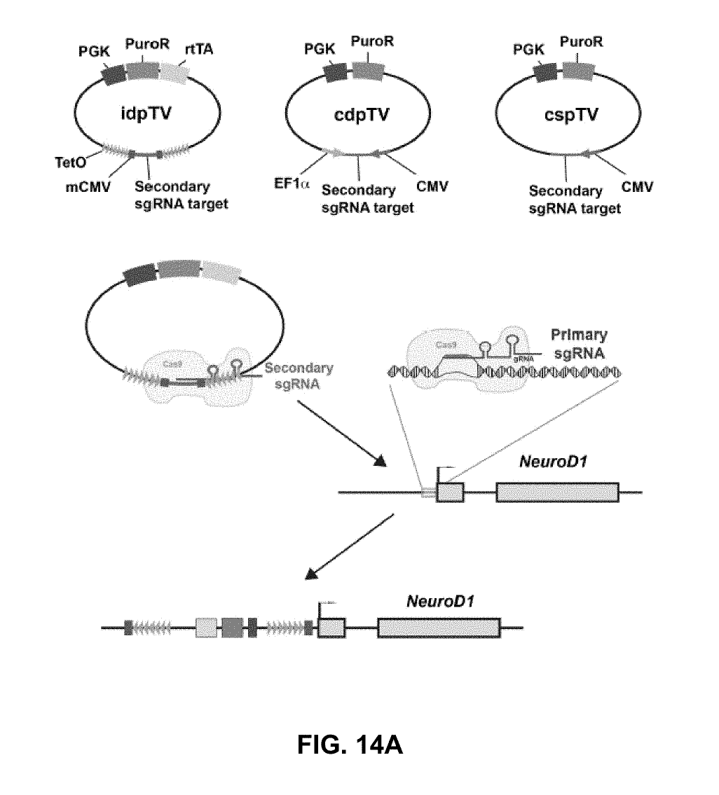

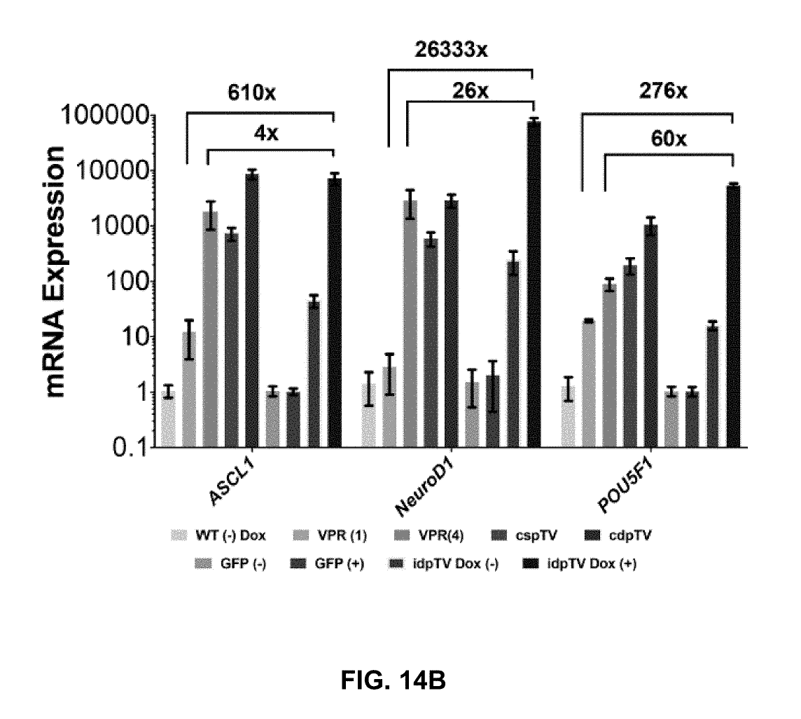

The two constitutive vectors contain either one CMV promoter followed by a target site for a universal secondary sgRNA (constitutive single promoter targeting vector, cspTV) or two opposing constitutive promoters separated by the secondary sgRNA target site (constitutive dual promoter targeting vector, cdpTV), each containing a cassette for expression of the puromycin N-acetyl-transferase gene. The targeting vector for inducible expression (inducible dual promoter targeting vector, idpTV) includes two identical promoters in opposite orientations, each consisting of seven TetO repeats and a minimal CMV promoter (mCMV). The idpTV also carries a puromycin N-acetyl-transferase gene linked with a reverse tetracycline transactivator (rtTA) via a T2A peptide. As in the cdpTV, the opposing promoters of the idpTV flank a universal secondary sgRNA target sequence. A DSB introduced in either idpTV or cdpTV by Cas9 generates a linear fragment of DNA with diametric promoters oriented towards the free ends of the vector (FIG. 14A). The architecture of the dual promoter TV ensures that there is always a promoter correctly positioned regardless of integration orientation, thereby addressing NAVI's lack of directionality.

In order to evaluate this gene activation architecture in the context of the human genome, three target genes were selected whose reported levels of activation utilizing CRISPRa are either high (ASCL1, ˜10³-fold), medium (NEUROD1, ˜10²-fold), or low (POU5F1, ˜10-fold). The primary sgRNAs targeting the genome were co-transfected into 293T cells with three plasmids containing (1) an expression cassette for active Cas9, (2) customized cspTV, cdpTV or idpTV, and (3) a universal secondary sgRNA. Following transfection, cells with integration of the TV were selected using puromycin and, in cells transfected with the idpTV, gene expression was induced with doxycycline. In parallel, one sgRNA or a mixture of 4 sgRNAs (previously validated for use with CRISPRa) were co-transfected into 293 Ts with dCas9-VPR for comparison of the NAVIa with CRISPRa. Gene expression using an individual sgRNA directing dCas9-VPR to target promoters was increased ˜10-fold for all targets tested but not statistically significant. Utilization of 4 sgRNAs simultaneously activated gene expression more effectively than 1 sgRNA (ASCL1: ˜1800-fold, NEUROD1: ˜2900-fold, POU5F:1 ˜90-fold). The levels of gene activation using the cspTV (ASCL1: ˜730-fold, NEUROD1: ˜600-fold, POU5F:1 ˜200-fold) or cdpTV (ASCL1: ˜8500-fold, NEUROD1: ˜3000-fold, POU5F1: ˜1000-fold) were superior to CRISPRa using 1 sgRNA but lower or not statistically different from activation obtained using 4 sgRNA for two of the three targets. However, the idpTV (ASCL1: ˜7200-fold, NEUROD1: ˜76000-fold, POU5F1: ˜5370-fold) surpassed activation obtained using dCas9-VPR using 4 sgRNAs (FIG. 14B). Interestingly, in this experiment, the improvement of NAVIa over dCas9-VPR was higher for targets branded as difficult to regulate with CRISPRa (POU5F1: ˜60-fold improvement, NEUROD1: ˜26-fold improvement) than for a target considered easy to activate (ASCL1: ˜4-fold improvement).

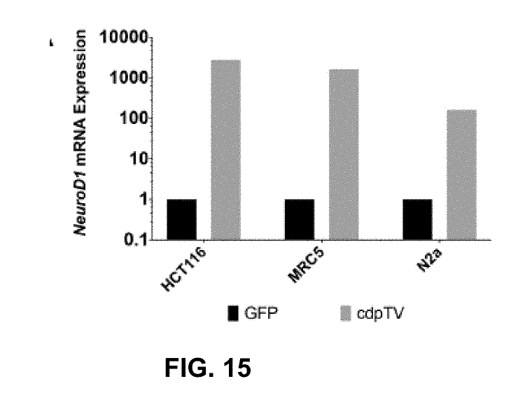

To further explore the trends we observed in 293T cells, NeuroD1 was targeted using the cdpTV in other cell lines. NAVIa effectively activated expression of NeuroD1 in the human colorectal carcinoma cell line HCT116, the primary human fibroblast cell line MRC-5, and the mouse neuroblastoma cell line Neuro2A (FIG. 15).

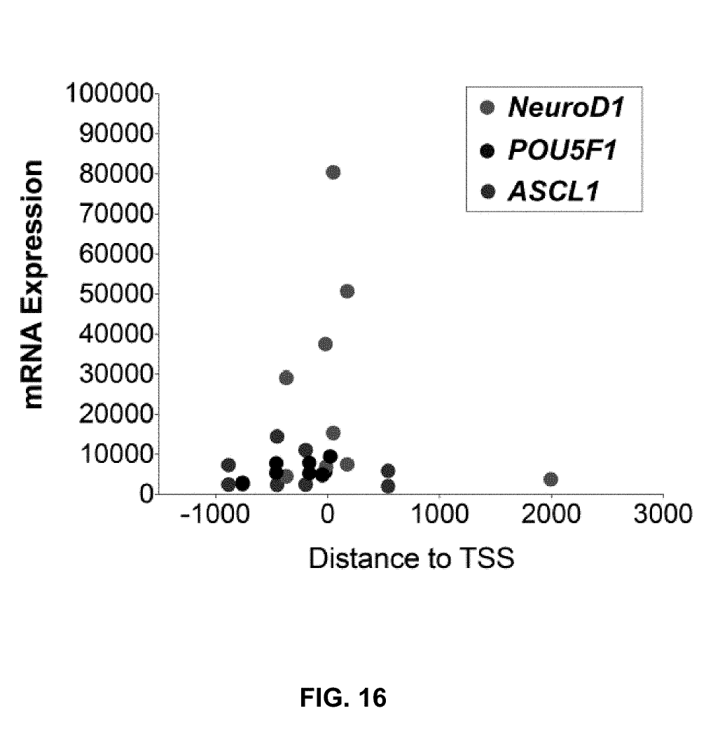

When using CRISPRa it is difficult to predict optimal sgRNA target sites for efficient gene activation. While it is generally accepted that proximity to the TSS of the target site is important, other parameters such as presence of enhancers or local chromatin structure are also critical and, perhaps, more difficult to predict. We investigated a potential correlation between gene activation using NAVIa and distance between integration site and TSS by measuring gene expression induced with sgRNAs that target DNA sequences between positions −1010 and +1995, relative to the TSS of 3 different genes (FIG. 16). Plotting these data for all 3 genes showed that NAVIa can activate gene expression efficiently from any integration site on this range, with the most activity being derived from sgRNAs between −500 and +200 bp relative to the TSS.

These results demonstrate a novel platform to activate native gene expression based on integration of heterologous promoters that overcomes some of the limitations intrinsic to CRISPRa. Promoter integration is accomplished by NAVI, which utilizes NHEJ and therefore overcomes some of the intrinsic limitations of DNA integration platforms that rely on Homologous Recombination (HR). For example, NHEJ is more effective than HR in non-dividing cells and has been exploited to integrate therapeutic transgenes in post-mitotic cells. In addition, we demonstrate that since this integration mechanism requires only one element that is variable, it can be adapted for genome-scale screenings.

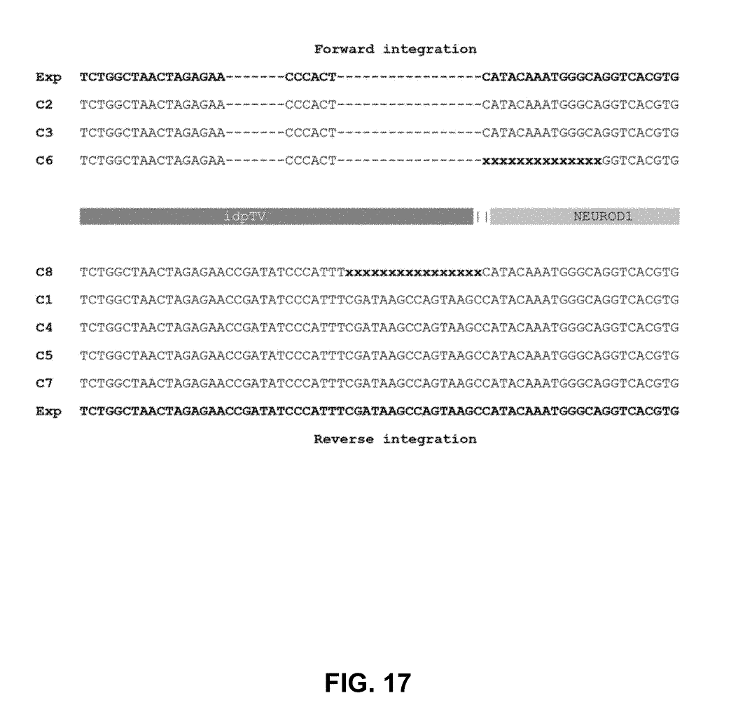

Although NAVI is subject to some shortcomings associated with its specific gene editing mechanism, such as the error-prone nature of NHEJ, only minor indels at target sites were observed (FIG. 17). Furthermore, as this system targets non-coding regions, supplanting basic functionality of the local sequence, imprecise genome editing is very unlikely to be prohibitive of endogenous gene activation.

One concern about the NAVIa system is that it is prone to Cas9 off-target nuclease activity. Such activity may lead to off-target vector integration and the inadvertent upregulation of additional genes. This problem could be lessened by using truncated sgRNAs or enhanced versions of Cas9 that have increased specificity. While CRISPRa is also susceptible to off-target activation, one fundamental difference between both systems is that, for sustained gene activation, CRISPRa necessitates the stable expression, or repeated introduction, of heterologous system components, which may have obvious negative implications on their own. In addition, it has been demonstrated that gene activation from viral vectors is less efficient than activation with episomal plasmids, presumably due to lower copy number. In contrast, NAVIa only necessitates transient nuclease activity to integrate a single synthetic element and is easily amenable to repeated customization to reduce or completely eliminate off-target effects.

### Example 6. Temporal Control of Gene Expression with the NAVIa System

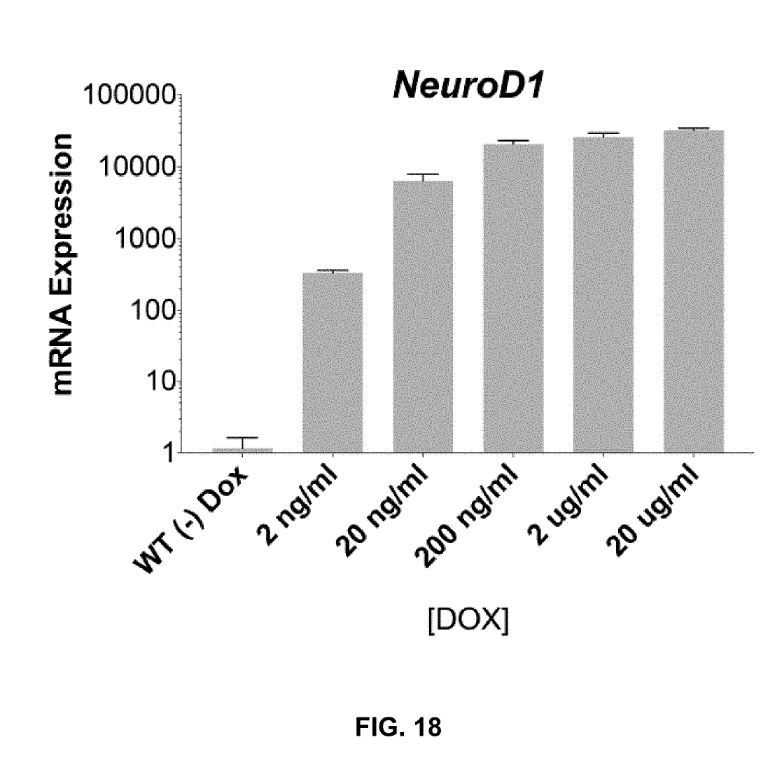

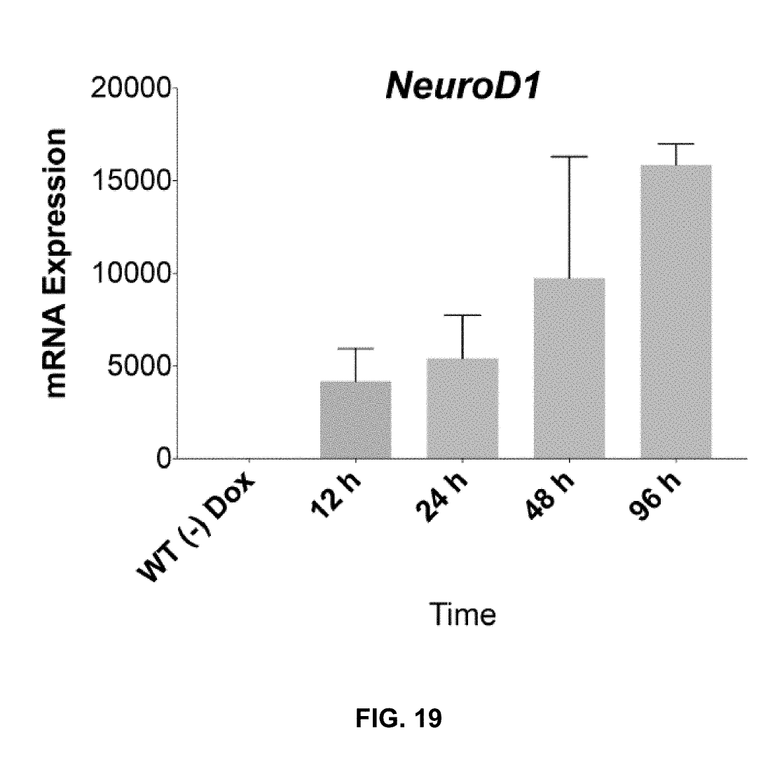

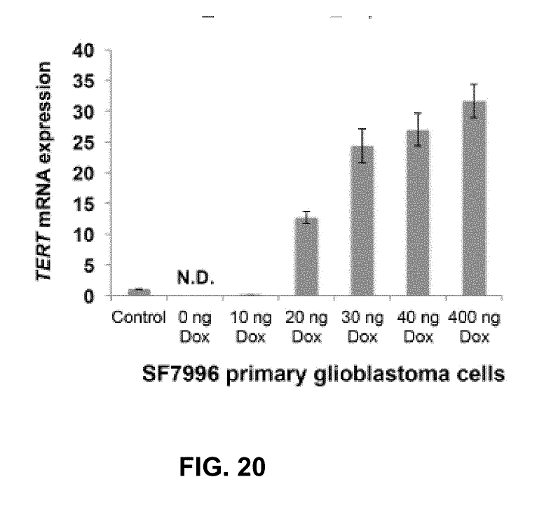

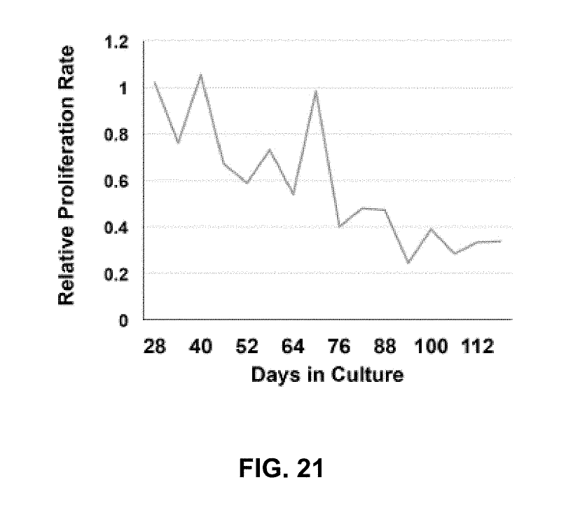

Since maximal gene activation may not be desirable in all experimental settings, CRISPRa has been adapted for tunable gene expression through combinatorial delivery of multiple sgRNAs. However, such efforts to modulate gene expression have proven unpredictable, with results that are difficult to reproduce. Alternatively, NAVIa enables facile customization of TV, including selection from a wide variety of gene regulatory mechanisms provided by existing artificial promoters. The idpTV used in these experiments introduces a doxycycline-inducible promoter and a precise temporal control of gene expression that could be tuned by the concentration of doxycycline in the growth medium. Induction of gene expression for 96 h with concentrations of doxycycline ranging from 2 ng/mL to 2 μg/mL led to a dose-dependent increase in gene expression ranging between ˜337-fold and ˜26015-fold (FIG. 18). Considering this result, 200 ng/mL doxycycline was used for a time course that demonstrated that induction of NEUROD1 is detectable 12 h after treatment (˜4000-fold) and continues to increase at 24 h (˜5000-fold), 48 h (˜10000-fold) and 96 h (˜15000-fold) (FIG. 19). In addition, a clonal population of SF7996 cells (primary glioblastoma cells) was derived in which expression of TERT is controlled by the idpTV and can be induced in a dose-dependent manner with doxycycline (FIG. 20). It is noteworthy that TERT expression could only be detected in the presence of doxycycline. Accordingly, since these cells depend on TERT expression for continued expansion, their proliferation rate in tetracycline-free medium decreased over time in comparison with the same cells treated with doxycycline (FIG. 21).

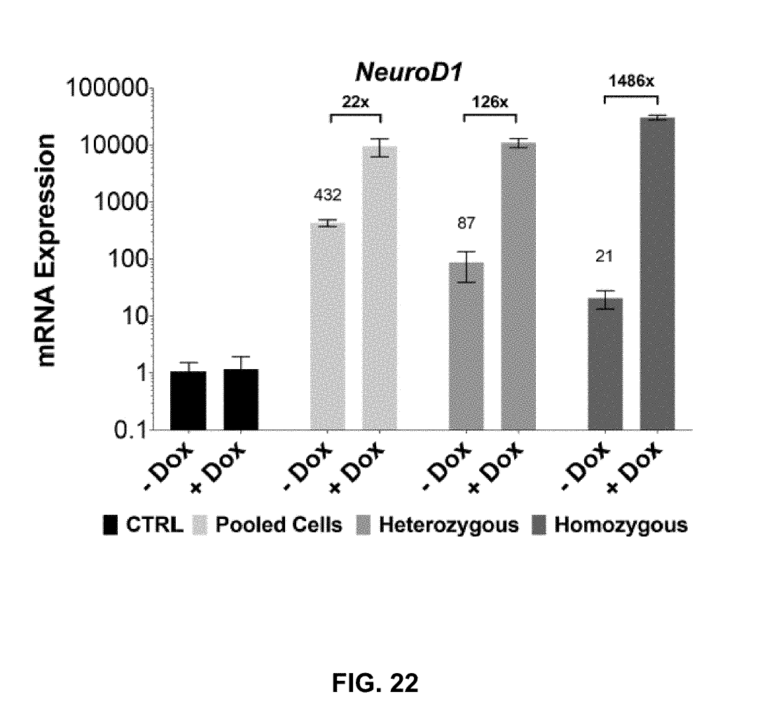

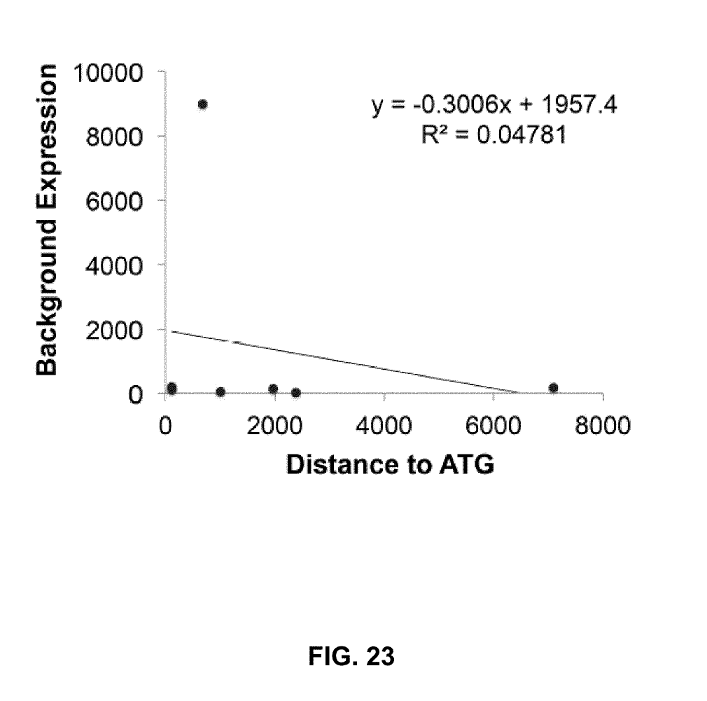

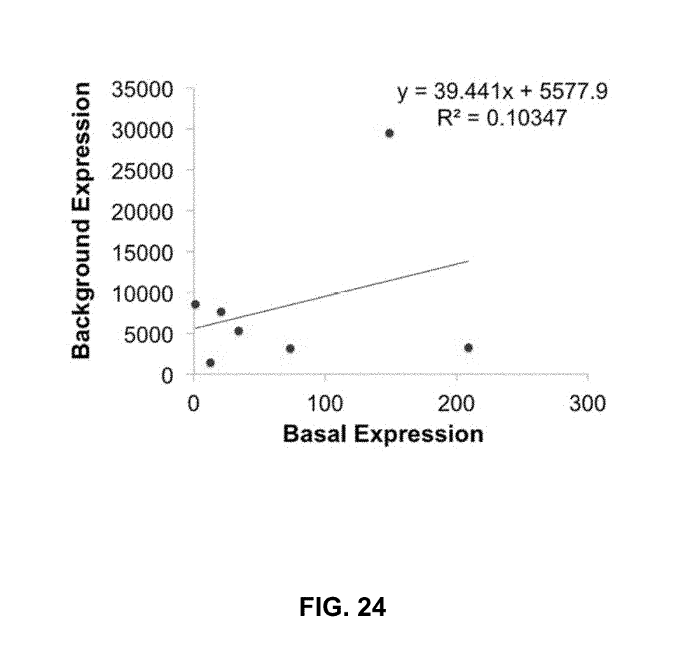

Tetracycline-inducible systems have been designed for high responsiveness to doxycycline, yet background expression in the absence of inducer, while low, continues to be a problem that hinders applications requiring precise control over gene activation. While inducibility is a significant advantage of NAVIa over CRISPRa, tetracycline-inducible promoters are typically used to modulate expression cassettes within a vector, and not in a genomic context where the surrounding transcriptional regulatory elements may contribute to undesired expression at steady state. Analysis of NEUROD1 activation within samples not induced with doxycycline revealed significant background expression (˜432-fold over basal expression, FIG. 22). While no correlation was identified between background and distance from the integration to ATG codons (FIG. 23) or between background expression and basal expression (FIG. 24), expression of rtTA from unintegrated plasmids still transiently present from the transfection might be partly responsible for high background levels of expression. Indeed, background expression in clones with heterozygous or homozygous integrations was significantly lower than in pooled populations, while gene induction in heterozygous clones was similar to that observed in pooled populations but significantly lower than activation in homozygous clones. The ratio of gene expression between samples with and without doxycycline treatment was improved from ˜22-fold induction in pooled cells to ˜426-fold and ˜1486-fold in heterozygous and homozygous clones respectively (FIG. 22).

One significant advantage of NAVIa over existing CRISPRa methods is the rapid and facile generation and screening of stable cell lines with tunable or programmable properties and a highly predictable pattern of integration. Inducible CRISPRa methods have been developed by integrating a tetracycline-inducible Cas9-based transcriptional activator at random genomic loci. Induction of target gene expression with these systems requires persistent expression of the sgRNA while expression of the ATF, and ultimately target gene activation, is controlled by treatment with doxycycline. Although these systems are tunable, they exhibit significant background expression in the absence of doxycycline. In contrast, NAVIa replaces native promoters via targeted integration of a tetracycline-inducible promoter to achieve a rapid response to the inducer while avoiding unpredictable lentiviral integration patterns. Further refinements of the minimal promoter, the positioning of TetO sites, and other attributes of the integrated vector will remove not only background expression but also basal expression, allowing generation of functional knock out or overexpression of a gene a single cell line by simply varying the concentration of inducer.

Another potential limitation of NAVIa in these experiments was the integration of two promoters in different orientations. While this approach ensures that one promoter is always positioned in the correct orientation for overexpression of the target gene, it is possible that the other promoter can modify expression in the opposite orientation. While this shortcoming also occurs with bidirectional gene activation induced by CRISPRa, it can be overcome in NAVIa by simply using a single promoter. This alternative strategy requires screening a few clones to identify those with the promoter in the correct orientation, but effectively prevents potential aberrant activation at the opposite end of the vector. Future iterations to enhance efficiency of this technique will require precise control over orientation by manipulating the DNA repair process.

### Example 7. Multiplexability of the NAVIa System

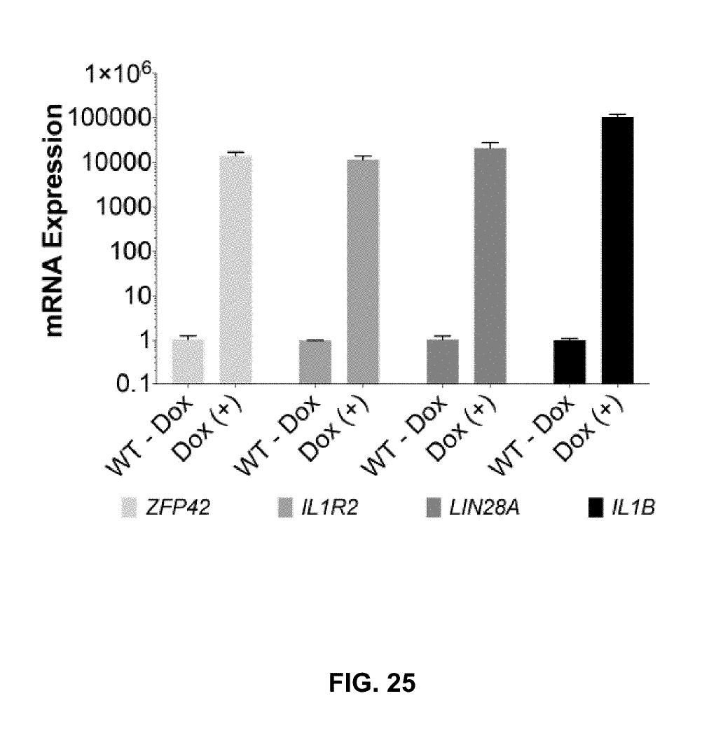

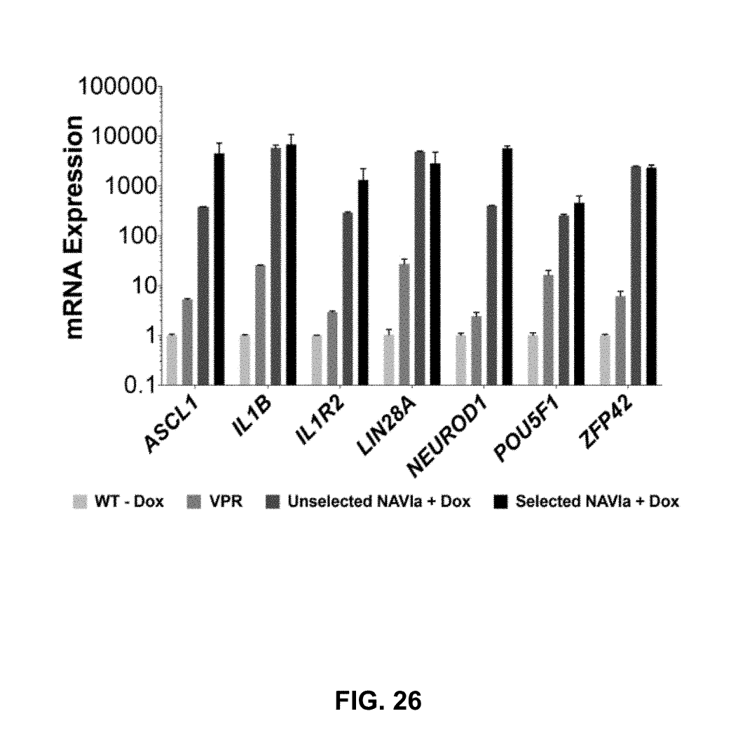

One important feature of CRISPRa architectures is multiplexability. Different genes can be activated simultaneously by delivering sgRNAs targeting different promoter. Two benefits of NAVI over other integration platforms, such as those utilizing HR, are the universal adaptability of the system to target different genomic loci, by simply providing additional primary sgRNAs, and facile clone isolation upon selection. Since activation of different genes using NAVIa can be accomplished using a set of vectors in which the only variable element is the primary sgRNA, this flexible architecture is also compatible with multiplexing. To demonstrate these capabilities, sgRNAs were first identified for targeting additional genes with NAVIa including IL1B, IL1R2, LIN28A and ZFP42 (FIG. 25). To facilitate multiplexing, a custom Golden Gate cloning plasmid was utilized to prepare two multi-sgRNA (mgRNA) vectors capable of delivering a total of 7 individual sgRNAs targeting genes and one sgRNA for linearizing the idpTV, each under independent promoter control. Co-transfection of these plasmids alongside the idpTV and Cas9 vectors into 293T cells was followed by induction of gene expression with doxycycline for two days. Analysis of mRNA expression across all targeted genes demonstrates that multiplexed gene activation with NAVIa surpasses CRISPRa for all targets tested (ranging from ˜45-fold to ˜400-fold) (FIG. 26). When selection with puromycin was applied prior to induction of gene expression with doxycycline, even higher levels of gene activation of all targets compared with unselected populations was observed (FIG. 26). Together, these results emphasize the multiplexing capabilities of NAVIa, as well as a clear advantage over CRISPRa when only one sgRNA is employed.

### Example 8. Genome-Scale Gain-of-Function Framework for the NAVIa System

CRISPRa gain-of-function genetic screenings rely on robust activation of native genes for efficient genome-scale interrogation. However, the required use of single sgRNAs, which are often insufficient for upregulating gene expression, may introduce important biases since only genes that are permissive for activation will be interrogated effectively. Previously, it was found that since shRNA and CRISPR-Cas9 knock down gene expression by different mechanisms, their application in parallel for genome-scale loss of function screenings generates results that are complementary. Unlike loss-of-function screenings, there are no alternative methods complementary of CRISPRa to perform gain-of-function screenings. However, since NAVIa requires only one sgRNA per target and achieves robust activation across targets, it was compatible with genome-scale activation screenings.

Transfection and Transduction of sgRNA Library

The human SAM library of sgRNAs, with 3× coverage of coding gene promoters, was prepared following the guidelines provided by Konermann et al., Nature, 517:583-588 (2015) and packaged into 2^{nd}-generation lentivirus within 293T cells. The resultant library was transduced into MCF7 cells.

Following a brief recovery period over a single passage, 10⁷ MCF7 cells were transfected with the NAVIa system plasmids (Cas9, TV, and secondary sgRNA) and selected by 1 μg/mL puromycin. Cells were split into two groups, which were either treated with 4-hydroxytamoxifen or not treated. The treated cells received 5 μM 4-hydroxytamoxifen for 14 days, replaced every two days. The untreated cells were handled identically receiving fresh media without 4-hydroxytamoxifen. After 14 days the cells were washed and recovered for isolation of genomic DNA.

NGS

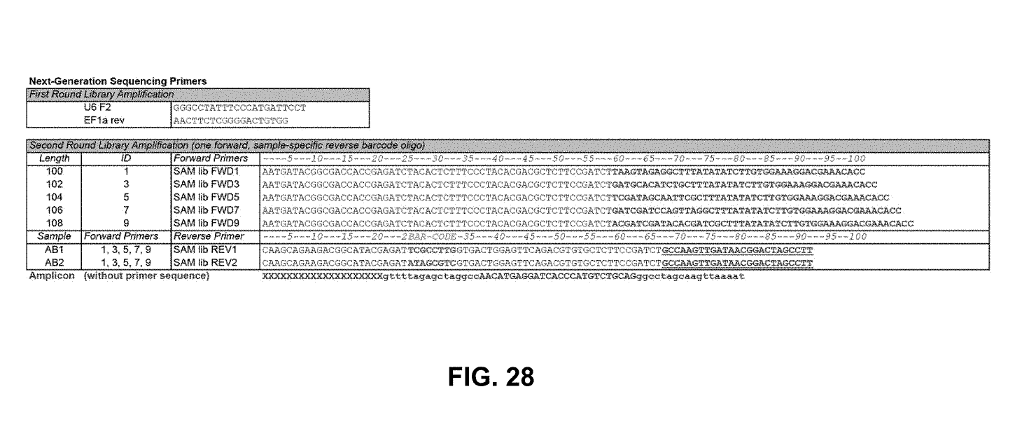

The sgRNA expression cassettes from library genomic DNA samples and controls were amplified in two rounds using KAPA HiFi HotStart polymerase (KAPA Biosystems). The first round reactions amplified the entire human U6 sgRNA expression cassette (552 bp) and were separated in 2% agarose gels, excised using the QIAquick Gel Extraction Kit (Qiagen), and used as template with the NGS primers (FIG. 28) for second round amplification. Second round products were also gel excised, cleaned, pooled, and submitted to the DNA Services laboratory at the W. M. Keck Center at the University of Illinois at Urbana-Champaign for HiSeq.

The final pool was quantitated using Qubit (Life Technologies, Grand Island, N.Y.) and the average size determined on the on an Agilent bioanalyzer HS DNA chip (Agilent Technologies, Wilmington, Del.) and diluted to 5 nM final concentration. The 5 nM dilution was further quantitated by qPCR on a BioRad CFX Connect Real-Time System (Bio-Rad Laboratories, Inc. CA).

The final denatured library pool was spiked with 10% indexed PhiX control library and loaded at a concentration of 9 pM onto one lane of a 2-lane Rapid flowcell for cluster formation on the cBOT, and then sequenced on an Illumina HiSeq 2500 with version 2 SBS sequencing reagents for a total read length of 100 nt from one end of the molecules. The PhiX control library provides a balanced genome for calculation of matrix, phasing and prephasing, which are essential for accurate basecalling.

The run generated .bcl files, which were converted into demultiplexed compressed fastq files using bcl2fastq 2.17.1.14 (Illumina, CA). A secondary pipeline decompressed the fastq files, generated plots with quality scores using FastX Tool Kit, and generated a report with the number of reads per barcoded sample library. Final fastq file data sets were first parsed using Cutadapt, to isolate sgRNA targeting sequences from leading and trailing sequence, and then analyzed using MAGeCK.

Following trimming, counting, and normalization of read counts, it was determined that the number of sgRNAs transduced into MCF7 cells was 4,292 (Table 11). Of the unique reads detected, ˜85% were found to be within the CRISPRa samples and ˜93% for NAVIa. In total, 77% of the unique reads overlapped between the CRISPRa and NAVIa libraries. In all, one or more sgRNA covering 3,817 genes were found to have been covered by these reads, with 100% overlap between the CRISPRa and NAVIa libraries, thus enabling a direct comparison between both methods.

The normalized read counts from the CRISPRa and NAVIa experiments were separately scored by gene association and assigned p-values according to the MAGeCK-RRA algorithm.

NGS Hit Validation

The top two hits from each the CRISPRa (CHSY1, GDF9) and NAVIa screen (MFSD2B, HMGCL) as well as the hit identified by both approaches (IPO9) were chosen for further tamoxifen resistance study. For each target, the primary sgRNA identified in the screen was co-transfected into MCF7 cells with Cas9, the cdpTV, and the universal secondary sgRNA followed by selection with 1 μg/mL puromycin. Ten thousand cells of each selected pool, and 10,000 wild type MCF7 cells, were seeded into 4-hydroxytamoxifen (5 μM) and tamoxifen-free media. The cells were cultured for 10 days, and were trypsinized every other day to refresh media and treat experimental cells with 4-hydroxytamoxifen in suspension. On day 10 cells were again trypsinized and counted. The cell culture and counting was done in duplicate by two independent researchers (n=4).

Statistics

Statistical analysis was performed by two-way ANOVA with alpha equal to 0.05 or with t tests in Prism 7.

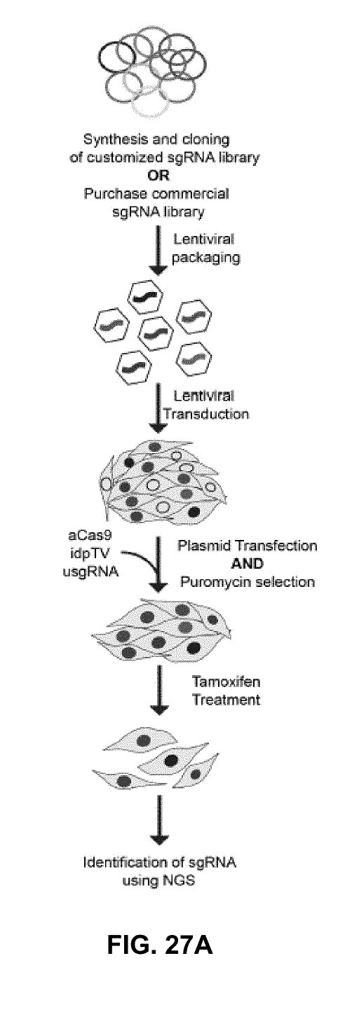

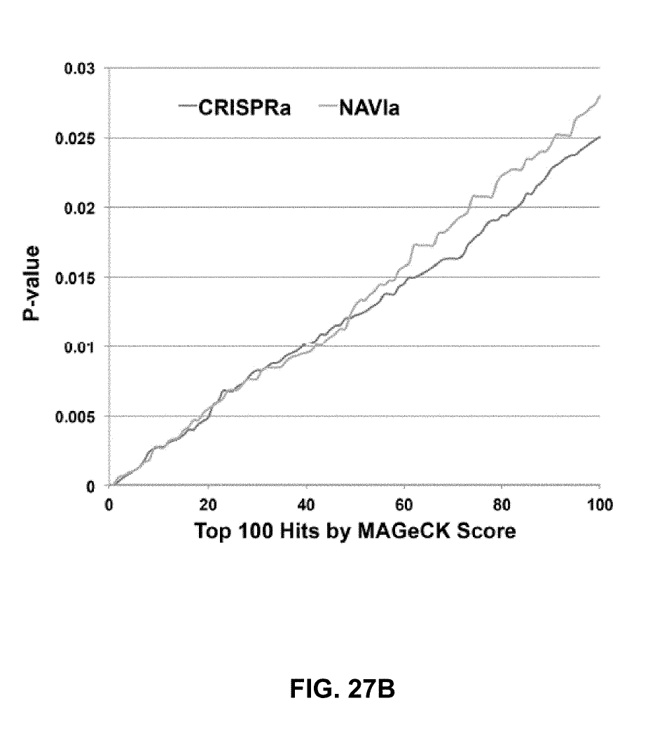

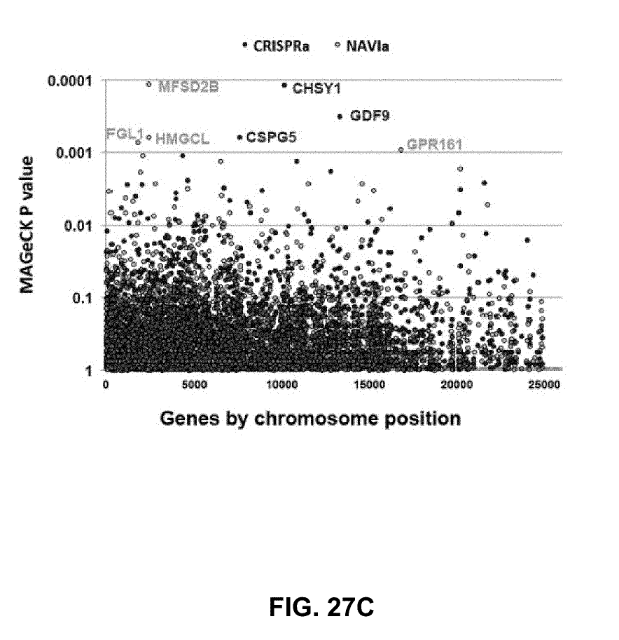

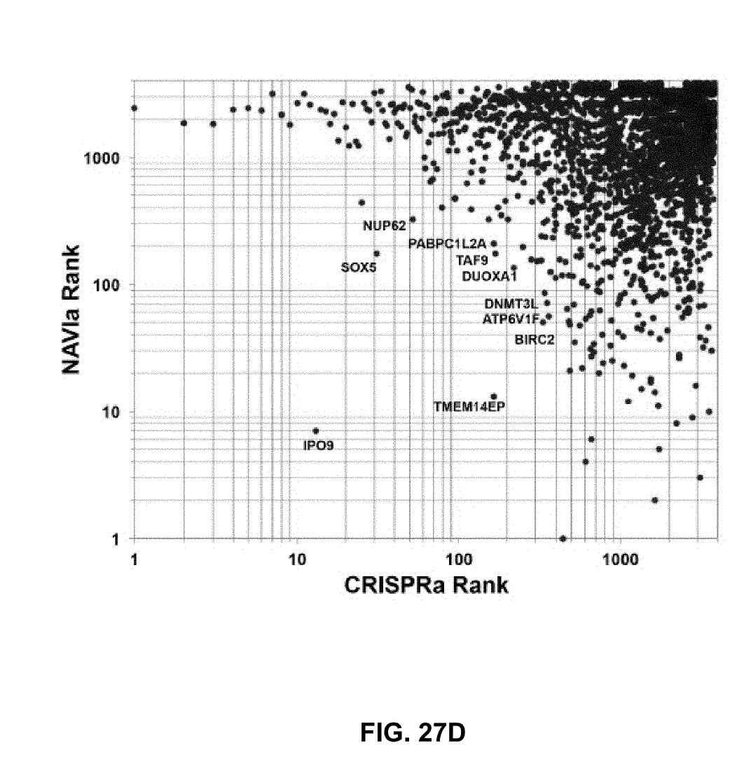

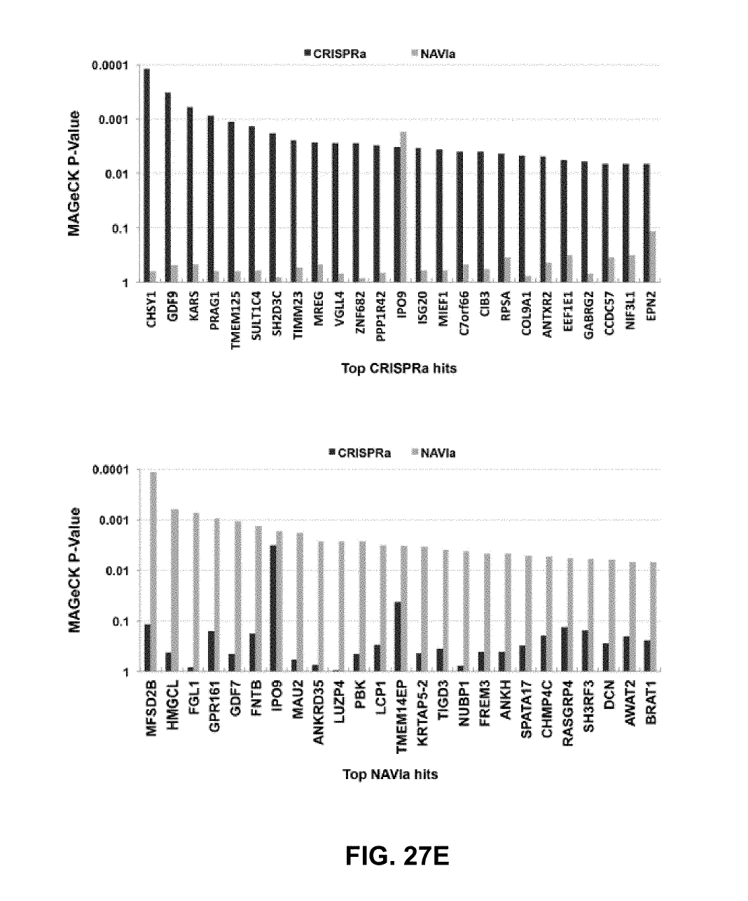

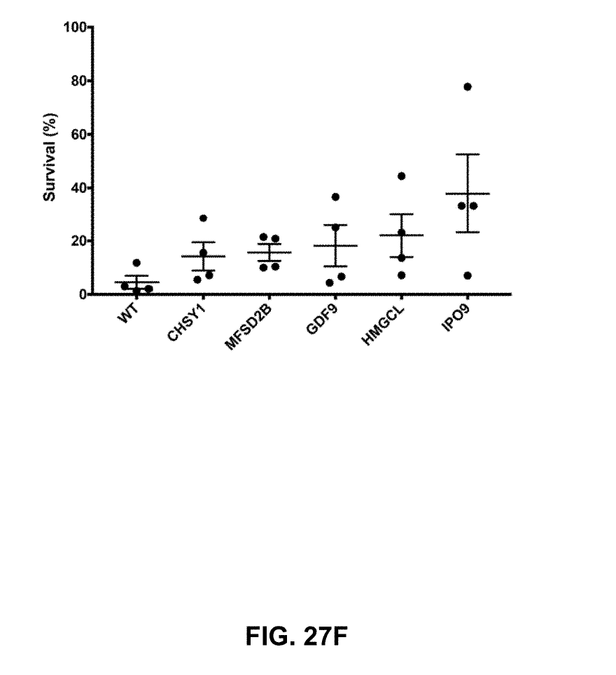

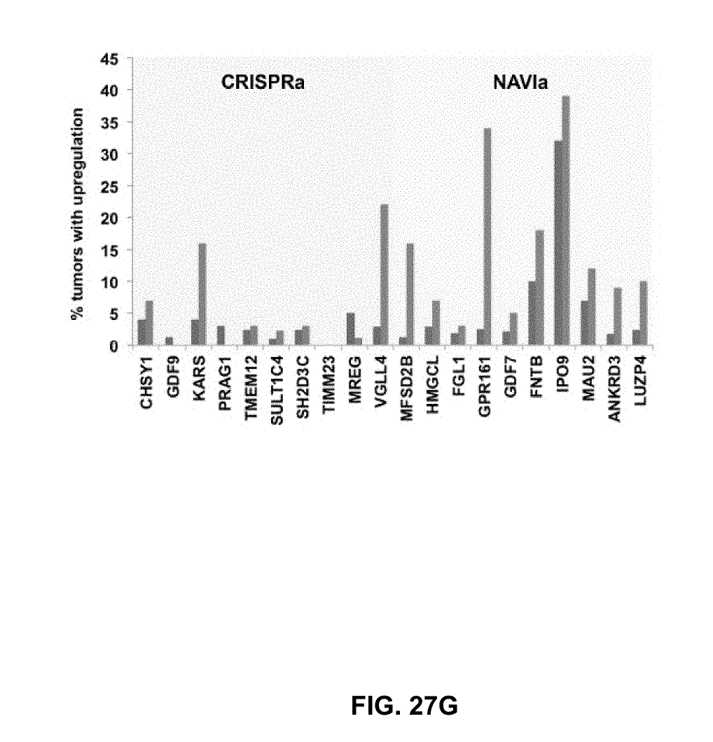

A genome-scale gain-of-function experimental framework for NAVIa was tested in which lentiviruses were first generated from a library of plasmids targeting the promoters of native transcription factors (library), which were transduced into 293T cells at MOI 0.2 (FIG. 27A). Recovery of the sgRNAs from the transduced cells followed by NGS demonstrated successful transduction of all sgRNAs (Table 11). These cells were transfected with plasmids encoding active Cas9, the cdpTV, and the universal sgRNA, and then selected with puromycin. In parallel, a CRISPRa screening was performed by transducing dCas9-VPR into the 293T cells pre-transduced with the sgRNA library.

Finally, side-by-side genome-scale screenings was performed between NAVIa and CRISPRa to evaluate their ability to identify transcription factors associated with rapid growth in 293T cells. While each method generated positive selection results, the enrichment observed with NAVIa was significantly more robust than that observed with CRISPRa. In addition, there is significant exclusivity, which highlights the differences between these approaches and suggests that NAVIa and CRISPRa could provide valuable complementary results. By combining results from each method, it is possible to identify a strong list of candidate genes with potential roles in the phenotype under investigation.

### Example 9. NAVIa Genetic Screening

To demonstrate the applicability of NAVIa genetic screenings, in comparison with CRISPRa, transcription that confer a proliferative advantage in 293T cells were identified. After 14 days of growth, next generation sequencing of the sgRNA expression cassette was performed for each of the gain-of-function screenings. Examination of FDR q-values from the top scores from each method reveals a different distribution for the top 350 hits, with a shift in significance for all hits skewed toward NAVIa (FIG. 27B). While CRISPRa yielded 3 candidate genes for which positive selection scores were highly significant (FDR q-value≤0.01), NAVIa yielded 161. Similarly, CRISPRa generated 74 hits with moderate significance (FDR q-values≤0.05), while NAVIa generated 302 (FIG. 27C). Comparison of FDR q-values from top scoring hits from either CRISPRa or NAVIa screenings demonstrates hits distributed throughout the genome (FIG. 27D). Interestingly, the results indicate little overlap for top targets between NAVIa and CRISPRa. More specifically, the screenings identified by one hit with FDR q value <0.01 that appeared in both screenings (out of 3 in the CRISPRa screening and 161 in the NAVIa screening) and 13 hits with q value <0.05 (out of 161 in the CRISPRa screening and 302 in the NAVIa screening). (FIG. 27E)

To verify the results from the tamoxifen 252 resistance screen, the top two gene hits from each screen were validated, as well as IPO9. Target-specific primary sgRNAs in combination with cdpTV, Cas9 and the secondary sgRNA were delivered to MCF7 cells, which, after selection with puromycin, were treated with tamoxifen. Each of the cell lines generated displayed increased resistance to tamoxifen compared with wild type, although not all the measurements were significant due to large variability across samples (FIG. 27F). The top hits in the NAVIa screening were validated, MFSD2B (p<0.05) and HMGCL (p<0.1), as well as IPO9 (p<0.1), which was identified by both screenings. However, the top hits in the CRISPRa screening were not statistically significant suggesting that the different mechanism of gene activation utilized by each system yields non-overlapping results. In addition to validating the top screening hits through individual gene activation, the expression profile of the top screening hits were analyzed using TCGA data sets (tcga-data.nci.nih.gov/tcga). Using cBioPortal, the available data from breast cancer samples was mined to identify those that exhibited upregulation of the top screening candidate genes. By this metric, it was found that all the top 10 hits from NAVIa and 9 out of 10 from CRISPRa screenings are overexpressed in ER+ breast cancers (FIG. 27G). Notably, expression of all NAVIa hits is higher in ER tumors (˜4.6-fold) but in only 7 of the top CRISPRa hits (˜1.8-fold).

In summary, the robust levels of activation, multiplexing capabilities, and adaptability for genome-scale gain-of-function screenings make NAVIa an attractive new platform for a variety of synthetic biology applications including metabolic engineering, drug screening, and signal transduction pathway analysis.

TABLE 11
Library of sgRNAs transduced into MCF7 cells

| | | | SEQ |
|---|---|---|---|
| | | | ID |
| Gene | Ref Seq # | sgRNA Sequence | NO: |
| AADAC | NM_001086 | ACTCAATACATGCTGTTTAT | 221 |
| AADAT | NM_001286683 | TCTCGAAGATCTCAGCATTT | 222 |
| AAGAB | NM_001271886 | ACTGAAAACCACGACCCTGT | 223 |
| AAR2 | NM_015511 | ATGGCTGGTGGCTGTGTTTC | 224 |
| AARD | NM_001025357 | TGCAGCATCCCACTTGGCAA | 225 |
| AARSD1 | NM_001261434 | GTTGTTTAACGACTGTTCTA | 226 |
| ABCA1 | NM_005502 | GGGGAAGGGGACGCAGACCG | 227 |
| ABCA12 | NM_015657 | CATCTGCATATGCAGGTCCT | 228 |
| ABCA3 | NM_001089 | ACATGCAGGGGGCACCGCGC | 229 |
| ABCA5 | NM_172232 | ACGCTCGGCCCCGCGCGTCC | 230 |
| ABCA6 | NM_080284 | ATTTTATTCCCAACCAACCA | 231 |
| ABCB9 | NM_001243014 | GTTTGCCACAGGTGAGCAGG | 232 |
| ABCC10 | NM_001198934 | GAGCGAATACTCCACGTGAG | 233 |
| ABCC4 | NM_005845 | GCCGGGACCGACGGGTGACG | 234 |
| ABCE1 | NM_002940 | TCAACTTCCTCTCAACTGTG | 235 |
| ABCG1 | NM_207627 | TCTGTTCCCTCACAAGTCAC | 236 |
| ABCG1 | NM_207629 | AACTATATCACTACCTCAAC | 237 |
| ABCG2 | NM_001257386 | GAAGAGGATCCCACGCTGAC | 238 |
| ABHD1 | NM_032604 | TGGGGGAGGCCGCTTGTCTC | 239 |
| ABHD14B | NM_032750 | TATCTGGCATTTACACAACG | 240 |
| ABHD17A | NM_031213 | AAACTTAGGTTTCATTCACT | 241 |
| ABI3 | NM_016428 | CAGGCTTGCTAACACCCCTC | 242 |
| ABL1 | NM_005157 | CCCGCGCCCGCCCATGGCCG | 243 |
| ABL2 | NM_001168239 | ATTGCTGGAAATTTTCCTTT | 244 |
| ABL2 | NM_001168239 | CGCAAAAGACTGAGTCAGAA | 245 |
| ABRA | NM_139166 | TGACAGCTCCAGTTTCATCA | 246 |
| ACACA | NM_198836 | TGAACGGCCTGGAGTAACCC | 247 |
| ACAT1 | NM_000019 | GCAAGAAGCCAACCGCAGCG | 248 |
| ACAT1 | NM_000019 | ACGAGCACCTGACACGCTGC | 249 |
| ACBD5 | NM_001042473 | CAATCTCAAGACACTTAAGC | 250 |
| ACBD6 | NM_032360 | CGGATCTGTTGCGTGCGCGT | 251 |
| ACIN1 | NM_001164817 | CTACAGAGGCTTAACCCCCC | 252 |
| ACIN1 | NM_001164817 | GGCCACAGGGAGCCGACTGC | 253 |
| ACKR2 | NM_001296 | CTCTGTCTCATTATATGCTT | 254 |
| ACKR4 | NM_178445 | AGAGAAGACAAGAATGAAGC | 255 |
| ACOT12 | NM_130767 | TCCCCCACTCGCGATAGTCC | 256 |
| ACOT6 | NM_001037162 | ACAGTCTCACTCTGTCGCCC | 257 |
| ACOT6 | NM_001037162 | TTCAATACCTTTTGGTGTAC | 258 |
| ACP2 | NM_001610 | AGACCTCATCTTGATTAAGA | 259 |
| ACP5 | NM_001611 | GCACACGTGTGCAGCAGCCT | 260 |
| ACRBP | NM_032489 | CCAGAGCCCATCCAGATGGT | 261 |
| ACSL1 | NM_001286708 | GTTCTATGAATATATCCTCA | 262 |
| ACSL1 | NM_001286711 | TATGAAATCCGAGGCAGTCT | 263 |
| ACSL1 | NM_001286712 | GCTTAAGCAAATCTAACTTT | 264 |
| ACSL4 | NM_022977 | GAGGAAGGCGAGGCGGCTAA | 265 |
| ACSL5 | NM_203379 | GTTACTACAAGTGTTTGAAC | 266 |
| ACSL6 | NM_001205251 | GGGTCGCGGTTACCTGTCCT | 267 |
| ACSM4 | NM_001080454 | GAGACTGGGAGGTGGATTTG | 268 |
| ACSM4 | NM_001080454 | GGAAGGATGAGGTGTTTTTC | 269 |
| ACTL10 | NM_001024675 | CCTACCTTATGACAACTCCC | 270 |
| ACTL6B | NM_016188 | CTAAGGAACTGGCGGCAGAG | 271 |
| ACTL8 | NM_030812 | TGCTGATATTTCATTGTTGC | 272 |
| ACTN4 | NM_004924 | CAAGGCCGCGCTCCGGAGCT | 273 |
| ACTR3 | NM_001277140 | CTAGGACTGACAGCCGGCGG | 274 |
| ACTR6 | NM_022496 | GGGGGCGTTCTACAAATTCC | 275 |
| ACVR2A | NM_001616 | GTTGTTGGCTTTTCGTTGTT | 276 |
| ACVRL1 | NM_001077401 | TGTTTAAGTGACTGAGAGCT | 277 |
| ACY1 | NM_001198895 | ACGGGACCGTCCTGAGCTCC | 278 |
| ACYP1 | NM_001107 | GATTTCAGGACGCGGTTGTC | 279 |
| ADAM2 | NM_001278114 | TTGCAGGACAAGCACTCCAC | 280 |
| ADAMTS14 | NM_139155 | GCCCCGGGCTGTCGGAGCAC | 281 |
| ADAMTSL3 | NM_207517 | ACGGCGTCTCTTCGCGCCCC | 282 |
| ADAMTSL3 | NM_207517 | GGCAAGTGCACGGCGCGCCC | 283 |
| ADAR | NM_015841 | GAGTCTCGCTCTTTTTGCCC | 284 |
| ADAT1 | NM_012091 | AGATACGTCATTCTAGTTGA | 285 |
| ADAT2 | NM_001286259 | TGGCAATTTAGGTGGAATGG | 286 |
| ADCY1 | NM_021116 | GGCTGCCCCGCGCGCGCGCC | 287 |
| ADGB | NM_024694 | ACTGAAATCCCACATCCCCG | 288 |
| ADGRB1 | NM_001702 | AGCTTAGCCTGCTACCAACG | 289 |
| ADGRE2 | NM_013447 | TCAACAGAGAATCATGTGAT | 290 |
| ADGRF1 | NM_153840 | ATTCTCCCAGCAGACATAAA | 291 |
| ADGRF3 | NM_001145168 | GCCTGTGACTCTGAGTGAAA | 292 |
| ADGRF3 | NM_153835 | AGAGGAATTTGTGAAGCGCT | 293 |
| ADGRG1 | NM_001145770 | AGGGGAGTCCTTGGGTTCTC | 294 |
| ADGRG1 | NM_001145771 | GGAGCACTGAGAGGGGAGAC | 295 |
| ADGRG1 | NM_001290142 | TCAGGTGTCCTGCAGGAGCC | 296 |
| ADGRG5 | NM_153837 | AGCAGAGAGAAGTGCAGTGG | 297 |
| ADGRG7 | NM_032787 | TGGTTGCCAGTAGTCACCTA | 298 |
| ADGRL1 | NM_014921 | GATCGGGTCTGCGCCCCTCC | 299 |
| ADH1B | NM_000668 | TTTATCTGTTTTGACAGTCT | 300 |
| ADIG | NM_001018082 | AGCATGCAGGGGACACTTTG | 301 |
| ADIG | NM_001018082 | GGCTGAGAATTAAAAAGCCC | 302 |
| ADIPOR2 | NM_024551 | CGCACGGCGTGTGGTCTTAT | 303 |
| ADNP | NM_001282531 | TGTGGGAGAGGCGGCTTCAC | 304 |
| ADORA1 | NM_000674 | AAAAAATGTGAGCTTTTCGA | 305 |
| ADORA2A | NM_001278500 | TCACTGCAACCTCCACCTCC | 306 |
| ADPRHL1 | NM_199162 | GACTGGGGCTGCCTCCTTCC | 307 |
| ADRA2C | NM_000683 | CTGGGCGCCGCGGTCCCCGG | 308 |
| ADRB3 | NM_000025 | ACGTTTCCTTTAGCTAAATC | 309 |
| ADSS | NM_001126 | AATCCCAGCATGCAACGCTC | 310 |
| AFAP1 | NM_001134647 | TACCCAGCTCAACGTCTACC | 311 |
| AFF2 | NM_001170628 | GTTTGATAGTTTGAGTATTC | 312 |
| AFF3 | NM_001025108 | TAGAACCGGAAGCCCCTCCA | 313 |
| AFM | NM_001133 | GAGTTGGAACAAAAGTCCAC | 314 |
| AFM | NM_001133 | TATTGTGCATACTTAGCCTG | 315 |
| AGAP6 | NM_001077665 | GCATCATAAGCCACAGGGTG | 316 |
| AGBL3 | NM_178563 | AGAGAGGCTTTGGGGTCTGT | 317 |
| AGFG1 | NM_001135187 | GAGGCCGCAGTGACTCCTCC | 318 |
| AGMO | NM_001004320 | ATACAGTGCAGTTTGACTGT | 319 |
| AGT | NM_000029 | GGAAGTTTCCAGTGTAGCTG | 320 |
| AHNAK | NM_024060 | CAGGTCCGGGACAGGACAGG | 321 |
| AIMP1 | NM_001142415 | GTCTCAAATAGATAGAAACC | 322 |
| AIMP1 | NM_001142416 | TCTCGCTATATGTCCTTTCG | 323 |
| AIPL1 | NM_001285402 | GACGGTGGGGGCGGTGACCT | 324 |
| AK3 | NM_001199855 | AGGTAGGCCCTCTCGGCTCA | 325 |
| AK4 | NM_203464 | TGCAGTAGACCGCGGTCCCC | 326 |
| AK8 | NM_152572 | AGGGTGGGGAGGCCCGTTCC | 327 |
| AKAP2 | NM_001136562 | AGGCCGGGCCTGCTCTGGCT | 328 |
| AKAP4 | NM_003886 | CAACTAGATCAGCCTTTCTC | 329 |
| AKAP8 | NM_005858 | CCGTGGCCTAATGGGAGTTG | 330 |
| AKAP8L | NM_014371 | GGGGGCGGAGCTGTGCACTA | 331 |
| AKR7A3 | NM_012067 | AAATGGCTGTGGCTTCGTAC | 332 |
| AKT1 | NM_001014432 | TCGGGAGCTGCCCCTCAGCC | 333 |
| AKT1S1 | NM_001278159 | ACGGCCCAGGTAGAGATCCC | 334 |
| AKT2 | NM_001243028 | CTGCGCACATTAGACAACTT | 335 |
| AKT3 | NM_005465 | AAGTCTGGCTCTTCAAACTG | 336 |
| AKTIP | NM_022476 | GTGTGAGAGCCAGTTGGCGC | 337 |
| ALDH3A1 | NM_001135168 | CGTGGTTTACACACCAAGCC | 338 |
| ALDH3A1 | NM_000691 | ATCAGCAGCCCCCACGCCCA | 339 |
| ALDH5A1 | NM_001080 | GCGGTGCAGCGAGAAAGACG | 340 |
| ALG11 | NM_001004127 | TTACTGGTAGCCGCTTCCCA | 341 |
| ALG12 | NM_024105 | CAATCCGAGTTCGCCACGAG | 342 |
| ALG14 | NM_144988 | AGGTAAAATGGATTGTGACT | 343 |
| ALKBH4 | NM_017621 | CCGCGGTAACTGAGCCCAGG | 344 |
| ALKBH4 | NM_017621 | GCAGCCCGCGCTGACCCAGT | 345 |
| ALMS1 | NM_015120 | CCCCGGAAGGCGCCCAGTCC | 346 |
| ALMS1 | NM_015120 | CTGTAAGCTCACAATAAACC | 347 |
| ALOX5AP | NM_001629 | CAAGCCCTGCTTCTCCTGGT | 348 |
| ALPK1 | NM_025144 | TCCTAAAGGGGTGTGTCTTA | 349 |
| ALPK3 | NM_020778 | CAGGAGAATGGCATGAACCC | 350 |
| ALS2CR12 | NM_001127391 | TCCACTTTCGTCATCAGTCA | 351 |
| AMBRA1 | NM_017749 | ACTAAAATAGTGGGAGAATG | 352 |
| AMD1 | NM_001634 | TGACAGGCGGCAGCAGCTAT | 353 |
| AMER2 | NM_152704 | GAATCTCAGACCCACTCCAC | 354 |
| AMOTL1 | NM_130847 | GGCGGCGGGTGTCTGCAGAC | 355 |
| AMOTL2 | NM_001278683 | GTGTCTGCCCTGTCCATCTA | 356 |
| AMPD2 | NM_004037 | GACAGAGACCCTAGCCTCTT | 357 |
| AMPD2 | NM_004037 | TCCTCTGTCTCTGCACACTC | 358 |
| AMPD3 | NM_001172430 | TATTGCAGTTCCAAACCCTC | 359 |
| AMTN | NM_212557 | TCATTTCCCAACACTTCATT | 360 |
| AMY1A | NM_001008221 | CTACTGGGTTTAGGCCAACC | 361 |
| AMY1A | NM_001008221 | CTGGAATCTATGAATAACAT | 362 |
| AMY1B | NM_001008218 | ACTTGTTGCTGATTTTGGCC | 363 |
| ANGEL1 | NM_015305 | GCAGAAGTGGGAATAAACTG | 364 |
| ANGPT4 | NM_015985 | ACTGAGGAAGGAGGAAGGGA | 365 |
| ANK1 | NM_020475 | TCTTGTAATCTGCGGTCCCC | 366 |
| ANKFY1 | NM_001257999 | AGAAGTGCGCGGCTCAACCG | 367 |
| ANKH | NM_054027 | AGGCGACGGCACAGGAAAGG | 368 |
| ANKRD13A | NM_033121 | CTTGGCCAAAGATCTCCACG | 369 |
| ANKRD16 | NM_019046 | GAAAGTTTCCCGCTCCGCCC | 370 |
| ANKRD17 | NM_001286771 | ATTTAACACGTCTGGCTTCC | 371 |
| ANKRD23 | NM_144994 | GCCCCTGGGCCAGATGACTC | 372 |
| ANKRD26 | NM_014915 | GGCCCAGACCTCGCAAATCT | 373 |
| ANKRD27 | NM_032139 | CGTGCCCAGAACGTGAGGGG | 374 |
| ANKRD35 | NM_001280799 | GATTTGAAGGGCGAGGTTCG | 375 |
| ANKRD46 | NM_001270378 | GCTGCAGCGCGAGACCGCTC | 376 |
| ANKRD50 | NM_020337 | GCCCAGGCACGGGATGCTGC | 377 |
| ANKRD52 | NM_173595 | CTCCCCGCGCAAACGGACCC | 378 |
| ANKRD54 | NM_138797 | ATGTCTGTCAGTCACGTTGC | 379 |
| ANKRD55 | NM_024669 | TTGGAGAACGGAGCTGAAAG | 380 |
| ANKRD62 | NM_001277333 | GCTGAGGTGCGCATGTGCCC | 381 |
| ANKS1A | NM_015245 | AGTCCACCTGCGCTGGTCCG | 382 |
| ANKS1B | NM_001204065 | ATTGTTCCGCGGCTGCTGCC | 383 |
| ANKS1B | NM_181670 | AAAAAATCTGCCTTATCTGA | 384 |
| ANO3 | NM_031418 | TCAACGCCCACCCCTCACTG | 385 |
| ANO6 | NM_001142679 | TGTGTGTCCACAGACGACCT | 386 |
| ANP32A | NM_006305 | AATCTAAAGGGGTCCGTCTC | 387 |
| ANP32E | NM_001136478 | TTAATTTTGATAGGTCCAGG | 388 |
| ANP32E | NM_001136479 | GCCTTCGCCCTGGGTAGGTG | 389 |
| ANTXR2 | NM_001145794 | CCCATGGAATCCTTAGTCTT | 390 |
| ANTXRL | NM_001278688 | GAACAAACAGCAGGGTCTAG | 391 |
| ANXA10 | NM_007193 | TTGAAAAAGCTGATGACTTA | 392 |
| ANXA13 | NM_004306 | CAGATAAACTTAGACTGCCC | 393 |
| ANXA3 | NM_005139 | TTAGACTGTCCCTATACCTA | 394 |
| ANXA6 | NM_001155 | TCAGTCTCAGATCCGGGGGC | 395 |
| ANXA8 | NM_001271703 | TGAGTGGGGCTTTCGCAGGC | 396 |
| AOC2 | NM_009590 | GCATGTGGAAGCAGTGCCCT | 397 |
| AOC2 | NM_009590 | TGTTCCAATTTTCTGTCCTG | 398 |
| AP1G2 | NM_001282474 | TCATCTCCTTTGGGGTGCGA | 399 |
| AP1G2 | NM_001282475 | AAAAAGCAATGGCTGAGCTA | 400 |
| AP1S2 | NM_003916 | CCTCCTATCATTAAACAAGC | 401 |
| AP2B1 | NM_001282 | ACATCCTCTGAGGCCCAGAT | 402 |
| AP2B1 | NM_001282 | GGCTAGCTTGCCGGGACCAA | 403 |
| AP2M1 | NM_004068 | CTTGCAATTTGAAGCGCTCT | 404 |
| AP3M1 | NM_207012 | GGCACAGAATGGGCGGAGTC | 405 |
| AP4E1 | NM_007347 | GTAGACCTCCTTTCTCGCGA | 406 |
| AP4S1 | NM_001254729 | TCATAATGTGAACCTTTGAT | 407 |
| APBA2 | NM_005503 | TCAGCTGCTCTGGAGAGCCT | 408 |
| APBB3 | NM_133172 | AGGCACTTCCGGAGCATTTT | 409 |
| APCDD1 | NM_153000 | GGAGACTTGAAAGGGCGCGT | 410 |
| APEH | NM_001640 | CAATGAGTCTTTGAGGATGA | 411 |
| APEX1 | NM_001641 | CACACAATGTGCTGTGCATC | 412 |
| APITD1- | NM_001243768 | ATTCTCTTACCAACAGGTAC | 413 |
| CORT | | | |
| APITD1- | NM_198544 | CCTGTTCCACTCGCTGAATG | 414 |
| CORT | | | |
| APLF | NM_138964 | TGTCTTTCAAAGGTTTAGAA | 415 |
| APOA1 | NM_000039 | CAGTGAGCAGCAACAGGGCC | 416 |
| APOBEC3D | NM_152426 | GAGCGGCCTGTCTTTATCAG | 417 |
| APOBEC3G | NM_021822 | CCAGGCGTCTGCCTCCCCCC | 418 |
| APOBEC3G | NM_021822 | CTGGGATGATCCCCGAGGGC | 419 |
| APOC4- | NM_000483 | GGAACCTTCTCTCAAGTGAC | 420 |
| APOC2 | | | |
| APOD | NM_001647 | TCATTTCCTGAAGTGGAACA | 421 |
| APOM | NM_001256169 | CCGTGGGAAGGCAGTAGACG | 422 |
| APP | NM_000484 | CCCACAGGTGCACGCGCCCT | 423 |
| APP | NM_001136016 | GGCTGTGGAGAAGGAACTGC | 424 |
| APRT | NM_000485 | TCTTAAAATCGATGGCGCCT | 425 |
| AQP6 | NM_001652 | TCAGATCCCCGGCCTGCTTC | 426 |
| AQR | NM_014691 | TCTCTCTGCCGCCCGCTAGA | 427 |
| AR | NM_001011645 | GGCAGTAATTGGCATCAGGA | 428 |
| ARAF | NM_001256196 | AAGCAGAACACAGGTCATTT | 429 |
| ARAF | NM_001256196 | ATACGTCTATGCCACTGTTG | 430 |
| AREG | NM_001657 | CTAGCTGCAAGCCGTTTTTG | 431 |
| ARFGAP3 | NM_001142293 | TGCTTCCATGGAAAGGTCAG | 432 |
| ARGFX | NM_001012659 | CTACCTTTGACAACCCTTCA | 433 |
| ARGLU1 | NM_018011 | GGAGACTCTCCTTTTCGCCT | 434 |
| ARHGAP18 | NM_033515 | GATCAGACTAACTTGGGGGT | 435 |
| ARHGAP20 | NM_001258416 | ACTTTGCGGGGCTGGTTGAC | 436 |
| ARHGAP31 | NM_020754 | GGAGTCGCAGAACTGCTCTC | 437 |
| ARHGAP45 | NM_001282334 | AGACTACTGCCAACAATCAC | 438 |
| ARHGAP6 | NM_006125 | GTTCTGCTTTCTCCTGCTCC | 439 |
| ARHGEF2 | NM_004723 | TGGCGCCCAGAAAGCAGGCG | 440 |
| ARHGEF25 | NM_182947 | AAGCGCTGGGGACGTGGAGT | 441 |
| ARHGEF4 | NM_015320 | CTGCGGGACAAACTCGGGCC | 442 |
| ARHGEF6 | NM_004840 | GGGAGATGTGCTGGCACAAC | 443 |
| ARID4A | NM_002892 | TTTCCGAAAACCAACTTTAT | 444 |
| ARID5B | NM_001244638 | CACGTTCCATGAATTTGACA | 445 |
| ARL14EP | NM_152316 | ATGATTCAAGGCGAGGCAAG | 446 |
| ARL17A | NM_016632 | AATCACAGTTAAACGAATTC | 447 |
| ARL4D | NM_001661 | GCTGCAGCCCCCACCATACG | 448 |
| ARMC9 | NM_001291656 | ACGAAAGTGGAGTGGTGGAG | 449 |
| ARMCX3 | NM_016607 | GGAAGGGAAACACAACTACA | 450 |
| ARMCX4 | NM_001256155 | TTTTCCCTGTACCAGAATTA | 451 |
| ARPC1A | NM_006409 | TACTGTCGGCGGCCCTTCAG | 452 |
| ARPC4 | NM_001198780 | CTTCCGGAAGTTTTCCACCT | 453 |
| ARPIN | NM_182616 | TTTTGTGCGTGTGCTGGGGC | 454 |
| ARRDC3 | NM_020801 | GAGCTAGGGGAAGGAGATAC | 455 |
| ARSB | NM_198709 | TTCAATAAGCACGTGACTAA | 456 |
| ARSB | NM_000046 | CTGTTTGACTCATTATGTCA | 457 |
| ARSF | NM_004042 | TGCTGTTGTTTTTCTTTTCC | 458 |
| ARSG | NM_001267727 | GGCGGCAGCACGCACGGCCC | 459 |
| ARSG | NM_014960 | GGGCCGCGTTGCTCCCTCTT | 460 |
| ARSK | NM_198150 | AGCCTCGGCGTTTGTAGAAG | 461 |
| ART1 | NM_004314 | TTCCTCCCTTAGAAGAACAC | 462 |
| ART5 | NM_001079536 | GGGAGGAAACTTGTGAGACT | 463 |
| ASAH2 | NM_001143974 | GAGCTAAGATATCTTAACCT | 464 |
| ASAP2 | NM_001135191 | GGGAAGCGGATCCCGCAGGA | 465 |
| ASB11 | NM_001201583 | AGGTTCTAATCTAACTGATT | 466 |
| ASB11 | NM_001201583 | TAGTTTATTTAACACTGCTG | 467 |
| ASB14 | NM_001142733 | ACATGTGGTTTAGCTCTTTT | 468 |
| ASB15 | NM_080928 | GGGTTTTACCCCACAGTCAC | 469 |
| ASB3 | NM_145863 | GGCGGGACTATAAAGCGCCC | 470 |
| ASCC1 | NM_001198798 | ACTAGAAAAATGGAGAAGGT | 471 |
| ASCL2 | NM_005170 | ACCCGTTTGGCCAATCGCGC | 472 |
| ASCL4 | NM_203436 | CTAATCTCACCCAGGATATA | 473 |
| ASF1B | NM_018154 | CTCCCTCTCCGCAGCGTGTG | 474 |
| ASH2L | NM_004674 | AGGAAGCTAGATGGTTAGTG | 475 |
| ASIC1 | NM_020039 | CCCCTCCTCGCGGCCGCTTT | 476 |
| ASMT | NM_001171038 | AGCACTCATTAATCGTCTTA | 477 |
| ASMT | NM_004043 | CACGGCCAGGCGCCCTCTCC | 478 |
| ASMTL | NM_001173474 | GGTCTCAGGGGAGATCAATG | 479 |
| ASNA1 | NM_004317 | TTCCTCATTACTTGCCTTTT | 480 |
| ASNSD1 | NM_019048 | GTTGAGATGCAGAAACGCTC | 481 |
| ASPH | NM_001164751 | TGGAGTTAGCTAGGACCAAC | 482 |
| ASPH | NM_001164756 | TCCAGTTTGTCTCGGTCCTT | 483 |
| ASPM | NM_001206846 | CGGCCGCCAATCGCTATCTG | 484 |
| ASTN2 | NM_198187 | TGAGCCACGGCCCACGACTC | 485 |
| ATAD2 | NM_014109 | GGACCTGAGCGGAGAGTCCT | 486 |
| ATAD2 | NM_014109 | TCCTCCCATTTGTAGAGCGA | 487 |
| ATAD3B | NM_031921 | CTATGGCGTCACTGCCCTCG | 488 |
| ATAD5 | NM_024857 | ATTCAAATTTCCAAACTCCC | 489 |
| ATCAY | NM_033064 | ATCTCCGAAAGCCACGCCAG | 490 |
| ATF2 | NM_001256093 | GACGGAATCACCTGACTCGG | 491 |
| ATF5 | NM_001193646 | AGCCTTTCCTTCCCACTCCT | 492 |
| ATF5 | NM_001290746 | CCCACCCCTCAACTAACGGT | 493 |
| ATF5 | NM_012068 | TTGAGTCTCATAAACCCACC | 494 |
| ATF6B | NM_004381 | CTTGGCGGTATGGCACTGTC | 495 |
| ATG16L1 | NM_017974 | AGTAAGCAGTCAGGCGGAAA | 496 |
| ATG16L2 | NM_033388 | ATCCCCGGCTTGTCCCAAGA | 497 |
| ATG5 | NM_001286106 | GACGCCCAGATTCCGCGCTC | 498 |
| ATG9A | NM_024085 | CACAACAATCCCCGTCACTA | 499 |
| ATP1A1 | NM_001160234 | ATTTCCAGAGACTTTCATTT | 500 |
| ATP1A4 | NM_001001734 | AGAGTCAGCTTTGAATCACA | 501 |
| ATP2C1 | NM_001199184 | CGCAGGCGCATTCGTGTTCA | 502 |
| ATP2C1 | NM_001001485 | GTGGCCCGCCTTGTTCTTGC | 503 |
| ATP5G3 | NM_001002258 | GTGGTTGTCGTTGTCCTTCC | 504 |
| ATP5G3 | NM_001689 | TCTGTTTAGTCCTCTCTGCC | 505 |
| ATP5S | NM_015684 | GGCTAAAGAGCGCGGGTCCT | 506 |
| ATP5SL | NM_001167867 | GTGGCTAGTGGGGGCCAGGA | 507 |
| ATP5SL | NM_001167867 | TCTGTGAGGGTCGCAGGCGG | 508 |
| ATP5SL | NM_001167871 | CCTGTGAACCCAGCACTTTG | 509 |
| ATP6AP2 | NM_005765 | GTAGGCAGCGATTGAAAAGT | 510 |
| ATP6V1E1 | NM_001039366 | GGTAGGAGGAAGAAAAGATA | 511 |
| ATP6V1E1 | NM_001039366 | TTCCTCTATCTGAAATTAGT | 512 |
| ATP6V1F | NM_001198909 | GTAAAGACAGGCCCGAACCA | 513 |
| ATP6V1G2 | NM_138282 | AGCATAAAGGGTTGTGAATG | 514 |
| ATP9B | NM_198531 | GTAACGAGCGGCGGCGCGGA | 515 |
| ATPAF2 | NM_145691 | GTAGTCTCCTCGCCGAGGCG | 516 |
| ATXN10 | NM_013236 | AACACAGGTCCCCCTCCCCC | 517 |
| ATXN1L | NM_001137675 | CCTCCCTTCCCGGGGAGTCC | 518 |
| ATXN7L1 | NM_152749 | CTGCTGCCCCTGGCGGCCGC | 519 |
| AURKA | NM_003600 | GCTGTTGCTTCACCGATAAA | 520 |
| AURKB | NM_001284526 | TCACGCTTGGCTTCCAGTTT | 521 |
| AVIL | NM_006576 | TGGTAATCCCCAGGCCAGCC | 522 |
| AVL9 | NM_015060 | CAGGGCTGGGCAAGGCCGGG | 523 |
| AVP | NM_000490 | ACTGCTGACGGCTGGGGACC | 524 |
| AWAT2 | NM_001002254 | AGTGGGCAGCTGGAAGGAAC | 525 |
| AWAT2 | NM_001002254 | TCTGTGGAGGGGTGGTACAG | 526 |
| B3GALNT1 | NM_003781 | GCCAAAATTAGACAACTTAG | 527 |
| B3GALNT1 | NM_003781 | GTCACCTTGCATTCCGAGCA | 528 |
| B3GALT1 | NM_020981 | TTAGGGTTTCAGCTGGTACT | 529 |
| B3GAT3 | NM_012200 | CGAGATTCTGCACCTACCCG | 530 |
| B4GALNT2 | NM_001159387 | CAGCGGAGGAGAAAAGTCCA | 531 |
| B4GALNT2 | NM_001159387 | GGAGAGAGAAGCCCGATCAC | 532 |
| B4GALNT2 | NM_001159388 | GTGTGGCTGAATCCTTCTAA | 533 |
| BAD | NM_004322 | CTCACACCTTGGGCGTGTGT | 534 |
| BAG1 | NM_001172415 | GCAAAAGGACTTGGTGCTCT | 535 |
| BAG6 | NM_080702 | ACCGTCCATAGCCCCTCTCG | 536 |
| BAIAP2 | NM_017451 | GGGCGGTGATGCGGGCGCAA | 537 |
| BAIAP2L1 | NM_018842 | TGCCCTGTCCGCCACAGGTG | 538 |
| BAK1 | NM_001188 | TCAGGGATGGGAAAAGCAGT | 539 |
| BAMBI | NM_012342 | ATCCGCCCCGCAGCGGGGGG | 540 |
| BATF | NM_006399 | AAGTCCGTCTTCTGTCAACA | 541 |
| BATF2 | NM_138456 | AGGAGGGAAGACCAAAGGCC | 542 |
| BAX | NM_138764 | TTGGACGGACGGCTGTTGGA | 543 |
| BAZ1B | NM_032408 | CTGCAACCCAACTACGCGAC | 544 |
| BBS5 | NM_152384 | AAGCCCAGCTGTGTCCGCCA | 545 |
| BCAN | NM_198427 | GATGACGATGTTGCAGCTGG | 546 |
| BCAP29 | NM_001008405 | CACGGACCCCGGTCAGGAAG | 547 |
| BCAP31 | NM_001256447 | CGTCCGTCCGCTCCGCAGCC | 548 |
| BCAR1 | NM_001170716 | CACCCACACAGAGATTCCCT | 549 |
| BCAR1 | NM_001170717 | ATTTGCATGGAGAGCGGCGG | 550 |
| BCAR3 | NM_003567 | ATGTCTCGGGGGGTTCCGCA | 551 |
| BCHE | NM_000055 | AGCACAGATTGAAGCTATAA | 552 |
| BCKDHA | NM_001164783 | AAGAAGAGGGCAACCTGACC | 553 |
| BCKDHB | NM_000056 | TTCTGCTCCTTGTGCGCATG | 554 |
| BCL10 | NM_003921 | TGTGTGACCAAAACAGTAAC | 555 |
| BCL2 | NM_000633 | CAGGCATGAATCTCTATCCA | 556 |
| BCL2L12 | NM_138639 | TAGCTGATTAGAGAGCCTCT | 557 |
| BCL2L15 | NM_001010922 | AAATACTTCCTCGACTTCTT | 558 |
| BCLAF1 | NM_001077441 | AAGTCGCGTGGCTGGTCTCG | 559 |
| BEND5 | NM_024603 | ATTGGCAGAACGGTGCTTTC | 560 |
| BEND6 | NM_152731 | GAGGCTGCGACTCGGCGGCT | 561 |
| BEST2 | NM_017682 | GGCAAGGGTCAGGACTGAAG | 562 |
| BEST4 | NM_153274 | TACCTTGTCCAACTCTAGCC | 563 |
| BFSP1 | NM_001195 | GAGCAGCGGCCCGCTTTGTG | 564 |
| BICD2 | NM_015250 | CGGGCGGGCGCCGGGCATGA | 565 |
| BID | NM_001244567 | GTGGTCATTCTAGGTCCTCA | 566 |
| BIRC2 | NM_001166 | TGAACCTCCGGGAAAGACGC | 567 |
| BIRC6 | NM_016252 | GGACGCTGCGGACGCGGAAC | 568 |
| BIVM | NM_017693 | CCTGAGAGAGAGGAGCAGCG | 569 |
| BLCAP | NM_001167820 | ATTCGGGCTTGAAGATCTCG | 570 |
| BLID | NM_001001786 | TTACAATTCAGAAATCAACG | 571 |
| BLK | NM_001715 | ATCAGCATTAAATGGTAGAA | 572 |
| BLK | NM_001715 | TAGGGTACTGTAAAACACAT | 573 |
| BLMH | NM_000386 | TGGCTTCTCACAAGGCTTCC | 574 |
| BLNK | NM_001258441 | AATAATGAAACCTATTGGGC | 575 |
| BLOC1S2 | NM_001001342 | TGAGTGTGTGGTGGCTCACC | 576 |
| BLZF1 | NM_003666 | TCCCACGCCTCGTGCGACAG | 577 |
| BMP4 | NM_001202 | TGGAGGGGAGGATGTGGGCG | 578 |
| BMPR1A | NM_004329 | GGGCGTCCGCGGGCCTTGCA | 579 |
| BMX | NM_001721 | AGTGGGTCCATCATACTCCC | 580 |
| BOD1 | NM_001159651 | AGTTGTAGTTTCTCTCGGCT | 581 |
| BOLA1 | NM_016074 | ACAGTTCCCATGAGCCCTCA | 582 |
| BOLL | NM_197970 | CCCTCTCGCCTTCTCTCAGA | 583 |
| BOLL | NM_197970 | GCGGAGCGAGGGCTCGGTTC | 584 |
| BOP1 | NM_015201 | CCGCCCTCCCGCGTCACCCC | 585 |
| BORA | NM_024808 | TGATTGCCTCGGAGAGAGGA | 586 |
| BPIFB6 | NM_174897 | ATGAGCACTGCCCTCTTCCA | 587 |
| BPY2 | NM_004678 | AGTCACATCACCTAGGTGAT | 588 |
| BPY2 | NM_004678 | ATATGTCACAATGCTCCATG | 589 |
| BRAT1 | NM_152743 | AGCTAAATGACCAAGGGCTT | 590 |
| BRD2 | NM_001199455 | TGTTTTAGACTGTGGGGCAT | 591 |
| BRD2 | NM_001199456 | TCGCGGAAACGTACTTATTG | 592 |
| BRI3BP | NM_080626 | AAATGATGAGAAGCCGCACC | 593 |
| BRINP3 | NM_199051 | AATCTGCAAAGAGAAGTAAA | 594 |
| BRPF1 | NM_004634 | CCATCTTAGAGTGGAGTTTC | 595 |
| BRPF3 | NM_015695 | TGCGGGCTCTCCCGCTGAAC | 596 |
| BRPF3 | NM_015695 | TGGAGGTGGCGGGGGGAGGC | 597 |
| BSDC1 | NM_001143888 | CCTAATGATGGCGCAGGGAG | 598 |
| BTAF1 | NM_003972 | CGGTAAGCAGGGGTCCAAGA | 599 |
| BTBD11 | NM_001018072 | CTGCAGCCTCGGTGTCCGCC | 600 |
| BTBD3 | NM_014962 | ATAGGTGTCACTGTTTTGCT | 601 |
| BTBD7 | NM_001289133 | CGGTGCGTTCGCTGGATCCA | 602 |
| BTBD9 | NM_052893 | AGGAAGGTTCTCCAAGGAGT | 603 |
| BTF3 | NM_001037637 | TGGGGCGCAGCCCGTACCTC | 604 |
| BTK | NM_000061 | AAGGGCGGGGACAGTTGAGG | 605 |
| BTN1A1 | NM_001732 | AAGAACTGTAGAGAGGACTT | 606 |
| BTN1A1 | NM_001732 | ATGACCAGAACACTTGCAGC | 607 |
| BTN3A1 | NM_001145008 | GAAATATCAGCAGAACACAA | 608 |
| BTNL3 | NM_197975 | ACTTGGAGGGACTTTGTTCT | 609 |
| BTNL9 | NM_152547 | GGGTCACAGAAGGAGGGGAA | 610 |
| BUD31 | NM_003910 | ATTCTATACAGGCATTGCTG | 611 |
| BVES | NM_007073 | AGCTGCTTGTTCTACGCGCC | 612 |
| BVES | NM_147147 | CGCAGAGCCTGCGTGCAGCC | 613 |
| BZW2 | NM_001159767 | ATGTGGCGAAATATTTGAAC | 614 |
| C19orf11 | NM_032024 | TGACTTCTAGTCCTCGCTGC | 615 |
| C10orf120 | NM_001010912 | TAAGACATTGAATGATCCCC | 616 |
| C10orf128 | NM_001288743 | AATACCCCAGCATGTACAAT | 617 |
| C10orf128 | NM_001288743 | TAATCACAGCCAGCTTCTGG | 618 |
| C10orf90 | NM_001004298 | CTCTTTGATGTTTACATTTG | 619 |
| C11orf54 | NM_001286071 | GGCTGGTTATCGGGAGTTGG | 620 |
| C11orf97 | NM_001190462 | TTTGGTGCGCGGAATACCTA | 621 |
| C12orf40 | NM_001031748 | TGAAAGCCTAAATTTTTGAC | 622 |
| C12orf65 | NM_152269 | GGCTGTCTCCGCCTCCTTCC | 623 |
| C12orf74 | NM_001178097 | AGGTTGTGAGATGCATTCTT | 624 |
| C14orf159 | NM_001102367 | TTCAAGCCAGATAGCACCTG | 625 |
| C14orf180 | NM_001286399 | TCTGGTCTTATCTGAAATCA | 626 |
| C15orf41 | NM_032499 | TAATCTTGAGGTTAAGGTTG | 627 |
| C15orf57 | NM_001289132 | AAGGAATCAACCTGGCCCTC | 628 |
| C15orf57 | NM_001289132 | GAGGGGAGGGGCAATGCTCA | 629 |
| C16orf45 | NM_033201 | ACACAAAGGAAGTGAGAACA | 630 |
| C16orf70 | NM_025187 | GCCAGCGCGAGGGAGGAGCC | 631 |
| C16orf74 | NM_206967 | CGGGTCCTGGCACGCTCCCC | 632 |
| C16orf74 | NM_206967 | GCGCCTGGCCCGTGCAATCC | 633 |
| C16orf95 | NM_001195125 | GATGAGTGGCTCCAGTGGCC | 634 |
| C17orf100 | NM_001105520 | AGAGCAAAAGCCCAGAGACG | 635 |
| C17orf105 | NM_001136483 | TGTGTTTTTAATGCTAACCT | 636 |
| C17orf50 | NM_145272 | TGGAAAAGGAAATTATTCCT | 637 |
| C17orf51 | NM_001113434 | TGAGGGGACGGGGCGGGGCT | 638 |
| C17orf80 | NM_001100621 | GCGAGCGCTTCTGCCACCCC | 639 |
| C17orf96 | NM_001130677 | GTGCGGAATGGGGACGGGGG | 640 |
| C18orf25 | NM_145055 | TGACGGTCTCAACAGAAGGA | 641 |
| C18orf63 | NM_001174123 | GCAAGGCTTGCAGGGCATGC | 642 |
| C19orf38 | NM_001136482 | ATCAGACCCGCGCACCTCTC | 643 |
| Cl9orf44 | NM_032207 | GGGGGTGTGCACTGCGCTTC | 644 |
| C19orf70 | NM_205767 | CCCAGCGCCGGAGCGTCGCC | 645 |
| C1orf111 | NM_182581 | TCTACTACATTCTTCTCTCT | 646 |
| C1orf123 | NM_017887 | TGCGAAAAGCCCAGTGGGCC | 647 |
| C1orf127 | NM_001170754 | CTTCTCCCCATCCCTCTGCA | 648 |
| C1orf131 | NM_152379 | GCAGAGGGTGCCGCCGCCCT | 649 |
| C1orf141 | NM_001276352 | TTTTAGTGACAAAAGTCTGT | 650 |
| C1orf159 | NM_017891 | GGCTGCACCAGGTTTGGCCG | 651 |
| C1orf185 | NM_001136508 | GATGATCCCTAGGGAAACCT | 652 |
| C1orf198 | NM_001136495 | GCTGTTGTAAGGATTAAATG | 653 |
| C1orf52 | NM_198077 | GCTGCTTTTGCTCATTTCTG | 654 |
| C1orf53 | NM_001024594 | GGCCCGCTGCGGAAATAAAA | 655 |
| C1orf54 | NM_024579 | CCTCTCAATCTGGGCAGCTC | 656 |
| C1QA | NM_015991 | AAGCAGACTTCAGCAAGACT | 657 |
| C1QL3 | NM_001010908 | AGTGGGGAAATCGGGGATTT | 658 |
| C1QTNF3 | NM_181435 | ACTTCAACAGAAACGTGCCA | 659 |
| C1QTNF4 | NM_031909 | GTCCTCTGGGTCTAGAGAGC | 660 |
| C1QTNF5 | NM_001278431 | AGGGGGAGAGAGACTTGAGC | 661 |
| C1QTNF6 | NM_182486 | ATTTCCTTTGCTTAACTCTT | 662 |
| C1RL | NM_016546 | TTAATTTTTGCCATGTGTGT | 663 |
| C20orf194 | NM_001009984 | ACCCCACTTCTTAAGCTGCG | 664 |
| C20orf194 | NM_001009984 | GCTCCCAACATCCGGTCCGG | 665 |
| C20orf196 | NM_152504 | GGCTTGTCGATAAATGTGCT | 666 |
| C20orf202 | NM_001009612 | CATCACATATTCTTGGCTTC | 667 |
| C21orf140 | NM_001282537 | CTGTAAGAAAGCCCTTTATG | 668 |
| C2CD2L | NM_001290474 | GAGGTTCCGGGGTTGAAAAT | 669 |
| C2CD4B | NM_001007595 | AGGCACCTTGTGGTCAGCTC | 670 |
| C2CD4C | NM_001136263 | TGGCAGGGAGGAGCCTCGCC | 671 |
| C2orf15 | NM_144706 | GGAGACGGGACGCTCGGCTC | 672 |
| C2orf57 | NM_152614 | CAGTTTGTTGCCAACTTTGC | 673 |
| C2orf68 | NM_001013649 | AAAACAAAAGCCCTCCGTCC | 674 |
| C2orf81 | NM_001145054 | ATGTCACCACCAAGGGATCA | 675 |
| C2orf83 | NM_001162483 | TGAGGCAGGCAGATCACTTG | 676 |
| C3orf30 | NM_152539 | GAGTACGCCATGTCCTGAGA | 677 |
| C3orf38 | NM_173824 | TTCTGCGGCCACTTCTGAGT | 678 |
| C4BPB | NM_001017366 | ATTTGGTTAACTCTGGACTC | 679 |
| C4orf3 | NM_001170330 | TCACACATGCTGGAGTGCAG | 680 |
| C5orf38 | NM_178569 | TGGCCGGGGACGGTGGGAGC | 681 |
| C5orf67 | NM_001287053 | AAGTCCTTGCCCTCATTCCA | 682 |
| C6orf10 | NM_006781 | AGGCAGAGGATCAAAAGGCT | 683 |
| C6orf48 | NM_001287484 | TTCTGTGTGGACAAACAATG | 684 |
| C7 | NM_000587 | CACAGATTAAGTACAAGGTC | 685 |
| C7orf50 | NM_001134396 | GCCATTAGCCGGCGGAGAGA | 686 |
| C8A | NM_000562 | TTTGAAAAACAATATCCGTG | 687 |
| C8B | NM_001278544 | TTTTGCACCAACCTAGTCAG | 688 |
| C8G | NM_000606 | TCAACTCGGACTTTGTACAT | 689 |
| C8orf22 | NM_001256596 | CTACATAAACCAGTTTCTTC | 690 |
| C8orf22 | NM_001256598 | GCTTGCTTGCTGCCTCTGGC | 691 |
| C8orf44- | NM_001204173 | ACAAGTACCGTGAGGCCAAG | 692 |
| SGK3 | | | |
| C8orf74 | NM_001040032 | CTGGTCACCTGCACCTGCTC | 693 |
| C8orf88 | NM_001190972 | AGCGCGCGCCACCCTTTTAA | 694 |
| C8orf89 | NM_001243237 | CTACAAGACAATGGAATACT | 695 |
| C9orf131 | NM_203299 | GAATTATGCTTCAGGCATTG | 696 |
| C9orf152 | NM_001012993 | GCCTCTGGATGTGTGCCCCG | 697 |
| C9orf3 | NM_001193329 | CATGAAAGAAAGCTGCATTA | 698 |
| C9orf57 | NM_001128618 | GTGCTGCTTTAAAGACTATA | 699 |
| C9orf64 | NM_032307 | AACTCACGGCCGGTGAACGC | 700 |
| C9orf72 | NM_018325 | CCAGAGCTTGCTACAGGCTG | 701 |
| CA1 | NM_001164830 | AACATGAGTGAAACAGGACT | 702 |
| CA1 | NM_001164830 | ACTCATGTTAGTAGAAGATA | 703 |
| CA11 | NM_001217 | TCATAGCGGCAAACACTCCT | 704 |
| CAAP1 | NM_001167575 | AAAACAAACTCTGACTAGAC | 705 |
| CAB39 | NM_016289 | TTGGCTTCTGCTTTTCTCTG | 706 |
| CAB39L | NM_001287339 | AGGCACAGGGAAAATCCAGC | 707 |
| CABIN1 | NM_001201429 | AGCAGCCCGCGGAGAGCGAG | 708 |
| CABS1 | NM_033122 | CAGCCTAGAAACAACCTCCA | 709 |
| CABYR | NM_138644 | ACCCACCGAGGCCTCAGATT | 710 |
| CACNA1F | NM_001256790 | TGTCATTTTCCAGTAGTATA | 711 |
| CACNA1H | NM_021098 | CTCGCTGCCTCACCGGTCCC | 712 |
| CACNA1I | NM_021096 | CAGCCCCACCTGAGCCCCAC | 713 |
| CACNA1S | NM_000069 | TTTCAAGCCTGGGGCAACAG | 714 |
| CACNB2 | NM_201590 | AGAACAACAGGTTGCATAAC | 715 |
| CACNG2 | NM_006078 | TTAAGGCATCTCACTTGGGG | 716 |
| CACNG5 | NM_145811 | CTTTACCCATCCATTGAGCC | 717 |
| CACNG5 | NM_145811 | GCACCTCTGTTGCAGTGACC | 718 |
| CACYBP | NM_001007214 | ACAGTCCATGACTGAAAGGA | 719 |
| CADM2 | NM_001167674 | AGAAGCCTGTTTGTTTTTCC | 720 |
| CALCB | NM_000728 | AGTGCGAGCTATGACGCAAT | 721 |
| CALCOCO2 | NM_001261393 | GGACTTAGGAGAGCCATCAA | 722 |
| CALHM1 | NM_001001412 | TGCTAGAGACCAGCTTTCTG | 723 |
| CALN1 | NM_001017440 | GCGCAACCTGAGGAACGCCT | 724 |
| CAMK2G | NM_001222 | GGAGGCCCCTCCCCGGGGGC | 725 |
| CAMKK1 | NM_172206 | AGCTCACCCAGCAGGTAGTG | 726 |
| CAMTA2 | NM_015099 | ACTCCACGTGTGCTGACCCC | 727 |
| CAPN1 | NM_001198868 | GCCCATGTGTCACCTTACCC | 728 |
| CAPN2 | NM_001748 | ATCCTAGCCTTCTTCCCTAT | 729 |
| CAPN3 | NM_173088 | GGCAGGACTGTGATAGGAGA | 730 |
| CAPN7 | NM_014296 | CGCCCGGGATTGAGCAGCTG | 731 |
| CAPN9 | NM_006615 | CACCTCTGCTTAGTGCGCTC | 732 |
| CAPS2 | NM_001286548 | GCAAGCCTTGTCCCGCCTCC | 733 |
| CAPZA3 | NM_033328 | TTCGAAGAAGACTGTTCAGG | 734 |
| CARD14 | NM_024110 | AAGGAAGCTTCAATAGTTAC | 735 |
| CARD19 | NM_032310 | GCCTATCCCAGGACGGCAAG | 736 |
| CARHSP1 | NM_001278260 | GAACGCAGAGCGCGGGACGT | 737 |
| CARHSP1 | NM_001278263 | GCCGCGCCAGCTGTGGCTCG | 738 |
| CARMIL1 | NM_017640 | AACGCAGGAGGAAGAGGAGA | 739 |
| CASP12 | NM_001191016 | CAACCCCGGAAGTGTGATTT | 740 |
| CASP8 | NM_033358 | AAACGACAACTCACAGTGCC | 741 |
| CASS4 | NM_001164116 | GGCCTAGTGGCCTCTCATCA | 742 |
| CATSPERG | NM_021185 | GCGCAACCCCTAAGGCACCG | 743 |
| CBFB | NM_001755 | GGGTGGCGCATGCGCGGCGT | 744 |
| CBL | NM_005188 | CTGCTCGAAGCCGGTGGCCC | 745 |
| CBR3 | NM_001236 | CTGGACTGAAGAAATTATTT | 746 |
| CBX1 | NM_006807 | GCAGCGCCCAAGAGCCCGAG | 747 |
| CBX1 | NM_001127228 | CCCATATGTTCTAATATTCT | 748 |
| CBY1 | NM_015373 | TGCTATCCCGAGGTGATTCA | 749 |
| CCDC105 | NM_173482 | TGGAAGAAGGGCCATGTTGC | 750 |
| CCDC110 | NM_001145411 | GGACCCACCGGGACCCCACC | 751 |
| CCDC114 | NM_144577 | GGGAGGGAGAGTGTCTGTCC | 752 |
| CCDC120 | NM_001163321 | TCACCCCTGGGGGCAGTTTC | 753 |
| CCDC144A | NM_014695 | TTGGCTTGGCCTTACCCACG | 754 |
| CCDC148 | NM_138803 | GGCGGCGTGCTGACGTTCCC | 755 |
| CCDC149 | NM_173463 | ATGTTAGTAAGGAGATGCTG | 756 |
| CCDC153 | NM_001145018 | GGACTGAGGGCTGGAAGGTT | 757 |
| CCDC155 | NM_144688 | GGTGGCTGCGCCCGCCATGC | 758 |
| CCDC159 | NM_001080503 | GTGCAGATCTACGACCCGAT | 759 |
| CCDC174 | NM_016474 | GTGTGGGCGCCATCTTGAGA | 760 |
| CCDC175 | NM_001164399 | AACGCAATGGAAATTGAAAG | 761 |
| CCDC18 | NM_206886 | GTGGGGGAAGCCATGGGAAC | 762 |
| CCDC184 | NM_001013635 | GGCTCTGGAGTCTGGACTAG | 763 |
| CCDC27 | NM_152492 | CAATATTGAAGGTTGCCTTC | 764 |
| CCDC33 | NM_001287181 | TCCTTGGCCACAGAATTGTA | 765 |
| CCDC38 | NM_182496 | ACATCTGCCCACAGGTTCTG | 766 |
| CCDC43 | NM_144609 | AGCGCGTCTTCGCATACGTG | 767 |
| CCDC57 | NM_198082 | CTTCGATCTGCGGCGGTGGT | 768 |
| CCDC68 | NM_001143829 | TGAAAACAACTACACTTCTT | 769 |
| CCDC68 | NM_025214 | TGTACAGGCGGGTGGGGGGA | 770 |
| CCDC80 | NM_199512 | AATTCTCAGATTTCTGCATC | 771 |
| CCDC90B | NM_021825 | AATTCGGCTTCCCTAAAGAA | 772 |
| CCK | NM_000729 | TTAGAAAGTGGAGCAGCAAC | 773 |
| CCL13 | NM_005408 | TGAATCTGCTGAGCTGGAGC | 774 |
| CCL14 | NM_032963 | AAATGGTCTTCCATCCCCAG | 775 |
| CCL15 | NM_032965 | GGTCTGCCAGCACTAGGGAG | 776 |
| CCL2 | NM_002982 | CCTACTTCCTGGAAATCCAC | 777 |
| CCL21 | NM_002989 | TGGGAATAGAAGGAAGGCTC | 778 |
| CCL26 | NM_006072 | CTGGGTGGACAATGAATTCT | 779 |
| CCL28 | NM_148672 | ATGTTTCTTTCCTTAAGACC | 780 |
| CCL3L3 | NM_021006 | TGCTGAGTGTTGCACAACTC | 781 |
| CCL5 | NM_002985 | AAGAAAACTGAAATAGCCTC | 782 |
| CCNA2 | NM_001237 | TTAAAATAATCGGAAGCGTC | 783 |
| CCND3 | NM_001136017 | TTGCCAACGCCGGGAGGCAG | 784 |
| CCND3 | NM_001760 | GTGGGCCTCCTACCCACCCA | 785 |
| CCNG1 | NM_004060 | GGAATTTGAGGCCAGATAAC | 786 |
| CCNJ | NM_001134376 | TGCGAAGCCGGCCTGATCGC | 787 |
| CCNK | NM_001099402 | CAGAGGGAGGAGCCAGCCAC | 788 |
| CCRL2 | NM_003965 | TGCCGCTCTGAGTGGTAGCA | 789 |
| CCRL2 | NM_003965 | TGGCATGTGACACTCTGAGT | 790 |
| CCS | NM_005125 | GGCCCTGCTTCGTCAGCCAC | 791 |
| CCSER1 | NM_001145065 | GAGCGCGAGATCCACCTCCC | 792 |
| CCSER2 | NM_001284243 | ACATAGCTACTGACTTAGGA | 793 |
| CCSER2 | NM_018999 | CAAGGTCAGTGGAGGGGGCG | 794 |
| CCT5 | NM_012073 | AGACACTTAGTGGAAATCTT | 795 |
| CCT6B | NM_001193530 | AGCAGCGTCTGAGCACCAGT | 796 |
| CCZ1 | NM_015622 | CGGCCAGGAAACAGCCACCC | 797 |
| CD101 | NM_001256111 | GGCTCACAGTATGTGTCATT | 798 |
| CD14 | NM_001174105 | GGAGTAGAGTGCCATGATCT | 799 |
| CD160 | NM_007053 | AGAAATAGACTAGGGTGCTG | 800 |
| CD1A | NM_001763 | AGGTGCTAAGAGAGACTGTT | 801 |
| CD1C | NM_001765 | GAATGGAGTGATGAGAAGAG | 802 |
| CD200R1 | NM_138806 | ATTGGGAAATTTACAAGGAT | 803 |
| CD200R1 | NM_138806 | CTGTGTACAGCAGAAGTGAG | 804 |
| CD200R1L | NM_001199215 | CAAAGGACACTTTGGAACAA | 805 |
| CD300E | NM_181449 | CAGATTTTCCTGTTTGTGCT | 806 |
| CD300LG | NM_001168324 | GTGGGCGCTCAGAAAAGGGA | 807 |
| CD33 | NM_001772 | GAGGGTCAATCTGTGTGGAG | 808 |
| CD3D | NM_000732 | CAATAGGGACGCTAAAGTTC | 809 |
| CD3D | NM_000732 | GCTGGCAGAGAATATGGAAA | 810 |
| CD3G | NM_000073 | TGCCTTTTGTTTTTCCGTTA | 811 |
| CD44 | NM_001202557 | CTCTCTCCAGCTCCTCTCCC | 812 |
| CD53 | NM_001040033 | TACCCAGTGTGAGGAGATCT | 813 |
| CD5L | NM_005894 | CCCCTTTGCTATGTAAACAG | 814 |
| CD63 | NM_001257389 | CGTCTGTGATAGCGAGGGCT | 815 |
| CD63 | NM_001780 | CCTCCGTGCCAACTCGGGGT | 816 |
| CD72 | NM_001782 | TGGGTTTAAGATGCATGGAG | 817 |
| CD79A | NM_001783 | CCTGCCCATGACACATGCCC | 818 |
| CD80 | NM_005191 | CATGAAACACCACGAGCACC | 819 |
| CD84 | NM_001184879 | TATTGCCAGCACCCAGAAGA | 820 |
| CD8A | NM_171827 | CTTAAACAGACCAGCATTCC | 821 |
| CDC20B | NM_001145734 | CTCTGACGACACCGCGGCGC | 822 |
| CDC40 | NM_015891 | CTCATATTCTTTAGTCAACT | 823 |
| CDC42BPA | NM_003607 | CTCCCCCTTCTTCACACCCC | 824 |
| CDC42EP3 | NM_006449 | AGAAACGCCTCCCTCTGGGT | 825 |
| CDC45 | NM_003504 | CCTCAGAGGTGACGCTTCTT | 826 |
| CDC7 | NM_001134420 | GTTTCCGACGGTTTGTTCCA | 827 |
| CDCA5 | NM_080668 | TCCGCTGCCACGTCTCTTCC | 828 |
| CDCP1 | NM_022842 | GTCCCTACTACTCCCCATTG | 829 |
| CDH18 | NM_001167667 | AAATTCCACAGCAAGCAAAA | 830 |
| CDH19 | NM_021153 | AATTCTCCCTTTATCAACTC | 831 |
| CDH2 | NM_001792 | TGGGTGCAGCACGCACGACC | 832 |
| CDH4 | NM_001252339 | GGACAGGGCTATTGTCTTGG | 833 |
| CDH6 | NM_004932 | TGGAACACTCCTTCAGCCCC | 834 |
| CDIP1 | NM_001199055 | GGCTGAGCACGTGGGATGGT | 835 |
| CDK10 | NM_052988 | CCTTATTTTAGGGTGAAGCC | 836 |
| CDK11A | NM_033529 | GTGAGCTGCACTTCCGACTT | 837 |
| CDK16 | NM_001170460 | AGTGTACACCAGCTCTTCTC | 838 |
| CDK17 | NM_001170464 | TCGGAGCGGGCAGTTTCCCG | 839 |
| CDK2 | NM_052827 | AGAGACATAGGTAGGAAACT | 840 |
| CDK2AP1 | NM_001270434 | GGGTTCTCCAGTGCTCCTCC | 841 |
| CDK2AP2 | NM_005851 | GCCACGTACCGTTCTTCCTG | 842 |
| CDK5RAP3 | NM_176096 | ACGCAGATTGAGACGTCTGC | 843 |
| CDKL5 | NM_001037343 | AAGCCTTCACTGTGACAGAA | 844 |
| CDS1 | NM_001263 | GGCCTGAGAAAAGGTGGGAG | 845 |
| CDYL | NM_001143970 | ACAGACGGCACCTGGAAAAT | 846 |
| CDYL | NM_004824 | GGGGAGCAGTGGGCTCCGCT | 847 |
| CEACAM21 | NM_001288773 | GGCAGCAAGACCCTCCCCAC | 848 |
| CEACAM21 | NM_001288773 | TCTAAGAGTGCAAATGTCAG | 849 |
| CEACAM7 | NM_001291485 | GCTGATGGACCCCTGTCCCC | 850 |
| CELA2A | NM_033440 | GGTGACATTTGGGAGGAAAT | 851 |
| CELF1 | NM_006560 | TCTTTGTCTCCGATCCCTAC | 852 |
| CELF5 | NM_001172673 | GCCCGCGCCCGCCCCGGCAT | 853 |
| CELF5 | NM_001172673 | TCAGTTTCCCCCCGCGGCCC | 854 |
| CENPA | NM_001042426 | AATATAGCGGCGATGATAGG | 855 |
| CENPL | NM_001127181 | GACTGTTACTCCTTGTTTTC | 856 |
| CENPM | NM_024053 | TTCCACGCTCCACAGTAAGC | 857 |
| CENPN | NM_018455 | ATCTAGCAATTGAGAATTTG | 858 |
| CEP152 | NM_014985 | GGATTCGAGAGCCAATTACG | 859 |
| CEP164 | NM_001271933 | AAGTGGATTGAAAGTGTAGA | 860 |
| CEP290 | NM_025114 | AGTCATGGTCTACCTCGTTC | 861 |
| CEP44 | NM_001040157 | CAAACTTTACTTGTCCACAC | 862 |
| CEP63 | NM_025180 | CAAATGAACTCACCCACATC | 863 |
| CEP76 | NM_024899 | AGGCCCGTCCAGCTAACTGC | 864 |
| CEPT1 | NM_001007794 | AGTTCTGGGTTCAGATACTT | 865 |
| CERS1 | NM_001290265 | GGTCTGCACAGCGGGCTACT | 866 |
| CERS1 | NM_001290265 | TCCCAGGCATCTTCTTCTGC | 867 |
| CERS1 | NM_021267 | AGAAACCCAGGCGCGGGGGC | 868 |
| CES1 | NM_001266 | GCCCAACTACTTGTTACATA | 869 |
| CES2 | NM_198061 | CCCCAGAGCGCTGGTAGATG | 870 |
| CETN1 | NM_004066 | GCGAGAATCCGCTGTCCCCT | 871 |
| CFAP100 | NM_182628 | ATGTCCTCCCTGACGCCGCC | 872 |
| CFAP43 | NM_025145 | GGTCTGTTTACCAGCAACAT | 873 |
| CFAP43 | NM_025145 | TTGGCTTGCCGCTCACCCAT | 874 |
| CFAP52 | NM_001080556 | TGCTATTTCTCTGGAAATTT | 875 |
| CFAP52 | NM_001080556 | TGGGGACTGGAAGAGAGATG | 876 |
| CFAP58 | NM_001008723 | GGGCGGTGCCCCTGAGAGGC | 877 |
| CFC1 | NM_032545 | CTTGTACTGGGAGATGGTGA | 878 |
| CFDP1 | NM_006324 | ATGCTGGAACTTGTAGTCTT | 879 |
| CFHR3 | NM_021023 | TTTGATTGCCTGATATGTAC | 880 |
| CGGBP1 | NM_001195308 | TGTCGCCCCTACGGCCCACT | 881 |
| CHADL | NM_138481 | CAGGCAAGCCAGGCTTCCCC | 882 |
| CHAF1B | NM_005441 | CCGCCCACTCATAGACGCCA | 883 |
| CHAT | NM_020986 | CTGGAAAAGAGGGTCTATCC | 884 |
| CHD2 | NM_001042572 | AGAGACAGATCCTCCATCCC | 885 |
| CHD4 | NM_001273 | GGGGGGGTTGGAGTTGGTTG | 886 |
| CHD8 | NM_020920 | TAGGTTGAGAGCGCACGGAG | 887 |
| CHFR | NM_018223 | GGCCATCTTTGATCCTGACC | 888 |
| CHID1 | NM_001142676 | GGAGCTGGTTATCAGGTTCC | 889 |
| CHL1 | NM_006614 | CCCACCACGCCCTTAAATGA | 890 |
| CHML | NM_001821 | ATGCAACAATGACAATCCAT | 891 |
| CHML | NM_001821 | TTTAAGACATGCTTTAGTAG | 892 |
| CHMP2A | NM_014453 | CTGGCTTGGGTCACTCGGGC | 893 |
| CHMP3 | NM_016079 | TACGAAAAGCACCGAATCCG | 894 |
| CHMP4A | NM_014169 | ATAGAAACTCCCCACACTGT | 895 |
| CHMP4B | NM_176812 | CTACAGCAAAAGACGCGCCG | 896 |
| CHMP4B | NM_176812 | GGCCGCGCCTCAAATCTAAT | 897 |
| CHMP4C | NM_152284 | GAAAAGACCGACAAAGACTG | 898 |
| CHODL | NM_024944 | ACTTCGTCTCTCCAGCCATG | 899 |
| CHP1 | NM_007236 | CATCGCCCCTTTAAGGCCGG | 900 |
| CHP2 | NM_022097 | GCACGGCTGGGATTCCAACA | 901 |
| CHRNA10 | NM_020402 | GGCAGAGGCCAGAAGAGGCA | 902 |
| CHRNB2 | NM_000748 | GGCAGGACCTGCAGCATGGT | 903 |
| CHST1 | NM_003654 | CGTGGCTGCCCCCGGCGGGT | 904 |
| CHST1 | NM_003654 | GGCTGCGGAGTGGGTGTCCA | 905 |
| CHST8 | NM_001127895 | TCGCTGGAGCGATCCCCGCC | 906 |
| CHSY1 | NM_014918 | GCGCAAAAGTGAATGAGGGG | 907 |
| CIAO1 | NM_004804 | ACCCGGGGCCGATGCACTTC | 908 |
| CIB3 | NM_054113 | AGGGAGATTTGCCCAGACAC | 909 |
| CIDEA | NM_001279 | GCGGGAGCCAGGACGACCGG | 910 |
| CIDEA | NM_001279 | GGATCGCGACTTCGCGCTCT | 911 |
| CILP2 | NM_153221 | GGACTGAGTGGGCTCGGGGA | 912 |
| CISD2 | NM_001008388 | ACGCTCGCGGCGGACTGCCG | 913 |
| CITED2 | NM_001168388 | ATGTGCTGCTGAGCCGGTCC | 914 |
| CKAP2L | NM_152515 | TGCACGTTCTTCCAATCAAA | 915 |
| CLASP1 | NM_001142274 | ACGCTCTCTATGGTGTACCC | 916 |
| CLASP2 | NM_001207044 | ATTAACTGCTCTCATTATGC | 917 |
| CLCN1 | NM_000083 | ACTGCCACATCTGATCTGCT | 918 |
| CLDN1 | NM_021101 | TGAGCCGCCCTGAAACCGCC | 919 |
| CLDN19 | NM_001185117 | AAAGCTCATGCCCAGCCCCC | 920 |
| CLDN23 | NM_194284 | AGGTGAGCGCAGGAAGCGGC | 921 |
| CLDN5 | NM_001130861 | CCGGGCATTCTTCTGCACAA | 922 |
| CLDN8 | NM_199328 | TAAACATACTGCTGTCTTCT | 923 |
| CLEC11A | NM_002975 | GATCTTTGGGCTACAGCAGA | 924 |
| CLEC11A | NM_002975 | GGAGACCCAAGGCGGGATCT | 925 |
| CLEC12A | NM_001207010 | AAATGCCAGAGGTTCAGCCT | 926 |
| CLEC12A | NM_201623 | AGACATAGTGTAGGATTTAT | 927 |
| CLEC17A | NM_001204118 | AGGAATAATGACAACTGGCC | 928 |
| CLEC17A | NM_001204118 | TTCTGTGCGTGAATCCAAAC | 929 |
| CLEC4D | NM_080387 | GGTTTCTACTAACTGTTGTT | 930 |
| CLIC3 | NM_004669 | GCTTCATCTGCCCGCCTAGG | 931 |
| CLIC5 | NM_001256023 | TGGTCCTGGCAAAGCCACCA | 932 |
| CLIP3 | NM_015526 | GGCCAGAGGCGGCGACTGAA | 933 |
| CLK1 | NM_001162407 | TCATGCACGGGGCGAGCAGG | 934 |
| CLN3 | NM_001286110 | GAGCCGTGACCTTAGATCAG | 935 |
| CLNK | NM_052964 | GCAATACGTGAAGCTTTCAG | 936 |
| CLNS1A | NM_001293 | GGAGGTCGGCTAAGAACGTG | 937 |
| CLPTM1 | NM_001282176 | ACTGACTGGATAAGATATCC | 938 |
| CLRN2 | NM_001079827 | ACACACTCCGCTACATAGTC | 939 |
| CLVS1 | NM_173519 | TGTGTGGGGAGTGATGACGC | 940 |
| CLVS2 | NM_001010852 | GGAGGCAATTTTGATGTAGA | 941 |
| CMSS1 | NM_001167924 | CTTAGGAACAGATGCCCAGA | 942 |
| CMSS1 | NM_032359 | TCCAAACTGCTTCTGCCTGT | 943 |
| CMTM7 | NM_138410 | CCTGGGATTTTGTGTGGGTG | 944 |
| CMTR2 | NM_018348 | GACGTGCTGGTTCCGCTCAC | 945 |
| CNBP | NM_001127196 | GATTTCCACCCAGTCTGGCC | 946 |
| CNDP2 | NM_001168499 | CTTAGTCCAGAAACAGCCAA | 947 |
| CNFN | NM_032488 | ATCAGACCGGCTTGGCTCCC | 948 |
| CNGA2 | NM_005140 | TCCCAAACTCAGTCCTTCAA | 949 |
| CNIH3 | NM_152495 | TGGCTGCAGCAGTGGGTTTC | 950 |
| CNN3 | NM_001286056 | ACGCCTCTCATCTCTTTCCC | 951 |
| CNNM4 | NM_020184 | CGCCGCGCGAGAGCCGCCAG | 952 |
| CNOT1 | NM_001265612 | CAATCACCGACAGGTGCCCG | 953 |
| CNPPD1 | NM_015680 | TCCGCGAGGTGAGCGTCGCA | 954 |
| CNPY1 | NM_001103176 | CGGCCGGAGGACTGGAAGCC | 955 |
| CNTD1 | NM_173478 | AACATGGCGTCTTCGGGAGC | 956 |
| CNTN4 | NM_175613 | ATGAAATGAGCATATCCTAT | 957 |
| CNTN5 | NM_001243271 | ACAGCGCGGGCGGCCGGGGA | 958 |
| CNTN6 | NM_014461 | CCAGTAACTCCTATTAGTGA | 959 |
| CNTNAP2 | NM_014141 | GCGGCGTCTCCTGCTCTCCG | 960 |
| CNTROB | NM_053051 | GCCGAGCGAGAACCCCCCTA | 961 |
| COA4 | NM_016565 | TCGAGATGGCGGCGCCTTTG | 962 |
| COL18A1 | NM_030582 | AGGCACCAGCCTTGGAATCA | 963 |
| COL28A1 | NM_001037763 | GGGATCAGTAAGCAATTTAA | 964 |
| COL4A1 | NM_001845 | AGCGCGGAGCCCTGGTGTCC | 965 |
| COL5A2 | NM_000393 | AGTTAAAGGGTGTGTGTCTG | 966 |
| COL6A5 | NM_001278298 | TAACGCACCCCTGATGCTAG | 967 |
| COL9A1 | NM_001851 | GAAATTCACCAGAAAGATCC | 968 |
| COLEC11 | NM_001255988 | TCCACTTGGTTTCCAACAGC | 969 |
| COLGALT2 | NM_015101 | TAGAACTCTACTCAGTCAAT | 970 |
| COLQ | NM_005677 | ACAGTTTAATGGGATATGGT | 971 |
| COMMD1 | NM_152516 | TCTGCAACACCCATCCCCTT | 972 |
| COMMD6 | NM_203497 | GAGAAGCGCTAATTAAATTT | 973 |
| COMMD7 | NM_053041 | TCAGTTTCTTCCACTCCAGA | 974 |
| COMT | NM_001135161 | GGAACATCAGTGGCTCCTTT | 975 |
| COMT | NM_001135162 | AGAGTCTTGCTCTGTCGCCC | 976 |
| COMT | NM_001135162 | TCTGAGGCGCTAAGAGTCCC | 977 |
| COMTD1 | NM_144589 | CAGGGGCGCAGTTCCCGGCG | 978 |
| COPS3 | NM_001199125 | CTGTCAAGCAAAGCGCCCGG | 979 |
| COQ10A | NM_144576 | GGTCACAGGACCCGATAGGT | 980 |
| COQ10A | NM_001099337 | AGAACTTAGAGGGCCAGGCA | 981 |
| COQ6 | NM_182480 | GTATAAAGTCCGAGAGGTTC | 982 |
| COQ8B | NM_001142555 | CCTGGAATTAAGGTGGGCAT | 983 |
| CORO1C | NM_001105237 | AAGTGGAGCCCAAGACCAGC | 984 |
| CORO6 | NM_032854 | GAAGAAAGCTCCCTGCTTCT | 985 |
| COX7A1 | NM_001864 | GTGCAGCACAGTTGTCCTAA | 986 |
| COX7A2 | NM_001865 | ACTAGTTTTCTTTGATAGCC | 987 |
| COX7A2 | NM_001865 | GATGAAGTCAATGTGAGACC | 988 |
| COX8A | NM_004074 | CGAGTTATGTTCCGCCTCCA | 989 |
| CPA2 | NM_001869 | TTGTTATCTTATCCTAGGAA | 990 |
| CPD | NM_001199775 | TGGGCTCCAGTGTCCCTCCG | 991 |
| CPE | NM_001873 | CAGTGACGTGGGTGGGTCAT | 992 |
| CPEB3 | NM_001178137 | ATACAGATTCTGAGGGGAAA | 993 |
| CPED1 | NM_001105533 | TTCAGACTCCAGATATACTT | 994 |
| CPNE1 | NM_003915 | TCAAGATCACCACATGAGGC | 995 |
| CPNE4 | NM_130808 | TTAGTTGTCTAGTTTGTCTA | 996 |
| CPNE6 | NM_006032 | CACATGCACCCACGACTCAC | 997 |
| CPNE7 | NM_153636 | ATTAGAAGCTGTCTCCTCCC | 998 |
| CPSF6 | NM_007007 | AAAAATTGGCCCCCACTCCC | 999 |
| CPSF7 | NM_001136040 | GTGCCCGCGCAGCCGGTTTC | 1000 |
| CPSF7 | NM_024811 | CCGCCACTTCCGGCATGCGC | 1001 |
| CPT1B | NM_152245 | ATGAAGACGACCCTGAGGTG | 1002 |
| CPXM2 | NM_198148 | CTGATTTACTTTAGGACCCT | 1003 |
| CRACR2B | NM_001286606 | GGAGATCTGATCCCAAGTGA | 1004 |
| CRAT | NM_001257363 | GGGCGAGTCATTGAGACCTG | 1005 |
| CRB2 | NM_173689 | GTCAGGAGGGAGAAACCAGT | 1006 |
| CRCT1 | NM_019060 | AGCATTGTAGGTGGTGCATG | 1007 |
| CREB3L2 | NM_194071 | CACTCCCCGGCTACATTCCA | 1008 |
| CREBRF | NM_001168393 | ACGTGACAGGGGTGCCCGGC | 1009 |
| CREG2 | NM_153836 | GTCCAGGCTCGCAGAAGACC | 1010 |
| CRIP1 | NM_001311 | CTTTGCATTTTAGTGATGTT | 1011 |
| CRISP1 | NM_001205220 | ATATGTTCAGTGATTCTTTC | 1012 |
| CRISP3 | NM_006061 | TTATTTGGTGATTCCTCAAA | 1013 |
| CRTAC1 | NM_018058 | GTAACCTTCAGGCGGCAGCG | 1014 |
| CRTC2 | NM_181715 | ATTAGCCCTGAGACTACGAA | 1015 |
| CRTC2 | NM_181715 | TTCCCAGCTTGCACCTCTCA | 1016 |
| CRY1 | NM_004075 | GCGCTCGGCGATTCCTCCCG | 1017 |
| CRYGN | NM_144727 | AGTGCAGCCCGCCCTGCCCG | 1018 |
| CRYL1 | NM_015974 | TGCTGACAGTCACAAGCGCG | 1019 |
| CS | NM_004077 | ACAACTGCTGTCAAGGGCTA | 1020 |
| CS | NM_004077 | CCCTTAATTAGCCCTAATCC | 1021 |
| CSDC2 | NM_014460 | ACGCAGCTGAGCCTCTCACC | 1022 |
| CSF1R | NM_001288705 | CCCTTCTAAAGCCATCTTCA | 1023 |
| CSF2RA | NM_001161532 | TGAACTCACGGAGCAATTAC | 1024 |
| CSGALNACT1 | NM_001130518 | CAGGGGCAGGGCAGGTCTGG | 1025 |
| CSGALNACT1 | NM_001130518 | CCCTGCAAGGCGCAATCTCC | 1026 |
| CSGALNACT2 | NM_018590 | CACTCTGCTGTCTCCACAAA | 1027 |
| CSH1 | NM_001317 | GACAAGTTGGGTGGAGTCTG | 1028 |
| CSMD3 | NM_198123 | TGGAGTTTATCAGAGAGCAG | 1029 |
| CSNK2B | NM_001282385 | CCAGGGGACTGGCCTATCCT | 1030 |
| CSPG5 | NM_001206945 | AACATATTTTACTTGGTCCC | 1031 |
| CSPG5 | NM_001206945 | TCATAGTTTCATGCTGCCTC | 1032 |
| CSRNP3 | NM_001172173 | CAAAAAATAGCTCCCAACTA | 1033 |
| CST11 | NM_080830 | TCAGCTGCTGATGAAGGGGG | 1034 |
| CST9 | NM_001008693 | TCATCTCCTGTTTAGGGGAG | 1035 |
| CST9L | NM_080610 | TCTTCGACGGGGTGAAGGAG | 1036 |
| CSTF2 | NM_001325 | GGAGTGAGAATATAGCCCTC | 1037 |
| CSTL1 | NM_138283 | GGGCATTCATGGGCTTTTGG | 1038 |
| CT47A1 | NM_001080146 | ATAGTGTTGCTCTGTTGCCC | 1039 |
| CT47A7 | NM_001080140 | CTTTGTCCAATGAATGATCA | 1040 |
| CT47A7 | NM_001080140 | TGAGTTGTCCTAGAGCTTAA | 1041 |
| CT83 | NM_001017978 | GGGATTTCTGGGAAGCCGAA | 1042 |
| CTAGE4 | NM_198495 | TTGTTACACTTCACATCCTG | 1043 |
| CTCFL | NM_001269051 | GGTATCTCAGTGCCTCCTGT | 1044 |
| CTH | NM_001190463 | TCCGCTTTGTGCACTGGGTG | 1045 |
| CTLA4 | NM_005214 | TACATTTTCCATCCATGGAT | 1046 |
| CTNNA2 | NM_001282600 | GAACATTTCAGTTTCCCACT | 1047 |
| CTNNBL1 | NM_030877 | CAATCAAGTTTGGTTTCTTC | 1048 |
| CTNND1 | NM_001206886 | GAGGAATTACTGCAGAGCTG | 1049 |
| CTRL | NM_001907 | CCTAAAGGGCCTGTCTTGCC | 1050 |
| CTSC | NM_001814 | CTGCAACTGGACCCAGAACT | 1051 |
| CTSD | NM_001909 | ATTCCCGTTTCGGCCTGGCC | 1052 |
| CTSD | NM_001909 | CAGACCCCAGAAGCTGGGCC | 1053 |
| CTSE | NM_148964 | GGGAGAACTTGGGAGTCCTC | 1054 |
| CTTNBP2 | NM_033427 | AGCCCGCGGCTGGCGCCACC | 1055 |
| CTU1 | NM_145232 | ACTTCCGCTGGATGCGCCTA | 1056 |
| CUEDC1 | NM_001271875 | GAAATGCAGCTGTCCCTGCG | 1057 |
| CUL3 | NM_003590 | CGCTCAGATCTCGCGAGAAG | 1058 |
| CUL7 | NM_014780 | ATGGAAATAAATGACGTCCA | 1059 |
| CUTA | NM_015921 | ACTCAGTGAGTGACGCCAAG | 1060 |
| CWC22 | NM_020943 | ATTCGCCTTCTTCCTACCGT | 1061 |
| CWC22 | NM_020943 | TTGACTCTGGTATTATGATA | 1062 |
| CWC27 | NM_005869 | CCCTCCAAAACTATCAGTAA | 1063 |
| CX3CR1 | NM_001171172 | ATACTAAGTTTGAGAAGCTT | 1064 |
| CXCL14 | NM_004887 | ACCTGAAAGGGTTTTGGAGC | 1065 |
| CXCL3 | NM_002090 | CATTTTCTGCCCCAAATTCC | 1066 |
| CXCL8 | NM_000584 | AATACTGAAGCTCCACAATT | 1067 |
| CXCL9 | NM_002416 | AAACCCTAGTCTCAGATCCA | 1068 |
| CXCR1 | NM_000634 | AGAGTGGAGAATTCAGATAA | 1069 |
| CXorf23 | NM_198279 | TCATTTCCATGTTAGAGATG | 1070 |
| CXorf49B | NM_001145139 | CAGGCACCTCGCCCCACAAA | 1071 |
| CXorf49B | NM_001145139 | CTCCATGCCCGTCATTTGAC | 1072 |
| CXorf56 | NM_001170570 | AGTCACTTCTCAATGAAGAT | 1073 |
| CXorf66 | NM_001013403 | CAGAAGCTTATGCTTCCCTA | 1074 |
| CYB561A3 | NM_001161452 | TCTCCCCTCACAGGACCAGA | 1075 |
| CYB561A3 | NM_001161454 | TCACCTCCAAACTCCAACGT | 1076 |
| CYB5R3 | NM_001171660 | ATTTCCTGTGAATGTAACTT | 1077 |
| CYC1 | NM_001916 | GGCAACAGAGAGACGCGACG | 1078 |
| CYFIP1 | NM_001287810 | ACCCAGGCCGGCAGGTAGCC | 1079 |
| CYFIP1 | NM_001033028 | TTCATTCTGTGTTTCTTGAT | 1080 |
| CYLC1 | NM_021118 | ACTTGAAGATGTCTTATTCT | 1081 |
| CYP11A1 | NM_001099773 | ATGTCACTGCACTCCCGCCC | 1082 |
| CYP11A1 | NM_001099773 | CAGGACACTCGCCCGAACCC | 1083 |
| CYP20A1 | NM_177538 | CACTGTAGCCTCTGCCTCCC | 1084 |
| CYP21A2 | NM_001128590 | TGGATGCAGGAAAAAGGTCA | 1085 |
| CYP2C9 | NM_000771 | TGGGTCAAAGTCCTTTCAGA | 1086 |
| CYP3A5 | NM_000777 | AAAGCTTAATCAGTGTTATC | 1087 |
| CYP4A22 | NM_001010969 | TGATCCACCTAGGGGAACAG | 1088 |
| CYP4F2 | NM_001082 | CTGATTCCTCTGCACCCAGC | 1089 |
| CYP4F8 | NM_007253 | AATTGGTTCTTCTACAGTTA | 1090 |
| DAAM2 | NM_001201427 | GGTTACTCTGAATTTTCCCT | 1091 |
| DAB2 | NM_001244871 | ACTCCTGACTTTTCTGACAA | 1092 |
| DAB2IP | NM_138709 | ACGGTTGCCCCCATCTGCCT | 1093 |
| DAG1 | NM_001177643 | AAAAATAAAATTGGCCAAGC | 1094 |
| DAO | NM_001917 | TGGCTGATCTCAAGCCCCTG | 1095 |
| DAOA | NM_001161812 | ATGTGTGTGTGAGTAGTCAT | 1096 |
| DAOA | NM_001161814 | TTGTATATCTGTGTGAACTA | 1097 |
| DAPK1 | NM_001288731 | TTCTCATATCCATACTGTCT | 1098 |
| DARS | NM_001349 | AAGAGAGCTGGCATTCGCCC | 1099 |
| DAW1 | NM_178821 | GGAGGTGTCTAGAGTGAAAG | 1100 |
| DCAF1 | NM_001171904 | GAAGAGAACGCCTGCACGAT | 1101 |
| DCAF10 | NM_024345 | CCTGATCTGGGTGGCAGAGT | 1102 |
| DCAF11 | NM_025230 | CTGTCTCTGATTCAGGAAGC | 1103 |
| DCAF11 | NM_181357 | ATCAGAGCGCCCCCTTACAA | 1104 |
| DCAF11 | NM_181357 | CTTCCGAGAGGGATTTCGAT | 1105 |
| DCAF15 | NM_138353 | GACAGGCATAGCGCGAGTGC | 1106 |
| DCAF5 | NM_001284206 | GCTGGCCGGAAGAACGCGGG | 1107 |
| DCAF7 | NM_005828 | AGGCGCTTTGGCAGCCCCAA | 1108 |
| DCAF7 | NM_005828 | TACTCGCCCCGCCCAACTCT | 1109 |
| DCAKD | NM_001128631 | CCCGCCCGCCCAACCTCTCC | 1110 |
| DCANP1 | NM_130848 | GCACTGATTGAATGCTTTAC | 1111 |
| DCBLD1 | NM_173674 | CGTTCCCAGGCAGTGACCGA | 1112 |
| DCC | NM_005215 | GGCAAAGATTCCACGGGAAG | 1113 |
| DCLK3 | NM_033403 | AGCAGTATGCGAAGAGGTTA | 1114 |
| DCLRE1A | NM_001271816 | CAACATGGAATAAGGCCTTA | 1115 |
| DCLRE1B | NM_022836 | ACTTCCGCAGAAAGCAAGAT | 1116 |
| DCN | NM_133503 | AAAAAATCAGACTGATTGCT | 1117 |
| DCP1A | NM_001290204 | AACGACTGGGTCCTGGGATC | 1118 |
| DCTN1 | NM_023019 | GTGGGCAAGGGAGGGAAGAG | 1119 |
| DCTN4 | NM_001135643 | CCACTGCCCTTACTGCCATT | 1120 |
| DCUN1D1 | NM_020640 | GGAGGCAGCCCCGGACCTCG | 1121 |
| DCUN1D5 | NM_032299 | CCGTCGACTGCGGCAGTCCG | 1122 |
| DCX | NM_001195553 | AGGTTTCATTTATAACCAAC | 1123 |
| DDA1 | NM_024050 | CAACCGAACTTGACCACAAT | 1124 |
| DDAH1 | NM_001134445 | TGGAGGTTGGGGATGGGGGA | 1125 |
| DDC | NM_001082971 | GGGCTCCAAACTTGAAATCA | 1126 |
| DDI1 | NM_001001711 | AGGATCTTATCCTGTCACCC | 1127 |
| DDI2 | NM_032341 | GGAAGCCAGGAGAGGATAGG | 1128 |
| DDN | NM_015086 | ATATATAGTTCCCAGTCCCC | 1129 |
| DDR1 | NM_001202521 | TAAGGGTTTAGGCCAGTGTC | 1130 |
| DDR2 | NM_006182 | AGACTATTTCTTTTGACCCA | 1131 |
| DDR2 | NM_006182 | AGCTTTGCCCATAGTCCCTT | 1132 |
| DDX1 | NM_004939 | GCCTTGGTGTGTGAATGACC | 1133 |
| DDX18 | NM_006773 | AAAATCTTTGCAGCGCCCCC | 1134 |
| DDX27 | NM_017895 | GTGGCAGTATTTGAGGAGGG | 1135 |
| DDX3X | NM_001193417 | TGGCCGGACACCTTCCTGCG | 1136 |
| DDX50 | NM_024045 | ACCCTGGCCAATCTCCATAA | 1137 |
| DDX53 | NM_182699 | TTGATGGCCTGACCAATCAC | 1138 |
| DDX54 | NM_001111322 | AGAGGACCCTCTCCATGTTT | 1139 |
| DECR2 | NM_020664 | TCCCAGCAGGCCGCGGGCGG | 1140 |
| DEFA1B | NM_001042500 | GGCTGACCAAGGTAGATGAG | 1141 |
| DEFA4 | NM_001925 | ATCAGGTGTCCTAATTTTTC | 1142 |
| DEFA6 | NM_001926 | TGTTTATTGAGTGTCTGTTC | 1143 |
| DEFB103B | NM_018661 | ATGAGCAAGTATGCCCCCTT | 1144 |
| DEFB106A | NM_152251 | GCTCATCATATTTCTGATTC | 1145 |
| DEFB108B | NM_001002035 | GAGTCTTTGTGTACCTCATT | 1146 |
| DEFB112 | NM_001037498 | TTCACCTCCTTGTCCCCTTT | 1147 |
| DEFB119 | NM_153289 | AATTCCTTTGTGGGTCTCAC | 1148 |
| DEFB129 | NM_080831 | AAATTCCTTGCTCTTGATCC | 1149 |
| DEFB136 | NM_001033018 | ACAGGGTTCTGCAGAATTCG | 1150 |
| DEFB136 | NM_001033018 | GAGGTAGCACTGAAAGGCCA | 1151 |
| DEFB4A | NM_004942 | GCAAGATAGGAGGAATTTTC | 1152 |
| DEFB4B | NM_001205266 | TTAGAATTCAGCCACTTACC | 1153 |
| DENND1A | NM_020946 | GTCCTCCGGGGCCCGCGCCC | 1154 |
| DENND1B | NM_001195215 | AGCGCTCCCCCTGCACCCTC | 1155 |
| DENND1B | NM_001195215 | TTTCTGGCTAGGTGGCAAAG | 1156 |
| DENND1C | NM_001290331 | CTGGTTCCCCCCATCGTGCC | 1157 |
| DEPDC5 | NM_001242897 | GTCGTGTGCGGCCTCTTCCT | 1158 |
| DEXI | NM_014015 | CGCCCCCTGCACGCGCTAAT | 1159 |
| DGAT2 | NM_032564 | AGCTCTGAGCCCTGCTTCCA | 1160 |
| DGKA | NM_201445 | AGAAAATGTGTCCAAAGCCC | 1161 |
| DGKH | NM_152910 | GAGCCGGGTGGACCCCTGCC | 1162 |
| DGKZ | NM_201533 | AATGGAGAGGAAAACCAGAC | 1163 |
| DGUOK | NM_080918 | TGCGAGTGGTTTTTGTTCAT | 1164 |
| DHDDS | NM_001243565 | CCCGCTCGGTCACGTGAGCC | 1165 |
| DHDH | NM_014475 | GTAGAAGCGACGTCAAGGTG | 1166 |
| DHFR2 | NM_001195643 | AATCTCAGCCCTCCAAGAGC | 1167 |
| DHFR2 | NM_176815 | ATGCTGACCCAGGTGAGACC | 1168 |
| DHRS11 | NM_024308 | GGCAGCGCTCACTGGGGAAG | 1169 |
| DHRS7C | NM_001105571 | CCTCCAAGCTGAACACCCAG | 1170 |
| DHX30 | NM_138615 | CGTCAAGTTGCTGCCTTTCT | 1171 |
| DIABLO | NM_001278302 | GAGGGCAGTTTGGGTTGAGA | 1172 |
| DIAPH3 | NM_001258368 | CGTCAGATTTGGAGAAGCGC | 1173 |
| DIDO1 | NM_001193370 | CGTCTTTCATACCTGCACTC | 1174 |
| DIDO1 | NM_022105 | CGCTCTCTTGCTGTCGCGAG | 1175 |
| DIO1 | NM_213593 | AGACCTTTGTGCACCTGGTT | 1176 |
| DIO2 | NM_013989 | GCCCATCAATTCATTCAATT | 1177 |
| DIO3 | NM_001362 | GGGGACCGGGAGCCCGACCA | 1178 |
| DIRAS1 | NM_145173 | TGGGAGAGGTCGCCAGGATC | 1179 |
| DISC1 | NM_001164538 | GGACTCGCTGAGGAGAAGAA | 1180 |
| DIXDC1 | NM_001037954 | TACACACACACACACTCACA | 1181 |
| DKK1 | NM_012242 | GGCGGGGTGAAGAGTGTCAA | 1182 |
| DKK2 | NM_014421 | CACTCTTGAATTGGGGGCGG | 1183 |
| DLG1 | NM_001290983 | ATACCTCTGAGTAGCTGTTA | 1184 |
| DLG4 | NM_001128827 | GCTGGCAGGAACCCGGATAA | 1185 |
| DLG5 | NM_004747 | GCGCTCCGGAGCCCGGGAGG | 1186 |
| DLGAP1 | NM_001242763 | AAGCTCTGCTTCTCTCTTTG | 1187 |
| DLGAP1 | NM_001242763 | TTTCTATAGAATCATGGCAA | 1188 |
| DLGAP1 | NM_001242764 | CAGCCGTAGAAACAGGAAAA | 1189 |
| DLGAP1 | NM_001242764 | TAAAATCTTGCTCTTCTGAA | 1190 |
| DLGAP3 | NM_001080418 | AGGCATCCTTGTATCCCTTT | 1191 |
| DLK1 | NM_003836 | GTGCACCCGTGTGCGCGAGC | 1192 |
| DLL4 | NM_019074 | CGCCCGACTGGCTGACGGGG | 1193 |
| DLX1 | NM_178120 | CCCGGCGCGCTCTGTTGCAG | 1194 |
| DLX5 | NM_005221 | TACTGTTGCTCCCGAGGCCC | 1195 |
| DLX6 | NM_005222 | GAGCTAAGGTGGCTGCAGAG | 1196 |
| DMBT1 | NM_004406 | AAAATTTCCAACTTCCCTCT | 1197 |
| DMC1 | NM_007068 | ACCGAAGGGCGGGGAACGAG | 1198 |
| DMGDH | NM_013391 | AACTCACCTTCTTGGCCCCC | 1199 |
| DMRTC1B | NM_001080851 | GACCGCTGCCACAACCATTT | 1200 |
| DMXL1 | NM_005509 | CTGGCCGGTGAGTCGGCCCC | 1201 |
| DMXL1 | NM_005509 | TCCCCTCACCGGCCACGACC | 1202 |
| DNAAF1 | NM_178452 | GGGGCGCGGTACCTGCAGGC | 1203 |
| DNAI2 | NM_023036 | TTAGTATGTTACCAACCTAT | 1204 |
| DNAJB2 | NM_006736 | AAAGTGACAGAGGAACCTGG | 1205 |
| DNAJB5 | NM_001135005 | GATTGGGTTCTGTGGGGCGG | 1206 |
| DNAJB7 | NM_145174 | GTTTCCCCTGTATGTTTCCC | 1207 |
| DNAJC15 | NM_013238 | GCCTCTTTAATTTCTCTCCC | 1208 |
| DNAJC19 | NM_001190233 | AGGCGTGCAGGTGTTGGCCG | 1209 |
| DNAJC22 | NM_024902 | ACGCCTTCATTTCAATGTCC | 1210 |
| DNAJC24 | NM_181706 | TTCACAGTTTGGGAACTTAC | 1211 |
| DNALI1 | NM_003462 | CCGGTTCGTCCCTGTACTCT | 1212 |
| DND1 | NM_194249 | AGTGGATACCTCCACCCCCC | 1213 |
| DNM1 | NM_004408 | GTCGTAGTTTTCACCTTCTG | 1214 |
| DNMT1 | NM_001130823 | AATGAATGAATGAATGCCTC | 1215 |
| DNMT3L | NM_175867 | TTCAGGGCAAGGGTGAAGAA | 1216 |
| DNTT | NM_001017520 | AATGTACTGAGGCCCTTCTG | 1217 |
| DOC2A | NM_001282062 | GACTTTCACTCTTGTTGCCC | 1218 |
| DOCK6 | NM_020812 | GCCCGCCCAGCCTGGATCCC | 1219 |
| DOCK9 | NM_001130050 | ACAGCGTGGGCCAAATCAAT | 1220 |
| DOCK9 | NM_001130050 | ACTGCCTCTCTGATAAAGAC | 1221 |
| DOK1 | NM_001381 | GAGGCCAGGCCTCTGCGGTC | 1222 |
| DOLPP1 | NM_020438 | CCCACGGCCTGCACGCTGAA | 1223 |
| DOPEY1 | NM_001199942 | CGGCCATGGCTACCAATTTC | 1224 |
| DOT1L | NM_032482 | CCTCTTTGTAGTCACAGGCC | 1225 |
| DPCR1 | NM_080870 | GCGTCATGGAGCCAGGCACC | 1226 |
| DPH5 | NM_015958 | AGTCGGCCGAGAGGAGTCCG | 1227 |
| DPH7 | NM_138778 | AATCCGCTCCTCCACAAAGC | 1228 |
| DPM2 | NM_003863 | CTCACCCATCCGGTCTCACT | 1229 |
| DPPA3 | NM_199286 | GGGTGTAGTTTAGACTCATA | 1230 |
| DPRX | NM_001012728 | AGCGGAGACCAACGACTCAA | 1231 |
| DPYSL2 | NM_001386 | CCTGGGCCACGCGGGGACAA | 1232 |
| DPYSL4 | NM_006426 | CAGCGGTTCCAGCGCTGGGG | 1233 |
| DRC1 | NM_145038 | AGACCTGACATCCCACGGGC | 1234 |
| DRD3 | NM_001282563 | AATTTCCAACACACAAACTT | 1235 |
| DRD3 | NM_033663 | ATTGCCTTTCCAGATTTTGG | 1236 |
| DRG2 | NM_001388 | GGCCATGCTGTACTGGCCCA | 1237 |
| DSC3 | NM_024423 | GGCGTGGGAGAACTGGCAGA | 1238 |
| DST | NM_001144770 | ACTTGAAGCGGAAAGGAGTT | 1239 |
| DTHD1 | NM_001136536 | ACAGAATACATTAATCACTG | 1240 |
| DTNA | NM_001198944 | GGTTCATACTTTTGTTTTCT | 1241 |
| DTNB | NM_001256308 | ACCCCTATGCTGAGTTTTGA | 1242 |
| DTNB | NM_001256308 | TATGCTCCAGGCACTATTCT | 1243 |
| DTNB | NM_021907 | GCGGGAAGCTGGCTCCATCC | 1244 |
| DTWD1 | NM_001144955 | GGTGTCGCACTTCTCCCGAG | 1245 |
| DTWD2 | NM_173666 | GGAGGTCCCACCCTGCCGCT | 1246 |
| DTX1 | NM_004416 | CGAGAAGCCCCACTGAAGCC | 1247 |
| DUOXA1 | NM_001276264 | GGCCCGGCTCGGCTCAGCCA | 1248 |
| DUOXA1 | NM_001276266 | CTAAAAGATGGGGAGATGGA | 1249 |
| DUOXA1 | NM_001276267 | GCAGAGGCACCGGACGAGAG | 1250 |
| DUXA | NM_001012729 | AAATATCAATTGACGGAAAG | 1251 |
| DXO | NM_005510 | GAAGAGGCATCACCTGATCC | 1252 |
| DYNC1H1 | NM_001376 | ACTCGCAGTGCGGAGGCTGC | 1253 |
| DYNC1LI1 | NM_016141 | GGGCTTCAGTTGCAGCATAG | 1254 |
| DYNC2LI1 | NM_016008 | TAACAAGGAGTTACTAACTT | 1255 |
| DYNLL1 | NM_003746 | AGACCACAATGCACCGCTCA | 1256 |
| DYNLRB2 | NM_130897 | cCCGGGAGGGAAGAGGGAAG | 1257 |
| DYRK1A | NM_001396 | AAGTAAATGGTGGAATATTC | 1258 |
| DYRK1A | NM_130436 | ACACTAGACCTACAACTAGC | 1259 |
| DYRK2 | NM_006482 | GCCGGGCGGGAGGTTGGGTG | 1260 |
| DYSF | NM_001130455 | CGCCGCGGGCAGGGCGGATC | 1261 |
| DYX1C1 | NM_001033560 | AGACTCTCACTCTGTCGCCC | 1262 |
| DZANK1 | NM_001099407 | CTTGGCCACCTCCCGCCGAA | 1263 |
| DZIP1L | NM_001170538 | GTCATCTCTGTTGAGGTCTC | 1264 |
| E2F4 | NM_001950 | GGAGGCTGGACATTTGCTAC | 1265 |
| EBF2 | NM_022659 | TTTTACAACTGATCCTGTTG | 1266 |
| EBNA1BP2 | NM_001159936 | GGGAGGAGCAAAGGGCGGGG | 1267 |
| ECH1 | NM_001398 | AAAGGGTCCATTTCTGAGCC | 1268 |
| ECHDC2 | NM_018281 | CCCAGCTCCTCTGTGTGATT | 1269 |
| ECI2 | NM_001166010 | CGCCATCGCCATCCCTTGGG | 1270 |
| ECT2 | NM_001258316 | GCCACCTCCTGGCCACATCC | 1271 |
| ECT2 | NM_001258316 | GGAGTTTGCAGAGAAGTGCC | 1272 |
| EDA2R | NM_001199687 | AAGAACAGTGACCCAGCCAC | 1273 |
| EDAR | NM_022336 | CCCCCCACTGAGATGGCTAC | 1274 |
| EDN1 | NM_001955 | ACGCCCGCCGTCTGACAATT | 1275 |
| EDN3 | NM_207034 | TGGATGGGGGGCTGCTACTC | 1276 |
| EEF1E1 | NM_001135650 | GGAGCTAGTTACTGGTAGAA | 1277 |
| EEF2 | NM_001961 | CCCCCGCCCGTTAACCCATT | 1278 |
| EFCAB12 | NM_207307 | ATCCACGCCCCGCCCAGTTC | 1279 |
| EFCAB12 | NM_207307 | CACTGGATTCAGGGACTACT | 1280 |
| EFCAB7 | NM_032437 | AGCGCGCGCTTTTCATGCCT | 1281 |
| EFCAB7 | NM_032437 | GCTGGGTTCGTTTTATTCAG | 1282 |
| EFNA5 | NM_001962 | CGCGCTGCAGCCGCCCGGCC | 1283 |
| EFS | NM_005864 | TTCCAGGGGTGCCTGCGTGC | 1284 |
| EGFL6 | NM_015507 | TCAACTAAATTCTTAAGTCC | 1285 |
| EGFR | NM_201284 | GACCCAAGGCCAGCGGCCGC | 1286 |
| EGR2 | NM_000399 | CTGATTTGCATACACGGGCT | 1287 |
| EHBP1 | NM_001142615 | GGCAGAGGTGGTCTGTGACC | 1288 |
| EHD1 | NM_006795 | GAAGGCGAGGAGCGGGCGTT | 1289 |
| EHD2 | NM_014601 | AATAGTAACAATAACAGGTC | 1290 |
| EHHADH | NM_001966 | TGGAAAACAGCTGTAATTGC | 1291 |
| EI24 | NM_001290135 | CGGGATCGGCGAGGAGGCGA | 1292 |
| EIF2AK4 | NM_001013703 | TCCGCGCCGGGAGCTAGCTC | 1293 |
| EIF3D | NM_003753 | CGAGACGCGAGAGGTGTGAT | 1294 |
| EIF3L | NM_001242923 | TCAGGCTGGTCTCAAACCCC | 1295 |
| EIF4E3 | NM_001134650 | GTAAAGGAGGAGACTGAGTT | 1296 |
| EIF4E3 | NM_173359 | GAGCAGGAAGAGCAGCGTGA | 1297 |
| EIF4EBP1 | NM_004095 | AGCAGACGGGAGTGGGTCGG | 1298 |
| EIF4G3 | NM_001198803 | TGGATTGAAAATCACGAACT | 1299 |
| EIF4G3 | NM_003760 | ATCCGTTGGTGCTCTTAATT | 1300 |
| EIF5 | NM_183004 | GGGAGGGGGCGAGGCCGGGC | 1301 |
| EIF5AL1 | NM_001099692 | ACCATGAATCAAGTAGTGTG | 1302 |
| ELAVL2 | NM_001171197 | CTGCAGCTTCGAGTCACAGC | 1303 |
| ELF3 | NM_001114309 | CACTTGGCCCGGATCTTAGC | 1304 |
| ELF5 | NM_001243081 | CCAATTAAGCATCTACACAT | 1305 |
| ELMOD2 | NM_153702 | TCTCCAGCGTTAGCAATAGG | 1306 |
| ELOF1 | NM_032377 | CTCAAATAGCAGCGCTCCGA | 1307 |
| ELOVL3 | NM_152310 | GGCGGGGTGTGCGAAACGCC | 1308 |
| ELOVL4 | NM_022726 | GAGGCGACTTGTGCGGGGAG | 1309 |
| ELOVL7 | NM_024930 | GGAGGAGCCGGGGCGGCGCG | 1310 |
| EMC1 | NM_015047 | GGCAGGCTGCAGTGCACATT | 1311 |
| EMC6 | NM_031298 | TTAACAAAGGCCGCCCCGCT | 1312 |
| EMCN | NM_016242 | CCTATGATCCATTCTCAAGA | 1313 |
| EMCN | NM_016242 | TTTGTTCTTCTTCAACAGAA | 1314 |
| EME2 | NM_001257370 | GGTGCGTCCGCGGCTGATCG | 1315 |
| EML4 | NM_001145076 | CGTCACGTGGGAGGCGGAGT | 1316 |
| EML6 | NM_001039753 | CGGCGGCGGCTTGTCTGCGG | 1317 |
| ENOPH1 | NM_021204 | AGAGCGCGCCCTCCGCAGAC | 1318 |
| ENPP1 | NM_006208 | GCCAAGGATCTGACCGCGAG | 1319 |
| ENPP3 | NM_005021 | AGTCTGAAATTTCTGTGACA | 1320 |
| ENPP3 | NM_005021 | GTGACAAGGCTTTTTGTTCG | 1321 |
| ENPP4 | NM_014936 | GGTTAGACAGGTGCTTGGAG | 1322 |
| ENSA | NM_207047 | AGTACTGTACTCTTCCTGAT | 1323 |
| ENSA | NM_207047 | TGCTTTGGCGCTGGTTAGTT | 1324 |
| ENTPD7 | NM_020354 | TGACCGAGCTGGTTCGCCCC | 1325 |
| ENTPD8 | NM_198585 | CTCCTGCCTCCCACCCCCCC | 1326 |
| EOMES | NM_005442 | AAAAAGGAAAAGAAAGTCAC | 1327 |
| EOMES | NM_005442 | GAGGTGACACTAATTCAATT | 1328 |
| EP300 | NM_001429 | GCCGCCGCACCGGCCCCTAA | 1329 |
| EPB41L2 | NM_001135554 | GAAAGACGTCCTCCACCCCC | 1330 |
| EPB41L5 | NM_020909 | CCGAAACCCAGTTCCCGCTG | 1331 |
| EPCAM | NM_002354 | TGCTGAGACTTCCTTTTAAC | 1332 |
| EPHA10 | NM_001099439 | TCCTGCAGATCTCCAAACCG | 1333 |
| EPHA5 | NM_004439 | TCGACGAAGTCACACACCCA | 1334 |
| EPHB6 | NM_001280795 | GGGGCAGTGAAGCAGTGAAG | 1335 |
| EPHX1 | NM_001291163 | TAAGTAGCCCGTTTTATCCC | 1336 |
| EPM2A | NM_005670 | ACCAAGTCACTTACTCTAGC | 1337 |
| EPM2A | NM_005670 | TAGGGAGCGCTCCAGAGACC | 1338 |
| EPN2 | NM_001102664 | CGCGCAGGGGCCACTAGGGA | 1339 |
| EPRS | NM_004446 | CACGATAGCCATGATTACGT | 1340 |
| EPSTI1 | NM_033255 | TTGGTCGGCTACAGGTGAGA | 1341 |
| EPX | NM_000502 | GGAGTTCTGAAACTTCTCTC | 1342 |
| ERAP2 | NM_001130140 | GCTAAATCTGGGTACTGGAA | 1343 |
| ERBB2 | NM_001289936 | CTCCCAGGGCGACCGTGAGC | 1344 |
| ERBB2 | NM_004448 | GTCACCAGCCTCTGCATTTA | 1345 |
| ERBB3 | NM_001005915 | GCTCACCCTAATTTTTCTGC | 1346 |
| ERC1 | NM_178040 | GAGCGTGACGCGGCGGCCCG | 1347 |
| EREG | NM_001432 | CACTACTCTCAGGTGCTCCA | 1348 |
| ERGIC1 | NM_001031711 | ATGAGTACTGGAGTCTTTGG | 1349 |
| ERI1 | NM_153332 | CAAGGATCTAGTCCAGTCAC | 1350 |
| ERICH4 | NM_001130514 | GAAGGAAAAAGAAAAGCACA | 1351 |
| ERLIN2 | NM_001003790 | CCCGCCCCTCGCGCTCCCAG | 1352 |
| ERMAP | NM_018538 | GAGGAGGCTCCCAAAAATGA | 1353 |
| ERMARD | NM_001278533 | TGGGGCTCGACTTCACGCCT | 1354 |
| ERN2 | NM_033266 | CCTCTGTAATCCCAGCACTT | 1355 |
| ESAM | NM_138961 | CTTCCCCCTCTACTCGTACC | 1356 |
| ESAM | NM_138961 | TGATGCCCCACGAGCCAGCC | 1357 |
| ESCO1 | NM_052911 | GTTTTTCACCCCGGCCCGGA | 1358 |
| ESCO2 | NM_001017420 | AGAGATTTTTCACCTCACCA | 1359 |
| ESF1 | NM_016649 | CGCATGCGCACAAAAAGCGC | 1360 |
| ESPL1 | NM_012291 | CAGAGCAGCAAGACCCTCCG | 1361 |
| ESR2 | NM_001437 | AATCTGAGACTGGGGCTGCG | 1362 |
| ESRRA | NM_001282451 | CGGACGAGTCGGGGCGGAGC | 1363 |
| ESRRG | NM_001243507 | GTCATTGCACTGGCAGTTAG | 1364 |
| ESRRG | NM_001243511 | ACAGCCCTGAGTGTATGTGT | 1365 |
| ESRRG | NM_001243511 | TGTGCTTAACTCTATTGCCT | 1366 |
| ETFBKMT | NM_001135863 | TCATTAAGAGAAATACCAAG | 1367 |
| ETFBKMT | NM_173802 | TTAACGTTCCCTTATTTTCC | 1368 |
| ETV1 | NM_001163149 | GGTTACCCTGGATACCCGTC | 1369 |
| ETV2 | NM_014209 | GATGTCAATATTGCTATGAT | 1370 |
| ETV7 | NM_001207037 | GTGCAGGACCCACGCCTCCC | 1371 |
| EVA1A | NM_001135032 | AACTAACTTGGCGCGGAGGG | 1372 |
| EVA1A | NM_032181 | GGACAAAGGTGAGCAATTCT | 1373 |
| EVA1B | NM_018166 | ACAAGAGCGCAGGAGCTCGC | 1374 |
| EVI2A | NM_014210 | TGACAGTATGCTCATTCTAT | 1375 |
| EVI2B | NM_006495 | CTGTTTACTTGTATGACCTT | 1376 |
| EXD3 | NM_017820 | GGCTGCGGGGTCTCCGAGGC | 1377 |
| EXO1 | NM_130398 | CCGTCTCGCTGGGTAGACAG | 1378 |
| EXOC7 | NM_001145299 | ACCGACGGCCATTTTGAGCG | 1379 |
| EXOSC10 | NM_002685 | GGGAAGCCTGCGATTAGGTT | 1380 |
| EXOSC8 | NM_181503 | ACCAGTGAAGAGGCAAGGCC | 1381 |
| EZH1 | NM_001991 | GCTTCCAAAGCGGCGCTGGC | 1382 |
| F11 | NM_000128 | GCTGGGGGAGAGCGGACGGA | 1383 |
| F13B | NM_001994 | ATCAGTTATCATGCTCTTAC | 1384 |
| F2RL2 | NM_004101 | TGCTGTTCAACATCTGTTTT | 1385 |
| F2RL3 | NM_003950 | ATCTTGCTGGCCTGGCACCT | 1386 |
| F8A2 | NM_001007523 | ACCTCATCAGGGCAAGGGGC | 1387 |
| F8A3 | NM_001007524 | ACCTCACCAGGGCAAGGGGC | 1388 |
| FABP3 | NM_004102 | GCTAGCAGGGCGCCACTGGC | 1389 |
| FAF1 | NM_007051 | GAAGCTTCAAGTCTCGCAAC | 1390 |
| FAIM | NM_018147 | CACAGGTGAGGCAGCAGACC | 1391 |
| FAM104A | NM_032837 | CGAGCGCTTCTGCCACCCCA | 1392 |
| FAM107B | NM_001282700 | CCTCCTGAGGCTGGGATTCA | 1393 |
| FAM110A | NM_031424 | TCAGGTTGCCCAGGTCGCCC | 1394 |
| FAM120B | NM_001286380 | TTACTTCTTAAAGCTGTCTT | 1395 |
| FAM122B | NM_001166599 | ATGCCATCGAGGAAGGCGCC | 1396 |
| FAM122B | NM_001166600 | ATCAGCTTTCAGGAGGAGTT | 1397 |
| FAM124A | NM_001242312 | ACACCGCATGCACAGACGCA | 1398 |
| FAM129A | NM_052966 | GGGGCATCCAAGAAACACCT | 1399 |
| FAM129B | NM_001035534 | GCAGGAAACAAAGTCTAGCA | 1400 |
| FAM129C | NM_173544 | ATGTGCAGGAGCCCAGCACA | 1401 |
| FAM129C | NM_173544 | TAGACTCTCTGGTGCTTTCA | 1402 |
| FAM131C | NM_182623 | CCCCACCTCCTGGGGTTGCC | 1403 |
| FAM133B | NM_001288584 | GCGAGAACCCTCGCTGTTCC | 1404 |
| FAM133B | NM_152789 | ACTGCAGCGATCTCTGGAGC | 1405 |
| FAM135A | NM_001162529 | TGCAGTCCGCAGTCTGGCCT | 1406 |
| FAM13A | NM_001265580 | TTGGCTCTTGCTGCAGTTAT | 1407 |
| FAM13C | NM_001166698 | AGGTGCTCCTCGCTGGATCC | 1408 |
| FAM156A | NM_001242491 | TTCTCGCGACCCACGCCGCT | 1409 |
| FAM156B | NM_001099684 | TAAGTTTTTTGTTGAGATGG | 1410 |
| FAM159B | NM_001164442 | GGGACAGGGCAGGTGGATTC | 1411 |
| FAM162A | NM_014367 | CGGCGCCAGGGGCACTAGGC | 1412 |
| FAM162B | NM_001085480 | AGCCTGCCTCTGTTTGAAAC | 1413 |
| FAM162B | NM_001085480 | AGTAGAAATGTATTCCCGCC | 1414 |
| FAM170A | NM_182761 | GGGAGAGTTGAATTCATTAG | 1415 |
| FAM170A | NM_182761 | TTCTGCCACATTTGAAATAC | 1416 |
| FAM170B | NM_001164484 | ACAGAAAAGGAGTTCCCATG | 1417 |
| FAM171A1 | NM_001010924 | TCTTCGGGGAAACCCGGCGC | 1418 |
| FAM174B | NM_207446 | GGCCAGCCCAAGTGTCATCG | 1419 |
| FAM177A1 | NM_173607 | CTGGCCAACTGCAGTCTGGG | 1420 |
| FAM178B | NM_016490 | TTCATGGTGAAGTGCCCTGC | 1421 |
| FAM178B | NM_001122646 | GCATCCACGTGCGCGGGAAT | 1422 |
| FAM185A | NM_001145268 | GCCCTTTGTCTCAAGACCAT | 1423 |
| FAM186B | NM_032130 | TCACTGCAACCTCCACTTCC | 1424 |
| FAM189A2 | NM_001127608 | ATATTTCCTCGGAAGTTTGG | 1425 |
| FAM193B | NM_001190946 | GGTCACCACCCGGAGTTCGC | 1426 |
| FAM198B | NM_016613 | TTCTGAGTCTGTTTGCGAAC | 1427 |
| FAM199X | NM_207318 | AGGGATTCAGGCCGCTAGAA | 1428 |
| FAM209B | NM_001013646 | CGGGGTGCCAATTCCCTGCC | 1429 |
| FAM20A | NM_017565 | ATCCTCAGGAGAGACGCCCC | 1430 |
| FAM214A | NM_001286495 | TTACAAACTCAGCTGTGTTT | 1431 |
| FAM217B | NM_022106 | TACAAGGCTGCAACTTGACC | 1432 |
| FAM219B | NM_020447 | TTGGGTTGAAGAGTCATATG | 1433 |
| FAM21A | NM_001005751 | GTTGGGGCGGAGGAAGCTGG | 1434 |
| FAM220A | NM_001037163 | GTCTTACCTGCCAAAAAGAA | 1435 |
| FAM227A | NM_001013647 | CCTGACGCGTCCCAGAAGCC | 1436 |
| FAM228A | NM_001040710 | TCACCGTCCAGCTGGCGTCG | 1437 |
| FAM229A | NM_001167676 | CCGCCGCGTCTGTGTGGACC | 1438 |
| FAM234A | NM_032039 | GGCCTTGAAATACGGTGCCA | 1439 |
| FAM24B | NM_152644 | GCATTTGAAATGATGTAAGC | 1440 |
| FAM25C | NM_001137548 | GCTGGACAGGTGAGTCAGTG | 1441 |
| FAM3D | NM_138805 | CCCTAAGCCACTCCTCAGCC | 1442 |
| FAM43B | NM_207334 | GGGTTCCCGAATGCGCCAAG | 1443 |
| FAM46A | NM_017633 | GTCGTCCCGCACTAACTGCT | 1444 |
| FAM46D | NM_152630 | ACTTAAGTTCAAGTATCTTG | 1445 |
| FAM47C | NM_001013736 | TAGAATCTGGGCTGCGCAGG | 1446 |
| FAM49B | NM_001256763 | GTGGCCACCCCCTTGCACCC | 1447 |
| FAM50A | NM_004699 | CGAGGCAGCGCGAGGGGCTG | 1448 |
| FAM53B | NM_014661 | GGGCCACTTCCCGCGTCCCG | 1449 |
| FAM71C | NM_153364 | AGTAGTCCCTGCCTCAGAGC | 1450 |
| FAM72C | NM_001287385 | CGTAGGCACCGCCCCAGTAA | 1451 |
| FAM72C | NM_001287385 | CTGAGATCAATTCGGCTTTC | 1452 |
| FAM83E | NM_017708 | GGCTGCTGCAGGGAGCCATT | 1453 |
| FAM84B | NM_174911 | GCGGGTGGATTATTTACAGG | 1454 |
| FAM96B | NM_016062 | TGACCGCGGCCCTGGCTGCT | 1455 |
| FAM98A | NM_015475 | AACGCGCATGTGCAAAACTG | 1456 |
| FAN1 | NM_001146096 | GGGAAAGGAAGGAGGTGCCC | 1457 |
| FANCM | NM_020937 | CAAAACACCGGAACCGCACC | 1458 |
| FARP2 | NM_014808 | ATATAAATCTGTGCAGCGCT | 1459 |
| FBLIM1 | NM_001024216 | ACAGGACCCACCAGGGAACT | 1460 |
| FBN3 | NM_032447 | GGGGCAGCCCCGGGGCCTCT | 1461 |
| FBP2 | NM_003837 | TACAGACTGCTGCGGCTCCC | 1462 |
| FBXL19 | NM_001282351 | GCAGGCTACCTAGCCTCTCC | 1463 |
| FBXL19 | NM_001099784 | GGGAGCCATCTCTCCCTTCT | 1464 |
| FBXL22 | NM_203373 | CCAGGACCCAGACACATGTG | 1465 |
| FBXL5 | NM_001193534 | TCGTCTTCATAAGCCGCAGA | 1466 |
| FBXO17 | NM_024907 | ACATCCCCAAGACGCCCCCG | 1467 |
| FBXO17 | NM_024907 | CCCAGTTGCCGCGAGGCCAG | 1468 |
| FBXO17 | NM_024907 | GCTCTCCCAGGGGTGGGCCC | 1469 |
| FBXO18 | NM_001258453 | GGGGGCGCGGCCACAGCTAC | 1470 |
| FBXO31 | NM_024735 | CGGAGCTCTACGTAGGGGCG | 1471 |
| FBXO41 | NM_001080410 | GGGTATCGCTGCTCCCACCC | 1472 |
| FBXO45 | NM_001105573 | CGGCTCCGCCATGCGGGTTG | 1473 |
| FBXO47 | NM_001008777 | TCCCAGAAGCCCTAGCGGGA | 1474 |
| FBXW2 | NM_012164 | GGCCCTCACGGTGCTTAGGC | 1475 |
| FBXW8 | NM_153348 | GCACGTGGTGGTCCGGCTTG | 1476 |
| FCGBP | NM_003890 | GGCCAGGGGGTATGGATCCA | 1477 |
| FCGR2A | NM_021642 | AGAACAGTAACCCCTCCCCG | 1478 |
| FCGR2A | NM_021642 | TACTCTAAGGAGGGGTATAC | 1479 |
| FCGR2A | NM_201563 | GGCTACACCAGATTTATTCT | 1480 |
| FCGR3A | NM_001127592 | GGGTCTCACTGTCCCATTCT | 1481 |
| FCGR3B | NM_001271037 | TTTACTCCCTCCTGTCTAGT | 1482 |
| FCGRT | NM_001136019 | CGAGACCAGCCTGGCCAATA | 1483 |
| FCGRT | NM_001136019 | GGCCTGTGGTCCCAGCTACT | 1484 |
| FCRL6 | NM_001004310 | TAATACTTCTTCAACCAAAG | 1485 |
| FDCSP | NM_152997 | GTTTCTAGGAAACTAAACAT | 1486 |
| FDXACB1 | NM_138378 | AGATAGGAGATTTAAGCACC | 1487 |
| FDXACB1 | NM_138378 | TGAGCAGCAGAGACACTGGG | 1488 |
| FDXR | NM_001258012 | CGACGGTGGGGCGTAGTTAA | 1489 |
| FERD3L | NM_152898 | TTCCATAAGCTTCGAGAGAA | 1490 |
| FERMT2 | NM_006832 | AGGCCGGCCGGACCCGCTCA | 1491 |
| FEV | NM_017521 | GGAGAAGAGGAGGAGGGAGC | 1492 |
| FGB | NM_001184741 | AAGATACACATCTCTCTTTG | 1493 |
| FGD2 | NM_173558 | CCCTGTTGCCACCTCTTAGG | 1494 |
| FGD2 | NM_173558 | GTGAAAGGTCAGCCCCCCTG | 1495 |
| FGD3 | NM_001286993 | CCACAAGTTAGAAGGTGAAG | 1496 |
| FGD5 | NM_152536 | AGCCTAAGACAAAGCACGGG | 1497 |
| FGF1 | NM_001257209 | AAGCAGATAGCACTGGAACC | 1498 |
| FGF1 | NM_033137 | TGAGTAAGCACAGCCTGCCC | 1499 |
| FGF18 | NM_003862 | GATGTGGGCTGGGCGCACCC | 1500 |
| FGF2 | NM_002006 | GGCAGGGCTTTGGCATTCCC | 1501 |
| FGFBP3 | NM_152429 | GACCGCTTCCATCATCCATC | 1502 |
| FGFR1 | NM_001174066 | AGCCACGGCGGACTCTCCCG | 1503 |
| FGFR1 | NM_001174066 | CGGAACCTCCACGCCGAGCG | 1504 |
| FGFR4 | NM_213647 | GGGGGGGGGGCGTGGAAGGA | 1505 |
| FGFRL1 | NM_021923 | CCGCTGCGGCTTCCTCCGCC | 1506 |
| FGL1 | NM_147203 | CCAGGATCCTGTAACTGCAT | 1507 |
| FGL1 | NM_201552 | AAGCTAAAAGAGAAGATTCA | 1508 |
| FHL1 | NM_001159699 | ACCGGAATAAAATTTGGACT | 1509 |
| FHL1 | NM_001159699 | TACAGGGATGACTTTCTATG | 1510 |
| FHL1 | NM_001159700 | CACGGGGGTTGAGCCTTAGA | 1511 |
| FHL1 | NM_001167819 | GTGACTTGTGCTCTACATTC | 1512 |
| FHL2 | NM_001450 | TTTCGGACGAGGCCTGGGCG | 1513 |
| FIG4 | NM_014845 | ATTTATCTCCTCCCTCTCTT | 1514 |
| FILIP1 | NM_001289987 | AAAACCGGCAGGCCCTTTTA | 1515 |
| FILIP1 | NM_001289987 | GCTCACCCTGTAAAAGATTG | 1516 |
| FILIP1L | NM_001042459 | GAAACTTCCCAAGCACAACC | 1517 |
| FKBP10 | NM_021939 | ATGAACCTTGCTTCTTTCGC | 1518 |
| FKBP11 | NM_001143782 | ACTAGCTCCTGACACACAGT | 1519 |
| FKBP14 | NM_017946 | ACCAGCGTGGATTTTGGGAG | 1520 |
| FKBP2 | NM_004470 | CCACAGCACTCCTGTTTTCC | 1521 |
| FKBP5 | NM_001145775 | AGGAAGAGACTCTGAACTCT | 1522 |
| FKBP6 | NM_003602 | GACACGTAACGGGACCACGC | 1523 |
| FKBP9 | NM_001284343 | GAAAGCCTTAAAAGTAACCA | 1524 |
| FLNA | NM_001110556 | CTTAATTGGTAAAATTGCCC | 1525 |
| FLNC | NM_001127487 | GCGGGGCGTCCTGTGCGGCG | 1526 |
| FLRT1 | NM_013280 | GGTTCCGACTCCCTGTTCGT | 1527 |
| FMN1 | NM_001103184 | TTTCAGAAGAGCAGCCTCCC | 1528 |
| FMR1NB | NM_152578 | AGCAGAAGACGTCATCGTGA | 1529 |
| FNDC3A | NM_001079673 | GCGTTCCGGTGAGAGAGCCC | 1530 |
| FNDC7 | NM_001144937 | GTATAACACCGTTGGTCGCT | 1531 |
| FNDC8 | NM_017559 | AGTCACACTGGCCCTTGGTC | 1532 |
| FNTB | NM_002028 | CGAGATGGCGTAGGACGCCT | 1533 |
| FOXB2 | NM_001013735 | ACTTTGCCCTCTCGCCCTCC | 1534 |
| FOXB2 | NM_001013735 | CCAGGCAATTCGGAGAAGGC | 1535 |
| FOXD3 | NM_012183 | GCAGGTGGCTTGGGGCCCGC | 1536 |
| FOXD4 | NM_207305 | CCTTTGCACGGGTTCTGTTA | 1537 |
| FOXD4L6 | NM_001085476 | TGACAATATTCCCAGGCTTC | 1538 |
| FOXG1 | NM_005249 | ACTGCTGCTGCGAGAGGAGG | 1539 |
| FOXJ3 | NM_014947 | GAAGCGACCGTGACCGCGCA | 1540 |
| FOXJ3 | NM_014947 | GTAGTGCCCTGAGACTCCCG | 1541 |
| FOXK2 | NM_004514 | GGCAGTGGGGCTACCGAAGC | 1542 |
| FOXN1 | NM_003593 | TCTCTCATCAGATGGCTGAC | 1543 |
| FOXP1 | NM_001244816 | ACAGAAAGCCTGAGAGCTGC | 1544 |
| FOXR1 | NM_181721 | GCAAGGGGCTTGGGCAAACG | 1545 |
| FOXR2 | NM_198451 | TATTTCTGAGTCTTCCTTAA | 1546 |
| FOXRED2 | NM_001102371 | GGGCTAGCGCGCACCCGCGA | 1547 |
| FPGT- | NM_001112808 | CATCCAAGTTCTCCACATCA | 1548 |
| TNNI3K | | | |
| FREM1 | NM_001177704 | CAACTGCGGTGACCTCACAG | 1549 |
| FREM3 | NM_001168235 | CTCTTGCTGGATCCGCAAGT | 1550 |
| FRG2C | NM_001124759 | TGGGGACCTAGACACAGTTA | 1551 |
| FRMD3 | NM_001244961 | AGATCAGTTAGATTTTGCTG | 1552 |
| FRMD3 | NM_001244962 | AATGATGAGGCATTTGGACA | 1553 |
| FRMD4A | NM_018027 | AGGCAGCCCTGTGGAGAGAT | 1554 |
| FRMD6 | NM_001267047 | AATACACTTGGTACTATGGT | 1555 |
| FRRS1 | NM_001013660 | TCTCGCTCTGTCCGCCAGGC | 1556 |
| FSBP | NM_001256141 | AGCTTTATGTAGGTCAGGCT | 1557 |
| FSD2 | NM_001281805 | TTTGGACCTTCACTCATGGC | 1558 |
| FSHR | NM_181446 | ATAGAACCATTAGGCATGTC | 1559 |
| FSHR | NM_181446 | TTGCTGTGTGCCTTAGGTCA | 1560 |
| FUBP1 | NM_003902 | ACCTCCTCTCCGCGCGTTCT | 1561 |
| FUBP1 | NM_003902 | CGCGAGAACAGAATTTCTTT | 1562 |
| FUNDC1 | NM_173794 | GTCCGTTGCCTTCCGCAACT | 1563 |
| FURIN | NM_001289823 | AGGCGATCCCAAAGTCCTCG | 1564 |
| FUT2 | NM_000511 | ATGGACTTTGTGGCCGGCAA | 1565 |
| FUT4 | NM_002033 | GCCTTCAGAGTCTCTGCATT | 1566 |
| FUT6 | NM_000150 | GCACTGAGATAGTAGAACTC | 1567 |
| FXN | NM_001161706 | TACACAAGGCATCCGTCTCC | 1568 |
| FXYD5 | NM_001164605 | GGGACTTACGTCGGAGCTGG | 1569 |
| FYN | NM_153047 | GGCTATTTCAGGCCTATTAG | 1570 |
| FZD6 | NM_001164615 | AGCAGTTCAACTTCCTATTA | 1571 |
| G0S2 | NM_015714 | GTCCCACTCCAGGCGAGCGC | 1572 |
| GABARAPL2 | NM_007285 | CTTCTTCGCCACCGCAGCCC | 1573 |
| GABPB1 | NM_005254 | CCTACCCACCGCAGAACAGG | 1574 |
| GABRA1 | NM_001127644 | GTTCATTCATATGCAGGCAG | 1575 |
| GABRA1 | NM_000806 | AGGTCTTAGTAAGCGCTCCC | 1576 |
| GABRA4 | NM_001204266 | AAGGCAGGTTCCGCCTCCCC | 1577 |
| GABRA4 | NM_001204266 | GAGCGAGAAAGGAGGGGGCG | 1578 |
| GABRA6 | NM_000811 | ATAATAAACGCTGAGCCTAT | 1579 |
| GABRE | NM_004961 | CCATCGGGGCGGGCCTGGGG | 1580 |
| GABRG2 | NM_000816 | TTTAAATACACACACCCACA | 1581 |
| GADD45A | NM_001924 | TGGGGTCAAATTGCTGGAGC | 1582 |
| GAGE1 | NM_001040663 | AAGATGGGGTGAGTTTTGAG | 1583 |
| GAGE1 | NM_001040663 | AGGAAACAGCAGAGGGAGGT | 1584 |
| GAGE1 | NM_001040663 | CTCCATGCCCATCCTCATTG | 1585 |
| GAGE10 | NM_001098413 | AAGATGGAGTGAGTTTTGAG | 1586 |
| GAGE10 | NM_001098413 | GCATAGGAAACAGCAGAGGG | 1587 |
| GAL3ST1 | NM_004861 | CCAGTGGAGGCAGAAGGCCT | 1588 |
| GAL3ST2 | NM_022134 | GGTTTTAACTGTTCTGTTCT | 1589 |
| GAL3ST3 | NM_033036 | TGGTTCCCTGGCTTGCCCGC | 1590 |
| GALNT10 | NM_198321 | ACGCGGGGGCAGGCGGCGCG | 1591 |
| GALNT4 | NM_003774 | TAGGAGGCTCTTGGCCGGGC | 1592 |
| GALNTL6 | NM_001034845 | GTGGGAGCTCCCAGCCTGCG | 1593 |
| GALR2 | NM_003857 | GAGCAAGAGACAGGAGGGCG | 1594 |
| GALR3 | NM_003614 | GTGACACTCAGCGATGACTT | 1595 |
| GAN | NM_022041 | CCCGCCTGACCAGCTGCGGC | 1596 |
| GAPT | NM_152687 | TTAATACTTGCAAAGTTTCC | 1597 |
| GARNL3 | NM_001286779 | AGCGGCCAGTGATGCGGGCT | 1598 |
| GART | NM_001136005 | CGGTCTCTCGCCTTCCTGAT | 1599 |
| GAS7 | NM_001130831 | TTGGGGAAGAGAGAACTTGC | 1600 |
| GAS7 | NM_201432 | TGGGCCTGCCCAAGCCCTGC | 1601 |
| GAST | NM_000805 | AAAGGGCGGGGCAGGGTGAT | 1602 |
| GATA2 | NM_001145661 | AAATGCCACCTCTTGCCCGG | 1603 |
| GATB | NM_004564 | GGAGGTGTGACTCCTCCTAG | 1604 |
| GATC | NM_176818 | GTTCGCCGAGAAATTTCTCA | 1605 |
| GBA | NM_001005742 | CTTCCTCTTTAGAGAGCCTC | 1606 |
| GBA3 | NM_001277225 | TCTGGACTCCTGCCTTGCAC | 1607 |
| GBP3 | NM_018284 | TGTGAATTGTCTCCTGTTAT | 1608 |
| GBP7 | NM_207398 | CTGACAGCTGTGCTAGTGAG | 1609 |
| GC | NM_001204306 | TTAGCATCATTCCACCTTTC | 1610 |
| GC | NM_001204307 | TATGCAGTGTAAAAGCAGCT | 1611 |
| GCDH | NM_000159 | GTAGCCTTGCCTGTGGAAAT | 1612 |
| GCFC2 | NM_001201335 | TCAGTCCACGCAACCTAACC | 1613 |
| GCFC2 | NM_001201335 | TGCAAAGCATTCCCTTTGCC | 1614 |
| GCH1 | NM_000161 | ACGGCCCTCGCCGCGCCCCT | 1615 |
| GCNT1 | NM_001097634 | GTAATTCCAGTGGGTAGCAA | 1616 |
| GCNT1 | NM_001097634 | GTTCCATAAGTAATTCCAGT | 1617 |
| GCNT2 | NM_145655 | GAAACTCGGCTCCAGTGAAA | 1618 |
| GCOM1 | NM_001285900 | ATGGGCGTCCAGGCTGTCCA | 1619 |
| GCSAML | NM_001281834 | TCGTTTCTTGTTCAGCAAAA | 1620 |
| GDF11 | NM_005811 | GGCCAGGCCCTTTATAGCCC | 1621 |
| GDF6 | NM_001001557 | CACCTCCGGCCCGCACCACC | 1622 |
| GDF6 | NM_001001557 | GGAGAGGGGCCGCGGTGCGC | 1623 |
| GDF7 | NM_182828 | AGGGAGGGCGAGGAGCTGAA | 1624 |
| GDF9 | NM_001288828 | AGCTGAGCCCTGTGCGTGAG | 1625 |
| GDPD1 | NM_182569 | AGGTGACAAACGCTCAGTCC | 1626 |
| GFIl | NM_005263 | CCTGGCTTGCCCCGGCAGGG | 1627 |
| GFI1B | NM_004188 | CATTTCTAACCCTCGACACT | 1628 |
| GFM2 | NM_032380 | CTTCACATTCGAGACACAGA | 1629 |
| GFOD1 | NM_001242628 | GGCATCTGATCTTCCTAGTT | 1630 |
| GFRA1 | NM_005264 | AAACTTTGTGTTCCGAAGAA | 1631 |
| GFY | NM_001195256 | GCAAGTCCCTTGGAGGCTTG | 1632 |
| GGA3 | NM_001172704 | GGAATATTATCGCAAGCCAG | 1633 |
| GGA3 | NM_001291642 | TGCGTTTCTCTCCACTGATC | 1634 |
| GGPS1 | NM_001037277 | GGTCGTCTAAGAGGCCATCC | 1635 |
| GGT6 | NM_153338 | GCATGTGAGCCTGCCCCATT | 1636 |
| GH2 | NM_022556 | AGGGTCACGTGGGTGCCCTC | 1637 |
| GHITM | NM_014394 | TCCCTGCAACAATCCTCAAC | 1638 |
| GHR | NM_001242462 | TAGGACAATATGAGACTCTG | 1639 |
| GHRL | NM_001134946 | ACGGAACAGAGGAGAGATGC | 1640 |
| GIN1 | NM_017676 | TCCTGAGGTGTAGTAGCCTG | 1641 |
| GJA9 | NM_030772 | AAGTGTTCAATAGCTACATT | 1642 |
| GJB1 | NM_000166 | CTATGGGGCGGGTGCGGCGA | 1643 |
| GJB1 | NM_000166 | TGTAGGGTGGGCGGAAGTCA | 1644 |
| GJB3 | NM_001005752 | TTTCCTTCCCAAGTCTAGGC | 1645 |
| GJC2 | NM_020435 | AGGCAGGCAGGGTGCCCGGC | 1646 |
| GK5 | NM_001039547 | GGAGTCTCACTCTGTCGCCC | 1647 |
| GKN1 | NM_019617 | CTTAGCAAGGAACTTTCACA | 1648 |
| GLB1L | NM_001286427 | CCAGCTTCATCGACATCACC | 1649 |
| GLB1L | NM_024506 | CTGCCGGACTGACCTGGCTC | 1650 |
| GLG1 | NM_001145666 | CTTACCCGGGGGGGTTGCTG | 1651 |
| GLIPR2 | NM_001287013 | CTCCTTATAAGGCGGGGGCC | 1652 |
| GLIPR2 | NM_001287013 | GAGGCCCACGGGGTGGCCCC | 1653 |
| GLIPR2 | NM_022343 | TCGCGGCACGAGGGGCGTTC | 1654 |
| GLIS1 | NM_147193 | TCTGGACAAATGGAATCATG | 1655 |
| GLP2R | NM_004246 | AGGCGGTCTAGAGCAATCTA | 1656 |
| GLRA1 | NM_000171 | TCGCCCAATCCAACGGTCCG | 1657 |
| GLRX5 | NM_016417 | GTTGCCGACGACCAATAGTA | 1658 |
| GLT8D1 | NM_001278280 | GCGAGGGCGACCGAGACTTA | 1659 |
| GLYAT | NM_201648 | ACAATGCTTTTTGTCCTCAC | 1660 |
| GNA12 | NM_001282440 | CAGCACTCCTCCCACGGGCC | 1661 |
| GNA12 | NM_007353 | GGCGAGATGAGCCAATCGAA | 1662 |
| GNA15 | NM_002068 | CCTGATTGGCTCCGAGGAGG | 1663 |
| GNAI2 | NM_001282620 | GCCTGACCTTGGGGGAAGCC | 1664 |
| GNB5 | NM_006578 | TCTCTCCTCCGGGAGAGGCA | 1665 |
| GNE | NM_001190388 | GGAATGGGAAATCCAAAACA | 1666 |
| GNG10 | NM_001198664 | CGGGTCCCCGCCTCGGTTCC | 1667 |
| GNG11 | NM_004126 | AAAACTCTTTGAGAGGTGAA | 1668 |
| GNG7 | NM_052847 | CAGGGTGACTTCGTGACGTC | 1669 |
| GNGT1 | NM_021955 | TTGGAATTGAAAGTAAGGAT | 1670 |
| GNPAT | NM_014236 | CACCAAAGTCGTAAAGGTTC | 1671 |
| GNRH1 | NM_001083111 | ACGTCCACGGTTGCACCTCT | 1672 |
| GOLGA3 | NM_005895 | GCCGCCCGGCCCGGATGCTC | 1673 |
| GOLGA6D | NM_001145224 | GGCAGGGACAGCAGTCGCAT | 1674 |
| GOLGA6L22 | NM_001271664 | AGCTTTCCTTGTGACAACAC | 1675 |
| GOLGA6L4 | NM_001267536 | AGCTTTCCTTATGATGCCAC | 1676 |
| GOLGA8K | NM_001282493 | CAGCTTTCCTTGTGAGCCAC | 1677 |
| GOLGA8M | NM_001282468 | GGTGCGGAGAGCGGTCGCAT | 1678 |
| GOLGA8N | NM_001282494 | AGCTTTCCTTGTGAGCCACA | 1679 |
| GORASP1 | NM_031899 | GCAGAATGGTTTTAAGGCGA | 1680 |
| GP5 | NM_004488 | CTATCTCAGAGCCCTTGTTC | 1681 |
| GPAM | NM_001244949 | GAGTACACACATTACACCCT | 1682 |
| GPANK1 | NM_001199240 | AATACAGTTTTGTGCTCACT | 1683 |
| GPATCH2L | NM_017972 | TCTAAGTGTAGCCAGATGAA | 1684 |
| GPATCH4 | NM_015590 | GGCAACCATACCGGCAAATT | 1685 |
| GPBP1 | NM_001127236 | GGATGACTGCAAGAAAGAAG | 1686 |
| GPC6 | NM_005708 | GGACTGGATCTCTTCCTAGT | 1687 |
| GPHB5 | NM_145171 | TGTGTTTAGTAGTTCCTGTA | 1688 |
| GPM6B | NM_001001994 | GAGTCTGCAGGCAAAGCTCG | 1689 |
| GPR101 | NM_054021 | GCAGAGTTAGTCACCCGTCA | 1690 |
| GPR107 | NM_001136558 | GGGACCCCTGATCTCAGGGT | 1691 |
| GPR135 | NM_022571 | CCGCGACACCGCCACTCCGG | 1692 |
| GPR146 | NM_138445 | CCACAGAGCGAGGCTGCCTT | 1693 |
| GPR150 | NM_199243 | GTTCCCAAAGTTAGTTGAAA | 1694 |
| GPR160 | NM_014373 | GGTCTCACTGAGCCCCCAAG | 1695 |
| GPR161 | NM_001267609 | CCTGATGCTGTGCTTAGAGC | 1696 |
| GPR161 | NM_001267609 | GGAAAGAAGGAAGGACAAAC | 1697 |
| GPR161 | NM_001267613 | GCCGAGGCGGGGAGGCGGCT | 1698 |
| GPR161 | NM_001267613 | GGAGCGAAGCGGGGCTCGGT | 1699 |
| GPR174 | NM_032553 | ATTTCTCTAGAGTAACTACA | 1700 |
| GPR3 | NM_005281 | GGCAGACTCGGGAGGGGGCG | 1701 |
| GPR33 | NM_001197184 | GTGAGGTCTTTTCCTCTTTT | 1702 |
| GPR37L1 | NM_004767 | ATGCTGTAGGGCCTGAGAAG | 1703 |
| GPR63 | NM_001143957 | GAGGAGGCAAGTAAAGAGGG | 1704 |
| GPR68 | NM_001177676 | AAGTCGCTGGAGGGAGAGCT | 1705 |
| GPR85 | NM_001146265 | CATTTCAGTATTACCAACAT | 1706 |
| GPRASP1 | NM_001184727 | TGGTGCCAACCCGCAGGCCC | 1707 |
| GPRASP1 | NM_001099411 | TCTGGCGCTGCTATAATATA | 1708 |
| GPRC5C | NM_018653 | GAGACAGTGGGACCTAACCA | 1709 |
| GPRIN3 | NM_198281 | CCCTGGAGACCAGAGACAGA | 1710 |
| GPRIN3 | NM_198281 | GGGCTGCAACACTTTCCCCC | 1711 |
| GPSM1 | NM_001145638 | GCCCTCTCCCCTGCATTCCC | 1712 |
| GPT | NM_005309 | TCTGTACCTACCCCCCATGT | 1713 |
| GPT2 | NM_133443 | AGTCCCACAGCGCCCCGCGC | 1714 |
| GPT2 | NM_133443 | CCTGGGCCCTGTAGTTCCCC | 1715 |
| GRAMD1B | NM_001286563 | ACTTCTGTCAGCATCCACTC | 1716 |
| GRAPL | NM_001129778 | GGGGAGTCTCCCTGAAGCTC | 1717 |
| GRASP | NM_001271856 | GGCCTGCCCGCTGGACACAA | 1718 |
| GRB2 | NM_203506 | CATGCGCCCTGACACCTAGC | 1719 |
| GRB7 | NM_001242443 | GGCCCCGGTAAAGCTTCGGT | 1720 |
| GREM2 | NM_022469 | TTGCAAGCGACTGAAGTGTG | 1721 |
| GRIA1 | NM_001258022 | TGGAAGCATCTTCGTTGGTT | 1722 |
| GRIA1 | NM_001258022 | TGTCAGTGTCGTTTGTGTCC | 1723 |
| GRIA4 | NM_000829 | TGAAAGGGTTCAGAGAGGGA | 1724 |
| GRIK2 | NM_001166247 | CAGTCTTTCTCACTTAATCT | 1725 |
| GRIK4 | NM_001282473 | AGTTTACAAATGGAATCCGG | 1726 |
| GRIN2A | NM_000833 | GCCCGGTCCTCTGAGCGCGC | 1727 |
| GRIP1 | NM_001178074 | CTTGATGCTGAGAAGGAAAG | 1728 |
| GRK2 | NM_001619 | TCAGACCCTGGCCGTGACCT | 1729 |
| GRPR | NM_005314 | GTCAATATTGCTATCAAATG | 1730 |
| GRSF1 | NM_001098477 | CTGGAGGCCACGCGTCTGGG | 1731 |
| GSDMA | NM_178171 | ACGTGTGCCCTGGCCTCCTG | 1732 |
| GSDMB | NM_001042471 | GGAGTCTTGCTCTATCGCCG | 1733 |
| GSDMD | NM_024736 | AGTTTGAGGCTACCAGGATG | 1734 |
| GSG1 | NM_001206843 | GGTAACTGGTGTGAATGGAT | 1735 |
| GSG1 | NM_001206843 | TCCACTGCCTGCCATTCCCT | 1736 |
| GSPT1 | NM_001130006 | GCGGTTTTCCCGGGGGCCGA | 1737 |
| GSTK1 | NM_001143680 | CCAGCCTACGGCCCCCAGCC | 1738 |
| GTDC1 | NM_001284233 | ATTCCACCATAGCAGTGAAG | 1739 |
| GTF2F2 | NM_004128 | GGAAATTTCTTGAGTGGGCG | 1740 |
| GTF2H5 | NM_207118 | ACCCTCCACCCGGCGGCTGG | 1741 |
| GTF2H5 | NM_207118 | TCTTTCCGCGGCTCCCGGCC | 1742 |
| GTF2IRD2 | NM_173537 | AATGCACAGCGCGGCTAAAT | 1743 |
| GTPBP1 | NM_004286 | TCAGGCGGGTAGCGGGGACT | 1744 |
| GTPBP3 | NM_001195422 | AACCCTAGAGTGACGTGCAT | 1745 |
| GTPBP3 | NM_001195422 | CGGGAAGGAGAATCGAGGTT | 1746 |
| GTSF1 | NM_144594 | GAGTTCACCTGTGAGCCCCT | 1747 |
| GUCA1A | NM_000409 | CAAGGTTAAAAGACCCTTCC | 1748 |
| GUCA2A | NM_033553 | CTGTCAGGCCTTATCAGATA | 1749 |
| GUCA2B | NM_007102 | AGCTGGCTCTCTGACAAGCC | 1750 |
| GUCY1A3 | NM_001130685 | CCTCCGCCTGGGTCTGTTCC | 1751 |
| GUCY1A3 | NM_001130687 | AACTTCCCCAGCAGAAATGT | 1752 |
| GXYLT1 | NM_001099650 | GCTAGCGCAGGCCGACGCGC | 1753 |
| GYPA | NM_002099 | TTAACTTTGCATCAGTTAAG | 1754 |
| GZF1 | NM_022482 | TTAACAACCTAGCTTTACTC | 1755 |
| GZMK | NM_002104 | GGAGTCTCTCTCTGTCGCCC | 1756 |
| H2AFB3 | NM_080720 | TGTGGTGACGGCCCCTCACA | 1757 |
| H2AFY | NM_138609 | CCAGGCACCAGCCCGCACCC | 1758 |
| H2AFY2 | NM_018649 | GCTCTGGGGAGAGTCTTCGA | 1759 |
| H2AFZ | NM_002106 | TTCTAATCTCAAGCCGCGAT | 1760 |
| H2BFM | NM_001164416 | CTGACATGATTCCAAGCAAC | 1761 |
| H2BFWT | NM_001002916 | TGTGTAACTTTCTCCGAGCT | 1762 |
| HABP2 | NM_004132 | CATGAAGTGGTTTCTCTTCT | 1763 |
| HABP2 | NM_004132 | GCTATGTCAGCTACTTTCTT | 1764 |
| HABP4 | NM_014282 | TCGCGTGACGTGACAGCAGC | 1765 |
| HACE1 | NM_020771 | AAACTGCTCCTGTACAACTT | 1766 |
| HAMP | NM_021175 | ATAAGCGGGAACAGAGCGAC | 1767 |
| HAPLN4 | NM_023002 | GGCGAGGCGGGGTGTATTAA | 1768 |
| HARS2 | NM_012208 | GGCGGCTCAAGTGGACAGCC | 1769 |
| HAS3 | NM_001199280 | TACTGTCGATAAGGTCAGTT | 1770 |
| HAUS3 | NM_024511 | AGGATGCCCGCAGCGGCCGG | 1771 |
| HAVCR1 | NM_001173393 | GCTATTACTGCATATGATGT | 1772 |
| HBE1 | NM_005330 | GAGATTTGCTCCTTTATATG | 1773 |
| HBZ | NM_005332 | CCCTCAGGGCCTGGTGGGAC | 1774 |
| HCLS1 | NM_005335 | ATTTAAGTGTCTAAAGCAGA | 1775 |
| HDAC9 | NM_001204147 | CAATGGTGGATACACAGAGT | 1776 |
| HEATR4 | NM_001220484 | TGGTAGTTTCATGGAGTTTT | 1777 |
| HEATR5A | NM_015473 | CTTCACCGTCGAAAGAGCGA | 1778 |
| HEATR5B | NM_019024 | TAGGAAACTGGTGGGAGCCG | 1779 |
| HECTD2 | NM_173497 | GCCTTCTCTCCGGGCCCTCG | 1780 |
| HECW1 | NM_015052 | GGGTGTTGGAAGGATGGGGC | 1781 |
| HEMGN | NM_018437 | AGAATTAGGGCTCAAAACTA | 1782 |
| HEPACAM | NM_152722 | GTTTTCCAGTCTTCTTCCTT | 1783 |
| HEPHL1 | NM_001098672 | AAATGACTGATGTCAGAGCA | 1784 |
| HERC3 | NM_001271602 | ACGGGTGTGTCAGCCGAAAT | 1785 |
| HERPUD1 | NM_014685 | GCCGCGTCTGCGTCACCCAG | 1786 |
| HHLA3 | NM_001036645 | GATGGCCGTGCCCTGTTTTT | 1787 |
| HIF1AN | NM_017902 | AGGCTCCACTGCTGAAGAAA | 1788 |
| HIF3A | NM_152795 | CCCAATCAGAGCCTCAGGCC | 1789 |
| HIF3A | NM_152796 | AACTCTATCCCACCCCTTTT | 1790 |
| HIGD1A | NM_014056 | CCGCCAGTACGCTAGAGCCG | 1791 |
| HIGD1A | NM_014056 | GGGCTTTGGCTCCTGGCCCA | 1792 |
| HIGD2A | NM_138820 | GGGAGTCGTAGTGCTCAGCA | 1793 |
| HINT3 | NM_138571 | ATGGAGCTTGTTGGGTGTTC | 1794 |
| HIPK1 | NM_181358 | CGAAACCAGCCTGGCCAACA | 1795 |
| HIPK1 | NM_198269 | TTTCTTCATCTGTAAAATGG | 1796 |
| HIPK3 | NM_001278163 | TGGGCTTTACTGTATAACCT | 1797 |
| HIST1H1A | NM_005325 | CTGAGACTGGGCGAAACCCT | 1798 |
| HIST1H1B | NM_005322 | TTGGCACTTTGAAGCTCCAA | 1799 |
| HIST1H1C | NM_005319 | ATTCCCCGCACCAAATCACT | 1800 |
| HIST1H2AB | NM_003513 | AACATAAACCTTACACCAGA | 1801 |
| HIST1H2AH | NM_080596 | CTTCACCTTATTTGCATGAG | 1802 |
| HIST1H2BK | NM_080593 | ACCAATGGAAGTACGTCTTT | 1803 |
| HIST1H2BN | NM_003520 | GAAGTTGTGCGTTTAACCAG | 1804 |
| HIST1H2BN | NM_003520 | TTTCAAAACCGCAATCCCAT | 1805 |
| HIST1H2BO | NM_003527 | GAAGCTGCAAGCTTAGCCAA | 1806 |
| HIST1H4I | NM_003495 | AGCAGGCCTGTTTCCCTTTT | 1807 |
| HIST1H4K | NM_003541 | AGATTTCCCCTCCCCCACCG | 1808 |
| HIST1H4K | NM_003541 | TAAAGGGCCAAACCGAAATA | 1809 |
| HIST2H2BE | NM_003528 | ACACCGACTCTTGACTTGAT | 1810 |
| HIST2H2BF | NM_001024599 | GTCTTGTTATCCTATCAGAA | 1811 |
| HJURP | NM_001282963 | AGCCACGCCCCAATGTCCGG | 1812 |
| HJURP | NM_001282963 | CAAATTTGCGTCCCACCTTC | 1813 |
| HK1 | NM_033498 | ACATGTTTGGCAGGTTAGGG | 1814 |
| HLA-A | NM_002116 | GAGACTCTGAGAGCCACGCC | 1815 |
| HLA-DPA1 | NM_001242525 | TTGTGTCTGCACATCCTGTC | 1816 |
| HLA-DRB5 | NM_002125 | TATTGAACTCAGATGCTGAT | 1817 |
| HLX | NM_021958 | TGTGCGCTACTAAGCCCACG | 1818 |
| HMG20A | NM_018200 | GGGATTATTTTGCCCCAATG | 1819 |
| HMGB4 | NM_145205 | GACCTTGGCTATGGATTTTT | 1820 |
| HMGCL | NM_000191 | CTCGGAATCAAAACGGAGAG | 1821 |
| HMX1 | NM_018942 | GGCTCAGCGGGCCGCCCTCC | 1822 |
| HMX3 | NM_001105574 | TCAACTACGGGGCGCAAAGT | 1823 |
| HN1 | NM_001288609 | GTCGACTCCCTTGAAGGTGG | 1824 |
| HN1 | NM_016185 | AAGGCGAATCTACCTCGCGC | 1825 |
| HN1 | NM_016185 | TTCTTGGGGAGTTACAACCT | 1826 |
| HNF4G | NM_004133 | AGAATATGGCCTGCTGAAGA | 1827 |
| HNRNPF | NM_001098208 | AGCGCTAGCTTGGCGGGCCG | 1828 |
| HNRNPH3 | NM_012207 | GCGCGCTGCAGCTCTTTAAC | 1829 |
| HNRNPK | NM_031263 | AAAAGTAAACGCAGCCTTTC | 1830 |
| HOMER1 | NM_004272 | ATGGAAGTGTGAAGAGGCGG | 1831 |
| HOMER3 | NM_004838 | CCCAGTGCAAAAAGCCGGCA | 1832 |
| HOOK3 | NM_032410 | CACTGCGCACGCTCGCGCCC | 1833 |
| HOXA4 | NM_002141 | GGCGCTGCACGTGGGGCACG | 1834 |
| HOXA5 | NM_019102 | CATCAGGCAGGATTTACGAC | 1835 |
| HOXA9 | NM_152739 | ATCACTCCGCACGCTATTAA | 1836 |
| HOXB2 | NM_002145 | AATGCTCTCTGTTTTCCACC | 1837 |
| HOXC8 | NM_022658 | GGGAGTCTGAGGAATTCGCC | 1838 |
| HOXD11 | NM_021192 | GATTTTTGCTTAGTTGATCC | 1839 |
| HOXD11 | NM_021192 | TTGCACGTCAGCGCCCGGTG | 1840 |
| HOXD12 | NM_021193 | GTGTTATCATAATACTCTGA | 1841 |
| HOXD4 | NM_014621 | GGGAGAATGAATCCTCCTAT | 1842 |
| HP1BP3 | NM_016287 | GCGTCCCAGCGCGCCTGCGT | 1843 |
| HPCAL1 | NM_001258358 | TTACTCTGTGATTAAAAGCC | 1844 |
| HPCAL4 | NM_001282397 | GGTGCAGCCCTCCCGCTTCC | 1845 |
| HPGD | NM_001256301 | CGGATACTGGAGATGAGAAG | 1846 |
| HPGDS | NM_014485 | CTTCGCAGGCTTGAACTGCC | 1847 |
| HPR | NM_020995 | CTTCACACTTGATTTTCCCG | 1848 |
| HPRT1 | NM_000194 | CAACTCAGTCTCCTATTCAG | 1849 |
| HPRT1 | NM_000194 | TTTTCTCCCAGAAGAAGCCG | 1850 |
| HPS1 | NM_000195 | AGAGAAGAAATAACTTGCTG | 1851 |
| HPS4 | NM_152841 | GCGTGTTTGCTCAGCAACCG | 1852 |
| HRASLS2 | NM_017878 | CGAGACCATCCTGACTAACA | 1853 |
| HRH1 | NM_001098211 | TGGGTTGTGGTCGGGTGCGG | 1854 |
| HRH2 | NM_022304 | AACCGCTCCAGGCAAGAGCC | 1855 |
| HRH4 | NM_001143828 | TTGTTGTTGTTGTTGTTGTT | 1856 |
| HS3ST2 | NM_006043 | ATGCAACCGCCTGTTCCCCG | 1857 |
| HSD17B12 | NM_016142 | TCAGAGAAGCCGCTAGTGAA | 1858 |
| HSD17B4 | NM_001199291 | TTAAGAGTGACTCCACTCGC | 1859 |
| HSD3B2 | NM_000198 | CACAGTGTGATAAAGAGTCT | 1860 |
| HSDL1 | NM_001146051 | GTTCGCGGCGGACGTCGCTA | 1861 |
| HSFX2 | NM_001164415 | GATGTGACCGCAGACACCCG | 1862 |
| HSFX2 | NM_001164415 | TTCTCTGGAGACACTGGCCA | 1863 |
| HSP90AB1 | NM_007355 | GGACATGACTCCATCAAGAG | 1864 |
| HSPA12A | NM_025015 | CGGGCCGGCCGGGAAAGGTC | 1865 |
| HSPA5 | NM_005347 | AGGGGGCCGCTTCGAATCGG | 1866 |
| HSPA6 | NM_002155 | TTCGCATGGTAACATATCTT | 1867 |
| HSPA8 | NM_006597 | GAGTCCTCAGTTACCCCGGG | 1868 |
| HSPB6 | NM_144617 | CGTGGCCAGACCCGGCCATT | 1869 |
| HSPB6 | NM_144617 | TAGAAACCCAAACAATGACT | 1870 |
| HSPBP1 | NM_012267 | GCCTTTCAGACTCTCCCAGT | 1871 |
| HSPD1 | NM_002156 | GAAAGTTCTGGAACCGAGCG | 1872 |
| HSPD1 | NM_199440 | AGAGACTCGCAGTCCGGCCC | 1873 |
| HTATSF1 | NM_014500 | CCGCTAGGTCCAGGGCGCTG | 1874 |
| HTN1 | NM_002159 | TGATCTATTGTAAAATCACC | 1875 |
| HTR1F | NM_000866 | GACTGTCAATCCGATTCATA | 1876 |
| HTRA1 | NM_002775 | GGACCGGGACCGCCCGCGGA | 1877 |
| HTRA2 | NM_013247 | GGTGGTGACTGTGTGGCCTC | 1878 |
| HUWE1 | NM_031407 | AGCGACCCTATCATCCTCTA | 1879 |
| HVCN1 | NM_001040107 | TGGGGAGAGGCTCACCTCCT | 1880 |
| HYAL1 | NM_153281 | ACGCTCCTCACTTTCCAGAC | 1881 |
| HYAL1 | NM_153283 | CCTGGCAAAGGGATCTTGGT | 1882 |
| HYAL2 | NM_033158 | AGATCCTACTCGGGAAGGGT | 1883 |
| HYAL2 | NM_033158 | GTCACCTGGCGCAGCTGGCG | 1884 |
| HYKK | NM_001083612 | GCAGCCTCCTAGGCGGGGCC | 1885 |
| ICA1 | NM_001136020 | CCACCTTCCCCCGGTCACCC | 1886 |
| ICA1 | NM_001276478 | ACTTGATTTCCAGGTACAGC | 1887 |
| ICAM2 | NM_001099789 | AGACTGAGTCTCAGTCACCC | 1888 |
| ICOS | NM_012092 | ATTGATGATTTTGAAGACAG | 1889 |
| ICOS | NM_012092 | GACATGAGTTAAACAATGCA | 1890 |
| ID3 | NM_002167 | CAGCAAATTGGGGAACAAGG | 1891 |
| IDNK | NM_001256915 | GGAGACGCGAGTGCCAGGCC | 1892 |
| IDO1 | NM_002164 | TCATTTTCTTACTGCCATAT | 1893 |
| IDO1 | NM_002164 | TGTTTTCCTTCAGGCCTTTC | 1894 |
| IER5L | NM_203434 | TGGCCAGCCGAGTAGCCCCG | 1895 |
| IFI16 | NM_005531 | AATCTCTGACTTCACCAATA | 1896 |
| IFI30 | NM_006332 | TGCGCCAGGGCTCACGTGCC | 1897 |
| IFIT3 | NM_001549 | GGTTAACTTTGGAATGCCCT | 1898 |
| IFITM1 | NM_003641 | ACTAGTGACTTCCTAAGTGT | 1899 |
| IFNA1 | NM_024013 | GCAAAAACAGAAATGGAAAG | 1900 |
| IFNA5 | NM_002169 | CTCTTTCTACATAGATGTAC | 1901 |
| IFNA8 | NM_002170 | ATGCAGTAGCATTCAGAAAA | 1902 |
| IFRD1 | NM_001550 | CCAGTCTTCCGTCCGCGCCC | 1903 |
| IFT122 | NM_018262 | CTTTCGCAACATTCAGACCT | 1904 |
| IFT27 | NM_001177701 | AGTTCAGTCTGCTTGACGAG | 1905 |
| IFT80 | NM_001190242 | AAAAATGCTTCATTTTGGCC | 1906 |
| IGF2 | NM_001007139 | GCTTTACTTAGAGTGACACT | 1907 |
| IGFBP1 | NM_000596 | AACAAGTGCTCAGCTGGGAG | 1908 |
| IGFBP4 | NM_001552 | AAGAAGGAAGCGGCGCAGTT | 1909 |
| IGFBP5 | NM_000599 | GCGCTGTTCAGGGAGCGAAG | 1910 |
| IGFL1 | NM_198541 | ACAATGACACGTACCCTGCC | 1911 |
| IGFL2 | NM_001135113 | GTTTTTTCTTATGCTTTCTG | 1912 |
| IGSF1 | NM_001170963 | TTGAAGGCCCGCTCCGATGT | 1913 |
| IGSF21 | NM_032880 | CCGCTAAGCCGATTTATTGC | 1914 |
| IGSF8 | NM_001206665 | GCCCGGGGCGGATCCAGGGC | 1915 |
| IKBKAP | NM_003640 | TCGGTAGCCATGGCGACCTC | 1916 |
| IKZF3 | NM_012481 | CCCGCGCACCGGCAGGTCGC | 1917 |
| IL10 | NM_000572 | GCATCGTAAGCAAAAATGAT | 1918 |
| IL11 | NM_000641 | AGGGTGAGTCAGGATGTGTC | 1919 |
| IL12RB1 | NM_001290024 | GCGCCTGACCCAGTCATTGC | 1920 |
| IL12RB2 | NM_001258214 | TATAGGTCCCGTGTTATAAG | 1921 |
| IL15RA | NM_002189 | ACCCCTGTCCCCGGGACGCA | 1922 |
| IL16 | NM_172217 | GGAGTGGGTGTTAACCGCTT | 1923 |
| IL17RE | NM_153483 | TCTTAAGCACTACTCAGCAC | 1924 |
| IL18BP | NM_005699 | GCTGCGTGTGAACCCACCAC | 1925 |
| IL18RAP | NM_003853 | AATAAACTACCTCTTTCAGT | 1926 |
| IL19 | NM_013371 | CCTCTGGGAGAACCAGAGAA | 1927 |
| IL1A | NM_000575 | CCCTGTAGTCCCAGCTATTC | 1928 |
| IL1R2 | NM_001261419 | ATTACGTACTTCCAGCCGAG | 1929 |
| IL1RAPL1 | NM_014271 | TCACATAGCAGTACTGTACA | 1930 |
| IL1RL2 | NM_003854 | CATCTAAGTCCTTCATCACC | 1931 |
| IL2 | NM_000586 | ACCCCCAAAGACTGACTGAA | 1932 |
| IL20RA | NM_014432 | TGTAAGAGGCTATACCATAT | 1933 |
| IL21 | NM_001207006 | ATGTGCTAATGTGTGGGGGC | 1934 |
| IL22RA2 | NM_181310 | TAAACGATTCGAGAAGCCAA | 1935 |
| IL27 | NM_145659 | GGAAATGTAATTTCCCTTCC | 1936 |
| IL3 | NM_000588 | GGAAGGATCTTTATCTGACA | 1937 |
| IL36A | NM_014440 | GACTGGGGTCACTGCTGGGC | 1938 |
| IL36G | NM_001278568 | TTTCTTCCTCCGAGCCTCAC | 1939 |
| IL37 | NM_173205 | ACTGATGTTACTGCTGCTGT | 1940 |
| IL4 | NM_172348 | CCAATCAGCACCTCTCTTCC | 1941 |
| IL9R | NM_002186 | GTCAGTTTAATGAATCTCAG | 1942 |
| ILDR1 | NM_001199800 | AGAGGGGGATACATTTGCAG | 1943 |
| ILDR1 | NM_001199800 | GGGACGGTGTTTCAGCGAGC | 1944 |
| IMPDH1 | NM_001142574 | GGCGGCGGTTTCCGCGGGAG | 1945 |
| IMPG2 | NM_016247 | TGGACTGCTTGTTAAAGGCA | 1946 |
| INA | NM_032727 | CGGAGCTCCTGCTCAGAGTC | 1947 |
| INO80B | NM_031288 | TGTCCCGACCTCAGAGGGAC | 1948 |
| INO80C | NM_194281 | GCGGGCGTTGTCCTGCCACT | 1949 |
| INO80D | NM_017759 | CTCTGGAAAAAAGTCCACAC | 1950 |
| INPP5J | NM_001284285 | GGAGAGTGTACCCATCTGCC | 1951 |
| INPP5K | NM_130766 | GGCGGGGGAGACCGGATCCC | 1952 |
| INSL6 | NM_007179 | GGGGCGTCGCCAGAACTTCA | 1953 |
| INSM1 | NM_002196 | GTACATCTGCCGCACCTACC | 1954 |
| INTS6L | NM_182540 | GGGAGTTGAAGTTTGAACCC | 1955 |
| INTS7 | NM_001199812 | CTTACAGTGGCGGGAGTTGG | 1956 |
| IP6K1 | NM_001006115 | TCAGCAGGAAGCACTTCCCC | 1957 |
| IP6K2 | NM_001005909 | GGACAATGCTCCGCCCTCTC | 1958 |
| IPCEF1 | NM_015553 | TGTCCTGGATATGGGCATCA | 1959 |
| IPO11 | NM_001134779 | AAGTTGTCCTCTATTTAAAG | 1960 |
| IPO8 | NM_006390 | CCAGCTCAAGTTTCCTCACC | 1961 |
| IPO9 | NM_018085 | GAAAGGTGCAGTTCTCGTTC | 1962 |
| IPO9 | NM_018085 | GTGAAAACTGAGCCCCAGAC | 1963 |
| IPPK | NM_022755 | CCCAGACACCCTGGCTACCC | 1964 |
| IQCK | NM_153208 | AAGGTGTAATACAATGATAC | 1965 |
| IQGAP2 | NM_001285460 | GTCCAAAGTTAACCCTTTCT | 1966 |
| IQGAP2 | NM_006633 | CCCCCGCACAGCTGGTGGCC | 1967 |
| IQGAP3 | NM_178229 | TTCCTCGTCTTGTTCCTTCC | 1968 |
| IQSEC2 | NM_001111125 | CACTGCGCAGCGCGGCCGCG | 1969 |
| IQUB | NM_001282855 | AGGCGACATGGGAAGTCCGC | 1970 |
| IQUB | NM_001282855 | GAATTTTCTCCCCTCTGCTC | 1971 |
| IRAK2 | NM_001570 | ACACGGGAATTCTGCCGCAG | 1972 |
| IRF5 | NM_032643 | CGCCGGGCGCGGACGCAGAG | 1973 |
| IRGM | NM_001145805 | CATTTTGACAGGGTGCTGAT | 1974 |
| IRX4 | NM_016358 | GTCGCCGCTGCGAGGCCGCT | 1975 |
| ISG20 | NM_002201 | CATCCCCAGGACTGGAGCTC | 1976 |
| ISL2 | NM_145805 | GGGATCCAGGGGCTGATGGG | 1977 |
| ISLR2 | NM_020851 | GCTTATATCAGCCCAGATCC | 1978 |
| IST1 | NM_001270976 | AAGTCATCTGCTCCCTGCTG | 1979 |
| ISY1 | NM_001199469 | CCGGTCCTCCCTTTCACTTC | 1980 |
| ISYNA1 | NM_001253389 | CGAAGCTCTGTGGGGCGGGA | 1981 |
| ITFG1 | NM_030790 | CTGTCGGGAGGCGCGCCTGC | 1982 |
| ITFG1 | NM_030790 | GCCGCCCTCACGCTCACTTC | 1983 |
| ITGA2B | NM_000419 | ATTCTAGCCACCATGAGTCC | 1984 |
| ITGA7 | NM_001144997 | CTGGCTGGGCCAAACAGGGC | 1985 |
| ITGA7 | NM_001144997 | GGAAGCTGCTGAGTTGTTAG | 1986 |
| ITGA9 | NM_002207 | ACTGAGGACGCCGCCGCTCG | 1987 |
| ITGAM | NM_001145808 | TTTGTCACCCACTTGTTTCT | 1988 |
| ITGB1BP2 | NM_012278 | GAGGCGTACACCTCCTAACA | 1989 |
| ITGB5 | NM_002213 | TCCCCTGCCAGGCCCTCGCC | 1990 |
| ITGBL1 | NM_001271755 | TGACAAGAGAATATTTGGAC | 1991 |
| ITGBL1 | NM_001271756 | CTCATCCCAAGCAGGACATT | 1992 |
| ITIH1 | NM_001166436 | TGATGTGCTCTTCTTGGGCA | 1993 |
| ITPKC | NM_025194 | CCCCGCCCCACCGGACGTGA | 1994 |
| IZUMO3 | NM_001271706 | ACTAAAGATTGCCCGATAGT | 1995 |
| JAGN1 | NM_032492 | TAATCCCCAGCCTCTTTTGC | 1996 |
| JARID2 | NM_001267040 | GCTCGGTTCCCCGACGCTCC | 1997 |
| JARID2 | NM_001267040 | GTCACAATGACAACAGAGTG | 1998 |
| JMJD7- | NM_005090 | CAGTCGCTCCACCGCTTCGG | 1999 |
| PLA2G4B | | | |
| JMY | NM_152405 | CCGCGCAGCCTCCAGTTCCC | 2000 |
| JOSD1 | NM_014876 | CTCCATCCCCTCGGGTACGG | 2001 |
| JOSD2 | NM_001270640 | AGGCTCTCGCGATAGCTTCC | 2002 |
| JPH2 | NM_020433 | ACATGTGCTTCCGAAAGCAG | 2003 |
| JRK | NM_003724 | GTGGCCGCGGAGGGCGTGGG | 2004 |
| JSRP1 | NM_144616 | CCCTGCCCTGCTGCAATGGC | 2005 |
| JTB | NM_006694 | AAGGACCAGCTCTGAGGAGT | 2006 |
| KANK2 | NM_015493 | CTATGAGTGGGTCCCAGACC | 2007 |
| KANSL1 | NM_001193465 | ACACAGAGACAGAGACGCCA | 2008 |
| KANSL1 | NM_001193466 | GGAGAGCGGCGGGCCCGGGC | 2009 |
| KARS | NM_001130089 | GTAGTGCTCGGCGTCAGACA | 2010 |
| KAT2A | NM_021078 | AGTGAAGAGGGGTCAATGTG | 2011 |
| KAZN | NM_201628 | CTTCGGAGACACACCCCCCG | 2012 |
| KBTBD3 | NM_152433 | TTGGCCAGTTCGTCTTTGCC | 2013 |
| KCNAB2 | NM_001199861 | TTGGCCAGAGCCTCGGGGTT | 2014 |
| KCNE4 | NM_080671 | AAGACAGTTGGAAGCAAGTG | 2015 |
| KCNF1 | NM_002236 | TGCGCCCGAGGAGGGGCCGG | 2016 |
| KCNJ10 | NM_002241 | CAGGCTCGAGCCGCCGAGAT | 2017 |
| KCNJ15 | NM_170737 | ACAGTCCTCTGGCATCATTA | 2018 |
| KCNJ6 | NM_002240 | AGCGCGTCGAGGACCGGGCT | 2019 |
| KCNK17 | NM_031460 | AGGAAATGTGAGGGGGCTCT | 2020 |
| KCNK7 | NM_033348 | TGAATGAATGAATGTGGTAT | 2021 |
| KCNMB2 | NM_181361 | TTCTATATGGAAAGCGAACT | 2022 |
| KCNMB3 | NM_171829 | AGAGAAAGAATTCACCAACC | 2023 |
| KCNRG | NM_199464 | ATGTTAGGAATGAGACAGCC | 2024 |
| KCTD1 | NM_001136205 | GGACCCTTCCCCACCCGCCC | 2025 |
| KCTD1 | NM_001258221 | AGAACAGCCGAGGTCCCCGG | 2026 |
| KCTD13 | NM_178863 | GGTCGGCCGCATCCTCGATC | 2027 |
| KCTD14 | NM_023930 | AAGGGGTCTGCTCCATTTCT | 2028 |
| KCTD21 | NM_001029859 | TCTCGACGCGCCGAGCTGCG | 2029 |
| KCTD6 | NM_153331 | GCTGAGGCAGGAGGATCACC | 2030 |
| KCTD8 | NM_198353 | GCTAACTACTCCTGGCAGCA | 2031 |
| KDELR1 | NM_006801 | GAAAGTGCCAAATCCAGCAC | 2032 |
| KDM4A | NM_014663 | CGATCCAGCTAGAGGCTCAC | 2033 |
| KDM5D | NM_001146706 | AGTAAACACTTTCACATGAA | 2034 |
| KDM7A | NM_030647 | GGCCCAGACTCGGCTGCTTC | 2035 |
| KDR | NM_002253 | TCCCCATTTCCCCACACAAC | 2036 |
| KERA | NM_007035 | TTTATTCCAAGTACCTGCTA | 2037 |
| KHDC3L | NM_001017361 | GGCCTGGGACCCAATAAGAA | 2038 |
| KHDRBS2 | NM_152688 | GCAGCTGCCTCCTGCCAGTC | 2039 |
| KIAA0100 | NM_014680 | CCAAGAGCTGAAACACGCCC | 2040 |
| KIAA0101 | NM_014736 | ACCCACTAGTCGGGTACCCC | 2041 |
| KIAA0141 | NM_014773 | GGGGCGGTGACGTGCGGCAA | 2042 |
| KIAA0586 | NM_001244189 | GAGATTTTAGAATTCGCTGA | 2043 |
| KIAA0907 | NM_014949 | ATCGGAATCGACATTTTCAC | 2044 |
| KIAA0930 | NM_001009880 | ACCGGGGCCGGGCCGGGCCG | 2045 |
| KIAA1109 | NM_015312 | ATACTCTGGCTCAAAATAAC | 2046 |
| KIAA1147 | NM_001080392 | GGAACCGCGAGCCTATTCGG | 2047 |
| KIAA1211 | NM_020722 | TCCTCCTCCATCCCCTGTAA | 2048 |
| KIAA1522 | NM_001198973 | TCCTCCTAATCATACTCTAC | 2049 |
| KIAA2013 | NM_138346 | GGACTTCACTCTTCCGGCCT | 2050 |
| KIDINS220 | NM_020738 | CTTGCCTGGGGCGCTTGTCC | 2051 |
| KIF12 | NM_138424 | CTTATCATACCTGCACCTAG | 2052 |
| KIF1BP | NM_015634 | ATCTCCAGATTGACCCTGTG | 2053 |
| KIF23 | NM_001281301 | CTCCATCACAAGAAGTTCAA | 2054 |
| KIF25 | NM_030615 | CTTCTTCTCTTTATGGGGGT | 2055 |
| KIF25 | NM_030615 | TTTCGTCGTTGAAGGCCACG | 2056 |
| KIF27 | NM_001271928 | CGCGTTGGTGGGACACAACT | 2057 |
| KIF2B | NM_032559 | CAGAGAAGCAACGGGAACCA | 2058 |
| KIF2C | NM_006845 | GGGGGTGTGGCCAGACGCAT | 2059 |
| KIF3B | NM_004798 | AGCGGGGGCCCAACACACCT | 2060 |
| KIF5C | NM_004522 | GTAGAGTGACTACAAGTCCC | 2061 |
| KIFC3 | NM_001130100 | GGGAGGCCCCGCGAAGGAGT | 2062 |
| KIR3DL2 | NM_001242867 | GGCTCTTTCTACCTTGCATG | 2063 |
| KITLG | NM_003994 | GCCAACCTTGTCCGCTCGCC | 2064 |
| KLF11 | NM_001177718 | GGGAACGCGGCACGGTTTTG | 2065 |
| KLF12 | NM_007249 | GGCTGCCGAGTTGCGAGCCC | 2066 |
| KLF14 | NM_138693 | AGGGGCGCGTCAGGCGGGGC | 2067 |
| KLF15 | NM_014079 | GGACGTGTGACGCGCAGCGC | 2068 |
| KLF7 | NM_001270944 | ACACGTGTGCAGCTGTGCTT | 2069 |
| KLHDC8A | NM_001271865 | TGGGAATCTCGCACCCACGC | 2070 |
| KLHL12 | NM_021633 | CGCCTATAATCCCGGCACTT | 2071 |
| KLHL13 | NM_033495 | ACCACTCCAAAGCTCAACAG | 2072 |
| KLHL14 | NM_020805 | TGGAGAGACTCGCAAAATTA | 2073 |
| KLK15 | NM_017509 | AGTAAACCTTCCAGAGATGG | 2074 |
| KLK8 | NM_144507 | CTCTACGATCTGAAACATAA | 2075 |
| KLRC1 | NM_002259 | CTTGGTCTATTAAAAGTACA | 2076 |
| KLRF1 | NM_016523 | TACCCTTAAAGTCAAGGGAA | 2077 |
| KLRK1 | NM_001199805 | AAAGGCAGCGAGGGTCACTT | 2078 |
| KLRK1 | NM_007360 | GAGTTAAGACCACCCATTGT | 2079 |
| KLRK1 | NM_007360 | TCAATTCCAGTTAATACCTC | 2080 |
| KMT2E | NM_018682 | GAGGCTCGAAGATAGCAAAC | 2081 |
| KNOP1 | NM_001012991 | CGGTAACCGCGTTCGCCGGA | 2082 |
| KPNA1 | NM_002264 | AGGTTTGCAGACCATGGCAA | 2083 |
| KPNB1 | NM_001276453 | AAAAGAAAAAACCCCAAGAG | 2084 |
| KPNB1 | NM_001276453 | AGAGGAATAACCGAGCAAAG | 2085 |
| KRAS | NM_004985 | GGGGAGGCAGCGAGCGCCGG | 2086 |
| KRIT1 | NM_194454 | GGCAGGCGACTAGGAGACTA | 2087 |
| KRT10 | NM_000421 | AAACCTCCTGTTTATTCTTA | 2088 |
| KRT2 | NM_000423 | GTCTGCCTGGGAGCTATTCC | 2089 |
| KRT23 | NM_015515 | CATCTGTCCAATTAGTGGCT | 2090 |
| KRT7 | NM_005556 | TGAGTCCGTTTCCAATGGGC | 2091 |
| KRT82 | NM_033033 | GGGCCAATGGTCAGTGCTGG | 2092 |
| KRT85 | NM_002283 | ATAACATCTTCAAGACTTCA | 2093 |
| KRT9 | NM_000226 | GTCTGGGATACGGAGGCAGC | 2094 |
| KRTAP10-10 | NM_181688 | AGAAATAATGAGGGTCCTCC | 2095 |
| KRTAP10-2 | NM_198693 | AACGCCCTCCACTTCCGTGT | 2096 |
| KRTAP1-1 | NM_030967 | TTACCAAGGACAAACACATT | 2097 |
| KRTAP13-1 | NM_181599 | CACCCTTCATCTTATATTTA | 2098 |
| KRTAP13-2 | NM_181621 | TAAAAAGTGAGCAAGGAGAA | 2099 |
| KRTAP13-4 | NM_181600 | CAGTTACACATATGTAAATG | 2100 |
| KRTAP1-5 | NM_031957 | TGTTTAAATTTGTTACTCCG | 2101 |
| KRTAP19-1 | NM_181607 | ATCTTACTGAGTGTTGTCAG | 2102 |
| KRTAP19-7 | NM_181614 | AACAAGGAAGAGAGTGGGAT | 2103 |
| KRTAP2-2 | NM_033032 | AGGAAGAATAAGTGAAAACA | 2104 |
| KRTAP2-3 | NM_001165252 | ATCCAGAGTTCTCATTTCAA | 2105 |
| KRTAP27-1 | NM_001077711 | ATAACATCTCATTACCACTT | 2106 |
| KRTAP29-1 | NM_001257309 | CATGCAAACATCTGATTAGC | 2107 |
| KRTAP3-1 | NM_031958 | TGAGGTGAGCAGTGTATCTT | 2108 |
| KRTAP4-2 | NM_033062 | GGTTAACTTATCCACATAGA | 2109 |
| KRTAP4-8 | NM_031960 | ATAACAAGGAAATAATGACG | 2110 |
| KRTAP5-1 | NM_001005922 | CCAGCCTCACACATGACCCT | 2111 |
| KRTAP5-11 | NM_001005405 | GTGTAAACAGTCACAAGGAA | 2112 |
| KRTAP5-2 | NM_001004325 | GTGTAAACAGTCACAAGAAA | 2113 |
| KRTAP5-4 | NM_001012709 | AAATGTAGTCACTTCCTCCT | 2114 |
| KRTAP5-7 | NM_001012503 | AAATAGCGTAAACAGTCACA | 2115 |
| KRTAP5-8 | NM_021046 | TGTGTTCAGTATAAACACCT | 2116 |
| KRTAP5-9 | NM_005553 | GTGCTAGCAACACCAGCCTC | 2117 |
| KRTAP6-1 | NM_181602 | GGTTTTCAATCGTGGCCTTG | 2118 |
| KRTAP6-3 | NM_181605 | GAAATCAGAGAGATACGTAA | 2119 |
| KRTAP9-3 | NM_031962 | AAACAATGTAAACAGCAACA | 2120 |
| KRTAP9-4 | NM_033191 | AGTCCGTTTGTGATTCTCAA | 2121 |
| KRTAP9-9 | NM_030975 | TGGTGGAAACTTTGGAAGCC | 2122 |
| KRTCAP2 | NM_173852 | ATGCGTCGAGGGGGCATCCT | 2123 |
| KSR1 | NM_014238 | ACTGAGGTGTGTAGGGACTT | 2124 |
| KXD1 | NM_001171949 | AGTCACACTATCTACAAAAT | 2125 |
| L2HGDH | NM_024884 | GCGCGCGCGTCGGAGGGCGA | 2126 |
| L3MBTL4 | NM_173464 | GTTCCACACCCCGGGGAGCC | 2127 |
| LACRT | NM_033277 | TGCGGAAGTCACACCTCTCC | 2128 |
| LAMA3 | NM_001127717 | CTCAGCTCTGGAACCTGCCG | 2129 |
| LAMB1 | NM_002291 | AACGTAAATGCGCGAGTCCG | 2130 |
| LAMB3 | NM_001017402 | ACAGGAGAAGGTTTGCCTCC | 2131 |
| LAMB4 | NM_007356 | ACCCACACACACACATAAAC | 2132 |
| LAMC3 | NM_006059 | CACGTCCAGCAGGTGGGAGT | 2133 |
| LAMP3 | NM_014398 | GAAGTCTCGCTCTGTCGCCC | 2134 |
| LAMP5 | NM_012261 | TGGCAACAGTTTCCTGAATT | 2135 |
| LAPTM4B | NM_018407 | CAGGAGAATCGCTTGAACCC | 2136 |
| LARGE2 | NM_152312 | ACAGCCTGAGCCCCCTTTCC | 2137 |
| LARP4B | NM_015155 | GGTGTTGCGGCGCGCTGATT | 2138 |
| LARS | NM_020117 | CAAGGGACTCCAACCTAACC | 2139 |
| LAS1L | NM_001170650 | GGCGCCGACCTAATGACATG | 2140 |
| LAYN | NM_001258391 | CTGGAGAGAGAGGCGATGCG | 2141 |
| LBR | NM_002296 | TCATCCCCGGCGCTGTCGAT | 2142 |
| LBX1 | NM_006562 | TCGGCAGTGGCTCCTGGCCC | 2143 |
| LCAT | NM_000229 | CGCCTTCTTCTCTTGGCGCC | 2144 |
| LCE1E | NM_178353 | CTTGCCCCCTGATACCCACG | 2145 |
| LCE2C | NM_178429 | GGAATGACCCAGCGTGTGCC | 2146 |
| LCE2D | NM_178430 | GAGCTTCTAGGACTCCTCTC | 2147 |
| LCE3D | NM_032563 | CAAGACTAGGTTTGTAGCTT | 2148 |
| LCE3E | NM_178435 | ATCTTGGTGAGTACACAGGA | 2149 |
| LCE3E | NM_178435 | TGCCTGGCTGTCACCTCCCC | 2150 |
| LCK | NM_001042771 | GTCAGGTCTCTCCCAGGCTT | 2151 |
| LCOR | NM_015652 | GCATTCTCTCTTCCATCTAC | 2152 |
| LCP1 | NM_002298 | AAAGACAGCTGGAGGAGAAA | 2153 |
| LCT | NM_002299 | CAGGTGTGAGCCACCACGCC | 2154 |
| LDB3 | NM_001171610 | CCTGGTTGGTGAGAATGCTC | 2155 |
| LDB3 | NM_001171610 | CTCCTTGCTCCTGTGTCCTC | 2156 |
| LDHAL6A | NM_144972 | ATTTCTAACCAAACCTTGTC | 2157 |
| LDLR | NM_001195803 | AAACATCGAGAAATTTCAGG | 2158 |
| LDLRAD1 | NM_001276395 | TTCCAAGCAGAGGCAAAGGC | 2159 |
| LDLRAD4 | NM_001276251 | AGCAGCAGGCGCGCCTCTGG | 2160 |
| LDLRAD4 | NM_001276251 | GCATTTCCCTCGCCCGCCAC | 2161 |
| LDLRAD4 | NM_181481 | GGCATCAAGTAATAAAGGGA | 2162 |
| LECT1 | NM_007015 | TGTTTGGGGGGCCAGTAGAC | 2163 |
| LEF1 | NM_001130714 | TTTCTTTTCCCAGATCCTGT | 2164 |
| LELP1 | NM_001010857 | GCTTGTTGTGCTGGGAGCTA | 2165 |
| LEPROTL1 | NM_001128208 | CCAGGTCTTGAATTCCTGTC | 2166 |
| LEPROTL1 | NM_001128208 | CCCCCTGCCTCTCTTCTCCG | 2167 |
| LETM2 | NM_001199660 | GTTTTGCTCCCGTGTGGTGA | 2168 |
| LEXM | NM_152607 | GGCCCTTCTTGTATTTAATA | 2169 |
| LGALS12 | NM_001142536 | TGGAGTCTTGCTCTCTTGCC | 2170 |
| LGALS12 | NM_001142538 | ACCTCTAATCCCAGCTACTC | 2171 |
| LGALS12 | NM_001142538 | TGCAACCTCCTCCATCTCCC | 2172 |
| LGALS3 | NM_002306 | CGACCTCCGCTGCCACCGTT | 2173 |
| LGALS4 | NM_006149 | AAGTCTGGGCAGGGTTTTAT | 2174 |
| LGMN | NM_005606 | AGTAGTTGCGCACTGAAGTG | 2175 |
| LGR4 | NM_018490 | GAGCTCATTACTATGCAGAG | 2176 |
| LGR6 | NM_001017403 | CGGTGCAGCCCGCCGGGACC | 2177 |
| LHPP | NM_022126 | CTTTCTTCCCAGGAGATCAG | 2178 |
| LHX2 | NM_004789 | GCACGCGCTGCCAGGGCCTG | 2179 |
| LHX3 | NM_014564 | CACCGCAGGTCCCGGCGCAA | 2180 |
| LHX5 | NM_022363 | GGCAACTTCTGCAAGTTCCA | 2181 |
| LHX6 | NM_001242334 | CAGGGAGAGGGGGAGAGAGA | 2182 |
| LIFR | NM_001127671 | GGAGGAACGCGGCCGCGCGA | 2183 |
| LIG4 | NM_002312 | ATCCGGTCGTGGGGGTGTCT | 2184 |
| LILRA2 | NM_001290270 | ATGACAGCCAGGCTCCTGAG | 2185 |
| LILRB1 | NM_001081637 | CAGTGTCCAACCCCACCCCC | 2186 |
| LILRB3 | NM_006864 | CTGCCCCCACTTCAGCCCTG | 2187 |
| LILRB4 | NM_001278427 | AACCAAAAACCTGCATTTTC | 2188 |
| LIM2 | NM_001161748 | ATTCGCTGAAGCAGGCATCC | 2189 |
| LIMCH1 | NM_001289124 | TTAACTGTGTAACAATTTGG | 2190 |
| LIMCH1 | NM_001112718 | ACCCGCGGGAGCGAGCGCGG | 2191 |
| LIMS4 | NM_001205288 | CAATGCCGTGCTTTTCACTC | 2192 |
| LIN54 | NM_001115008 | AAGGGCCGTGCAAGTGCACA | 2193 |
| LIPA | NM_001127605 | GAGCCCGTCCTCCGCCTCGC | 2194 |
| LIPF | NM_001198830 | TATTGGCCAAAGTAGTTCTG | 2195 |
| LIPH | NM_139248 | AGGAGTCAAAGATCCTGAAA | 2196 |
| LIPT2 | NM_001144869 | TCCAGCTTTTAACACGCACC | 2197 |
| LLGL2 | NM_004524 | GCTGCGCTCCTGCCAATCCG | 2198 |
| LMAN2 | NM_006816 | GGGGCGGATTCGCGAAGACT | 2199 |
| LMNB2 | NM_032737 | GACTCCAGAGACAGACTTCC | 2200 |
| LMNTD1 | NM_001145727 | AGTCAGCGGCAGGCACTTTA | 2201 |
| LMO1 | NM_002315 | AGCGTCTTTGCTCCGATCCC | 2202 |
| LMO3 | NM_001001395 | TAACAGATCATACAGTTGGA | 2203 |
| LMO7DN | NM_001257995 | GGCCGTTGGCTTATTGTCTG | 2204 |
| LMX1A | NM_177398 | CGTGTGGTGGCCGCGCAGCC | 2205 |
| LMX1A | NM_177398 | GCGTGTCCGAGAGCTCCCAG | 2206 |
| LONP2 | NM_031490 | ATACTCTGTAAGTGAGGCGA | 2207 |
| LOXHD1 | NM_001145472 | CAAACCCACAGCCCCCACCC | 2208 |
| LOXL2 | NM_002318 | AACCCGGGCGCGAGGAGCCT | 2209 |
| LOXL3 | NM_032603 | AGAGGAGGGAACTGGCCGGG | 2210 |
| LOXL4 | NM_032211 | ACCTGGCCTGTGTCCCGACG | 2211 |
| LPAR5 | NM_020400 | AGGCTGGTGGGTTAGTCATC | 2212 |
| LPIN1 | NM_001261428 | CTTCTGGAAGTTTTGCATCC | 2213 |
| LPP | NM_001167672 | GCTCTGCGCGGCGGCTTCGC | 2214 |
| LPP | NM_005578 | ACACGATGTCCAGCCCCCAC | 2215 |
| LPXN | NM_004811 | CATGAATCCAAGATGAATCC | 2216 |
| LRBA | NM_001199282 | CGGTGGCCGCTGGGTTTCTC | 2217 |
| LRCH3 | NM_032773 | AAAGCGCATCATGTGGGCGG | 2218 |
| LRFN5 | NM_152447 | GACTTTGATAACCTCCCTGC | 2219 |
| LRIG3 | NM_153377 | GCGTAGGCCCCCGGCTGGAG | 2220 |
| LRP3 | NM_002333 | CGGGCGGGGGTCTTCCCTGG | 2221 |
| LRP8 | NM_004631 | GTCTGCAGAGCCCAGCACTC | 2222 |
| LRRC20 | NM_207119 | GACGAGGTGCCATTGGCTGC | 2223 |
| LRRC23 | NM_006992 | GTTATTTTCAGGTAGACCTT | 2224 |
| LRRC29 | NM_001004055 | GTGCTTAGTGATTGCGGTTT | 2225 |
| LRRC30 | NM_001105581 | GTGAGAACCAACTTGTGACT | 2226 |
| LRRC32 | NM_005512 | CCAAAGGAATGTGGCTGTGA | 2227 |
| LRRC32 | NM_005512 | GAATTTCAGGCAGCTCGGCG | 2228 |
| LRRC36 | NM_001161575 | TTCCCTACAATTACTTTCCC | 2229 |
| LRRC55 | NM_001005210 | ACGTGCCCTTTAAAGATCCT | 2230 |
| LRRC61 | NM_001142928 | AATCTAGGCCGCCATCCGTC | 2231 |
| LRRC72 | NM_001195280 | CGGACGCATCACCATGAGCA | 2232 |
| LRRC75A | NM_207387 | GAGGGAGGCGCGCGACGCCG | 2233 |
| LRRN2 | NM_201630 | CGTTCGCAGGTGCCCGGAGC | 2234 |
| LRRN3 | NM_001099660 | TTCCCAACATTCCCTCAGAA | 2235 |
| LRRN4CL | NM_203422 | AGAGCTGGGAGACATCATTC | 2236 |
| LRSAM1 | NM_001005374 | CCGACGTCCAGCCTAGATGC | 2237 |
| LSM3 | NM_014463 | CGGGTGCGTCACTCGCGAAG | 2238 |
| LSM5 | NM_001130710 | GAGATCGACTCTGTGGGGCG | 2239 |
| LSM7 | NM_016199 | GCGGGCACCGGCCGACATGG | 2240 |
| LSM8 | NM_016200 | GGGTTTCCAATCCGAGTAAA | 2241 |
| LSMEM1 | NM_001134468 | TACAGACCCACCACAGGTGA | 2242 |
| LSMEM1 | NM_182597 | TTGCAAGTCAGTCATCATAG | 2243 |
| LSP1 | NM_001289005 | CCAGACATCCCCGTTTAAAG | 2244 |
| LSP1 | NM_002339 | CAGCTCTTCATGGCTCGGGG | 2245 |
| LTA | NM_000595 | GAACCACAGGCTGGGGGTTC | 2246 |
| LTA4H | NM_000895 | TACCTGGGAGCGTGTGTGTT | 2247 |
| LTN1 | NM_015565 | AGGACAGGATTTGGCGCCAC | 2248 |
| LUC7L2 | NM_001270643 | ACCAGAGTATCGCGAGATCC | 2249 |
| LURAP1 | NM_001013615 | CGCCCAGCCCCACGCAATCC | 2250 |
| LUZP4 | NM_016383 | GCTCGCTAGAAGAAAAAAAA | 2251 |
| LY6G5C | NM_025262 | TTCTGCCCCTCTGGCTGGTC | 2252 |
| LY6G6D | NM_021246 | GATGCTGAGAGCATGCTGTG | 2253 |
| LY6G6F | NM_001003693 | AGCCCAGCAGCATGTCTACT | 2254 |
| LY6G6F | NM_001003693 | TGACCACCACTTTTCTATCC | 2255 |
| LY86 | NM_004271 | GGACCTTGAATCTACAGGTG | 2256 |
| LY96 | NM_015364 | CAGGCATGAGCCACCGTGCC | 2257 |
| LYPD4 | NM_173506 | GGCTCAACTCGAAGCGCTAT | 2258 |
| LYPD5 | NM_182573 | AACCTGTGCTCCGAGTGCGT | 2259 |
| LYPD6 | NM_001195685 | TTTTGCACCAAACCCATAAC | 2260 |
| LYPD6B | NM_177964 | AACTAACTCACCTGCACCCT | 2261 |
| LYRM7 | NM_181705 | TGCTAAAGGCGTTTGCTAAA | 2262 |
| LYRM9 | NM_001076680 | AGCTTTCAACTGGGTGGGGT | 2263 |
| LYSMD2 | NM_153374 | TGAGGCTGTTGAGATGGACC | 2264 |
| LYSMD3 | NM_198273 | GCGGGTCCAATCCCCGGGCC | 2265 |
| LYSMD3 | NM_198273 | TGGTTGGACTCCCCCGTTTT | 2266 |
| M1AP | NM_138804 | ACCAACACCTGCCTGAGGAC | 2267 |
| MAFF | NM_001161574 | GTGTCATTGGCTCATTTTAC | 2268 |
| MAG | NM_001199216 | GGGTTCTCCTAGCTCTTTCC | 2269 |
| MAGEA12 | NM_001166386 | ATCCGGCCCCGTGACTTCCC | 2270 |
| MAGEA12 | NM_005367 | TTGGGGGTAGGGGTAGGGAT | 2271 |
| MAGEA4 | NM_001011549 | CGGTGGAGGGGGCGGGTTTT | 2272 |
| MAGEA9B | NM_001080790 | GGGGCCCTCAGTCATCCCTC | 2273 |
| MAGEB1 | NM_177404 | CACCTTAGTATCTAGCAGTC | 2274 |
| MAGEB1 | NM_177404 | GGTCCCTACGTCCCCACTAG | 2275 |
| MAGEB4 | NM_002367 | AATTCTAAAGGTAATCAGAG | 2276 |
| MAGED2 | NM_177433 | GGAGATGAGTGGCCTTTCAT | 2277 |
| MAGED4 | NM_001272062 | AGAGGTGAAGTGGATCTGGC | 2278 |
| MAGIX | NM_001099681 | GGATGTTGCTATTCCAGCAT | 2279 |
| MALSU1 | NM_138446 | AGTGACCCGGAAGAGCTACT | 2280 |
| MAN2A2 | NM_006122 | TGCTTGTGCTACTTGGAGCC | 2281 |
| MAP1A | NM_002373 | GCTGGTCCGTGACGAGGCAC | 2282 |
| MAP2 | NM_031847 | AAATAAGGCGAGTGGGAGAG | 2283 |
| MAP2 | NM_031847 | TTTTCCTGTTCGCCACTGCG | 2284 |
| MAP2 | NM_001039538 | GGCTGCGGCAGAAGGCGAAG | 2285 |
| MAP2K1 | NM_002755 | CCGCCGAGGCTTGCCCCCAT | 2286 |
| MAP3K15 | NM_001001671 | ATCGAGGGAACGGAGCGCAC | 2287 |
| MAP3K2 | NM_006609 | TGAATACCTGCTTTTCTTCT | 2288 |
| MAP4K4 | NM_001242559 | GGCTGCGCTCTCGGGCCGCT | 2289 |
| MAP7 | NM_003980 | GCTTCCTAAAGCGCAGATCC | 2290 |
| MAP7D2 | NM_001168466 | CAGTCCTCACACAGCGCGTA | 2291 |
| MAPK15 | NM_139021 | AGGTGGGGTGGGCCCACTGT | 2292 |
| MAPK7 | NM_139033 | GGAAGGAAAGGTTTTCTAAA | 2293 |
| MAPK8IP2 | NM_012324 | GGCGTCGGGCCCCGCCCTGG | 2294 |
| MARCH10 | NM_001288780 | AGGAGGCGGTTGGCTTTGTC | 2295 |
| MARCH10 | NM_001288780 | GGAACGAGGCGGGCTGCAGT | 2296 |
| MARCH7 | NM_001282807 | CTTCTGTTATCTCAGGCACT | 2297 |
| MARCH7 | NM_001282807 | GCTTCAGAGAAAAGAGGGTC | 2298 |
| MARK1 | NM_018650 | GGCGGGCAAGAGAGCGCGGG | 2299 |
| MARK2 | NM_017490 | ACAAAGCCTCCAATAGGGCT | 2300 |
| MASTL | NM_001172304 | CACTGCAACCTCTGCTCCCC | 2301 |
| MAT2A | NM_005911 | GGCCGGGATAGCTTTCCCGG | 2302 |
| MATK | NM_002378 | CTTCCGAGAGCCGCCTCTCC | 2303 |
| MATN2 | NM_030583 | GCGAGGGCGGCCCCACCCTG | 2304 |
| MAU2 | NM_015329 | TGTAAAAGGGCGACGCCGTT | 2305 |
| MAZ | NM_002383 | AGGCCCCGCGGGGCCGGGGC | 2306 |
| MBD2 | NM_003927 | ATTAATTGGGAAGCAAACAT | 2307 |
| MBNL3 | NM_001170701 | GGAAGGTGGAGTGGCTGCCA | 2308 |
| MBOAT2 | NM_138799 | GACGGGGGCGACGGCAGGAC | 2309 |
| MBTPS1 | NM_003791 | CGACGCGCAGAGCGGACCAA | 2310 |
| MC5R | NM_005913 | GTGTCCAGGGGCACTCTTCC | 2311 |
| MCF2L2 | NM_015078 | ACAGTCCCTGGAGGCGGCGC | 2312 |
| MCFD2 | NM_001171511 | TAACTCTGTCTACCGTGAAA | 2313 |
| MCHR2 | NM_032503 | AGTGTTTATTGATGTACCAA | 2314 |
| MCM3 | NM_002388 | GAGGCTGGTCATTGAGCAGC | 2315 |
| MCM4 | NM_005914 | GCAGGAGACCTTGTCCGCTG | 2316 |
| MCM5 | NM_006739 | TTTGGCGCGAAACTTCTGGC | 2317 |
| MCM9 | NM_017696 | GGGTTAATATGAAGGAAATT | 2318 |
| MCPH1 | NM_024596 | CCGTCGTCCTCCTTACTCCC | 2319 |
| MCRIP2 | NM_138418 | CAGGCAGCAACGGCCTTCCC | 2320 |
| MCRIP2 | NM_138418 | GCGGTGCCCCGACACTGACA | 2321 |
| MCRS1 | NM_006337 | ACGTTAAGGATTATAGGCAC | 2322 |
| MCRS1 | NM_006337 | GGAGAGGTAACCCGGCTTGA | 2323 |
| MCTP1 | NM_024717 | CTGAAGTCGCTGGGCACTCC | 2324 |
| MCTP2 | NM_001159644 | AGAGATATTATACCAGAACA | 2325 |
| MCU | NM_001270680 | CGGCGGCGACCAGGAAGGGA | 2326 |
| MCU | NM_001270680 | TGAAGGGCACGGCGGCTCCT | 2327 |
| MDGA2 | NM_182830 | TCCCTTAATGGTTTTCACGA | 2328 |
| MDH2 | NM_001282403 | TTCTAGCGTAGCCGTCTGTG | 2329 |
| ME3 | NM_001014811 | GCAGGCGGGGTGAGGAGCTG | 2330 |
| MECOM | NM_001105077 | CGACGGACAGAGACACACGG | 2331 |
| MECOM | NM_001105077 | GGGTTTCTCTGCCGGCTTGT | 2332 |
| MECOM | NM_001105078 | AGAGAACTCCTCACTTTAAA | 2333 |
| MECP2 | NM_004992 | GCTGCGAGCCCGCCCGTCAT | 2334 |
| MED12 | NM_005120 | CCCAGCTCATTCTGCGCCTC | 2335 |
| MED17 | NM_004268 | AAACGCAGGCTTAAAAAGCA | 2336 |
| MED21 | NM_001271811 | GGCTGGATCTTTTGAGTAAC | 2337 |
| MED24 | NM_001079518 | GGGTGTGGCGTTCAGCAATA | 2338 |
| MED29 | NM_017592 | ATCCGTGTGTGGTTCCGAGC | 2339 |
| MEDAG | NM_032849 | GAGGTGGGGAGAGTCCTCCC | 2340 |
| MEF2C | NM_002397 | GAAGACGGAGCACGAATGGT | 2341 |
| MEF2D | NM_001271629 | CTTGCCAGGGAGAAGAGGGC | 2342 |
| MEGF11 | NM_032445 | GAAGGAGAGGGAGGGGCCGA | 2343 |
| MEGF8 | NM_001271938 | CAAATGGGCGGGGATTTCCC | 2344 |
| MEIS2 | NM_002399 | GGAGGAAAAGACGGAGAGAG | 2345 |
| MEIS3 | NM_020160 | GGTGGGAGTCGGGGAGGGGC | 2346 |
| MEN1 | NM_130801 | CCCGGCCCGCCACTATTTCC | 2347 |
| MEN1 | NM_130804 | CACTGAAGCCTCCGCCTCCC | 2348 |
| MEOX1 | NM_001040002 | TCTGAAGTGAAATGTGAGAG | 2349 |
| MEPE | NM_001184694 | CAAAAGCAGACACTGAGACA | 2350 |
| MEPE | NM_001184694 | TTTTGAGAAAGCCTAACCTC | 2351 |
| MEPE | NM_020203 | TAAAATTACTTCACCCCCTA | 2352 |
| METAP1 | NM_015143 | ACGCAGGCACCGCCGGCGGG | 2353 |
| METTL22 | NM_024109 | CTCCTATTTAAGTCTTTTAG | 2354 |
| MFAP1 | NM_005926 | TTCCTTTGGGCTTTGCTGTT | 2355 |
| MFN2 | NM_014874 | AAGATTACAGAATGCAAATC | 2356 |
| MFNG | NM_002405 | CACAACAAACCCTCCGTGCC | 2357 |
| MFSD10 | NM_001120 | ATGGGGTGCACACCGGACGC | 2358 |
| MFSD2B | NM_001080473 | GGGAAACGCAGAAACCGCGA | 2359 |
| MFSD4B | NM_153369 | CTCTTGATTTCCCTGGTCCC | 2360 |
| MFSD8 | NM_152778 | TTCCTTGTGACGAAAGGAGC | 2361 |
| MFSD9 | NM_032718 | TCATCATTATCATCACAAAC | 2362 |
| MGAT1 | NM_001114619 | AGGTCCTCGCCTCCACGCAG | 2363 |
| MGAT4D | NM_001277353 | GCTCTAGTGTTTCTCAGCTT | 2364 |
| MGAT5 | NM_002410 | CTGTAAGCTGAGGGGAAATC | 2365 |
| MGST1 | NM_145764 | TCGAGAGATCAAGTCCATCC | 2366 |
| MIB1 | NM_020774 | GGCCGGGGGAGGCTAGCCCG | 2367 |
| MICAL2 | NM_001282667 | TGCCACATCGACAGGCCAAA | 2368 |
| MICB | NM_001289160 | CAGGAGACTCACTTGAACCC | 2369 |
| MID2 | NM_052817 | ACACACACGCACACCCGTCC | 2370 |
| MIEF1 | NM_019008 | CTCCGTGTGTGACCTCACCA | 2371 |
| MIEF2 | NM_148886 | CTTGGTTTATCCTGCGAACG | 2372 |
| MIGA1 | NM_001270384 | GTTTTTGCATCCACTTGACG | 2373 |
| MIIP | NM_021933 | GGAGTCTCACTCTGTTGCCC | 2374 |
| MINK1 | NM_153827 | GCGCACGCGCACCAGCTGGT | 2375 |
| MINPP1 | NM_001178118 | CATAATCATGCTTCAACTAC | 2376 |
| MIS18BP1 | NM_018353 | GCTACGGCGCACAGCCTGTA | 2377 |
| MITF | NM_198177 | TGCTGTTGCAGACAGAAACC | 2378 |
| MKL1 | NM_001282662 | GCCTGACTTCCTGTGACTGA | 2379 |
| MLC1 | NM_015166 | GGGTTCATGGTTTAAGGAGC | 2380 |
| MLYCD | NM_012213 | CGGCTGGGGACGCGGCCAAT | 2381 |
| MMADHC | NM_015702 | GAGGACTATCAAACGCATCA | 2382 |
| MMD | NM_012329 | ACGCTGCCATTCATTCCCGC | 2383 |
| MMD | NM_012329 | CGGGGTGCCGATTGGCTGAC | 2384 |
| MME | NM_007288 | GCTCTCCTGGGACTCACCAG | 2385 |
| MMP11 | NM_005940 | CTGAACTCTCCTAGCAGCCG | 2386 |
| MMP17 | NM_016155 | GGCGTTTCCCCGGGTGTCTT | 2387 |
| MMP20 | NM_004771 | CTCATTTCTCTCCCTGATGA | 2388 |
| MMP24 | NM_006690 | TGGCTCCCCGACCAGCCCTG | 2389 |
| MMP27 | NM_022122 | TGTGTTTACTAAACAATTGC | 2390 |
| MMRN2 | NM_024756 | GTCCCTGAGCCAAGTCCTCA | 2391 |
| MOCS3 | NM_014484 | ATTGATCGCTAGTTCTTCTA | 2392 |
| MOK | NM_014226 | AAGGCTATCGTCCACGTAGT | 2393 |
| MOK | NM_014226 | CAAATCCCCGCCTTTGACAC | 2394 |
| MON1A | NM_032355 | AAATGAACTGCTAGCTGGCT | 2395 |
| MON1B | NM_001286640 | GGAGACGTCAATCAATGGAT | 2396 |
| MORC3 | NM_015358 | GGGAAGATGAATTGCCTGAC | 2397 |
| MORF4L2 | NM_001142421 | CTTCTGTAAATAGCACTAGT | 2398 |
| MORF4L2 | NM_001142421 | GAGCAAAATTATTTGGATCT | 2399 |
| MOSPD2 | NM_001177475 | TTGAGTTCCCCTTATGATTC | 2400 |
| MPDU1 | NM_004870 | AAGACAAGATGGCGCCCAGC | 2401 |
| MPHOSPH10 | NM_005791 | GGCACCGGCGACCTTCGCCA | 2402 |
| MPP2 | NM_001278374 | CGAGAGCCTCTTTTAGGTCT | 2403 |
| MPP2 | NM_001278376 | GTGCAGAGCAGGCGGTAACC | 2404 |
| MPP6 | NM_016447 | GCGGCGGCGGCTGGAGGAGG | 2405 |
| MPP7 | NM_173496 | AAGCGGGCAGCCACATTTGC | 2406 |
| MPZL3 | NM_001286152 | CTTTTGCTTGAAAATGAAGT | 2407 |
| MRE11 | NM_005590 | TGGGTTGTTATTCCCTGTCC | 2408 |
| MREG | NM_018000 | CCCTGGAGCCACAGAGCACG | 2409 |
| MRFAP1L1 | NM_203462 | GATGGACGTGCGCGCGCCCG | 2410 |
| MRGBP | NM_018270 | TTTCTTACTGTGCTTTAAAG | 2411 |
| MRM2 | NM_013393 | AGACTAGGGGAGCTGAGCCA | 2412 |
| MRNIP | NM_016175 | AGGGGCGGGGCCGCGGCGGC | 2413 |
| MROH5 | NM_207414 | GAGAAGGAAGGGGCAGGCCC | 2414 |
| MRPL12 | NM_002949 | CGGGCGACCCTCGTCCCGCC | 2415 |
| MRPL18 | NM_014161 | TAAGCAACAAGCGTGGTCTT | 2416 |
| MRPL27 | NM_016504 | CTGCAGAGCGGTGTTCAGGA | 2417 |
| MRPL3 | NM_007208 | GAATAAGGACAGACTTCCTG | 2418 |
| MRPL35 | NM_145644 | GTAAAACGACTGCCTATAGA | 2419 |
| MRPL37 | NM_016491 | CCAGGTTCCTCCCAGTCTCT | 2420 |
| MRPL38 | NM_032478 | AGGGGTGCGAGCTCCGATTC | 2421 |
| MRPL38 | NM_032478 | CGCTGCGTCCTGATTTCCCC | 2422 |
| MRPL52 | NM_181306 | GAGAGACAAAACTGCAGTAC | 2423 |
| MRPL58 | NM_001545 | ACCGTCTTCCCCAGCCAACC | 2424 |
| MRPS18C | NM_016067 | AGCTCTCAGGGCTCGCGGAC | 2425 |
| MRPS28 | NM_014018 | GAAGAGACTTAAGCTAAAAT | 2426 |
| MRPS33 | NM_016071 | GATGGCTGCGAAGTCTACGG | 2427 |
| MRPS33 | NM_016071 | TCATTAGTGACCAGCTCGGG | 2428 |
| MRPS35 | NM_001190864 | ACTGATTCACTCGATTTTTA | 2429 |
| MRVI1 | NM_001206880 | GATTGCCAGAGAGAATGGCC | 2430 |
| MS4A14 | NM_032597 | AAGATAACTACGTGAGGTGA | 2431 |
| MSANTD1 | NM_001042690 | GCCGGGGCGGCACTGAACTG | 2432 |
| MSANTD3 | NM_001198805 | CGCCTCGCCGGCCCCTCCCC | 2433 |
| MSANTD3 | NM_001198806 | GAATGAATGTTATCACGGAC | 2434 |
| MSH5 | NM_172165 | TCTGCCGTTGCTTAGCAGCC | 2435 |
| MSL3 | NM_001193270 | GGGCTGGGGGACCCGGGACC | 2436 |
| MSLN | NM_001177355 | CAGGAAGGCAAAGCTGCCCT | 2437 |
| MSMB | NM_002443 | AGGTAAACACATAACTTGGG | 2438 |
| MSMO1 | NM_001017369 | CTGCAGAGCCAGCCAATGGT | 2439 |
| MSR1 | NM_138715 | CACACCACTGCACTCCACCC | 2440 |
| MSTN | NM_005259 | GACTGTAACAAAATACTGCT | 2441 |
| MSX1 | NM_002448 | GCGGGCCCGGAGCGATCCAT | 2442 |
| MT1B | NM_005947 | CAGGTCACTGCTCATGGCCC | 2443 |
| MT3 | NM_005954 | TGCGCGCTTCCACGCAGTGG | 2444 |
| MTIF3 | NM_152912 | TGTCGAATTTCTGCAGCAAT | 2445 |
| MTMR4 | NM_004687 | ACCCCACTCATTGGTCGAGT | 2446 |
| MTNR1A | NM_005958 | GCGGGCTCGCGGCGGACACC | 2447 |
| MTR | NM_000254 | AGGCTTACACTTCCGGATCC | 2448 |
| MTRNR2L10 | NM_001190708 | TCGTCTGGTTTTGGGGAACT | 2449 |
| MTRNR2L7 | NM_001190489 | TATTCACAACAGCAAAGACA | 2450 |
| MTTP | NM_000253 | TCCCTGTCAACTCTTCAGCT | 2451 |
| MTUS1 | NM_001166393 | AGGCTCAGAGATGTTGTCAC | 2452 |
| MTUS1 | NM_001001931 | TGTTGTGGCAACAGAATTTG | 2453 |
| MTUS1 | NM_020749 | ACTTTAATTCCCACATGCTG | 2454 |
| MTUS2 | NM_015233 | TATTGATTTGCCTCACCCTG | 2455 |
| MURC | NM_001018116 | AGTCAGTCAGCAAGCATGTT | 2456 |
| MUS81 | NM_025128 | ACTGGTCTTGAAAAGAGTCC | 2457 |
| MUSTN1 | NM_205853 | ACTGGGATGAACCCTTGCAG | 2458 |
| MUSTN1 | NM_205853 | TTCAGATGGTCACACATTCC | 2459 |
| MUTYH | NM_001048172 | ATGGCCGCCGACAGTGACGA | 2460 |
| MVB12A | NM_138401 | CCTCGCCACCACGCGTCGCC | 2461 |
| MXI1 | NM_001008541 | TGGTGGCCACGCCGGAGCCC | 2462 |
| MXI1 | NM_130439 | GGCTTCCCTGCCTCTCCCCA | 2463 |
| MYADM | NM_001020818 | GCTCTCAGCCCATGTTTATA | 2464 |
| MYADM | NM_001020820 | ACAGACCCTCTTTGTCACTC | 2465 |
| MYCN | NM_005378 | GGCTTTTGGCGCGAAAGCCT | 2466 |
| MYCT1 | NM_025107 | CCTAAAAGCAGTTTTGGAGG | 2467 |
| MYD88 | NM_001172569 | GTGGAGCCACAGTTCTTCCA | 2468 |
| MYF6 | NM_002469 | GTGATTCTCTCTGTGTAACC | 2469 |
| MYH1 | NM_005963 | AATATGAGGGGAATTAGGCT | 2470 |
| MYH13 | NM_003802 | TTACTTGGATAAATGACCAG | 2471 |
| MYH14 | NM_001145809 | GGCCAATCAGAAGTTGTCGA | 2472 |
| MYH8 | NM_002472 | AATGTCTTGCCCTAACAAAG | 2473 |
| MYH8 | NM_002472 | GTCACTACAAACTATGCTGA | 2474 |
| MYL10 | NM_138403 | ACAAAGGGCTTTTTGTATCC | 2475 |
| MYL10 | NM_138403 | TACACCAAGGCAAGAACCCC | 2476 |
| MYL3 | NM_000258 | GGAGGGCATTGTTCAGGCTC | 2477 |
| MYL7 | NM_021223 | TTGAGGACATGAAGGTCATC | 2478 |
| MYL9 | NM_006097 | CAAGGCCCTCTGTGCAGCCC | 2479 |
| MYLK4 | NM_001012418 | CAGGTAAGGAGAGGATGAAC | 2480 |
| MYNN | NM_001185119 | ACATACATGGTTAAGAATGA | 2481 |
| MYO16 | NM_015011 | TCCAGAAAACACATCAGCTC | 2482 |
| MYOCD | NM_001146312 | CCAATCAGGAGCGGCGAGCG | 2483 |
| MYRF | NM_013279 | CCCAGCCCACCACCGGCACA | 2484 |
| N4BP1 | NM_153029 | GTCACCCTCAGTCGCCATGT | 2485 |
| N4BP2L1 | NM_052818 | GTGCGTCACCCTTGTTTTCC | 2486 |
| NAA35 | NM_024635 | CTGTCGGAGTCCTGGGTAGT | 2487 |
| NABP1 | NM_001031716 | TGCTTCCCCTCCCCAGCACC | 2488 |
| NACAD | NM_001146334 | CACCCACTGCCCCCACCGCC | 2489 |
| NADSYN1 | NM_018161 | TTGCCCGCAAGGGCCGGGCC | 2490 |
| NAE1 | NM_003905 | GGGCAAATTGGCAGGCTAGC | 2491 |
| NAGS | NM_153006 | CGGGGTCCGGACAGGGGACC | 2492 |
| NANOG | NM_024865 | AGAGTAACCCAGACTAGGTG | 2493 |
| NAP1L5 | NM_153757 | GATGTCAGGGTAGCAACAGG | 2494 |
| NARS2 | NM_001243251 | AGATTATCGCTGAAAGAACG | 2495 |
| NASP | NM_152298 | CACCTCCTGCCCTCTCCATA | 2496 |
| NAT8B | NM_016347 | CTACCTTCTCCCAGTGGCAG | 2497 |
| NAT8L | NM_178557 | GGGCGGCCGGGGCGCGCGCA | 2498 |
| NAV2 | NM_001111019 | AAAATATGCATTAATTCCGC | 2499 |
| NAXE | NM_144772 | GGTCCAGCTTCCCTTCCACT | 2500 |
| NBL1 | NM_005380 | ACGGGCCAGGGCGCCCGGCT | 2501 |
| NBL1 | NM_005380 | TTCGGCGCGCTCCGACGGCG | 2502 |
| NBPF1 | NM_017940 | CAGGTTAGGGGCCGCGCAGG | 2503 |
| NBPF11 | NM_001101663 | AGCTTCTCTCAGGCCACACA | 2504 |
| NBPF12 | NM_001278141 | CGAATTGCAGGGTCAAGGGC | 2505 |
| NBPF20 | NM_001278267 | CATCTTCAAATAAGTACACA | 2506 |
| NBPF3 | NM_001256417 | CGAGCAGGTTAGGGGCCCTG | 2507 |
| NBPF4 | NM_001143989 | CACCCTTGTGACAATGCTAC | 2508 |
| NBPF6 | NM_001143987 | CACCCTTGTGACAATGCTAT | 2509 |
| NCALD | NM_001040629 | GGGGGGCCAAGATGAGGCGC | 2510 |
| NCK2 | NM_001004720 | AGTGTGGCTTCCAGTGCTCC | 2511 |
| NCK2 | NM_003581 | CTCCGGCCTGACGATCCCCG | 2512 |
| NCKAP1L | NM_001184976 | AAAACAAATCACCAGGAACA | 2513 |
| NCMAP | NM_001010980 | CCCCGCTCCTGGGTCCTTTT | 2514 |
| NCMAP | NM_001010980 | CTCTACTGGACTGAGTGCCC | 2515 |
| NCR3LG1 | NM_001202439 | GCGCAACCTCGTGCCGCGGG | 2516 |
| NCSTN | NM_015331 | GAATTTGGTTAACATCTCTC | 2517 |
| NDFIP1 | NM_030571 | TCGTCGGAGCAACTACACCA | 2518 |
| NDN | NM_002487 | CATGGCGAGGCTTCACCTGC | 2519 |
| NDP | NM_000266 | TTGGAAATACAAAGGCAGTG | 2520 |
| NDRG2 | NM_201538 | GGACGCTTCCAGGCTCTGCT | 2521 |
| NDUFA12 | NM_018838 | GCTTCCCAAGTAGGCAGAAT | 2522 |
| NDUFB2 | NM_004546 | GGGCTTTGCTCTCGGGAGAG | 2523 |
| NDUFB3 | NM_002491 | GTAGGCGGCGGTGCTGTCTT | 2524 |
| NDUFB7 | NM_004146 | CCTGTCCGCGAGGTGACGCC | 2525 |
| NDUFS1 | NM_001199984 | GAGGTCTTGTATGGATGGGA | 2526 |
| NDUFS6 | NM_004553 | ACAGTACTCGGTGTAATCAG | 2527 |
| NDUFV2 | NM_021074 | GGCGGGGACCAGTCCGTGCT | 2528 |
| NECAB3 | NM_031232 | TGGGTAGGCCCGCAGCCCCT | 2529 |
| NECAP1 | NM_015509 | TGGAAATCTCTGTCCTGGAG | 2530 |
| NECAP2 | NM_001145277 | ACAGACCCCTCTGTAACCCG | 2531 |
| NEDD4 | NM_006154 | CATGGCGTGGGGAGCGCGCG | 2532 |
| NEDD4 | NM_198400 | AAGTCGGCTGGAGAAAGTAT | 2533 |
| NEDD4L | NM_001144970 | ACACACGTCTCATGGCAAGT | 2534 |
| NEIL1 | NM_024608 | GAAGTGCAGACTCCACACGG | 2535 |
| NEK8 | NM_178170 | CCGCCACGCGTCCGTATTTG | 2536 |
| NELFE | NM_002904 | TCGCTCTGTCTCCATCATCC | 2537 |
| NENF | NM_013349 | GGCTACTCGGGCCACGCAGC | 2538 |
| NET1 | NM_005863 | TCGGGAATGCATTTTAAATC | 2539 |
| NETO1 | NM_001201465 | GGCGGTCGCAGGGCGAGCCC | 2540 |
| NETO2 | NM_001201477 | GCCGGTCACTGCCCCGGCGC | 2541 |
| NEU1 | NM_000434 | TTTTGATTGGCCGCGGCACC | 2542 |
| NEURL1 | NM_004210 | GGCGGAGCGCGGGGCGTTCT | 2543 |
| NEXN | NM_001172309 | GCGAGCTGACCCCCTAACTT | 2544 |
| NFATC4 | NM_001198967 | GACTGGGGGGGTGGTCCCCT | 2545 |
| NFIA | NM_001145511 | CGACTGGCGGGGAGACAGAC | 2546 |
| NFKBIL1 | NM_001144962 | ATGAGATTGGGAGAGACACT | 2547 |
| NFRKB | NM_001143835 | TTGCGCGTCTCACCTGATTT | 2548 |
| NFYB | NM_006166 | GCTCCGGATGCCGCTCCTCT | 2549 |
| NGEF | NM_001114090 | GCCCGGGTCGCGCCCAGCCC | 2550 |
| NGLY1 | NM_001145294 | TGAATGTAAAGGAGGAAAGG | 2551 |
| NHLRC2 | NM_198514 | ACATCCCCAACCCTCCACAT | 2552 |
| NHLRC3 | NM_001017370 | ACATCCTATTCCTACCATCC | 2553 |
| NHLRC3 | NM_001017370 | AGGCATCCATAGCGGATGCC | 2554 |
| NHLRC4 | NM_176677 | GAAGCTTCAGGGGCCAAGGC | 2555 |
| NID2 | NM_007361 | TCCCGGGTCATCCTCTCATC | 2556 |
| NIF3L1 | NM_021824 | AGTGTAAGGCGAAACTACCT | 2557 |
| NINJ1 | NM_004148 | CGCGACGCCGATGGCCCCAG | 2558 |
| NINJ2 | NM_016533 | TGAGCTAGTAGCTTTATGAC | 2559 |
| NIPA1 | NM_144599 | GTGCCAGGGACCGGCGCCTT | 2560 |
| NKIRAS1 | NM_020345 | ACAGCTCTTTCCTTTCCGTC | 2561 |
| NKIRAS1 | NM_020345 | GGAAGACGATCAAAGGCGGA | 2562 |
| NKIRAS2 | NM_001001349 | GAGCTGCTCTATGCTCCAAC | 2563 |
| NKRF | NM_001173488 | ATAAAAAATGATCATCAGGC | 2564 |
| NKX2-2 | NM_002509 | GCGGGAGAAGGGTGGAAAAA | 2565 |
| NLGN4Y | NM_014893 | ACTGCCTGGGGTGCTTCTTT | 2566 |
| NLRC3 | NM_178844 | GCCCCCGTGCAAGTTAAGTG | 2567 |
| NLRC4 | NM_001199139 | CCTCCGGAGTATAAACAGCC | 2568 |
| NLRP12 | NM_144687 | ACTGTTTTGTCAAGAGATCC | 2569 |
| NLRP14 | NM_176822 | CGAGTGTCTACTCCAAGACC | 2570 |
| NLRP3 | NM_001127461 | GTTCACCTTGCTCTCCTCTG | 2571 |
| NLRP6 | NM_138329 | GTGGACCCGGGGAATGGACC | 2572 |
| NLRP8 | NM_176811 | GGATTAGTCCATTAGACTAA | 2573 |
| NM_000645 | NM_000645 | AGAGAAAGCTAGTTTCTCTA | 2574 |
| NM_ | NM_001001435 | CCTCTCAGCTTCTCTTCCCC | 2575 |
| 001001435 | | | |
| NM_ | NM_001004727 | AAGGGAAGAGCATTCCAAGA | 2576 |
| 001004727 | | | |
| NM_ | NM_001004727 | TTGGAAATTGAAAGGTGAGT | 2577 |
| 001004727 | | | |
| NM_ | NM_001014444 | AGGGTCCCTCCCATAACACG | 2578 |
| 001014444 | | | |
| NM_ | NM_001017436 | TGCAGAACCTTCTCACCCAG | 2579 |
| 001017436 | | | |
| NM_ | NM_001024607 | CACACTGTAACTCCCATTGT | 2580 |
| 001024607 | | | |
| NM_ | NM_001033019 | CAGTCCTATACAAACCTCTC | 2581 |
| 001033019 | | | |
| NM_ | NM_001039517 | GTGCCCTCTTCATCCCGCGT | 2582 |
| 001039517 | | | |
| NM_ | NM_001039841 | ACTTACAGCGACCTTCTTTC | 2583 |
| 001039841 | | | |
| NM_ | NM_001040282 | CGTGCGTGCACACGTGTATG | 2584 |
| 001040282 | | | |
| NM_ | NM_001042389 | GGTATAGCATATTTAAGCTC | 2585 |
| 001042389 | | | |
| NM_ | NM_001042391 | GTGGGTTGTGGCCCTGGCCC | 2586 |
| 001042391 | | | |
| NM_ | NM_001042395 | CTCTCAGTGCCTTGGAAGAC | 2587 |
| 001042395 | | | |
| NM_ | NM_001042395 | GAGGCAGGTTCTGTCTCTCC | 2588 |
| 001042395 | | | |
| NM_ | NM_001042402 | CGCGGGGCCGCTAAGGGTTG | 2589 |
| 001042402 | | | |
| NM_ | NM_001077685 | GACCAGCCGGCTTATTTAAT | 2590 |
| 001077685 | | | |
| NM_ | NM_001079809 | CCGGCACCCGCGAATCAAGC | 2591 |
| 001079809 | | | |
| NM_ | NM_001080826 | TTAAGAGCCTTGTGACAAAT | 2592 |
| 001080826 | | | |
| NM_ | NM_001097616 | AATCATTGACTGTTTACTCT | 2593 |
| 001097616 | | | |
| NM_ | NM_001099414 | AGCAAATGCCAGCCTTCCAG | 2594 |
| 001099414 | | | |
| NM_ | NM_001099435 | TCACTGCAACATCCATCTCC | 2595 |
| 001099435 | | | |
| NM_ | NM_001101337 | CTAAATCCTAATTCAGTGCC | 2596 |
| 001101337 | | | |
| NM_ | NM_001101337 | GTTAACACTTCCTAGAAGCC | 2597 |
| 001101337 | | | |
| NM_ | NM_001103169 | GTGGCTGGATCCGGCTGGAT | 2598 |
| 001103169 | | | |
| NM_ | NM_001104548 | GGGTGTGGGTTCTGAGAGGT | 2599 |
| 001104548 | | | |
| NM_ | NM_001123065 | AGAGCAGAGCTCCTATACCC | 2600 |
| 001123065 | | | |
| NM_ | NM_001123228 | TAGTCTTATGAACAGAGTGA | 2601 |
| 001123228 | | | |
| NM_ | NM_001123228 | TGTTTCATTTCTTGTCCCAA | 2602 |
| 001123228 | | | |
| NM_ | NM_001127386 | CTCCACCCCTTCATGAATGG | 2603 |
| 001127386 | | | |
| NM_ | NM_001129826 | CTGACTTAAGACATAACTTC | 2604 |
| 001129826 | | | |
| NM_ | NM_001129895 | CCCCCCTCAGAGGCTCCACG | 2605 |
| 001129895 | | | |
| NM_ | NM_001139502 | ATATTGTGGGAGAGACCCGG | 2606 |
| 001139502 | | | |
| NM_ | NM_001142861 | AATGTGCTATCAACACTACT | 2607 |
| 001142861 | | | |
| NM_ | NM_001163391 | ACAATGGCTGGGTAAAGAAG | 2608 |
| 001163391 | | | |
| NM_ | NM_001164182 | CTAGCTTCATAATTGCAGTA | 2609 |
| 001164182 | | | |
| NM_ | NM_001170721 | TCAGCCCCACTGCTAATCAC | 2610 |
| 001170721 | | | |
| NM_ | NM_001184963 | CTGGGCCGAAGACCCTCTTT | 2611 |
| 001184963 | | | |
| NM_ | NM_001190943 | AAGAGCTGTCCCTGGGCAGT | 2612 |
| 001190943 | | | |
| NM_ | NM_001193523 | AGGACGATCCTCTCCGGCTT | 2613 |
| 001193523 | | | |
| NM_ | NM_001195017 | GGAAAAAGTTAAGCAGAATC | 2614 |
| 001195017 | | | |
| NM_ | NM_001195150 | GGGCATGGCAAGTAGAACCC | 2615 |
| 001195150 | | | |
| NM_ | NM_001195190 | GCATATTTTGCTGACTGGCA | 2616 |
| 001195190 | | | |
| NM_ | NM_001195257 | GGACATAAAACAGCTTCCGT | 2617 |
| 001195257 | | | |
| NM_ | NM_001199053 | GCCAACGCCAGCGCTGGACC | 2618 |
| 001199053 | | | |
| NM_ | NM_001199057 | GCGCTGTGTGGCTCCCGAGT | 2619 |
| 001199057 | | | |
| NM_ | NM_001207030 | CAATCCATCTTGAATCCTAT | 2620 |
| 001207030 | | | |
| NM_ | NM_001242348 | GGACCAATCTTGAGGTGGCA | 2621 |
| 001242348 | | | |
| NM_ | NM_001242473 | AGCTACCTGTGGGTGACTTC | 2622 |
| 001242473 | | | |
| NM_ | NM_001242668 | TTAGTCTCTTAGTGATCAAT | 2623 |
| 001242668 | | | |
| NM_ | NM_001242713 | TGGGGAGCGCATAGGCTCAT | 2624 |
| 001242713 | | | |
| NM_ | NM_001242812 | AGGGAGGGGGATGCAGAACT | 2625 |
| 001242812 | | | |
| NM_ | NM_001242853 | GAGTGATTATTGAACCTTTC | 2626 |
| 001242853 | | | |
| NM_ | NM_001242885 | GATGCTGTCAAGACCGGCCC | 2627 |
| 001242885 | | | |
| NM_ | NM_001243466 | TAATGGGAATGAAAACAATG | 2628 |
| 001243466 | | | |
| NM_ | NM_001243476 | TCTTCCCCTAAGAGGTGCCC | 2629 |
| 001243476 | | | |
| NM_ | NM_001244193 | AATGGCAGTCTGGCCAGGCG | 2630 |
| 001244193 | | | |
| NM_ | NM_001247987 | GCCGGAGCCTTCCAGGTGGA | 2631 |
| 001247987 | | | |
| NM_ | NM_001253913 | AGACTGAATAGCTTTGTGGG | 2632 |
| 001253913 | | | |
| NM_ | NM_001257177 | ACCATGGGTGAGATAGGTTT | 2633 |
| 001257177 | | | |
| NM_ | NM_001258300 | GTGCTAGGAGGCGAGGCGAG | 2634 |
| 001258300 | | | |
| NM_ | NM_001278082 | CGGAGATCCGTTTTCCATGC | 2635 |
| 001278082 | | | |
| NM_ | NM_001278094 | GAGATTCTAACAGTTGACAC | 2636 |
| 001278094 | | | |
| NM_ | NM_001278319 | GGAAGCAGAACTACCCTACC | 2637 |
| 001278319 | | | |
| NM_ | NM_001278420 | GGCACCTGTTCTTCCGGGGG | 2638 |
| 001278420 | | | |
| NM_ | NM_001278502 | AAGGACTGATTGATCAGCTG | 2639 |
| 001278502 | | | |
| NM_ | NM_001278606 | ACAACATCACATCTTGCAAT | 2640 |
| 001278606 | | | |
| NM_ | NM_001278606 | GTTTGCCTCATTTACACGTA | 2641 |
| 001278606 | | | |
| NM_ | NM_001278674 | AGTTGACATTGGGGGAGGCT | 2642 |
| 001278674 | | | |
| NM_ | NM_001281518 | AGTTAGGAACAGGTAATTAA | 2643 |
| 001281518 | | | |
| NM_ | NM_001282503 | AGCTTTCCTTATGATGCTAC | 2644 |
| 001282503 | | | |
| NM_ | NM_001282507 | ACATTCATTTTAAGCATGCA | 2645 |
| 001282507 | | | |
| NM_ | NM_001282578 | GTGGGGACTTGCAGGTTGCT | 2646 |
| 001282578 | | | |
| NM_ | NM_001282670 | GACAAAGCTCTCCGTGGCTG | 2647 |
| 001282670 | | | |
| NM_ | NM_001284235 | ACCTCGCGCCAGCGGAGTCC | 2648 |
| 001284235 | | | |
| NM_ | NM_001284235 | GCGGGAGCGCCGCTGACTCA | 2649 |
| 001284235 | | | |
| NM_ | NM_001286517 | CGAAGCACAGGGGACACGCC | 2650 |
| 001286517 | | | |
| NM_ | NM_001287428 | TCTGGTGAGAGCACAGAGCC | 2651 |
| 001287428 | | | |
| NM_ | NM_001287430 | GAGGAAGGTGGGGGCGGGCG | 2652 |
| 001287430 | | | |
| NM_ | NM_001287601 | GGAGCTGGCTGAGAGGGGAC | 2653 |
| 001287601 | | | |
| NM_ | NM_001287807 | ACACTGGGAGATACAAATTA | 2654 |
| 001287807 | | | |
| NM_ | NM_001287807 | CTTTGATTATGTCACAGGCT | 2655 |
| 001287807 | | | |
| NM_ | NM_001287812 | GGGGAATGTGGACATATACC | 2656 |
| 001287812 | | | |
| NM_ | NM_001289922 | ACTGGGCAGGTGCCCAGATC | 2657 |
| 001289922 | | | |
| NM_ | NM_001289933 | CGGTGCCTTCATGTCCCCGC | 2658 |
| 001289933 | | | |
| NM_ | NM_001290021 | GTCTGTGGCATGGTTGCTAT | 2659 |
| 001290021 | | | |
| NM_ | NM_001290031 | GAGATGGGTGTCCCTGGTAG | 2660 |
| 001290031 | | | |
| NM_ | NM_001291410 | GAACCGCTGACTGCGAAGTC | 2661 |
| 001291410 | | | |
| NM_ | NM_001291420 | GCTGGCGTCTCTGAGGACCT | 2662 |
| 001291420 | | | |
| NM_ | NM_001291717 | ATTGTTTTATCAGTCAGGCC | 2663 |
| 001291717 | | | |
| NM_004542 | NM_004542 | CACAAGTAGAGGCGAAAGCA | 2664 |
| NM_004542 | NM_004542 | TCTGTGCGACGGCCCGCTTT | 2665 |
| NM_006250 | NM_006250 | AGTGTATCCCTCATTTCTTT | 2666 |
| NM_014577 | NM_014577 | CCAACAGGGGAGCCCTGTAC | 2667 |
| NM_014577 | NM_014577 | CCTGTCCATCCTCTATAGAC | 2668 |
| NM_015372 | NM_015372 | TAAAATGAAACGTGACTTCT | 2669 |
| NM_018232 | NM_018232 | ATAACAAGCATGTTGTACTT | 2670 |
| NM_022896 | NM_022896 | AGGGCGCCCTTTGGCCTCGG | 2671 |
| NM_025170 | NM_025170 | AGACAGAAGACTTTACATGC | 2672 |
| NM_130387 | NM_130387 | TAGACAATATGGGAAGCCTC | 2673 |
| NM_138464 | NM_138464 | GAGTCGGTGGCAGGTCCTGA | 2674 |
| NM_144728 | NM_144728 | AGAAGCTTCTAGACATTTCC | 2675 |
| NM_144729 | NM_144729 | GGGTCCTCGGTGTTAAAACA | 2676 |
| NM_145813 | NM_145813 | CCTGTTCAAGGAGGGACTCG | 2677 |
| NM_173600 | NM_173600 | TAGAAGATGTCATAGGAGTA | 2678 |
| NM_173687 | NM_173687 | GTTCTTATCTCCCTTGTATT | 2679 |
| NM_175895 | NM_175895 | GCCGGGAGTAGCCGAGCCGC | 2680 |
| NM_178342 | NM_178342 | ACTGGGTTTCAGGCAAGTTC | 2681 |
| NM_207313 | NM_207313 | GCATTCATTTGCACCTGACC | 2682 |
| NMD3 | NM_015938 | ACTGACGGCAAATGAGCCCC | 2683 |
| NME1 | NM_000269 | TGAGTCAGAGAACCCGGGGG | 2684 |
| NME4 | NM_001286440 | AGCGCAAGGAAGGCAGAGGC | 2685 |
| NME5 | NM_003551 | TCATCCTTCTTCCCGTTTGA | 2686 |
| NME7 | NM_013330 | ATTTGTTTACCCTGCTCTTT | 2687 |
| NMRAL1 | NM_020677 | CAGGAGAATCTCTTGAACCC | 2688 |
| NMU | NM_006681 | CGAGGTAGGCCGGGGGCGGC | 2689 |
| NOC3L | NM_022451 | CTCTCGCGGTGACTGTCTCG | 2690 |
| NOD2 | NM_022162 | GGGACAGGCCACAAGTAAGT | 2691 |
| NOL6 | NM_022917 | GCCTCTTCGCGACGCTAGAA | 2692 |
| NOMO2 | NM_173614 | CTCTTCTGGGGCTGTGAACG | 2693 |
| NONO | NM_001145408 | CTAGATGCTTCTCCTGTTGC | 2694 |
| NOP2 | NM_001258310 | AGACGCGCAGCTTACACCCG | 2695 |
| NOS1 | NM_001204218 | CAGGGCAGGGCAGGTCTATT | 2696 |
| NOS1AP | NM_014697 | CAGCGCGGGGGCGGACCCGG | 2697 |
| NOS3 | NM_000603 | AACTACTTACCCTGCCAATC | 2698 |
| NOSIP | NM_015953 | GTTCCGGATATTGAAACTGG | 2699 |
| NOSTRIN | NM_001171631 | ATCTCAGGTGTTAGGTAAGT | 2700 |
| NOTO | NM_001134462 | TGATAAGTACATTTTCCATC | 2701 |
| NOX1 | NM_007052 | GGAAGGCAATGCTTCACATT | 2702 |
| NOX5 | NM_001184779 | CCCACAGTCCCTCATAAAAC | 2703 |
| NPAS4 | NM_178864 | GGGAGCCGCTGACTGGGGAG | 2704 |
| NPIPB5 | NM_001135865 | ACTTGTCGAATCAATGCATG | 2705 |
| NPIPB9 | NM_001287251 | AAAGTACAGGAATTTGAACT | 2706 |
| NPM2 | NM_001286681 | CAAGCCCGGGCTAAGAAGCC | 2707 |
| NPS | NM_001030013 | GAACAATTAGTCATATAGGA | 2708 |
| NPTXR | NM_014293 | CCCCGCCCCACTCGCTTCCC | 2709 |
| NQO1 | NM_001286137 | TTGACTTCCACCAGTTGCTC | 2710 |
| NR0B1 | NM_000475 | GGCGGGTGCTCTTTAAAAGC | 2711 |
| NR1H4 | NM_001206993 | TCCAGTTTAAGAACTTTTAG | 2712 |
| NR2F2 | NM_001145157 | GCTTTCGCTCTGCGCGAGTT | 2713 |
| NR3C1 | NM_001018074 | TTCCTAATTTCTCATTCCCA | 2714 |
| NR3C1 | NM_001018076 | CTCGCTGGAGGTTTTGCATT | 2715 |
| NR4A1 | NM_001202233 | TAGAGTCCCAAGGATCTGTG | 2716 |
| NRAP | NM_006175 | ACAACAGCATCATGTTTATG | 2717 |
| NRARP | NM_001004354 | CGGTGCCGTGCGCAGGGGTC | 2718 |
| NREP | NM_001142480 | TGGGGACGGCGCGGCGAGCG | 2719 |
| NRF1 | NM_005011 | CACGGAGCGCTTCAGAGGTT | 2720 |
| NRF1 | NM_001040110 | GATTCTTCAAGTCATCAATG | 2721 |
| NRIP1 | NM_003489 | GGCGAGGCGCAGGGACGACC | 2722 |
| NRL | NM_006177 | CCTGAGGCCTCCAACCAATA | 2723 |
| NRM | NM_001270709 | TCTAACATTCCCTTCTGTGA | 2724 |
| NRN1L | NM_198443 | CTCAGAGAGCAGAAATTCGC | 2725 |
| NRTN | NM_004558 | GGGTGGTGTTTAGGACAGTC | 2726 |
| NT5C1B | NM_001199088 | AATGACTTTGCCATTCATTT | 2727 |
| NT5E | NM_001204813 | TCGTGCGTTCTCAACCCAAC | 2728 |
| NTAN1 | NM_173474 | AAATCCAGGACATGGCCGCA | 2729 |
| NTHL1 | NM_002528 | GGAAGTGCGGGTCGCGCTTC | 2730 |
| NTM | NM_016522 | CAGCCCGCACCGGAGCCGCG | 2731 |
| NTNG1 | NM_014917 | TGGACGGCGGCAGAAGTGGG | 2732 |
| NTNG2 | NM_032536 | GGCGTCTCGTCGGGGAGCCG | 2733 |
| NTSR1 | NM_002531 | CCGCGCGGCGCGCCCAGCAG | 2734 |
| NUBP1 | NM_002484 | ATGATAGGAAATCTCTGAAA | 2735 |
| NUDCD3 | NM_015332 | GATTTTTGTCACGTTGTCTG | 2736 |
| NUDT1 | NM_002452 | GCGCTCGCTGAGTGCGGGGA | 2737 |
| NUDT12 | NM_031438 | AGATGTAGTTTGAAGCCCAC | 2738 |
| NUDT13 | NM_001283014 | GGGAGAGGATGAAGCAGGGG | 2739 |
| NUDT22 | NM_032344 | GGCGGCGGGGACAAACCTCC | 2740 |
| NUDT22 | NM_032344 | TGCGCCCCGCAGGGTGGTCC | 2741 |
| NUDT9 | NM_198038 | GGAACTGGAACGGGAATAAG | 2742 |
| NUMA1 | NM_006185 | CTTGGCGTCCCACTGCCTCA | 2743 |
| NUMB | NM_001005744 | GGTAAAGAGCGATGACGGGC | 2744 |
| NUMBL | NM_001289980 | GGCCCTGGAAATAGGGATCC | 2745 |
| NUP205 | NM_015135 | GGATTATTCCCATTCAAATA | 2746 |
| NUP54 | NM_017426 | TCACTGTTAAGGTAAAATGC | 2747 |
| NUP58 | NM_014089 | ACTGACATAATCCGCACTTT | 2748 |
| NUP62 | NM_016553 | GGGGCAGGGAGGGTGGAGGA | 2749 |
| NUP93 | NM_001242796 | ACTTGAGGAGCTGTCAATTG | 2750 |
| NUP93 | NM_001242796 | CAGGAGAGCTGCTCAGCAGA | 2751 |
| NUTM1 | NM_175741 | AACCGGAAGTCTCTCTCTCC | 2752 |
| NWD1 | NM_001007525 | TGCCCAATTCTCCCAGCAAC | 2753 |
| NXF5 | NM_032946 | AAAATTGGAGCGAGGGGTTG | 2754 |
| NXN | NM_022463 | CGAGGGCAGCCGAAGGGGCG | 2755 |
| NXT2 | NM_001242618 | GACCTTGTAGCAGTGTGTTC | 2756 |
| NYAP1 | NM_173564 | CGGGGGAGCCGCGGAGCCTG | 2757 |
| OARD1 | NM_145063 | AACGAAACTGCCCCACGAGT | 2758 |
| OAZ3 | NM_001134939 | AACTATTGTGATTGTGACAC | 2759 |
| OBSCN | NM_052843 | AGCCCAGCCCCAAAATAGCC | 2760 |
| OCM2 | NM_006188 | TGTGCCACTGCACTCCAGCC | 2761 |
| ODF3L2 | NM_182577 | CGTGGCCCCGTTTCTACACC | 2762 |
| ODF4 | NM_153007 | GGGATGCAGTGGCACAACCT | 2763 |
| OGFR | NM_007346 | TCCCCCAACGTCCGCCCGGG | 2764 |
| OGN | NM_014057 | AGCAGATTGTTTGATCTCCT | 2765 |
| OLFM3 | NM_058170 | CCTTCTGCTGTCATTGACAG | 2766 |
| OLFML1 | NM_198474 | ACAGGGCTACATCGCCCCTT | 2767 |
| OLFML2A | NM_001282715 | TTCATTCTCGCCTGCGGAAT | 2768 |
| OLFML2A | NM_182487 | GCGCGGGCAGGGATGCCCTT | 2769 |
| OLIG2 | NM_005806 | TTCATTGAGCGGAATTAGCC | 2770 |
| OMA1 | NM_145243 | GGCGCTCTAGCGCCTCCGTG | 2771 |
| ONECUT3 | NM_001080488 | ACCAGGATGTGGCAGGGGAG | 2772 |
| OPRK1 | NM_000912 | GGGAGCTGGGGGCTGACTCC | 2773 |
| OPRM1 | NM_001145286 | TGAGCCTCTGTGAACTACTA | 2774 |
| OR10A2 | NM_001004460 | CAAGGCACTTCCTCTGCCTG | 2775 |
| OR10A6 | NM_001004461 | AAGAAAATTTCTGTCAGGAT | 2776 |
| OR10C1 | NM_013941 | AAGGGTGGAATATGGACTCC | 2777 |
| OR10H2 | NM_013939 | TCACCTTAAGTGCTTTGTGC | 2778 |
| OR10W1 | NM_207374 | TATCACTTATTCAATACCCC | 2779 |
| OR11G2 | NM_001005503 | GAAATCATTGCAGCTTTTTG | 2780 |
| OR12D3 | NM_030959 | CTAGGAAGTGCAAGATTTGA | 2781 |
| OR13A1 | NM_001004297 | CAGTTTTCTAGATTTTATGC | 2782 |
| OR14I1 | NM_001004734 | ATGCAGAATTTCAAGTCTCA | 2783 |
| OR14I1 | NM_001004734 | GTTACTCAACTCATAGTCTT | 2784 |
| OR1B1 | NM_001004450 | CCATCTACTCTCCCTCCCTA | 2785 |
| OR1E2 | NM_003554 | GAGTGTTTTAGAAAGAAAGG | 2786 |
| OR1J4 | NM_001004452 | ATAATTCGCCAAGAGAGTAG | 2787 |
| OR1K1 | NM_080859 | ATAAATTGTTCAAGGCTTCC | 2788 |
| OR1L3 | NM_001005234 | AGTTCTGATTCTCCATGCTC | 2789 |
| OR1S1 | NM_001004458 | AAAATGCCTTAGAAAAAGAC | 2790 |
| OR1S1 | NM_001004458 | ATTTCAGCAGTGCAGAGATT | 2791 |
| OR2A7 | NM_001005328 | CAGGCGTGAGCTACCGCACC | 2792 |
| OR2AE1 | NM_001005276 | GTGCTTTTCCTTGGGTATAC | 2793 |
| OR2AP1 | NM_001258285 | ATTCAAATGGGCCACTGGTC | 2794 |
| OR2B6 | NM_012367 | TTTGGGGAACAGGAGGTGTT | 2795 |
| OR2C1 | NM_012368 | AGAGTCTCTCACTGTCACCC | 2796 |
| OR2G2 | NM_001001915 | CAATACTTTTTTGGGTAGGC | 2797 |
| OR2J3 | NM_001005216 | AATAAAATCACTGGTTATGG | 2798 |
| OR2M2 | NM_001004688 | TAGGAACTATCTGTTTGCTT | 2799 |
| OR2T10 | NM_001004693 | ATCTGATTCCCCATCTAGAA | 2800 |
| OR2T12 | NM_001004692 | CAGGAAAAGCTGTGCCTACT | 2801 |
| OR2T3 | NM_001005495 | TGTCTTACCAGAAAAAGGTC | 2802 |
| OR2T6 | NM_001005471 | TCATTCATCTTCATCCCATG | 2803 |
| OR3A1 | NM_002550 | TAAGGAATTTTGCGCTCCTT | 2804 |
| OR4A47 | NM_001005512 | CACTAAATCAAACTAGGATC | 2805 |
| OR4D2 | NM_001004707 | CAAGACAGCACCTAGTATAA | 2806 |
| OR4D9 | NM_001004711 | GCAAGTCAGTATGCCACCAC | 2807 |
| OR4F15 | NM_001001674 | ATAGTTATTTTCATGGCTGG | 2808 |
| OR4K14 | NM_001004712 | TGTATTAAGTGAAATAAGCC | 2809 |
| OR4K5 | NM_001005483 | AGAGGCCATAATAGTATGTC | 2810 |
| OR4N2 | NM_001004723 | TTTTTTGTTGTATCTCTGCC | 2811 |
| OR4S1 | NM_001004725 | ATTTTTTGTGATGGGGATGA | 2812 |
| OR51B4 | NM_033179 | ATTGTAAGCCTGTACTCACA | 2813 |
| OR51B5 | NM_001005567 | CCACAGAGCCAAATCATATC | 2814 |
| OR51E1 | NM_152430 | ACCCCCAGGCATATCCTCCC | 2815 |
| OR52D1 | NM_001005163 | AATGATGTGCAGGATATGGA | 2816 |
| OR52H1 | NM_001005289 | ATTTGTATCTGGAACAATCT | 2817 |
| OR52K1 | NM_001005171 | CCTAGCAGCCTTCATAGACA | 2818 |
| OR5A2 | NM_001001954 | GACTGTTTGTATGATCTTCT | 2819 |
| OR5D16 | NM_001005496 | ATCTCTGTTAATATCCTGAT | 2820 |
| OR5D18 | NM_001001952 | AACAACAAACTCATAGATTC | 2821 |
| OR5M11 | NM_001005245 | GGATAGATAGATACAGGTGT | 2822 |
| OR5M8 | NM_001005282 | TTTCTACTGAACTTTGTTTC | 2823 |
| OR5T2 | NM_001004746 | AACAGCTTAATACAATTCAG | 2824 |
| OR5V1 | NM_030876 | ATCTGTGTTGCATGGTAGGT | 2825 |
| OR5V1 | NM_030876 | GTATTTATATCTGTGTTGCA | 2826 |
| OR5W2 | NM_001001960 | TTTGAAAGTGACACTCACCT | 2827 |
| OR6C1 | NM_001005182 | AAAGGACCACTGTTATTATC | 2828 |
| OR6C6 | NM_001005493 | GCAAATTTTGAATTCACCTA | 2829 |
| OR6K6 | NM_001005184 | GCAAGTATTTCAGATGATTT | 2830 |
| OR6P1 | NM_001160325 | GTCTGTTAACTTTTCCTATA | 2831 |
| OR6S1 | NM_001001968 | TAAGTGCTTCAGATCTTAAC | 2832 |
| OR7D2 | NM_175883 | CTACTGATGTAGCATAAATC | 2833 |
| OR7D4 | NM_001005191 | TAGAAATCTCTCTCTTTGGC | 2834 |
| OR7G1 | NM_001005192 | GAATCTACCCCTTTTCAAGA | 2835 |
| OR8B12 | NM_001005195 | AGAGAGATTTGAACTTTGGT | 2836 |
| OR9A2 | NM_001001658 | GTGACATGTCCCTGCTACTG | 2837 |
| OR9Q1 | NM_001005212 | GTCACAGCTTCATTGCCATC | 2838 |
| ORC4 | NM_001190882 | GGAACGGAAGTGGGCGTGGA | 2839 |
| OSBPL1A | NM_018030 | GTTCCAAAACCAAGACTGAA | 2840 |
| OSBPL1A | NM_018030 | TGAAGACTGCCTTTCAGTCT | 2841 |
| OSBPL6 | NM_001201481 | ATGCTGCGCACCCGCCCTAC | 2842 |
| OSGEP | NM_017807 | AGGAGGAGCTAGGCTGCCAT | 2843 |
| OSMR | NM_003999 | CACAACCCGGACTTTGCGGG | 2844 |
| OSR2 | NM_001142462 | CCACTCTGTTTACTTCTGTT | 2845 |
| OTOA | NM_001161683 | CACTGGGCATGTCTGTTTAA | 2846 |
| OTOA | NM_001161683 | GATTTGCATGTGGCTTGTCT | 2847 |
| OTUD1 | NM_001145373 | AGCGCGTCCCGCCGGCGAGG | 2848 |
| OTX2 | NM_001270525 | AGATTGTAATTGCTTTCTTC | 2849 |
| OXGR1 | NM_080818 | AGAACACGCACTTGCTCGCT | 2850 |
| OXSM | NM_017897 | AGCTACCCAGCCGCCTCCCA | 2851 |
| OXT | NM_000915 | CAACGCGGTGACCTTGACCC | 2852 |
| P2RX6 | NM_005446 | AGGGACACTTCCACTAAAGC | 2853 |
| P2RY14 | NM_001081455 | TGGTTTTCCAACTAATTTCA | 2854 |
| P2RY6 | NM_176797 | GGGGAGGTGATGTCTGGAAG | 2855 |
| P2RY8 | NM_178129 | CACAGCGACGTTACTCCAGT | 2856 |
| P3H2 | NM_018192 | CGGTTTGATTCAGTCTGAAA | 2857 |
| P3H4 | NM_006455 | AGACACTCGGAGGGTGCAGG | 2858 |
| P4HB | NM_000918 | GCTTTCGCCTGCACCTTCCA | 2859 |
| PAAF1 | NM_001267803 | TCAGGAACCAGCCCCTCGTG | 2860 |
| PABPC1 | NM_002568 | CTCCGCGTCTCCTCCTACTC | 2861 |
| PABPC1L2A | NM_001012977 | CGCCCGGGTGGCAACGGTGG | 2862 |
| PACRG | NM_001080378 | TCGTTCACAAACTTGCACCT | 2863 |
| PACS2 | NM_001100913 | GCGGGAAAGTGTCGAGGCCG | 2864 |
| PACSIN1 | NM_020804 | GGCGGGTGGCGGGTGGGGTC | 2865 |
| PACSIN3 | NM_001184974 | TTCTCTGCTTCGCCCGTGTG | 2866 |
| PADI3 | NM_016233 | CAGGTTCGTATACAAATACT | 2867 |
| PADI4 | NM_012387 | TCTCAAAATCTCCTCTGCCC | 2868 |
| PAEP | NM_002571 | AACCTCCTCTGTGTCCGGGC | 2869 |
| PAGE1 | NM_003785 | ATGAAACAGCAGAGGGAGGT | 2870 |
| PAK2 | NM_002577 | GAGACGAGCGCCACCTCCCA | 2871 |
| PAK5 | NM_020341 | TGTTGGGGGAGAGGGCGTGC | 2872 |
| PAK6 | NM_001276717 | CCGCCTCCCGACTGAACTCC | 2873 |
| PAK6 | NM_001276718 | GAGGAGGAAGGGCTGCCTGC | 2874 |
| PAK6 | NM_001276718 | TATCTGCCTTTCTTTGCTGA | 2875 |
| PALB2 | NM_024675 | TCAGAGATTCCGGCTACTTC | 2876 |
| PALLD | NM_001166108 | ATAAAGCCACTTAACATAGA | 2877 |
| PALLD | NM_001166108 | GGTGCTTCCCAGCCCGCTGC | 2878 |
| PALM2 | NM_001037293 | AATTGGATAATGTTGTTCGC | 2879 |
| PALM3 | NM_001145028 | GACTCTTCCCAGGTGCAAAG | 2880 |
| PALMD | NM_017734 | AAATCCAATCAGTGGAAGAA | 2881 |
| PAMR1 | NM_001001991 | CTTTTGCAACTACAGGCTAC | 2882 |
| PANK2 | NM_153640 | CTCGGCTGAGGGCACGAGGC | 2883 |
| PAPD5 | NM_001040285 | ACAGCCTATAACACTTTTTC | 2884 |
| PAPOLB | NM_020144 | GGATTCACGTTGTTGATGAC | 2885 |
| PAQR7 | NM_178422 | AGAGGGTGAACCAAATTAGC | 2886 |
| PARD6B | NM_032521 | TGGGTGTGGGCGGAACGCGA | 2887 |
| PARM1 | NM_015393 | TGTCCAGCAGAGGCCGCTCT | 2888 |
| PARP10 | NM_032789 | AATACCTCCTGGTCAGCTGG | 2889 |
| PARP15 | NM_152615 | TGAGTAAACTAACACTGTCC | 2890 |
| PARP3 | NM_005485 | GTCACGTTCCAGAACGCGAA | 2891 |
| PARP4 | NM_006437 | CAGGAGGGATTTTGTCAATG | 2892 |
| PARVA | NM_018222 | ACTGCCCCTTGCAGGACAGG | 2893 |
| PARVB | NM_001243385 | AGCTATCGCTGGAAACACCC | 2894 |
| PARVB | NM_001003828 | CTGATGAAACCGTTTGTTAA | 2895 |
| PASK | NM_001252120 | TGGCCCGCACCTTGCAGCCA | 2896 |
| PAWR | NM_002583 | AAAGGCCGAGGCGGCGCGCG | 2897 |
| PAX3 | NM_001127366 | CCACTTTCTCTTCCCATCTC | 2898 |
| PAX4 | NM_006193 | ATCAGGACGGTGAGGAGCCT | 2899 |
| PAX6 | NM_001258463 | TGTGTGTGTGTGTGTCCCAC | 2900 |
| PBK | NM_018492 | ATCTGCTCCCCAGGAGGGGA | 2901 |
| PBX4 | NM_025245 | GAGGAGGAGCAGGAACTCTG | 2902 |
| PBXIP1 | NM_020524 | AGACCTCCCTTCCCCTCCCC | 2903 |
| PCBD2 | NM_032151 | GGAAGCGCCCAGCCTTCCCG | 2904 |
| PCDH10 | NM_032961 | TGTCTGTTTGGCGGCCAGTT | 2905 |
| PCDH11Y | NM_032971 | AACTGCTGAGTACCCCCCTC | 2906 |
| PCDH11Y | NM_032971 | GGTTCTCCGTCAGCGGGGAG | 2907 |
| PCDHA2 | NM_018905 | TTTACTCATAGCTTTCATCT | 2908 |
| PCDHB14 | NM_018934 | GAGAACATGAATCATTATAC | 2909 |
| PCDHB7 | NM_018940 | AGCATTACTGTGACCATTTG | 2910 |
| PCDHB9 | NM_019119 | GTGTTAGATTTAGCTGTGTT | 2911 |
| PCDHGA12 | NM_003735 | AGATTGTGCAGTAATTGGTT | 2912 |
| PCDHGA7 | NM_018920 | GTGTATTGTGTGCATCAATG | 2913 |
| PCID2 | NM_001127203 | GGGCCCGGGGTCTTTCTGCC | 2914 |
| PCIF1 | NM_022104 | GGAAGGGGAGACAGCTTTGT | 2915 |
| PCNA | NM_002592 | CGGTCCGGAATATCCACCAA | 2916 |
| PCNA | NM_182649 | CCCGGACTTGTTCTGCGGCC | 2917 |
| PCNP | NM_020357 | ATGTCATCGAGTAGCCGCCT | 2918 |
| PCNX2 | NM_014801 | GCGAAGGCTAAGGAGGGACT | 2919 |
| PCNX4 | NM_022495 | AGACAGCCTGACCCGACCTC | 2920 |
| PCOLCE2 | NM_013363 | GGAGTGGCACCCCAGCGGCC | 2921 |
| PCSK1N | NM_013271 | GCGGTTGCCATGGCAGTCGG | 2922 |
| PCYOX1 | NM_016297 | GAGGCGGCAGGATGTGCTTA | 2923 |
| PCYT1B | NM_001163264 | TGACATAGTTAATTCACCAA | 2924 |
| PCYT2 | NM_001282204 | CCCGCGCCCGTTCCGGATCA | 2925 |
| PDCD2 | NM_001199461 | AAGACATGTGCAGAGGTGAG | 2926 |
| PDCD2 | NM_001199464 | CAGAACCATCCCAGAGCACC | 2927 |
| PDCD2 | NM_001199464 | GAGGCACCAGGAAAGCGGCT | 2928 |
| PDCD6IP | NM_013374 | ATATTTTGCAGCACAGTACA | 2929 |
| PDCD7 | NM_005707 | CCGTTCTTATTGAGCATCCT | 2930 |
| PDCL2 | NM_152401 | CCCAACACAGGGGATGGTTG | 2931 |
| PDDC1 | NM_182612 | GAACCCGCCGGGGCCAAAGC | 2932 |
| PDE1A | NM_001003683 | AAAAACCTTGGCATTTAAAC | 2933 |
| PDE4D | NM_006203 | AGGTATGGGTCCATCCATTT | 2934 |
| PDE4DIP | NM_001195261 | TAAATGACTTGTGGCTGATT | 2935 |
| PDE5A | NM_033437 | GGGTTTTGCTGATTGGATTT | 2936 |
| PDE7A | NM_001242318 | GCAGTGCAAGAAAAGACAGC | 2937 |
| PDE7A | NM_001242318 | GGCCGAGAGGAGCAGGTACC | 2938 |
| PDE7A | NM_002603 | TAGAACTGCCTAAGTAATGT | 2939 |
| PDGFB | NM_033016 | GCTTCCTCTGGCTTTGCTAA | 2940 |
| PDGFRB | NM_002609 | GGGGAAAAGAAAGAGAGAGG | 2941 |
| PDIA6 | NM_001282705 | TTTGGGGAGCTTGAGGAGGC | 2942 |
| PDIA6 | NM_001282706 | ACACTAAAAAATCGGGGCTG | 2943 |
| PDK1 | NM_002610 | ATGGGACTGGGGACACTAAG | 2944 |
| PDK4 | NM_002612 | ACCACGGAGTGCCCTGGCAC | 2945 |
| PDP2 | NM_020786 | ATCTCAGGCACGTGACTGCC | 2946 |
| PDSS2 | NM_020381 | GGAGCTGAACCTCCCAACCC | 2947 |
| PDXK | NM_003681 | GCTGCAGAGCCCTCTCCAGG | 2948 |
| PDYN | NM_001190892 | AAACAAGCTCTTTCGATTAT | 2949 |
| PDZD11 | NM_016484 | ATTGGTTGGCGTCTCCGGGA | 2950 |
| PDZD8 | NM_173791 | GTCAGAGGCGTGCTCGCTCC | 2951 |
| PDZRN4 | NM_013377 | CACTATTAATATTCATGAGC | 2952 |
| PECR | NM_018441 | AGTCTCACCCACACCTGCCC | 2953 |
| PEG10 | NM_001172438 | GCCCGCCGCTAGAGGGAGTA | 2954 |
| PEG3 | NM_001146185 | TGTGGCAACCGCAGCCTGAT | 2955 |
| PERP | NM_022121 | AACACGCGCCTGGAGAGGCC | 2956 |
| PEX2 | NM_000318 | CATCGCGAAGGGCCTCTGGC | 2957 |
| PEX26 | NM_001127649 | ACAAACTGGTGCTACAGCTT | 2958 |
| PEX5 | NM_000319 | ACCGACCTCCCTCGAACTCC | 2959 |
| PEX5L | NM_001256753 | CGGCAAGGCGAGGTGCCGGC | 2960 |
| PFKFB1 | NM_001271804 | CGAGAGGTTGGGCAGAGGTC | 2961 |
| PFN3 | NM_001029886 | ACGCCCCACGTGCCCCAGCC | 2962 |
| PGA3 | NM_001079807 | GCTGGAAAGATCTCAGAATG | 2963 |
| PGA5 | NM_014224 | GCTGGAAAGGTCTCAGAATG | 2964 |
| PGAM1 | NM_002629 | CAGAGCGAGTGGAAAGATTT | 2965 |
| PGAP2 | NM_001145438 | GTGGACGCGGCCGCCACTCT | 2966 |
| PGAP2 | NM_001256235 | CCGCAACGAGCCTCTGACGC | 2967 |
| PGF | NM_002632 | CACCTGGGATGGGGGCATCC | 2968 |
| PGK1 | NM_000291 | GGAAGGTTCCTTGCGGTTCG | 2969 |
| PGK2 | NM_138733 | AAGAAACCCCAGAATAAGAA | 2970 |
| PGLYRP1 | NM_005091 | GAACTTACATCGCAGAGGCC | 2971 |
| PGM1 | NM_001172818 | CTTCAGCTGTAAACACCAGG | 2972 |
| PGR | NM_000926 | CAAAACGTAATATGCTTATG | 2973 |
| PHACTR2 | NM_014721 | GATTCAAGTACCCACTTGAT | 2974 |
| PHC2 | NM_198040 | AATATTTTTGATCCTGTGGT | 2975 |
| PHF11 | NM_001040444 | AAGTTCGTCCAGCGCCGCCC | 2976 |
| PHF11 | NM_001040444 | GTGCCTGTTGGTGGGGGAGG | 2977 |
| PHF19 | NM_001286843 | GCGGCCACTAGCCAGGACCC | 2978 |
| PHF20 | NM_016436 | TCGTGTTCCTGCTAGGGCGC | 2979 |
| PHF21A | NM_016621 | GTCCCTCTCGCCCGGCTCTC | 2980 |
| PHF21B | NM_001284296 | AGTGCGAATAGGCCCCCTTC | 2981 |
| PHF23 | NM_001284517 | CAAAGTTCCGGAGGTTCATG | 2982 |
| PHF24 | NM_015297 | GGACGGCTCCGATGAGCAGA | 2983 |
| PHLDA2 | NM_003311 | CTTGGGGAGGGTATGGCCCG | 2984 |
| PHOX2A | NM_005169 | GATGCGCGGGACCCTATCCC | 2985 |
| PHYHIPL | NM_001143774 | TTGCCGCAGTCCGGATTTCC | 2986 |
| PI4K2A | NM_018425 | GCGTAGGAGCAGGTTCTGAT | 2987 |
| PI4K2B | NM_018323 | GCCACCTGCTTCCGTGAGCG | 2988 |
| PICALM | NM_001206946 | CCGCCCTCCCTCGCTCAGCG | 2989 |
| PICALM | NM_001206947 | AGACCATAGAAGGAAGTGAG | 2990 |
| PIDD1 | NM_145887 | TGCGCGGGCGGCTCGGCAGA | 2991 |
| PIF1 | NM_025049 | ATTGGTACAGCCCAAGCTCC | 2992 |
| PIGA | NM_002641 | ACATCTCGCGCTTAAGGGTG | 2993 |
| PIGR | NM_002644 | CAGAGTCTCCCCAAGGTCAA | 2994 |
| PIGS | NM_033198 | CCTCCGTGTTTGAGGCTTTG | 2995 |
| PIGS | NM_033198 | CTAGTATGTTTTAGCACAAT | 2996 |
| PIGV | NM_001202554 | GGCGTCTGTCTCATTTCTAC | 2997 |
| PIK3C2A | NM_002645 | ACCCCATTTCCTGACACAAC | 2998 |
| PIK3C2B | NM_002646 | TGCAGGATAGGTCCTTTCAC | 2999 |
| PIK3C2G | NM_001288772 | TTTGGCAGGTTGGGCGTGTT | 3000 |
| PILRB | NM_178238 | CCTTCTCTTGTTCCTGATCT | 3001 |
| PINK1 | NM_032409 | AAAGGGAAAGTCACTGCTAG | 3002 |
| PINLYP | NM_001193622 | TCCTCTCTCAGATCCTGCCA | 3003 |
| PITPNB | NM_012399 | AGGCTGCGCAACCGCAGTGG | 3004 |
| PITPNC1 | NM_012417 | GGCTGCTCCGGAGCGGAGCC | 3005 |
| PITRM1 | NM_001242307 | GCAAGGCGAGGGGCGTGGTA | 3006 |
| PITX1 | NM_002653 | AAGGTGGCTGCGGAGGGGGA | 3007 |
| PKD1 | NM_000296 | CCAGTCCCTCATCGCTGGCC | 3008 |
| PKD2L2 | NM_001258449 | GCCAACTTCTGGGAATAACC | 3009 |
| PKD2L2 | NM_001258449 | GCTGCTGGGGTCTGGTGCGG | 3010 |
| PKIG | NM_001281445 | TTTCCTTTGGACAATGAGCC | 3011 |
| PKLR | NM_181871 | TGGCTAGGTGGGTTTTGGAG | 3012 |
| PKN1 | NM_213560 | TCCCTTAGATGCCCTGGAGT | 3013 |
| PKN3 | NM_013355 | CTCTTTGTCTCGCACGTTGT | 3014 |
| PLA2G12A | NM_030821 | GCGGGGCCTCCATGCCCACG | 3015 |
| PLA2G15 | NM_012320 | TCAGCGTGGTCCAGGAAGCA | 3016 |
| PLA2G2D | NM_012400 | GCCTCCATGAGAGTGGGGGC | 3017 |
| PLA2G4A | NM_024420 | GAAATCCACAACAGCACTCA | 3018 |
| PLA2G4B | NM_001114633 | AAGGCTGGCGAGTGCCACAG | 3019 |
| PLA2G4D | NM_178034 | CGGAGCACCTCTTCCAGACC | 3020 |
| PLA2G7 | NM_005084 | GACACCACCCAGGCATTGCC | 3021 |
| PLAC1 | NM_021796 | CTCTGCAGCATTTCCCAGTT | 3022 |
| PLAGL1 | NM_001080956 | GCGCTGTACCTGGGCGACCT | 3023 |
| PLAUR | NM_001005376 | TTTGACGGTAAATATGAATG | 3024 |
| PLB1 | NM_153021 | CCGCCACTACCCCCTTTCAA | 3025 |
| PLCG1 | NM_002660 | CCCCAGACAGGCCGCAGGCG | 3026 |
| PLCH1 | NM_001130961 | CATTATGCACATTTAATGTC | 3027 |
| PLCL1 | NM_006226 | AGACTTGTTTTGACAGCCCT | 3028 |
| PLCXD1 | NM_018390 | ACAGGTGTGGTTGCTTCTCT | 3029 |
| PLD3 | NM_001291311 | GGCATTGAGACGGGCTGAGG | 3030 |
| PLD3 | NM_012268 | CCACCCGTCCCTACCGCAAC | 3031 |
| PLEC | NM_000445 | GATCTCGGGAGCGGCGGGGC | 3032 |
| PLEC | NM_201378 | ACGGGAAAGGGCGTGCGTGC | 3033 |
| PLEK | NM_002664 | TGGTAGTAAGAATTTCCCTT | 3034 |
| PLEKHA1 | NM_001195608 | ATAGCAGTATTAGTCATAAC | 3035 |
| PLEKHA5 | NM_019012 | CGCGCCCCAGACCCCTCCCT | 3036 |
| PLEKHB1 | NM_001130033 | GTTCTTGAGTCGGCTAAGAG | 3037 |
| PLEKHG1 | NM_001029884 | GGACGAGCGATCCACTGCTC | 3038 |
| PLEKHG1 | NM_001029884 | TTGGCAAGGCTCCAGAGACA | 3039 |
| PLEKHG4 | NM_001129727 | CCCCCAGGAGCCCTAAGAGC | 3040 |
| PLEKHG4B | NM_052909 | CTCAGACAGGGACTTCGAAA | 3041 |
| PLEKHG5 | NM_001265593 | GAGGGAGGTGTCCGCCTTCC | 3042 |
| PLEKHG5 | NM_020631 | GGTGCTCACTACCTCCACTT | 3043 |
| PLEKHG6 | NM_001144857 | GGTGTGATATCCCTGGAGCC | 3044 |
| PLEKHO1 | NM_016274 | GGAGCTGCGGGGTGCGGACT | 3045 |
| PLIN3 | NM_001164189 | GGACCCTGTGAAGTTGGCCC | 3046 |
| PLK4 | NM_001190799 | TAAACTCTCCGCAGCGCTTC | 3047 |
| PLK4 | NM_001190801 | CTCGATCTTCTCCCCGATGC | 3048 |
| PLOD1 | NM_000302 | TGCCCTAATAAGGAGAGGCC | 3049 |
| PLOD2 | NM_000935 | TGCAGTCACTTCAGACTGGG | 3050 |
| PLP1 | NM_001128834 | TATTTTCCAAGGAATCGGGA | 3051 |
| PLPP1 | NM_176895 | GCCTCATCCCTCCCGACCTG | 3052 |
| PLPP4 | NM_001030059 | GCACGCACGTGGGCATGTAG | 3053 |
| PLPP6 | NM_203453 | TTCCAATGTGAGGAGAGCAG | 3054 |
| PLS1 | NM_001172312 | ATAGGAAAAGGGAAGGGCTG | 3055 |
| PLSCR2 | NM_001199979 | TGCTGCCATTCCAACACCAT | 3056 |
| PLTP | NM_001242921 | AGTGGCCTTCTTTGCCCCGC | 3057 |
| PLTP | NM_001242921 | ATCTCTGAGTAAGTGGGGGG | 3058 |
| PLXNA4 | NM_020911 | GTTGGACATTACGCCCACCT | 3059 |
| PLXNC1 | NM_005761 | GGAAGAGAGGATGAGGAAGG | 3060 |
| PMEPA1 | NM_020182 | GCTCTTAAAGGGCCAGAGCT | 3061 |
| PMEPA1 | NM_199170 | CCAAGGGGCCTCCGGCTGGG | 3062 |
| PMM2 | NM_000303 | CATGCTCGAATGTACAAGGC | 3063 |
| PMP22 | NM_153321 | TGAGAAAGCTCAGCCGCCTC | 3064 |
| PMP22 | NM_000304 | ATAATCCCAAGAGGCCCTGC | 3065 |
| PMPCA | NM_001282944 | CAGCGGCGGCTCCATGGCCC | 3066 |
| PNISR | NM_032870 | GGTGTTGACCAGAGTAGAGA | 3067 |
| PNKD | NM_015488 | CAGCCAACCTTCGTAGCTAT | 3068 |
| PNKP | NM_007254 | CAGCAAGAGAGATGAAGGTC | 3069 |
| PNLIPRP1 | NM_006229 | GTATTAAGTGCGCACAGCAT | 3070 |
| PNMA6A | NM_032882 | ACGTGACCCGCCCGCGGCAA | 3071 |
| PNPLA1 | NM_001145717 | GCTGGGTAGGGAGTTCCTAC | 3072 |
| PNPLA6 | NM_001166113 | TGGAAGATACTGAGAGATGC | 3073 |
| PNRC1 | NM_006813 | GCGCTGCCAGCGAGCTCTTT | 3074 |
| POC1A | NM_001161581 | GGCCTTAAGGATCCCGGAAG | 3075 |
| POC5 | NM_152408 | TCTTCATACACTCTGTACAA | 3076 |
| POLD4 | NM_021173 | TGAAGTCGGGGCATCCCGAC | 3077 |
| POLE3 | NM_017443 | TTTAGCAACCCTAAGCGGTT | 3078 |
| POLI | NM_007195 | GCTTTCAATCTCTCCGCTTC | 3079 |
| POLL | NM_013274 | CTCCTTCGTTTTTTTCCCTC | 3080 |
| POLR1D | NM_015972 | AAAGGTACCAGAGTTGAGCC | 3081 |
| POLR2F | NM_021974 | TCCACATAGAAGTGGGCTCC | 3082 |
| POLR2L | NM_021128 | CCGCTCGTTCTCCGCTGTTC | 3083 |
| POM121 | NM_001257190 | TGGGGAGCGCGTAGGCTCAT | 3084 |
| POM121C | NM_001099415 | GGGGGAGCGCGTAGGCTCCT | 3085 |
| POM121L2 | NM_033482 | GAACAGCAAAGCAAGTTACT | 3086 |
| POMGNT2 | NM_032806 | CCCGCGCCGCCACCAGCCTG | 3087 |
| POMGNT2 | NM_032806 | GAGTGATAATTTGCGCCGAG | 3088 |
| POMP | NM_015932 | GGGAGGGAAGACACGGACTC | 3089 |
| POMZP3 | NM_012230 | CAGAAACAGGCGTTGAAGGC | 3090 |
| PON2 | NM_000305 | CACATCATGAGCCTAATGTA | 3091 |
| POPDC2 | NM_022135 | TTCCTTGGTTCCATGTTTCT | 3092 |
| POR | NM_000941 | TTTGCGCTCTTGGTACGGCC | 3093 |
| POU2AF1 | NM_006235 | TTTTGGGCTCATCACTGGCC | 3094 |
| POU2F3 | NM_014352 | CATACATGGAGCTGGGGACC | 3095 |
| POU3F4 | NM_000307 | AATCAATCTTTCAGCTCCAT | 3096 |
| POU4F2 | NM_004575 | CGGCGTTTCCTGGCAAGGGA | 3097 |
| POU4F2 | NM_004575 | GCAGAAAGGACTCAAGCCTG | 3098 |
| PPA2 | NM_176869 | GCATAGTGCGCACAACTGGC | 3099 |
| PPARG | NM_138711 | ACTTCGCCTTTCCAGCCCCC | 3100 |
| PPEF2 | NM_006239 | ACTCTGCTATTTCAGGGCTA | 3101 |
| PPEF2 | NM_006239 | AGGCTTCTCAGATGTGGCCT | 3102 |
| PPIAL4A | NM_001143883 | ACTGAATAATATTCCACTGT | 3103 |
| PPIAL4A | NM_001143883 | ACTGTGGTATATTCCTACAG | 3104 |
| PPID | NM_005038 | CGAGAAGAATAATGAGAACT | 3105 |
| PPIL1 | NM_016059 | GAATTTCTTAGTCTCACAAT | 3106 |
| PPIP5K1 | NM_014659 | AAGAAGAGGTTTAAGGGGAA | 3107 |
| PPM1B | NM_002706 | ACGAAGTACGGAGGTGCCGA | 3108 |
| PPM1H | NM_020700 | TGCATGGAGCGGGCCGACCG | 3109 |
| PPM1K | NM_152542 | GGACTGTAGTTGTGACAGCC | 3110 |
| PPM1N | NM_001080401 | CCGCCTAAAGAGCAGGTCAA | 3111 |
| PPDX | NM_000309 | AGGCGGCGAGCGCTTAATGC | 3112 |
| PPP1R3D | NM_006242 | CTCCCTGGCTGAGCTGAGGC | 3113 |
| PPP1R3E | NM_001276318 | TTCACTCGGGACCGCAAAGG | 3114 |
| PPP1R42 | NM_001013626 | AACAGGACTCTAGTCGGAGT | 3115 |
| PPP1R9A | NM_001166162 | TTATCATTCTGATTGGTCTT | 3116 |
| PPP2R2B | NM_181678 | ATGGTTGAGCGGCCAGTAAG | 3117 |
| PPP2R2D | NM_001291310 | TCTGCACCAGAACCAATAAG | 3118 |
| PPP6R3 | NM_001164164 | GCCAATCGGAATGTAGTCAA | 3119 |
| PPY | NM_002722 | GCCAGTACTGAGGCCAGAGA | 3120 |
| PQLC2 | NM_001040126 | AGCAGCGGCGCCTGCGCGTT | 3121 |
| PRAME | NM_001291715 | GAGAGGAAGTTGGAGAGCAG | 3122 |
| PRAMEF12 | NM_001080830 | AGAATGTCTTCCAAACAATG | 3123 |
| PRAMEF15 | NM_001098376 | GGAGAGCCAAAAACCCAATC | 3124 |
| PRAMEF15 | NM_001098376 | TGACTCAATCCATTAATCTG | 3125 |
| PRAMEF17 | NM_001099851 | AGGGCAGAACTATGCCTCTG | 3126 |
| PRAMEF20 | NM_001099852 | TCCACCCAGTTAATCCTGAT | 3127 |
| PRAMEF6 | NM_001010889 | TTTGGCTCTCCCCAGATTAC | 3128 |
| PRCD | NM_001077620 | TGTGGCATTGAGCACGTATT | 3129 |
| PRDM16 | NM_022114 | CCGCGCCGAGGCGGCGGCGG | 3130 |
| PRDM2 | NM_001007257 | CGATGGCAAACAGCTGTCGG | 3131 |
| PRDM2 | NM_012231 | GACCTATGTTAAACTCTGGT | 3132 |
| PRDX1 | NM_001202431 | CTTTGGGAGGCCAAGGCGGG | 3133 |
| PRDX1 | NM_001202431 | TAAGCGCGAGCCACCGCACC | 3134 |
| PRELP | NM_002725 | GAGGAGAGAGGGAGGGAGCT | 3135 |
| PREPL | NM_001171603 | GACTCGCGACTCCATCTCAC | 3136 |
| PREPL | NM_001171613 | AGCTCGAGATGAAGCACAGA | 3137 |
| PREPL | NM_001171613 | ATTTCGAGACTAAAGAACCC | 3138 |
| PREPL | NM_006036 | CAGTTGCTATTATTTACGAC | 3139 |
| PRG2 | NM_001243245 | AATGAATGAGTGGGCTCCCC | 3140 |
| PRG3 | NM_006093 | CAAACAAGGCAGTAGGCCCC | 3141 |
| PRG3 | NM_006093 | GACTGCAGGGACCTGCCTCC | 3142 |
| PRH2 | NM_001110213 | AGTGTATCCCTCATTTCTTC | 3143 |
| PRH2 | NM_001110213 | GTTGGGGAGGATGTTGTTTG | 3144 |
| PRIM2 | NM_001282488 | TTTGAGATGCTATGGTTCAG | 3145 |
| PRIMA1 | NM_178013 | GGCTTTAAATGGGGGCTGTC | 3146 |
| PRIMPOL | NM_152683 | GGAGCACATCTCCCGGCGGC | 3147 |
| PRKAA1 | NM_206907 | AGGGCGGTGACTCGGCTCGG | 3148 |
| PRKACB | NM_001242860 | TACTAGTGATATCTCATGCT | 3149 |
| PRKAG3 | NM_017431 | AGGATCGGTTTCTCTCTGAT | 3150 |
| PRKAR1A | NM_212471 | TCGGCAGGGCTCAGGTTTCC | 3151 |
| PRKAR1B | NM_001164761 | GGCAGGTGAGTGCAGGACCC | 3152 |
| PRKAR1B | NM_001164762 | AGGTGGGAAAGAATTTAGGA | 3153 |
| PRKCSH | NM_002743 | CTTAGAGAGGATAGTTCTGA | 3154 |
| PRKCSH | NM_002743 | GGGCGGTGCCAGAGCCGAGA | 3155 |
| PRKCZ | NM_001033582 | AGCCCAGGCAGGGAGCATCC | 3156 |
| PRL | NM_001163558 | TTTTCAAAGGGCAAGCAGTT | 3157 |
| PRLR | NM_001204314 | AACATTGGCCCCTCAGTGAT | 3158 |
| PRLR | NM_001204314 | ATGAGACAGCTCTAGTGTTC | 3159 |
| PRLR | NM_001204314 | TACGTAGCATGGCTGAACAT | 3160 |
| PRM3 | NM_021247 | GCAGGATGCTGACATCACAA | 3161 |
| PRMT9 | NM_138364 | TCACTGCTGCCCATTCCCGC | 3162 |
| PRODH2 | NM_021232 | CACTGCACCCTTGACCTCCC | 3163 |
| PROSER1 | NM_025138 | GATGTTTTGATTTTGCCCTC | 3164 |
| PROSER2 | NM_153256 | CCCGGCCCTTTAAGCGCCGC | 3165 |
| PROX1 | NM_001270616 | GATAGCAAGGCAAGAGAACT | 3166 |
| PROX1 | NM_002763 | CGTGTTTTCCTCTCTCTGCC | 3167 |
| PRPF38B | NM_018061 | TTCAGCGTGCAGAGAACGCG | 3168 |
| PRPF40B | NM_001031698 | CGACTGCGAAGCCAGGACGC | 3169 |
| PRPH | NM_006262 | GTGGGTAGAGGCCTGCAACC | 3170 |
| PRPSAP1 | NM_002766 | GGTTGACCGCAGTACTGAAG | 3171 |
| PRR14 | NM_024031 | TCTTCCGCAGCTCCCACCTC | 3172 |
| PRR20D | NM_001130406 | CCAGTCCCCTGCCAGTCAAA | 3173 |
| PRR20D | NM_001130406 | GAAATGGCGGCATCTCAGAA | 3174 |
| PRR21 | NM_001080835 | GAGACATGGGATTTAATGGG | 3175 |
| PRR5- | NM_181334 | GCGGAAACTCCGGCGAGAGC | 3176 |
| ARHGAP8 | | | |
| PRR9 | NM_001195571 | GAGGTCTGGTGAGGACCCAC | 3177 |
| PRRC2B | NM_013318 | GTGGTGAGAGCAGTTTTCTA | 3178 |
| PRSS21 | NM_006799 | GAGGTTGTAGGTGGAGGACG | 3179 |
| PRSS3 | NM_002771 | GCTGCAGGTGTGTTTGTGCT | 3180 |
| PRSS3 | NM_002771 | TGATGCAAGACCCTGGCAAG | 3181 |
| PRSS53 | NM_001039503 | GAGCTAGGAACTGCTGGCTA | 3182 |
| PRSS55 | NM_198464 | TTTTCTGGCTGCTTTGTTTC | 3183 |
| PRSS56 | NM_001195129 | TGATGAGACTTCAGAGGTGA | 3184 |
| PRSS57 | NM_214710 | GAAACGCCCGCCTGGGCTCC | 3185 |
| PRTG | NM_173814 | GGCCGCTCGCGAGAAGCAAG | 3186 |
| PRTN3 | NM_002777 | TGGCTGTCACCCACCCAAGT | 3187 |
| PRX | NM_181882 | CGGGGGTGTGACGTCACCAG | 3188 |
| PSD3 | NM_015310 | GGCCGACGCCTCGGGGAGGG | 3189 |
| PSENEN | NM_172341 | GACGTAAGAGCAGCCAGACC | 3190 |
| PSMB2 | NM_002794 | CAGGCGTGAGCCACTGCGCC | 3191 |
| PSMB4 | NM_002796 | ATGCGATGCGAAGCGATGTT | 3192 |
| PSMD1 | NM_002807 | GGAACACTGGTCTGCACCTG | 3193 |
| PSME4 | NM_014614 | AACGAACTGAGAGCCGCGTG | 3194 |
| PSORS1C2 | NM_014069 | CACTGTCCCAGCTGCATCCC | 3195 |
| PSPH | NM_004577 | CGCCGCCGCCATTGGGCCAC | 3196 |
| PSRC1 | NM_032636 | GTTCCCAGAAGACTGCATCC | 3197 |
| PTAFR | NM_001164723 | CTTGTTCCTCTCATCTCTCC | 3198 |
| PTGDR2 | NM_004778 | CACCCATCCCCGCTTCATGA | 3199 |
| PTGES | NM_004878 | TTTCTCTTCACAGGAGAAGG | 3200 |
| PTGFR | NM_000959 | GAGCAGTACTGGGAGAGAAG | 3201 |
| PTGIS | NM_000961 | GGGTTTCTAACAGAGCGCCC | 3202 |
| PTGS1 | NM_001271166 | TCTGCCAGAAATGAAAAGAC | 3203 |
| PTGS2 | NM_000963 | GCGTAAGCCCGGTGGGGGCA | 3204 |
| PTH1R | NM_001184744 | CGAGGCCCGGAGTCTTACGG | 3205 |
| PTH1R | NM_001184744 | GGGGGGCGGAAGGCTCCTCT | 3206 |
| PTHLH | NM_002820 | AGGGTTGACTTTTTAAAGCC | 3207 |
| PTK2B | NM_173174 | CGTGCGGGGGGGATGGCGAG | 3208 |
| PTP4A2 | NM_001195101 | CAGGCATCAGCCACCACACC | 3209 |
| PTPDC1 | NM_001253830 | GGGGACCCTAAGTAAGGGGA | 3210 |
| PTPN12 | NM_001131008 | ACGCGAAGGGAGCGGCCGCG | 3211 |
| PTPN5 | NM_001278236 | ATGAAATGGAGTGCTAGTGT | 3212 |
| PTPRA | NM_080840 | CGTTCTCCTGGTAGCTCCAG | 3213 |
| PTPRE | NM_006504 | TGTGGGCATCCGTTTACTCA | 3214 |
| PTPRH | NM_001161440 | ATCTCCAGTGTCAGAGCTAG | 3215 |
| PTX3 | NM_002852 | TACGCTGCAGTCAGATTAAT | 3216 |
| PUS1 | NM_025215 | GTGCTGGATGCAGGAGGGCC | 3217 |
| PUS7 | NM_019042 | CTCTGCCGCTGGTGCGACTC | 3218 |
| PVRIG | NM_024070 | GGATGTGACCTCAGAAACAG | 3219 |
| PXMP2 | NM_018663 | ACCGGGGAAAAGTGTGTGGT | 3220 |
| PXYLP1 | NM_152282 | TGCTGAGAGGACACTGCCTC | 3221 |
| PYGM | NM_005609 | GGGAAGGGCTCAAAGCTGTG | 3222 |
| PYROXD1 | NM_024854 | TTCATGGAATAACTACATTC | 3223 |
| QPCTL | NM_001163377 | ACGTCAGTAACGCGTCCCAG | 3224 |
| R3HDM4 | NM_138774 | AAACCCAGGCGCGCGGGGAG | 3225 |
| RAB10 | NM_016131 | TTTCTCTGCACAGCGCTTGT | 3226 |
| RAB11FIP4 | NM_032932 | GTCGCGGAGGACGCGGCCGT | 3227 |
| RAB14 | NM_016322 | AGAACTAGGGTTGTCGCTCG | 3228 |
| RAB1A | NM_015543 | GACTTCGCTCGGACTCCCCC | 3229 |
| RAB27A | NM_183234 | AACAGCTGAGACTAATTAGC | 3230 |
| RAB28 | NM_001159601 | GAGGCGCTGCGTTTCCCTTC | 3231 |
| RAB2B | NM_032846 | CCCTTATCCCTCCAAACTCC | 3232 |
| RAB30 | NM_001286061 | AGAAAGCCTTGAGAACTAAG | 3233 |
| RAB31 | NM_006868 | CCCGGGACCTGCGGCGTCGC | 3234 |
| RAB33A | NM_004794 | GACCCGAGGGAAGAAGCCTC | 3235 |
| RAB33B | NM_031296 | GGCGTGTACCTGGAGAGCAA | 3236 |
| RAB39A | NM_017516 | AGGCGGGGCCAGGCCCGGCT | 3237 |
| RAB40A | NM_080879 | GCTTCATTTGTGAAAACAAA | 3238 |
| RAB43 | NM_198490 | GTCGGGGGCGGGGACGTAGG | 3239 |
| RAB44 | NM_001257357 | CTTCCTGTGGAAGCGACCAC | 3240 |
| RAB4A | NM_001271998 | GCTGAGTCCCGATTTCCCTG | 3241 |
| RAB6A | NM_001243718 | TGGCTTGCCCCGCCTCCTCC | 3242 |
| RABAC1 | NM_006423 | CCTGACGGTGACTAAGAGGA | 3243 |
| RABGAP1L | NM_001243763 | TTTGATAGAACCTATCGAAT | 3244 |
| RABL2A | NM_013412 | GTGTGGTACTGAGGCTTCAG | 3245 |
| RABL2B | NM_001130920 | GTGTGGTACCGAGGCTTCAG | 3246 |
| RABL6 | NM_001173988 | CCCAGCGTCCGCAGCAGTCC | 3247 |
| RACGAP1 | NM_001126103 | ATGGCATCCTGAATGACTTC | 3248 |
| RAD17 | NM_133338 | ACACATTTCCGTCGCAAAGT | 3249 |
| RAD23B | NM_001244724 | GCTCCACGCCATCTGCCACC | 3250 |
| RAD50 | NM_005732 | CCAAAAGTCAGTGCCTCTCC | 3251 |
| RAD51 | NM_001164269 | CTAATTCAAACTTTATGCCG | 3252 |
| RAD51D | NM_133629 | CAGAAGGCTCTTTAGAAGGT | 3253 |
| RAD52 | NM_134424 | AAGAGCCGCAAAGCCTTCTG | 3254 |
| RALA | NM_005402 | AGCTCAGAGAGCCGGGGGTG | 3255 |
| RANBP1 | NM_002882 | GCAACGTCATCGTCACGCGC | 3256 |
| RANBP6 | NM_012416 | AAACAAATGGAGGATGCCAT | 3257 |
| RAP1B | NM_015646 | AGAGGCCGGCGCCGAGGACC | 3258 |
| RAP2C | NM_001271186 | TTACAAGCACGGCTGGTGGA | 3259 |
| RARA | NM_000964 | TGTCTCAAATACACAGCATA | 3260 |
| RARB | NM_001290216 | GACCTTGCTTCTTCCCAGCA | 3261 |
| RARS | NM_002887 | AGGAGAACCCGCGGGGATTT | 3262 |
| RASA3 | NM_007368 | GTTGGCAGGGACGGCGCTGG | 3263 |
| RASAL2 | NM_004841 | ACCCTTCCTTACTCACTCAC | 3264 |
| RASGEF1B | NM_152545 | TGACGCGCTGCGGGAGTCTG | 3265 |
| RASGEF1C | NM_175062 | CGCAGCGCCGCGTTGCTCCG | 3266 |
| RASGRP4 | NM_001146203 | TATTGAAGTATGACAGTGAC | 3267 |
| RASL10A | NM_006477 | AGGGGCTTCTATTTTGGAGC | 3268 |
| RASSF1 | NM_001206957 | GGAGATACCCGTGTTTCTGG | 3269 |
| RASSF5 | NM_182665 | AAGTGGACTCAGGGAACTGC | 3270 |
| RASSF6 | NM_001270391 | TTAACATCAGTCAAATCCCG | 3271 |
| RAX2 | NM_032753 | TTGAGGCGGCCCCTCCCACT | 3272 |
| RBBP7 | NM_002893 | AGGGCTCGCCCGGCGCTCCC | 3273 |
| RBBP9 | NM_006606 | AAGCTCGCAGGCTTTGTTCT | 3274 |
| RBFOX1 | NM_001142334 | GCATTTGTGTGTGTATGTGT | 3275 |
| RBFOX2 | NM_014309 | GAGGGGCAAGCGCCATGTGC | 3276 |
| RBM12 | NM_152838 | TTGCACAGTCTTGCAGTGAA | 3277 |
| RBM19 | NM_001146699 | CGTCTCACAGAATCCGCCCA | 3278 |
| RBM3 | NM_006743 | GAGAAGGTTCCTTTGTGGAA | 3279 |
| RBM39 | NM_001242600 | GTCTCTAGGGCAAAGACAGT | 3280 |
| RBM48 | NM_032120 | TCTTCGCACGCAGGAAACGA | 3281 |
| RBMS1 | NM_002897 | TTAACCACTCCTCACCTCCC | 3282 |
| RBMY1J | NM_001006117 | CCTGCGGCTCCATCATCTCG | 3283 |
| RBMY1J | NM_001006117 | TGAGGCCGCTCCGCCCCAGC | 3284 |
| RCAN1 | NM_004414 | CGGTGGCCGGCCCTAGGGGC | 3285 |
| RCBTB1 | NM_018191 | GTTGTAGGGCCCGAAGAGCA | 3286 |
| RCCD1 | NM_033544 | GGTTGGTGGCCAGCTGAGCC | 3287 |
| RCN2 | NM_002902 | TGCTTTTAGAAGCGTTTCGG | 3288 |
| RDM1 | NM_001163130 | AGATTTTTAGAGTCCCGGAG | 3289 |
| REEP1 | NM_001164730 | TCTTTTCCCTCCAGGCATCT | 3290 |
| REG4 | NM_001159352 | ACATAAGGGGAGAGGAAGAT | 3291 |
| RELB | NM_006509 | TGGGGGTTTTCCCGTTCCCC | 3292 |
| RELT | NM_152222 | GTTCCCAGGGGCGCGAGAGA | 3293 |
| REM1 | NM_014012 | CGCCCCATTAGGGCAGCCCC | 3294 |
| RENBP | NM_002910 | CCTTGGCCCTACCAAGCCTG | 3295 |
| REP15 | NM_001029874 | CTTTAACTTAATAAACCAGC | 3296 |
| REPS1 | NM_001128617 | GATCTCAGCAGCAAGACCCC | 3297 |
| REST | NM_005612 | GCTCGCCTGGGGGCGCGTCT | 3298 |
| RET | NM_020975 | GGAGCTCAGTGCGGGACGCG | 3299 |
| RETNLB | NM_032579 | TAATACACCTGGTATTAACC | 3300 |
| REXO2 | NM_015523 | TGCTAAGTTTGTTTGCTTCC | 3301 |
| REXO4 | NM_001279350 | ACCCGGTAGGGCAGCTGAGC | 3302 |
| RFC2 | NM_002914 | GCGACGCCTTCCGAGAAAGC | 3303 |
| RFK | NM_018339 | AAGCCCGGGATCCAGACATT | 3304 |
| RFPL4A | NM_001145014 | AACACAGTCGTCTTCCTTTA | 3305 |
| RFPL4A | NM_001145014 | TGAGATTGTTACTATTGGAC | 3306 |
| RFPL4B | NM_001013734 | ATCATCATAAACGGAAGGGT | 3307 |
| RFWD2 | NM_022457 | ACAGACAGACTCCCTTCGCC | 3308 |
| RFX1 | NM_002918 | CAGATCGCCGGGAAGTCCAG | 3309 |
| RFX4 | NM_032491 | TGAATAGTCAAGAAGTGGTC | 3310 |
| RFX7 | NM_022841 | AAAGCGACTCACTCGAGCCC | 3311 |
| RFX7 | NM_022841 | CCCCCTTCGTCCTCCCCTCC | 3312 |
| RGL3 | NM_001035223 | CAGATATGTCCTTTCTTCTG | 3313 |
| RGL3 | NM_001035223 | GAAGAGCCAGGACCTCTCCT | 3314 |
| RGL4 | NM_153615 | GTAACACCATGGACCACCAG | 3315 |
| RGMA | NM_001166287 | CCCTTACACCGTGTGCGGGC | 3316 |
| RGMB | NM_001012761 | GAGAGAACTGATCCAGGACC | 3317 |
| RGPD1 | NM_001024457 | AATGTCCACAGTGCTCCAGT | 3318 |
| RGPD1 | NM_001024457 | CAGTTCAGATGCTTGTCAAG | 3319 |
| RGPD4 | NM_182588 | GCAAGACACCCTCAGAGCAC | 3320 |
| RGPD5 | NM_005054 | ACAGTGCTGAGGCAGAACGC | 3321 |
| RGR | NM_002921 | TGAATGGGTTCCTTCTGCTT | 3322 |
| RGS10 | NM_002925 | GGAGGCTACAAATAACAGTT | 3323 |
| RGS19 | NM_001039467 | GTGGGGGCCGACGCGCGGGC | 3324 |
| RGS5 | NM_001195303 | AAGTGGGCTAAACGATCTCC | 3325 |
| RHBDD1 | NM_001167608 | TTACTGCCATAAATAGCCAC | 3326 |
| RHBDL3 | NM_138328 | CGCGCCCGCCCCCATGGCCC | 3327 |
| RHEB | NM_005614 | TTGAAGCCTTCAAACCTAGC | 3328 |
| RHOQ | NM_012249 | GCCGCGGGAGGGGCCCGGGT | 3329 |
| RHOU | NM_021205 | AGGAGCATTCACAATGGAGC | 3330 |
| RHOV | NM_133639 | TGCCTGCCTTTCCTCCTCCC | 3331 |
| RHPN1 | NM_052924 | CAACCAGAGTTCCAGGAAGG | 3332 |
| RIBC1 | NM_001031745 | CGGAAGGCGAAAATCCCGTT | 3333 |
| RILP | NM_031430 | TAAGCTTTCTGTGTCAGTCC | 3334 |
| RILPL1 | NM_178314 | GGGATCCGAGTTGCGCTCAA | 3335 |
| RIMS2 | NM_001100117 | GGGAAATGTTTCTTCTTCCC | 3336 |
| RIOK3 | NM_003831 | AACAAGTGGCAAAGCTAATA | 3337 |
| RIOK3 | NM_003831 | GAGGTCACACAGATAACAAG | 3338 |
| RIT1 | NM_001256821 | GTCATGTGACTGAACTGTCT | 3339 |
| RIT2 | NM_002930 | GGGGTAGGCAGGAAAGAGAA | 3340 |
| RLF | NM_012421 | CGTAGGCCACTGAGAGCACC | 3341 |
| RLIM | NM_016120 | GATTCCTCGAAAAGGCTCCG | 3342 |
| RMDN2 | NM_001170791 | CACACGGTCCGGCGCGAGCC | 3343 |
| RNASEH2A | NM_006397 | CTATGGCCGAACACTCAGCT | 3344 |
| RNF123 | NM_022064 | ACATGCTAACCGGAATCCCT | 3345 |
| RNF130 | NM_018434 | ACCAGCACCAGCGGCTGACC | 3346 |
| RNF14 | NM_183399 | GACATCATGTCAGAGGTCAC | 3347 |
| RNF14 | NM_183399 | GTCAATTTTGAGGACAAGAT | 3348 |
| RNF146 | NM_001242846 | CTTCGCTGCTTGCATTCTTC | 3349 |
| RNF146 | NM_001242851 | GGAGGAAGTAAAACGTGTGT | 3350 |
| RNF151 | NM_174903 | GGGTCTCTGGGTCCTGAACC | 3351 |
| RNF20 | NM_019592 | TACTCTTAGAGGTCGTAGCC | 3352 |
| RNF212 | NM_194439 | ACCTGAGGACCGCCAAGACA | 3353 |
| RNF214 | NM_001278249 | CGCCGCCAGAGGGCGCCGTC | 3354 |
| RNF217 | NM_001286398 | CAGTGGCTCGGCTCGACTCG | 3355 |
| RNF225 | NM_001195135 | ACGCTAGCTACACCCTTCTC | 3356 |
| RNF32 | NM_001184997 | CACGTCCTCCCCATGTGCTG | 3357 |
| RNF6 | NM_183043 | TGGGCTCGAGGGAAAGATCT | 3358 |
| RNF6 | NM_183044 | TAAGAAGGCAGTTAACCAAT | 3359 |
| RNF7 | NM_183237 | TCAGCGGCGTCGCCCCATAA | 3360 |
| RNPEPL1 | NM_018226 | CGGCGGGGCGCGGGCACAAC | 3361 |
| ROCK1 | NM_005406 | CCTGCATGGCTCCTCAGAGC | 3362 |
| ROS1 | NM_002944 | AGCTCAGAGAAGTAAGGTGG | 3363 |
| ROS1 | NM_002944 | TGACACATGCAGTCTGAAAC | 3364 |
| RP1 | NM_006269 | AGGCAAGAAAGAAGATGCAA | 3365 |
| RP9 | NM_203288 | CTGAGACTTCGGGGCCGCCG | 3366 |
| RPAP3 | NM_001146076 | GGAACCAGCTTGGTGGCTTG | 3367 |
| RPE | NM_199229 | AAGATCCAAACAGCACAAGA | 3368 |
| RPF2 | NM_001289111 | AAATCCGTAACCAAGACAAC | 3369 |
| RPGR | NM_000328 | CGGAGGCCGGGTGGCTGGTA | 3370 |
| RPGRIP1 | NM_020366 | ATTTCTCAGCACTTTCATGA | 3371 |
| RPL10 | NM_001256577 | GCGGGCTTCTCGCGACCATG | 3372 |
| RPL13 | NM_033251 | CGGCAACATGTCTGCGACGG | 3373 |
| RPL15 | NM_001253380 | AGAACCAGAACTGAGCACCA | 3374 |
| RPL17 | NM_001199340 | GCCATTTACAAACCACTTTC | 3375 |
| RPL17 | NM_001199342 | CGAGATCTGAGGAGGCAGGA | 3376 |
| RPL26L1 | NM_016093 | AAGCAGGCCCTTGTACTCAC | 3377 |
| RPL28 | NM_000991 | ATTCGGAACTCTTCGGTTAG | 3378 |
| RPL32 | NM_001007074 | CTACCGGAAGGACCATCTGG | 3379 |
| RPL35A | NM_000996 | TGTAAGAGTGCTATTGAATG | 3380 |
| RPL36 | NM_015414 | ACGCGCATGCTCAGGGAGCT | 3381 |
| RPL36 | NM_033643 | CTCATTTCACAGGCAGAGGG | 3382 |
| RPL36AL | NM_001001 | GTTGTCATAACGGTCCCCGC | 3383 |
| RPL7 | NM_000971 | AGTTCTTTGCGTCTGCAAGG | 3384 |
| RPL7 | NM_000971 | TTTAGTTCTGGATTCTTTTC | 3385 |
| RPL7A | NM_000972 | CTGACTAGGTTTTCGGACCG | 3386 |
| RPL7L1 | NM_198486 | TGGCAGGAATCGGGGTTAGC | 3387 |
| RPN1 | NM_002950 | TATCCCGAGCAGCTCTGAGA | 3388 |
| RPP38 | NM_006414 | GTATGTATCGCGAGACCATG | 3389 |
| RPP40 | NM_006638 | GAGCAGTTCTTAGACTTCTT | 3390 |
| RPRD1B | NM_021215 | GCTACTTAGCGCGTCACTTC | 3391 |
| RPS15A | NM_001019 | TCGATGGAATCGACCTCCCC | 3392 |
| RPS17 | NM_001021 | CTCCCCCATCTGATTTTTAA | 3393 |
| RPS20 | NM_001146227 | ACCTGAGAAACTCCTCTGTC | 3394 |
| RPS24 | NM_033022 | GAGTTGTTCTGGTTCTGGAT | 3395 |
| RPS27 | NM_001030 | AGTTAAAGACCTTCCGAAAA | 3396 |
| RPS29 | NM_001030001 | GTATGGTGACGTCATCAACT | 3397 |
| RPS6 | NM_001010 | TGGGTCTGAGGTTGTGCCAG | 3398 |
| RPS6KA2 | NM_001006932 | GCCCCAGCCCGAGCGGGAAG | 3399 |
| RPS6KA4 | NM_003942 | GGAGACAGGGCGGCCCCAGC | 3400 |
| RPS6KL1 | NM_031464 | CTTCTACCCCCCATCCAACG | 3401 |
| RPSA | NM_002295 | CTGAAGAAAAAGCCCAGTCC | 3402 |
| RPTN | NM_001122965 | AAGCTGGGCTGAGCTGGGCT | 3403 |
| RPUSD2 | NM_152260 | TAACGTCGTATCTCCCTAAT | 3404 |
| RRAS2 | NM_001177314 | AAGATGGCTTTTCTGTTCTA | 3405 |
| RRAS2 | NM_001177315 | TCGCGCTCCTGCCTCCTCCC | 3406 |
| RRM1 | NM_001033 | ATTAACCGCCTTTCCTCCGG | 3407 |
| RRM2 | NM_001034 | CGCAGCGCGGGAGCCTCCGC | 3408 |
| RSBN1L | NM_198467 | TCCACCTAAGAGCCAATCAA | 3409 |
| RSC1A1 | NM_006511 | CTGTTTAGATTTGTATCCTC | 3410 |
| RSC1A1 | NM_006511 | TAAAATAAGGTCCTCAAACT | 3411 |
| RSF1 | NM_016578 | TTGCCACTGCCTCGTGTGAC | 3412 |
| RSL24D1 | NM_016304 | AGACCTGTTCGCTGTTACTT | 3413 |
| RSPO2 | NM_178565 | AAGAGGATTCGCTCCAAGTT | 3414 |
| RTBDN | NM_001080997 | GAGCCCTGCCACACCAGCCT | 3415 |
| RTF1 | NM_015138 | CTTCCCCCGTCGCTGGTTCC | 3416 |
| RTKN | NM_033046 | GGGGCAAGGGGACGCGACAA | 3417 |
| RTL1 | NM_001134888 | ccCCAAGTGACCAGCCAAAG | 3418 |
| RTN4RL2 | NM_178570 | TTAACCCTTTCTCGACCACT | 3419 |
| RTP2 | NM_001004312 | TTTCCTGATCTGATCTGCTT | 3420 |
| RTP3 | NM_031440 | CCCCAAGGACAAAGGTCAGT | 3421 |
| RTP3 | NM_031440 | GTGTCTTTTGAAATTCCTTG | 3422 |
| RUNX1T1 | NM_175635 | TCAGAAGTAAAAGCCTTGTC | 3423 |
| RUSC2 | NM_014806 | GGAAAGCTCTGCGCGTGACT | 3424 |
| RXFP1 | NM_001253729 | TCCTATTCCTGTGTCATTAG | 3425 |
| RXFP2 | NM_001166058 | CTCACTGGCATGAAGGGAGA | 3426 |
| RXRG | NM_001256571 | TCAGATGGAAGCTTTGGTCC | 3427 |
| RXRG | NM_006917 | TTCTATCTGTCCAATGTACT | 3428 |
| RYK | NM_002958 | CGGACGATGCAGCGAGGAGG | 3429 |
| S100A10 | NM_002966 | GGCGGCACCTCCCCAGAAGC | 3430 |
| S100A13 | NM_005979 | GGTGTTCGTCTGTGAAGGGG | 3431 |
| S100A4 | NM_019554 | TGGGCTGGTGGAGGGTGCTG | 3432 |
| S100A7L2 | NM_001045479 | GGATTTCTGGCCAGAATCCC | 3433 |
| S100B | NM_006272 | AAGCAGCCCCGGGGACTTGC | 3434 |
| S100PBP | NM_001256121 | ACTGTCACGCGAGTCCAGCC | 3435 |
| S1PR4 | NM_003775 | CCCGGGTGGGGGCCGACCGT | 3436 |
| S1PR5 | NM_001166215 | GTCGGGGGAACACGGAATCC | 3437 |
| SAAL1 | NM_138421 | TTATGAGTATGTTCGTGCCA | 3438 |
| SAC3D1 | NM_013299 | GTCCCTTCCACCCAATAAAC | 3439 |
| SALL1 | NM_002968 | GGGGCTCTTTGAAAGGCGAT | 3440 |
| SAMD13 | NM_001010971 | ACCCCAATGAAGTTTTAAGC | 3441 |
| SAMD3 | NM_001258275 | CTGGAGCTCCCCAGCCGCTC | 3442 |
| SAMD7 | NM_182610 | CCTTGCAGGGCACTTTCCTT | 3443 |
| SAMHD1 | NM_015474 | CCGGCACCGCACCCCCAATT | 3444 |
| SAMSN1 | NM_001256370 | GTAAAATTCAGGAACAGATG | 3445 |
| SARAF | NM_001284239 | GCGCGGCGGCGACAGGCCCT | 3446 |
| SARS2 | NM_017827 | TGGTAGATTTGGAGGACCCC | 3447 |
| SART1 | NM_005146 | GTGCAGTCGAGCGCTGATCC | 3448 |
| SCAF1 | NM_021228 | GGGGTCCGCGCGATGCACGC | 3449 |
| SCAP | NM_012235 | TATGGACGGCCGGGCCGGGC | 3450 |
| SCAPER | NM_020843 | ATGCTATATTATACCCCAAC | 3451 |
| SCARA3 | NM_016240 | GGGATGCGCGCTCTGGGCGG | 3452 |
| SCARA5 | NM_173833 | CTGAGGATGAATGTGACTCC | 3453 |
| SCARF1 | NM_145350 | CTGACTGGCCTGGGCCTGGA | 3454 |
| SCD5 | NM_001037582 | GGCCGAACTGGGGAGCCCGC | 3455 |
| SCEL | NM_144777 | TCAGTTAAAAGGGTGATCAC | 3456 |
| SCG2 | NM_003469 | AATGTGTCCTCCATTCATCT | 3457 |
| SCG5 | NM_003020 | GAGGAGGTGAATGACTTACA | 3458 |
| SCGB1A1 | NM_003357 | TGGCATTGGCTTGGTGGGAT | 3459 |
| SCGN | NM_006998 | TTTAACTTGCTTCTCAGACT | 3460 |
| SCIMP | NM_207103 | TCTGGCTTCTGGACAGCCGT | 3461 |
| SCML2 | NM_006089 | TGGTCCGCCACTGCCTGCGG | 3462 |
| SCML4 | NM_001286408 | GTTCTTTAAAAGCCAGTGGT | 3463 |
| SCN11A | NM_001287223 | AATCATAGTTCACACATGTC | 3464 |
| SCN1A | NM_001165964 | TCTGTGACACACCCAGAAGA | 3465 |
| SCN1A | NM_001165964 | TGAACCACTTTTAAAACTCA | 3466 |
| SCN1B | NM_199037 | ACCCCGGTCCCGCTCCGGCT | 3467 |
| SCN2A | NM_001040143 | TAGATCTCCATGTGAGCAAA | 3468 |
| SCN4A | NM_000334 | GTGGGCGTGCAGACTCTATC | 3469 |
| SCN4B | NM_001142349 | CGCCCTGCGCGTCCTGGAGT | 3470 |
| SCN4B | NM_174934 | GCGGTGGCCGCCGCGTAGGC | 3471 |
| SCN5A | NM_001099405 | CCAAGCCCCAGGCCGAACCC | 3472 |
| SCN5A | NM_001099405 | CGCGCCCAGGGCTCCGCACG | 3473 |
| SCNM1 | NM_001204848 | TTGACCTTTGTCTTATTTCT | 3474 |
| SCP2 | NM_001193617 | CAGTGGGGCCTAAGACTGAG | 3475 |
| SCRN1 | NM_014766 | CTCGACGGTGAGCAGCGCCG | 3476 |
| SCUBE1 | NM_173050 | CCTCCGGCCCTCCGAGGAAG | 3477 |
| SDC4 | NM_002999 | CCGCAGGCCTCGCTTCCACT | 3478 |
| SDCBP | NM_005625 | CTCCAGGTATCCGGCAAAGT | 3479 |
| SDPR | NM_004657 | CGTTACAATAACTTGTATCC | 3480 |
| SDSL | NM_138432 | ATGAGTCATAGGCAGTGCCC | 3481 |
| SEC13 | NM_001136026 | CGCAGTTACCCTGACCCGGA | 3482 |
| SEC14L1 | NM_003003 | ATCCAGCAGTGCGACGGGGC | 3483 |
| SEC16A | NM_001276418 | CGATGGCTGCCGCCAGTCCC | 3484 |
| SEC24D | NM_014822 | GTTAAAGGCTTTGACCTGTA | 3485 |
| SECISBP2 | NM_024077 | TTGGATCTGCCTTTTAGTGC | 3486 |
| SEL1L3 | NM_015187 | GCGCCCGCTGCTCCGAGGGG | 3487 |
| SELENOT | NM_016275 | GTCCTGACTCACCACCATCT | 3488 |
| SELPLG | NM_003006 | CTCCCCAGAAAGCTTCTACT | 3489 |
| SEMA3B | NM_001290060 | CTAGGCTGGCATGAAGTGGG | 3490 |
| SEMA3B | NM_001290061 | ACGCCACTGGGCACACCCTC | 3491 |
| SEMA4D | NM_001142287 | AGAACAAAGCTTCCACAGTG | 3492 |
| SEMA4G | NM_001203244 | ATTGTGAGTCGATCCTGGCG | 3493 |
| SEMA4G | NM_001203244 | CTATCGCTTTGCTCTGATGC | 3494 |
| SEMG2 | NM_003008 | GTCCCCATGCTAAGTCCCTG | 3495 |
| SENP1 | NM_001267595 | CGCTAGGTGGCTGAAGAGGA | 3496 |
| SEPT10 | NM_144710 | GCGTCTGAGGCCAGAGGACT | 3497 |
| SEPT11 | NM_018243 | CGGAGACGGTCGTTTGGGGA | 3498 |
| SEPT8 | NM_001098813 | GTTTTGAGCAGTGACATTAG | 3499 |
| SEPT9 | NM_006640 | TAAGCAGCCTCTGAGGACCC | 3500 |
| SERF1B | NM_022978 | ATTCAACAAGCTCGGAGCCC | 3501 |
| SERF1B | NM_022978 | TTAGTGCTAATGTAGCATGA | 3502 |
| SERF2 | NM_001018108 | TTCACATTTAAAGTTTCTGG | 3503 |
| SERINC1 | NM_020755 | ACTGCTGGCTGGAAACTTAA | 3504 |
| SERINC1 | NM_020755 | CTTTCCTGGAGAATTTCTCA | 3505 |
| SERPINA10 | NM_016186 | CAGGACCCAAGGCCACACAC | 3506 |
| SERPINB11 | NM_080475 | TGCACCATGTGCACTGACAC | 3507 |
| SERPINB12 | NM_080474 | TAATTTCTTATGGCAGCCCC | 3508 |
| SERPINB2 | NM_002575 | AATACTTGTTTGTAAAGGCA | 3509 |
| SERPINB2 | NM_002575 | GCATGGTTTAAGAAATTTTG | 3510 |
| SERPINB6 | NM_001271825 | CACATGAGTTTCACTGTGTC | 3511 |
| SERPINB6 | NM_001271825 | TGAACTGGAGAAACCAAAGC | 3512 |
| SERPINB7 | NM_001040147 | GTGCAGTCTGGGATGAAGGA | 3513 |
| SERPINE3 | NM_001101320 | TTTCTAATGCTGAAACAAGA | 3514 |
| SERTAD3 | NM_203344 | GTGGAAGGAAGCGGTTCTGT | 3515 |
| SESN1 | NM_001199934 | TTCTGCCCAGGGACGACTCA | 3516 |
| SESTD1 | NM_178123 | GGGTCGCGCGGACGCGGCTC | 3517 |
| SET | NM_001248000 | GGTTGTGGTGGAGCCTTCCT | 3518 |
| SET | NM_001248000 | TAGGTCTGGCTCATAGGGGA | 3519 |
| SETDB1 | NM_012432 | GCGGAGACTCGGTAATATAC | 3520 |
| SETDB2 | NM_031915 | ACTTACCGCTGGCACCGCAG | 3521 |
| SETDB2 | NM_031915 | GCGACCAATCAATGGGCTCC | 3522 |
| SF1 | NM_201995 | CCGCGACTCTCGCTTAATCC | 3523 |
| SF3B2 | NM_006842 | CCCTCGGCGGTCTGGTCGCG | 3524 |
| SF3B2 | NM_006842 | GCAGACGCACCTTTCTCTAG | 3525 |
| SFRP2 | NM_003013 | AAGTAGTGACCAGCCCTCCT | 3526 |
| SFXN3 | NM_030971 | GCGGCGCCACACCAGCGACC | 3527 |
| SGCE | NM_001099401 | GCAGACTGTGAGCCTTATAT | 3528 |
| SGIP1 | NM_032291 | GTGACAAGCGGGAGGCGATG | 3529 |
| SGK2 | NM_170693 | CACAACTTGTTATGTGACCA | 3530 |
| SGMS2 | NM_001136257 | TGTGAAGAGCTTTGTGCCCC | 3531 |
| SGO1 | NM_001012413 | CGGAGCCTGCGGTCGGGTCT | 3532 |
| SH2B1 | NM_015503 | TCCTTCAGCGACGGGAAAGG | 3533 |
| SH2B3 | NM_001291424 | ATTATTTATCTGATCCTGGG | 3534 |
| SH2D3C | NM_001142533 | GCGGAGCGGAGGACCTGCCA | 3535 |
| SH3BGRL | NM_003022 | CAGAAAAATCACTACGTAAT | 3536 |
| SH3BGRL3 | NM_031286 | CAACACGCACCACTAACCCT | 3537 |
| SH3D19 | NM_001009555 | AAATTTTTGATCGTCACAAC | 3538 |
| SH3D19 | NM_001009555 | TGGGAAGAAGGGAACTCTCA | 3539 |
| SH3D21 | NM_001162530 | GCTGCACAGGCCAGAGACCC | 3540 |
| SH3GLB2 | NM_001287046 | GGGGCGGAGCGAGAGGGCAG | 3541 |
| SH3RF2 | NM_152550 | AAAATATAAGCCAGTCCCTA | 3542 |
| SH3RF3 | NM_001099289 | AAGAAAGTCACGGCGGAGCC | 3543 |
| SHANK1 | NM_016148 | CTACCCCCACTGCCCAAGAT | 3544 |
| SHBG | NM_001146281 | GAGTCTTGTGACTGGGCCCC | 3545 |
| SHC1 | NM_003029 | GTTTGAAAGCGAGGCCAAAG | 3546 |
| SHC2 | NM_012435 | ACATCACCGGGCCGGGGGGC | 3547 |
| SHC3 | NM_016848 | TATAGTGTGCTGTCAGCGGG | 3548 |
| SHFM1 | NM_006304 | AACTACACGGATCTCAACTT | 3549 |
| SHFM1 | NM_006304 | TTGGTCTCTACCTTGTTATT | 3550 |
| SHISA4 | NM_198149 | GGGCATTCGGAGGTGGCACC | 3551 |
| SHISA5 | NM_001272082 | GGTCGCCCTCTGGGCCTAGA | 3552 |
| SHMT2 | NM_001166357 | GCATCAGGCAGGGGTCCCGG | 3553 |
| SHOC2 | NM_007373 | AGGAACTGAGGAAAGGACAA | 3554 |
| SIGLEC10 | NM_001171156 | CACAGTGAGCTACCCTTATC | 3555 |
| SIGLEC12 | NM_053003 | TCTCTGGCCTCAGGGTCCCC | 3556 |
| SIGLEC8 | NM_014442 | CACCACCCCATTTCCACTCC | 3557 |
| SIGLEC8 | NM_014442 | TCTCTGGCCTCAGGGTTCCC | 3558 |
| SIMC1 | NM_198567 | GCCTCGGCGTCTCGCACGCC | 3559 |
| SIPA1L1 | NM_001284245 | GAGTTTCACTCTTGTTGCCC | 3560 |
| SIRPA | NM_001040022 | TACAAAAATAGCGTGTGTGT | 3561 |
| SIRPB2 | NM_001134836 | AATCTTGCACAGCCAAGAAG | 3562 |
| SIRT5 | NM_012241 | CTCGCGAGCGGAGGTGGCAC | 3563 |
| SIVA1 | NM_006427 | TCGACGCCGCGGGAAAGGCC | 3564 |
| SIX1 | NM_005982 | AGCGTCCCCGGCACGCTGAT | 3565 |
| SIX5 | NM_175875 | ACGCCACGCGCATCCGCTCC | 3566 |
| SIX6 | NM_007374 | TGACTGACAGGGGGTCTCCA | 3567 |
| SKAP1 | NM_003726 | GGTGCACGTGGCGCTCACGC | 3568 |
| SKIL | NM_001248008 | AAAAAATTAGCCGGGTGTCG | 3569 |
| SKOR1 | NM_001258024 | CTGGAGTCAGCAGCGGAACC | 3570 |
| SKOR2 | NM_001278063 | GGTTAAGACACGATTATTAC | 3571 |
| SLAIN2 | NM_020846 | TGGCGGCAGGGGCCGGATAT | 3572 |
| SLBP | NM_006527 | AGACCATCGGGCCACGCCGC | 3573 |
| SLC10A1 | NM_003049 | GAGGAGTACAAGTAGCACCC | 3574 |
| SLC10A3 | NM_001142392 | CGCTGCCTGGACCAATCGCT | 3575 |
| SLC10A4 | NM_152679 | TTCTGTTATCGAGTGTAGCC | 3576 |
| SLC10A5 | NM_001010893 | TTGTAGGATCAAAGTCCAGT | 3577 |
| SLC11A2 | NM_000617 | GGCCAACGCAAGCAGCAACT | 3578 |
| SLC12A3 | NM_000339 | ATCAAATGGTGTTCTGCCTC | 3579 |
| SLC12A8 | NM_024628 | GCAGAGGCTTTCCCTCCGCA | 3580 |
| SLC13A3 | NM_022829 | CGGGAACGTTGGAGAAAGTT | 3581 |
| SLC15A5 | NM_001170798 | CTCCATGCTAGAATTTCATA | 3582 |
| SLC17A2 | NM_005835 | AGGGCTCCTGAAATCAGTGA | 3583 |
| SLC17A3 | NM_006632 | ATGCTTCTTCAAAGCCTATT | 3584 |
| SLC17A8 | NM_139319 | TAGGCCACGGATACTGCTGC | 3585 |
| SLC1A2 | NM_004171 | CCCAAGCCTTCCCGGACGAG | 3586 |
| SLC1A5 | NM_001145145 | ACACTGTCACACAAGAGTAA | 3587 |
| SLC1A6 | NM_001272088 | CCCCTTCTCCCACACGGCTG | 3588 |
| SLC1A6 | NM_005071 | GGACTCTCAGAAGGCGGGGG | 3589 |
| SLC22A1 | NM_003057 | GCTGAACTTCAATTCTCTTC | 3590 |
| SLC22A14 | NM_004803 | CCCCCCTGGCCCAACCATCC | 3591 |
| SLC22A17 | NM_001289050 | TAGGAAGGCAGTCAGGGGCG | 3592 |
| SLC22A18AS | NM_007105 | GCTTCCAGAGCCACACACTG | 3593 |
| SLC22A2 | NM_003058 | GTGGAGCACCGACAAGCCTG | 3594 |
| SLC22A3 | NM_021977 | GGCCGCGAGCCGGACGCACC | 3595 |
| SLC22A7 | NM_006672 | GGTCACTGGCTCGTGGCTCT | 3596 |
| SLC23A2 | NM_005116 | GGGAGCGCTGCCGGGTGCCA | 3597 |
| SLC24A5 | NM_205850 | AATCTGCCCTTAGAGACTGT | 3598 |
| SLC25A18 | NM_031481 | TCCAGATGCCTTCGCCTTCT | 3599 |
| SLC25A18 | NM_031481 | TGGCTAGTATTTTTCACTGA | 3600 |
| SLC25A19 | NM_001126122 | CCGTCCAGCTGTCCTGCCCT | 3601 |
| SLC25A24 | NM_013386 | CCAGTCCCGCTGTCAGCATG | 3602 |
| SLC25A28 | NM_031212 | AAGGGGAAAAGGTGGGATGT | 3603 |
| SLC25A34 | NM_207348 | ACTGGAGGGAGAGCGTGGAT | 3604 |
| SLC25A41 | NM_173637 | TCACGCTGCCCACCACACCT | 3605 |
| SLC25A42 | NM_178526 | ATTGGCGAGTATGAAGCAGA | 3606 |
| SLC25A43 | NM_145305 | AGCAAGATGTCTAGCAGGCT | 3607 |
| SLC25A45 | NM_001077241 | TCAGTCAGCCTTCTGTCTCC | 3608 |
| SLC25A48 | NM_145282 | GGCTCATCCCAGACACAAAG | 3609 |
| SLC25A51 | NM_033412 | GTCGGTTTTAGGGGCCTTGT | 3610 |
| SLC25A6 | NM_001636 | CATACCTAGGGGTGCGGGGC | 3611 |
| SLC25A6 | NM_001636 | GCGGGACGCAGCGGGATTCC | 3612 |
| SLC26A5 | NM_206885 | AGCACGCTTTGGAAAGTTCT | 3613 |
| SLC26A7 | NM_052832 | TGGGCTATGCTAATGAAACC | 3614 |
| SLC27A6 | NM_001017372 | GGTCCCGGAGAACTGCTCCT | 3615 |
| SLC29A2 | NM_001532 | GTCCCGGATCCCTGCGGCGG | 3616 |
| SLC2A10 | NM_030777 | GGGGAGCCCAGGACCGCCCC | 3617 |
| SLC2A14 | NM_001286237 | TCACTGCAACCTCTGCCTCC | 3618 |
| SLC30A5 | NM_022902 | GGAATCCGCTGTACTTCTGA | 3619 |
| SLC32A1 | NM_080552 | GGGGACGTGAGGAAGGGGCT | 3620 |
| SLC34A2 | NM_001177999 | AGAATGGAAGACGGCAGCCC | 3621 |
| SLC35A1 | NM_006416 | ATCCAAGCTACACCCCAAAA | 3622 |
| SLC35A5 | NM_017945 | GTGCGTCCGCTTCTCACCTC | 3623 |
| SLC35B1 | NM_005827 | GAAGTGGTTGCTGGGTTCTG | 3624 |
| SLC35B2 | NM_001286511 | CTGAGGAGTATCATCTCAAC | 3625 |
| SLC35C1 | NM_001145266 | CCTGTGGTCTGCCACTCACC | 3626 |
| SLC35E1 | NM_024881 | AAGCGCATCTACAGTGGACT | 3627 |
| SLC35E1 | NM_024881 | AATGGGAAACGGCGTAGACC | 3628 |
| SLC38A1 | NM_001278389 | AGTCTATTTCCCCCTGAGAA | 3629 |
| SLC38A1 | NM_001278390 | ACACAGGAAATTTTCACCAA | 3630 |
| SLC38A1 | NM_030674 | CCAACGCTGCCCGTAGTCCC | 3631 |
| SLC38A10 | NM_001037984 | AGCTGTCCGGTTCGCCAAGC | 3632 |
| SLC38A11 | NM_173512 | ACTCTTCCCTGGAGCTGCAG | 3633 |
| SLC38A11 | NM_173512 | AGGAACGGACTGCAACGAGG | 3634 |
| SLC38A11 | NM_173512 | AGTTAGCTTCTCCTTTGCTG | 3635 |
| SLC39A1 | NM_014437 | TCCAATCAGGACTCAGCTTT | 3636 |
| SLC39A5 | NM_173596 | AAAATAGGTTACAGGTAAGG | 3637 |
| SLC39A5 | NM_173596 | AACTAGGCATTTGGGAAGGT | 3638 |
| SLC39A9 | NM_001252148 | TCTGATGTCACTGTCTATAC | 3639 |
| SLC43A1 | NM_001198810 | TGAGACCGAGGAAAGCGGAG | 3640 |
| SLC45A3 | NM_033102 | AAAGCGGGAGGTCTCGAAGC | 3641 |
| SLC45A4 | NM_001286648 | ATTGACCCCTGAGCTTAGCC | 3642 |
| SLC45A4 | NM_001286648 | CAGGCCATGTCCTGCAGCCC | 3643 |
| SLC46A1 | NM_080669 | GGTGAGGTCATCCCGCGGGC | 3644 |
| SLC46A3 | NM_001135919 | CGCGGCCCACCACTCAACAG | 3645 |
| SLC4A11 | NM_001174089 | GGCGGCCGGGTCCCAGCCCT | 3646 |
| SLC5A10 | NM_001270649 | CTCCCTGACTCCTGCGCTCT | 3647 |
| SLC5A5 | NM_000453 | ACAGGCCAGGACAGGCTATC | 3648 |
| SLC6Al2 | NM_003044 | AGGTGGAAGGAGAAGTGGAC | 3649 |
| SLC6Al2 | NM_001122847 | GTCTCCAACTGCTGCTCAGA | 3650 |
| SLC6A17 | NM_001010898 | GGCAGCGAGCGAGGCTCTGA | 3651 |
| SLC7A8 | NM_001267037 | TTGGACAGGCCAAGCCGAAG | 3652 |
| SLC8A3 | NM_033262 | CAGATCCAACCCCTGCCCCG | 3653 |
| SLC8A3 | NM_182936 | CCTTGGCTGTGGACTGTTCC | 3654 |
| SLC9A1 | NM_003047 | CTTCTTTCCCTCGGCGACAG | 3655 |
| SLCO1C1 | NM_001145944 | TATAAACTTCCGCCCTCCTC | 3656 |
| SLCO2B1 | NM_001145211 | GGGGTCAGCTGGTCACTGAA | 3657 |
| SLCO4A1 | NM_016354 | GGAACGCGCGGCGGGGGACC | 3658 |
| SLCO5A1 | NM_001146008 | GAAAATGCCCAAAAGAACAA | 3659 |
| SLCO5A1 | NM_030958 | TTGGGCCCCCGCAGCCACGC | 3660 |
| SLF2 | NM_018121 | CAACAAGAACCGTCGTAGAA | 3661 |
| SLITRK4 | NM_001184749 | GGAAAGGGGGTTGGAGAACA | 3662 |
| SLITRK6 | NM_032229 | TCTCTTGTGTTATATGACAC | 3663 |
| SLU7 | NM_006425 | TAGGAGCTTTCTTTTAGAAT | 3664 |
| SLU7 | NM_006425 | TGCGTATCGCGCTATTTACC | 3665 |
| SMAD1 | NM_001003688 | GGCCGAGAAGAAAACCCGTG | 3666 |
| SMAD3 | NM_001145103 | TTAGCGACAGAGAAAATAGG | 3667 |
| SMAD4 | NM_005359 | GAGCGACCCTCCCCGTCACT | 3668 |
| SMAP2 | NM_001198980 | GATTGCATAAGCCTTTATTT | 3669 |
| SMAP2 | NM_001198980 | TGCAAGTGTTCTGAAAGCAG | 3670 |
| SMARCA2 | NM_001289398 | GAAATTTCTTCCATGTGCAA | 3671 |
| SMARCAL1 | NM_014140 | TTTGGAAACCTCAACGTCCT | 3672 |
| SMARCAL1 | NM_001127207 | CAGAGCCTCCCGAGCGGGAC | 3673 |
| SMARCB1 | NM_003073 | CCAGTCCTGGCTGTAAGACT | 3674 |
| SMARCD1 | NM_003076 | GGAAGACAAGGACCTGGAAA | 3675 |
| SMC3 | NM_005445 | CAGTCCTCCACAGCGTTTTT | 3676 |
| SMG8 | NM_018149 | TAGGAGAGGAGAAGAGGAGG | 3677 |
| SMIM1 | NM_001163724 | GGTGGCGGGGCTAGAGTGGT | 3678 |
| SMIM19 | NM_001135675 | GCCACTCACGCTGCCGGCTC | 3679 |
| SMIM22 | NM_001253791 | CAGCTCCTGGAAGCTCCACC | 3680 |
| SMOX | NM_175842 | GGCAGGGATCCAGCAGTCTC | 3681 |
| SMTNL2 | NM_001114974 | TCCGGGACACCCCCCTGCCC | 3682 |
| SMYD3 | NM_022743 | GGTATGAGTCATGGTCCAGA | 3683 |
| SMYD5 | NM_006062 | ACACTCCCGTCAACAAACCA | 3684 |
| SMYD5 | NM_006062 | CTGCCTTTGTGCTTTTACAT | 3685 |
| SNAI1 | NM_005985 | CGTGGCGGTGAGAGCCCGGG | 3686 |
| SNAP47 | NM_053052 | CACGGTCCATGCCATCTCCC | 3687 |
| SNAP91 | NM_001256717 | TCTCGGGTTCTACTCTGTGA | 3688 |
| SNCA | NM_001146055 | GTCTGATTCTTGCGCTAATT | 3689 |
| SNRNP35 | NM_180699 | CAGGCGTGAACCACCGCGCC | 3690 |
| SNRPA1 | NM_003090 | GGGTGTGTTTCGGAGTCTGG | 3691 |
| SNTB1 | NM_021021 | AGGAGGCACGCTGGCGGTGA | 3692 |
| SNUPN | NM_001042588 | TGCCAGGGTGTAGCCTCTGC | 3693 |
| SNURF | NM_022804 | TAGACATGTCCATTGATCCC | 3694 |
| SNW1 | NM_012245 | ATTATTCCTTGATAACCGCT | 3695 |
| SNX1 | NM_003099 | ATATCTCAGCATCGCGAACC | 3696 |
| SNX13 | NM_015132 | TCGGCTTGGCGCTGACTTGT | 3697 |
| SNX18 | NM_052870 | TCGCGGCACCGGCCACTAGA | 3698 |
| SNX21 | NM_001042633 | GATGACTCTGCGGCAGGCCT | 3699 |
| SNX24 | NM_014035 | AGATCAGCTGGGCCCGAAAG | 3700 |
| SOAT2 | NM_003578 | CTCACTCTGCTGTCTGTCGC | 3701 |
| SOBP | NM_018013 | GCCACGCCCGCTCGAGAGCC | 3702 |
| SOCS2 | NM_001270471 | GGTGACTATTTGCTCTTCCT | 3703 |
| SOCS2 | NM_003877 | AGAATTATGTACTCAAAAGC | 3704 |
| SOCS5 | NM_144949 | AATAGCAGGCAGGGCTTTAG | 3705 |
| SOGA1 | NM_199181 | AATAGAGGGGTTATTACTGG | 3706 |
| SON | NM_138927 | ATGGCGGACATAGTCGTGCG | 3707 |
| SON | NM_138927 | GCAGGGCCGTGCTCACTGAT | 3708 |
| SORBS2 | NM_001145672 | ACTCGGAAAGGAGGTGTGAA | 3709 |
| SORBS2 | NM_001145674 | TCTATTGCCCTAAGCCTCCT | 3710 |
| SORBS3 | NM_001018003 | GCCCTGTATTTTATTTATGG | 3711 |
| SOS1 | NM_005633 | CCAGCCGTGGAGAACGGACG | 3712 |
| SOS2 | NM_006939 | AGCGCGGCGACCCGCAAGCC | 3713 |
| SOST | NM_025237 | GCAAACTTCCAAATTGCTGC | 3714 |
| SOWAHB | NM_001029870 | AGGTGACACTCGCCCGGCCA | 3715 |
| SOWAHC | NM_023016 | ACGGCGCGAGGAATGCAGGC | 3716 |
| SOX13 | NM_005686 | GGGGACTTGCAGAAGAAGGG | 3717 |
| SOX14 | NM_004189 | GCGCTCTCTGTTTCTTGCAC | 3718 |
| SOX5 | NM_178010 | CTCACACCTGTCCTTCTCCA | 3719 |
| SOX5 | NM_178010 | GTGTATGTGTGTGTGTTTAA | 3720 |
| SOX6 | NM_033326 | TGCAGTGTTTGTTCTACCTA | 3721 |
| SP110 | NM_004510 | GATGTGGTTAGGGAAGCATT | 3722 |
| SP110 | NM_004510 | GGTACAGCCCCAGCGGCAAT | 3723 |
| SP4 | NM_003112 | GGCCGACTCCCCACCCCCCT | 3724 |
| SP6 | NM_199262 | CAGGAAGAGGGGATGGAATT | 3725 |
| SP7 | NM_152860 | AGCAAATGGAGCAGGAAATT | 3726 |
| SPACA1 | NM_030960 | CTCCTTGAGCCTTCCGGGTG | 3727 |
| SPAG11B | NM_058203 | TGAGAAGCGTTTGAGGACAT | 3728 |
| SPAM1 | NM_001174045 | AGAGTCTCACTCTGTCACCC | 3729 |
| SPAM1 | NM_001174045 | CATGCCACTACACTCCATCC | 3730 |
| SPANXA1 | NM_013453 | TGTGATGTGAAGCCACCCTA | 3731 |
| SPARC | NM_003118 | GGCACTCTGTGAGTCGGTTT | 3732 |
| SPATA17 | NM_138796 | AAAGCAGCATGAGAGAAAAG | 3733 |
| SPATA20 | NM_001258373 | GGGGAGGACAGCCCTTCTCA | 3734 |
| SPATA31D3 | NM_207416 | CCAGGAAGGTGGAGTCAGCT | 3735 |
| SPATA32 | NM_152343 | GAGGAAGGAGTTCTGGCTTC | 3736 |
| SPATA5 | NM_145207 | TCAGGAATTTACAATCTAAG | 3737 |
| SPATA6 | NM_019073 | CGTCAAACTGCGCCCAAAGC | 3738 |
| SPATA6L | NM_001039395 | CACACGTTTGTTATTGACGG | 3739 |
| SPATC1 | NM_198572 | TCACGGAAGAGGCACCATGA | 3740 |
| SPC25 | NM_020675 | GGATTGGTTGAACTCACCCT | 3741 |
| SPDYC | NM_001008778 | GGAGAGGCTTTCAAACCCTG | 3742 |
| SPDYE3 | NM_001004351 | CACTGTCCAAAAGCATCTTC | 3743 |
| SPEF2 | NM_144722 | CCAGCGCAGGAGGAAGCCGT | 3744 |
| SPG21 | NM_016630 | GGAGAGGGCTGAGTTACGTC | 3745 |
| SPHAR | NM_006542 | TGTTGGTTATATTGCACAAT | 3746 |
| SPI1 | NM_003120 | AGGGCTGGCCTGGGAAGCCA | 3747 |
| SPIN1 | NM_006717 | CGCCTGCCGCCGCCCATTCC | 3748 |
| SPIN2B | NM_001006683 | GAAGGGGCCACAGGGTTCCG | 3749 |
| SPINK2 | NM_021114 | TTCTTGTATGTCGGAGGGAG | 3750 |
| SPINK4 | NM_014471 | CAGCGTGCAAAGATTAACTC | 3751 |
| SPINK9 | NM_001040433 | TTGGGGACTAGCTATTAAAA | 3752 |
| SPIRE1 | NM_001128627 | CACAACAAATTTTCACATAC | 3753 |
| SPOCK2 | NM_001134434 | TCTGACCATTTCATCTGCCT | 3754 |
| SPON1 | NM_006108 | AGCAGCAGCCTCCTAGGCGA | 3755 |
| SPON2 | NM_012445 | GTGGCACCTAGGGAGGCACC | 3756 |
| SPRED2 | NM_001128210 | GATTGGTAATCATAACTTAC | 3757 |
| SPRED3 | NM_001042522 | AGACATGGAGAAGAAGATAG | 3758 |
| SPRR1A | NM_005987 | GAACACCACCTGATATTTTT | 3759 |
| SPRR2D | NM_006945 | GTATCCATATCTGGCATGAG | 3760 |
| SPRR2E | NM_001024209 | CTATCCATAACTGGCATGAC | 3761 |
| SPRYD4 | NM_207344 | AACAGAAACCACTACCTTGG | 3762 |
| SPTB | NM_001024858 | CTGTCAGGATCTACTCACGT | 3763 |
| SRC | NM_005417 | TGGTTCTTGCAAGTAGGTAA | 3764 |
| SRCIN1 | NM_025248 | CCGCGCGCTGCGGGATCACG | 3765 |
| SREK1 | NM_139168 | CCGGGTGCCCTAATCAAATA | 3766 |
| SRF | NM_003131 | TATCATTCTCGGGTTCAGGG | 3767 |
| SRGAP1 | NM_020762 | GACTAGATTAGCCCGGGCGC | 3768 |
| SRGN | NM_002727 | TTTGAAAAAGCAGGCCTGGG | 3769 |
| SRI | NM_001256891 | ACGAAGAAGCGCGCAGGCAG | 3770 |
| SRI | NM_001256891 | GCACTGCATTAGCGCCGTAA | 3771 |
| SRI | NM_198901 | ATTTCCAATTAGCCCTATAG | 3772 |
| SRI | NM_198901 | TTTCATAGAGGGCCTCTATA | 3773 |
| SRP68 | NM_001260503 | GAAGCTCTCATGATTCTCCC | 3774 |
| SRP68 | NM_001260503 | TATATTGAAGGCTTCCTGTT | 3775 |
| SRR | NM_021947 | ACGACGGTGGCCGCGCTGGG | 3776 |
| SRRD | NM_001013694 | GCGGGGCGGCGCGTGACCTA | 3777 |
| SRRM2 | NM_016333 | GGGAGACGATATCCCAGCCG | 3778 |
| SRRM3 | NM_001110199 | GCCTGGAGGAACGCCCGCAG | 3779 |
| SRRM4 | NM_194286 | TCTGCATAACAAAAGCCCGC | 3780 |
| SRRM5 | NM_001145641 | GGTGAGTGGTATGAAGTCAG | 3781 |
| SRRT | NM_015908 | GGAACTACGGGACCTCGGCT | 3782 |
| SSBP3 | NM_145716 | GAGCCGCTGCCTGCTCCTGC | 3783 |
| SSH2 | NM_033389 | GGTGGTGGGTGCGGAGTCTG | 3784 |
| SSR3 | NM_007107 | GGGCGAGCGGGCCAGACTTC | 3785 |
| SSSCA1 | NM_006396 | GCTGCTACCGAGAACCTGCT | 3786 |
| SSTR1 | NM_001049 | CTGAGGCTTGATTTGTGAGC | 3787 |
| SSTR2 | NM_001050 | GAGACCGGCTGAAACGCCTG | 3788 |
| SSUH2 | NM_001256748 | TGGTCAGTAGAAGGCTCTTG | 3789 |
| SSX2B | NM_001164417 | CTACTGTTCTGACTTCTAAT | 3790 |
| SSX2B | NM_001164417 | GGCAGTTAGTGAACTCCATC | 3791 |
| SSX5 | NM_175723 | CGGAACAAGCGAAGCTGATG | 3792 |
| ST6GAL1 | NM_003032 | AGAGTCTCGCTCTGTCGCCC | 3793 |
| ST6GAL2 | NM_001142351 | GCCCGCTAGAGCTGGGACCC | 3794 |
| ST6GAL2 | NM_001142351 | GGCGGGAGTCGTCCTGCCGC | 3795 |
| ST6GALNAC6 | NM_001287001 | CCGAAGCCGAGCTCCGGATG | 3796 |
| STAG2 | NM_006603 | TCCTTTCTCCCCTCCCCCCT | 3797 |
| STAM2 | NM_005843 | CTAAATTCGTGACAAGAACT | 3798 |
| STAMBP | NM_201647 | GAACGACACAGCGGCCATCT | 3799 |
| STAP1 | NM_012108 | AGGTGTAGACTGACTTTCAG | 3800 |
| STARD8 | NM_014725 | AATGTTCAGGGAATTTCAAT | 3801 |
| STAT6 | NM_001178079 | GGGATCCTCGTCCGCCCGCT | 3802 |
| STIM2 | NM_001169118 | CTTTAGCGAGCCGCGAAGAT | 3803 |
| STK10 | NM_005990 | CTTCCCCAAAGCCCAGCCCG | 3804 |
| STK19 | NM_004197 | AATGTTTCAAGGCCAGAGCC | 3805 |
| STK19 | NM_004197 | TCTGTACCCCTGCTTGTCTT | 3806 |
| STK25 | NM_001271978 | CTCTGTTCGCCCGGGGACCC | 3807 |
| STON1- | NM_001198594 | TCTCTTGGATAACATTTGCA | 3808 |
| GTF2A1L | | | |
| STOX1 | NM_001130161 | AAGTCGAGGGCATCGCCAGG | 3809 |
| STPG1 | NM_178122 | ATCACAAGATTTTTGAAGCA | 3810 |
| STRADB | NM_018571 | GACTTCACAACATCATCACT | 3811 |
| STRBP | NM_018387 | CGCGCGGCGAACGAGGGGGC | 3812 |
| STUB1 | NM_005861 | GGGGCCTCTGCTGATGGGGC | 3813 |
| STX4 | NM_001272096 | CATCATGGGACCTTGAAAAT | 3814 |
| STX6 | NM_001286210 | TGGCTTGTTCCCTCAGAACT | 3815 |
| STXBP2 | NM_006949 | GGACTCAACTTCCTGGGCCT | 3816 |
| SUCNR1 | NM_033050 | TGGCTGCAGGATATGCAAAT | 3817 |
| SUGCT | NM_001193312 | CAGACCAAGGGCACTCAGAC | 3818 |
| SUGT1 | NM_006704 | GTAACGTACTGTCATCCCTA | 3819 |
| SULF2 | NM_018837 | GGCCATCGATCAGGTCCACT | 3820 |
| SULT1A1 | NM_177529 | AGCAAACTCAGTCGTGGCTT | 3821 |
| SULT1A2 | NM_001054 | GTGATCTCCAAAGCCACGAC | 3822 |
| SULT1C2 | NM_176825 | AGGCTAAGGAGGAAGGAAAA | 3823 |
| SULT1C3 | NM_001008743 | TTCCCGATTAACAAGTAATA | 3824 |
| SULT1C4 | NM_006588 | GGAACGGGACCCAGCCAGCA | 3825 |
| SULT2A1 | NM_003167 | AAGATCGAATAACAAACACG | 3826 |
| SULT2A1 | NM_003167 | AGCTCAGATGACCCCTAAAA | 3827 |
| SULT2B1 | NM_004605 | TTTTGTCTTTTTAGTAGGGG | 3828 |
| SUN1 | NM_001171945 | ATTGGCCAGAACGCTTCGGG | 3829 |
| SUN2 | NM_015374 | CCTCCCACGCGCGGACTCCT | 3830 |
| SUN5 | NM_080675 | ATTGAGGCATCAAGACAGGA | 3831 |
| SUPT20H | NM_001278482 | CCAAGACGGCGCCGCCTGCT | 3832 |
| SUPT20H | NM_017569 | CCAGGATCTCTGCTCAATCC | 3833 |
| SURF4 | NM_001280788 | AGGAGGTGAGCAGCAGGCAG | 3834 |
| SURF4 | NM_001280788 | GGGTGGTAATGCGAGCCATG | 3835 |
| SURF4 | NM_001280792 | CGCGTTCCGCCGGGCCGGGA | 3836 |
| SVIL | NM_021738 | TGGGCTCCTCTGAATTTCCA | 3837 |
| SVOP | NM_018711 | TTACTGAGCACCTATGTGCC | 3838 |
| SWSAP1 | NM_175871 | GAACTGTACCGATGCGGCCA | 3839 |
| SWT1 | NM_017673 | AACTGCGCAGAAGCGTACTG | 3840 |
| SWT1 | NM_017673 | CGGTTTCTACGGTGCGTCTC | 3841 |
| SYBU | NM_001099748 | CAGAGTCTCACTCTGTCGCC | 3842 |
| SYBU | NM_001099751 | TTCGAGCACTTTGAGAGGCC | 3843 |
| SYCE2 | NM_001105578 | TTCTCAAAGAGGGCGGGGCC | 3844 |
| SYCP2L | NM_001040274 | CAGGCGTGAGCCACCGCGCC | 3845 |
| SYK | NM_001174167 | AAAGAGGCCCCGTGCTGCTG | 3846 |
| SYNE1 | NM_033071 | GGAACCGGTCGCGGAGGGCG | 3847 |
| SYNPO2 | NM_001128933 | CTGTTAGTGCAAGATAACTT | 3848 |
| SYT12 | NM_177963 | TCGAGCGCTGTCTCTGCTCC | 3849 |
| SYT4 | NM_020783 | AACTGACAGGGATCAGTTTC | 3850 |
| SYT7 | NM_004200 | GCGCGCAGGCCGGAGGGAGG | 3851 |
| TAB2 | NM_015093 | TTAGAAGCGAACGCCCCGCA | 3852 |
| TAC4 | NM_001077506 | TTAAGCTGAAGGAAGGAATC | 3853 |
| TACC2 | NM_206862 | ACTCTGACATTTTGCCCCTT | 3854 |
| TACO1 | NM_016360 | AACAAAGTCCGGCGCTCTCT | 3855 |
| TAF12 | NM_001135218 | GAGCTCTGCGTATTCCAACC | 3856 |
| TAF13 | NM_005645 | GGGAGGACGGTGGTGCTTTC | 3857 |
| TAF13 | NM_005645 | GGGATTACAGGGAGGCGCTC | 3858 |
| TAF1L | NM_153809 | GCCTGTAGTCCCAGCTACTC | 3859 |
| TAF4B | NM_005640 | CCCTCCTTGCTGGCGATTCT | 3860 |
| TAF6L | NM_006473 | TTATTTCCTCGTTACTATTG | 3861 |
| TAF7L | NM_024885 | CTACAATCTTGAACCGGCAC | 3862 |
| TAF9 | NM_003187 | GAAATGTGTCATCGAAAGCC | 3863 |
| TAF9 | NM_001015892 | CCTGTAATCAGTGGGGTGCC | 3864 |
| TAGAP | NM_138810 | AAGGCTCTGATTAATGTCAT | 3865 |
| TAGLN3 | NM_001008273 | CTGCAGTTCAACATGAAAGG | 3866 |
| TAL1 | NM_001287347 | GCTTCTAAGTGTGGTCTTCT | 3867 |
| TAL1 | NM_001290404 | CTCGGTTCCTTTCGATGGCC | 3868 |
| TAL1 | NM_003189 | GAGCGTTGGACGCGCTGTCT | 3869 |
| TANC2 | NM_025185 | TACATGAGATGTTTTGATAC | 3870 |
| TANGO2 | NM_001283179 | ATTTGCTGTCAGATGGGGCG | 3871 |
| TANGO6 | NM_024562 | GGCTTAGTCCGGGGGGTAAG | 3872 |
| TAOK1 | NM_025142 | TGAGGGCGCCTCCTCGACCC | 3873 |
| TAOK1 | NM_025142 | TGGGCTCAGTTAAGATGGCG | 3874 |
| TARBP2 | NM_004178 | AAGGAAGGTTGTGATTGGTC | 3875 |
| TARM1 | NM_001135686 | GGAAACTGAAAGGCTAGGAA | 3876 |
| TAS2R16 | NM_016945 | TTTGTTTATGCTTTGCTTGC | 3877 |
| TAS2R20 | NM_176889 | ACTCATTCATTAGTTTAAGC | 3878 |
| TAS2R41 | NM_176883 | TTCCTAGGAGTGCTAAAGAG | 3879 |
| TAS2R43 | NM_176884 | GGTTTATTGAGAAGAGAGAA | 3880 |
| TAX1BP1 | NM_001206901 | GACATTAGCTTTGATAACAT | 3881 |
| TBC1D12 | NM_015188 | AGAACTGTCACGCTTAGAGC | 3882 |
| TBC1D12 | NM_015188 | AGCGAGCAATACCCGCGCTT | 3883 |
| TBC1D14 | NM_001113361 | AGACGGCCCGGGCCCCGCCG | 3884 |
| TBC1D14 | NM_001113363 | CAACACGTTTCTCAGCTCTC | 3885 |
| TBC1D16 | NM_001271844 | TGTCAGCTGCAGTTTTGCCC | 3886 |
| TBC1D22A | NM_014346 | GGAGTCCGTTGCGGGCAGGT | 3887 |
| TBC1D25 | NM_002536 | GCTCCTGGCAACAGCACTCT | 3888 |
| TBC1D26 | NM_178571 | GAGGGTGCTGGCTCTGGTCC | 3889 |
| TBC1D3F | NM_032258 | TGCACAAACACGTTGCAAGC | 3890 |
| TBC1D3H | NM_001123392 | TGCACAAACACGTTGCAGGC | 3891 |
| TBCCD1 | NM_018138 | GGGTCGAGAGTCCGCAACAG | 3892 |
| TBCCD1 | NM_018138 | TCAAGCGTCTGAGAAAATCT | 3893 |
| TBL1x | NM_005647 | CTCGCGGCAGCTCCCCGTGG | 3894 |
| TBR1 | NM_006593 | TTTAGGAAGATTCAAAGATG | 3895 |
| TBX1 | NM_080646 | GTCGCAGGGTCTGATTCCTC | 3896 |
| TBX21 | NM_013351 | GAGTACTGCAGGGCCCCCCA | 3897 |
| TBX22 | NM_001109878 | AAGTTGCTGGAGTCCAACCC | 3898 |
| TBX6 | NM_004608 | CCGACCGCGAGGGGGCTGCG | 3899 |
| TC2N | NM_001128596 | AGGCCTAAGATACTACTAAG | 3900 |
| TC2N | NM_152332 | GCGCGGCTCAGGTACGCGGG | 3901 |
| TCEA2 | NM_003195 | ACACTTAACTCCAGTTTCAC | 3902 |
| TCEA2 | NM_198723 | GTCGAGTGTGGAGGACACCC | 3903 |
| TCEAL1 | NM_004780 | GGCAGGGCCGCAGATCAAAG | 3904 |
| TCEB3B | NM_016427 | ATTAACCTAATCAACCTCTG | 3905 |
| TCEB3B | NM_016427 | CGTTGACCTTCCATGTTCGC | 3906 |
| TCEB3CL | NM_001100817 | GGTGGCCGGTCCTCGCTGCC | 3907 |
| TCF15 | NM_004609 | CGAGGGAGGGGCCAATGGCA | 3908 |
| TCF25 | NM_014972 | CCGGAACTTTCCCGCTTCAG | 3909 |
| TCF3 | NM_003200 | GGGTCGCGCGTGGGCGGCGG | 3910 |
| TCF4 | NM_001243226 | CATTTTCCTCCTACCATTTC | 3911 |
| TCF4 | NM_001243235 | ATCGATCTCGCGTATGCATT | 3912 |
| TCF4 | NM_001243235 | GGAAGGCAGCCCGGCCCTGA | 3913 |
| TCF7 | NM_003202 | CCTTAAAGGGCTCGCTCTTC | 3914 |
| TCHP | NM_001143852 | ACGTCGCTGCTCCTTGAAAT | 3915 |
| TCP10 | NM_004610 | ACTCTCTCCAGTGTCCTTTG | 3916 |
| TCP11L1 | NM_001145541 | ATCTCTTCGCCTCTTCCCGT | 3917 |
| TCTEX1D1 | NM_152665 | GGGTTGGCGGCGAGCTGGAG | 3918 |
| TDG | NM_003211 | CGCTCCTAGTCCCCGTCTTC | 3919 |
| TDGF1 | NM_001174136 | CTTGTTAATGAAGTGTGGCC | 3920 |
| TDP2 | NM_016614 | GAGCAGCGCATTTCCCCGCC | 3921 |
| TDRKH | NM_006862 | CTAGCCGCTGCCCAATTACC | 3922 |
| TEAD2 | NM_003598 | GCTGGTAGGAACTCAGGATT | 3923 |
| TECRL | NM_001010874 | CTGTCTAAGGTAAAGAGAAG | 3924 |
| TECTA | NM_005422 | CATGAAGTGTTGAACTTCGG | 3925 |
| TEFM | NM_024683 | CGGACGACCCACTGCTCAGC | 3926 |
| TEK | NM_000459 | CAGGTTGTATTTTCTCATCA | 3927 |
| TEK | NM_000459 | TTTTCTCATTTTAACCCACA | 3928 |
| TEN1-CDK3 | NM_001258 | CCTCCTCTGAAGGCAGAGCC | 3929 |
| TENM3 | NM_001080477 | CTACCATCCCAGATTGAGAA | 3930 |
| TENM4 | NM_001098816 | AGCTGCAATCCCGAGGCTTC | 3931 |
| TENM4 | NM_001098816 | GCACGACCGGCTCCCGCTCC | 3932 |
| TERF2 | NM_005652 | GTAGCTGTTTTCTGTAAATT | 3933 |
| TERF2IP | NM_018975 | ACTCACTTCTTGCTCAGTTT | 3934 |
| TESC | NM_001168325 | GCAGGTGTGCGGAAGGGACG | 3935 |
| TESPA1 | NM_001098815 | AGGTCTTATGGGCCACATCA | 3936 |
| TEX101 | NM_031451 | TCTTTGAAAGGCAGGCATCC | 3937 |
| TEX13B | NM_031273 | GAAGGCCTCTGCCATTCCAC | 3938 |
| TEX2 | NM_001288733 | TAGTCAGCTGATGTGCACTC | 3939 |
| TEX22 | NM_001195082 | TGGGCTCCGTTGCGGCGGGT | 3940 |
| TF | NM_001063 | GACTGCGCAGATAGGACTGG | 3941 |
| TFAP2E | NM_178548 | GTCTCTTTAATGCGCGCCCC | 3942 |
| TFDP2 | NM_001178139 | TGCACTCAGCCACCGCCCCT | 3943 |
| TFEB | NM_007162 | GTCCTGCTTCCCTCTCCTGC | 3944 |
| TFEC | NM_012252 | AGTGCTCTTTCTCAAATTAG | 3945 |
| TFPI | NM_006287 | ACTGATTACAAAAACAATCC | 3946 |
| TFPI2 | NM_006528 | CGGAGCGGGATTCGTTGCAA | 3947 |
| TGDS | NM_014305 | TCGCCCGGATGGTAGGGGTA | 3948 |
| TGFB3 | NM_003239 | GAGCGAGAGAGGCAGAGACA | 3949 |
| TGFBI | NM_000358 | TAGGTCCCTTAGGCCTCCTG | 3950 |
| TGFBI | NM_000358 | TGGCAGTGAGGGCAAGGGCT | 3951 |
| TGFBR1 | NM_001130916 | CTGCGGATTGGCTGCCTGGC | 3952 |
| TGFBR3 | NM_001195683 | ACAGGCTCGAGCAGCATTCG | 3953 |
| TGIF1 | NM_170695 | GGTTGTAAGTGCAAAGAGCA | 3954 |
| TGIF1 | NM_173207 | TCAGATACCAGCAATTGCTT | 3955 |
| TGIF1 | NM_173209 | GGAACTCGCAGCTTTAGCCC | 3956 |
| TGIF2LX | NM_138960 | CTGCGTGAAATCAAGTGCAT | 3957 |
| TGIF2LY | NM_139214 | CTGCGTGAAATGAAGTGCAT | 3958 |
| THAP2 | NM_031435 | GGCCGCTTGGTGTCCGAGTA | 3959 |
| THAP5 | NM_182529 | CCTGCATCCGTCGCCGGCCC | 3960 |
| THBS2 | NM_003247 | AAGTTGCCAACATTTATCTC | 3961 |
| THEM6 | NM_016647 | GCGAGGGTGCACGCGCGCCC | 3962 |
| THEMIS | NM_001164687 | ATTGCAGGAAATACTGAATC | 3963 |
| THEMIS | NM_001010923 | TTCTGACATTGAAGTTGAAC | 3964 |
| THG1L | NM_017872 | CTGATTTGCCGCAGGACGGG | 3965 |
| THOC2 | NM_001081550 | CCCTTTGCGAGGTTACTACA | 3966 |
| THOC2 | NM_001081550 | CCTTGCCTCGGGTTTCCGCT | 3967 |
| THOC3 | NM_032361 | TATTACTAAGTAAGCAGACG | 3968 |
| THOC5 | NM_001002879 | GTAAGGAAGGGGCGGCCGAC | 3969 |
| THOC6 | NM_024339 | CCTGGACGCCAGGTGCGTGT | 3970 |
| THPO | NM_000460 | GATCCATCTTTTCCTGGACA | 3971 |
| THSD1 | NM_199263 | TAATACCAATTCTGACCCCA | 3972 |
| THUMPD2 | NM_025264 | GAGGGGACAGATGGTCAACC | 3973 |
| TIAF1 | NM_004740 | TTTGGGAGAAAGAAAAGAGA | 3974 |
| TIAM2 | NM_012454 | TGCTTCTCCAGTTAGGATGT | 3975 |
| TICRR | NM_152259 | CTCCAGGAACTGCTGCTATT | 3976 |
| TIGAR | NM_020375 | CCTGCGCGCCGGCCTGTGAT | 3977 |
| TIGD3 | NM_145719 | ACGTCCAATGAAACTTAGCC | 3978 |
| TIGIT | NM_173799 | AACAAATACACAAACTGCAT | 3979 |
| TIMM10 | NM_012456 | ACCAAAGTACCATAGAAGCT | 3980 |
| TIMM10B | NM_012192 | GCGACGGGAACTGGAGCCCG | 3981 |
| TIMM22 | NM_013337 | GTCTCGCTGGTGTGCGCACC | 3982 |
| TIMM23 | NM_006327 | GCCAGTGGAAGAGAGAAAGC | 3983 |
| TIMM44 | NM_006351 | GTGACGGAATACACGCCCCT | 3984 |
| TIMM50 | NM_001001563 | GATCATTCTTGGGTGTTTCT | 3985 |
| TIMM9 | NM_012460 | CGCATGCGTGTTGTGTCTCA | 3986 |
| TJP2 | NM_001170415 | ATGCTCTAGTTCCCTGGCAA | 3987 |
| TJP2 | NM_001170416 | ACGTAAGGCGGATACAATAG | 3988 |
| TK2 | NM_001272050 | GCGTCTTGGTCCCGCCTCCC | 3989 |
| TKTL1 | NM_001145934 | ACAGACTGAGAAATTTGTCA | 3990 |
| TKTL2 | NM_032136 | TACTAAAAATCCATTCAGCT | 3991 |
| TLDC2 | NM_080628 | AAGGGCAGCTGGCGTGGGCA | 3992 |
| TLE2 | NM_003260 | CCTTAAGGCGGCTCAGCCCG | 3993 |
| TLE6 | NM_024760 | ACGCGACCCACGTGCGTAAA | 3994 |
| TLL2 | NM_012465 | GATTGGCTGCTTAGGGCCCC | 3995 |
| TLR10 | NM_030956 | CACACCACTGCACTCCAGCC | 3996 |
| TLR2 | NM_003264 | GCGAGGTCCAGAGTTCCCTC | 3997 |
| TM4SF18 | NM_001184723 | CAACAACTGAAGAGCTGAGC | 3998 |
| TM4SF4 | NM_004617 | CATGGGCACTGTCAGATTAA | 3999 |
| TM4SF5 | NM_003963 | ATCAGAATGATAAGGGAGAG | 4000 |
| TM9SF2 | NM_004800 | TGGAATTGGAACGTGAGCGC | 4001 |
| TMBIM4 | NM_016056 | GTTTCACTTCAGATGACGCC | 4002 |
| TMBIM6 | NM_001098576 | GTACGTCTGAACCTAGTACT | 4003 |
| TMC2 | NM_080751 | TCTTGGTTTGAGATTGAATG | 4004 |
| TMC3 | NM_001080532 | TGCTCTGCCCGCTAGTTCTC | 4005 |
| TMC5 | NM_001105248 | AGAATTGAGCCAGTTCCTGC | 4006 |
| TMC7 | NM_024847 | TGCTTGTCGCCACCGCTGGA | 4007 |
| TMCO1 | NM_019026 | GCTGGCGCGCGCCTTTTTCT | 4008 |
| TMCO2 | NM_001008740 | AATGAACTGAAAACCCAGGC | 4009 |
| TMED1 | NM_006858 | AAAGGCTTCGGCTCTCTTCT | 4010 |
| TMEM100 | NM_018286 | AAAAGCTGGCTCCTGTCTCT | 4011 |
| TMEM107 | NM_032354 | AGTACATTCTCCGGCTGCTG | 4012 |
| TMEM123 | NM_052932 | AGGGGATGGGATTCACTCTA | 4013 |
| TMEM125 | NM_144626 | GAACTCTTGAGTTCAAAAAC | 4014 |
| TMEM126A | NM_001244735 | AAACGAGCACACTCTACGCC | 4015 |
| TMEM128 | NM_032927 | CACACTTGCCGACATGAGAG | 4016 |
| TMEM132D | NM_133448 | GGGTGGCCGGGCTCGCTGGG | 4017 |
| TMEM135 | NM_001168724 | GTACGCGAGGGAGCGCAGCT | 4018 |
| TMEM143 | NM_018273 | AGGGAGTCGGCGGTGAGAAA | 4019 |
| TMEM150B | NM_001085488 | GAGTTTCGCTCTTGTTGCCC | 4020 |
| TMEM154 | NM_152680 | ACAGCTTCTTCCTAGGGTCT | 4021 |
| TMEM154 | NM_152680 | AGTGAGAATGCGTGTGGTCC | 4022 |
| TMEM155 | NM_152399 | GGAAGGCTTTGGTGCCAGCT | 4023 |
| TMEM161B | NM_001289007 | CTGCGCTTGCGAGGACCCTG | 4024 |
| TMEM185A | NM_001174092 | GATCTGCCCGCCAGACTCCC | 4025 |
| TMEM196 | NM_152774 | ATCTTCGCACCACCGAACCC | 4026 |
| TMEM203 | NM_053045 | CGAAGAGCACCAGAAGCTGC | 4027 |
| TMEM208 | NM_014187 | GGTGAGAGGAAGCCGCCCTC | 4028 |
| TMEM218 | NM_001258241 | CCATCTCTCCGTAACTCATT | 4029 |
| TMEM251 | NM_001098621 | CCGGGCTGGAGCCGGAGCTC | 4030 |
| TMEM256- | NM_001201576 | TCGCTGCGAGGTGCCCGTGT | 4031 |
| PLSCR3 | | | |
| TMEM257 | NM_004709 | TAAATACAGAATACAGAGGT | 4032 |
| TMEM266 | NM_152335 | TCGGCCAAGCCGCCGGCGCG | 4033 |
| TMEM42 | NM_144638 | CCACGCTCCGGCAGGCCCCT | 4034 |
| TMEM61 | NM_182532 | TGCCCGAGGACGCGGAGGAG | 4035 |
| TMEM67 | NM_153704 | AGAGTTCCTCTACTTACGAT | 4036 |
| TMEM79 | NM_032323 | AAGGGGTAAGTTCACATTCT | 4037 |
| TMEM8B | NM_016446 | TGCTTGGGGTGAGAAAGGCA | 4038 |
| TMEM9 | NM_001288571 | ACGTCAGCCTTCCAAACTCC | 4039 |
| TMEM95 | NM_198154 | TGGCACTGCCCATCCTCAGC | 4040 |
| TMEM99 | NM_145274 | GGCTACGGTGGTGGCAGTTC | 4041 |
| TMIGD3 | NM_001081976 | TCATGAGTTTTAGGAGCTTA | 4042 |
| TMOD2 | NM_001142885 | AGAGGACACCTGTCGGGGAA | 4043 |
| TMOD4 | NM_013353 | TCAGCCAGTTCCTCCTTATT | 4044 |
| TMPRSS15 | NM_002772 | GTGAGTTGTGTATGTCTCTT | 4045 |
| TMPRSS2 | NM_001135099 | ATCTCAGGAGGCGGTGTCCC | 4046 |
| TMX2 | NM_015959 | GTCGCCTTATGAGAACGTTC | 4047 |
| TNC | NM_002160 | GCCATAAATTGTATGCAAAT | 4048 |
| TNFAIP2 | NM_006291 | TGTTTCACCCATTCAGCCAC | 4049 |
| TNFAIP3 | NM_006290 | CCGCCCCGCCCGGTCCCTGC | 4050 |
| TNFAIP8 | NM_001286813 | GAGGAACTGGAGGCTCAGAG | 4051 |
| TNFAIP8L1 | NM_001167942 | CAGAGCAGAGCCCCACGCCA | 4052 |
| TNFRSF12A | NM_016639 | TCTGCGTCCCTGCGGGGTCC | 4053 |
| TNFSF18 | NM_005092 | TTTATGTTCTGAGTTTGTGT | 4054 |
| TNIP1 | NM_001258456 | GGCAGTCCCCCACTTTAAGC | 4055 |
| TNIP3 | NM_001128843 | TCTAATACATAGAGCATGAA | 4056 |
| TNIP3 | NM_024873 | AATCGTCATTCTTCCTTTAC | 4057 |
| TNNI2 | NM_001145841 | GAAGTGATTCCCCTGTGACC | 4058 |
| TNNI2 | NM_003282 | CCGCCCAGTCCAAGAAGTCT | 4059 |
| TNNT2 | NM_000364 | TGTTCCTGTAGCCTTGTCCC | 4060 |
| TNPO1 | NM_002270 | AGCACCAGACTTCACCGGCC | 4061 |
| TNPO2 | NM_013433 | CTGAGTGAGGCCCACTTACC | 4062 |
| TNRC6A | NM_014494 | TAGCAACTGGACCCGCAGAT | 4063 |
| TNS2 | NM_170754 | GAGGGGGGAGGATGTGGGGG | 4064 |
| TNS3 | NM_022748 | ATTGTTAGGGTGATGAGGCC | 4065 |
| TNS3 | NM_022748 | CGCCTCCAGGCGCCCTTCAC | 4066 |
| TOM1 | NM_001135730 | CCTTTAGACCTCGCCCTAAA | 4067 |
| TOMM6 | NM_001134493 | AGGCGGCGAGGTGACAAGTT | 4068 |
| TOP1MT | NM_001258447 | CAGCCACCGGACGCCCCGCG | 4069 |
| TOPAZ1 | NM_001145030 | AGTGGGGCTCATCACATAAC | 4070 |
| TOPAZ1 | NM_001145030 | CCGCGCCCGATTGCATTGCG | 4071 |
| TOR1A | NM_000113 | GCGGAGCAGAACCGAGTTTC | 4072 |
| TOR1AIP1 | NM_001267578 | AAATTTTTGCCACGAAAACA | 4073 |
| TOX2 | NM_001098798 | GAGATGGATTTTGATAAAAG | 4074 |
| TP53 | NM_001126117 | GGTCTTGAACTCCTGGGCTC | 4075 |
| TP53I11 | NM_001258320 | ACTCGGTTTCCCCTCTCCCC | 4076 |
| TP53I11 | NM_001258321 | AGCCTTCAGGCTTCCAGCCT | 4077 |
| TP53I11 | NM_001258321 | TGTGCTTAGTCCCATTTTAC | 4078 |
| TP53I11 | NM_006034 | ACTTGCCAGGAAAGTCATCC | 4079 |
| TP53I11 | NM_006034 | CAAGGCTATTTAAGATGGTG | 4080 |
| TP53RK | NM_033550 | CGAGAGTCACCGAAGATTTC | 4081 |
| TP53TG3C | NM_001205259 | CAAGGGGATTAAATCAGGAG | 4082 |
| TP53TG3C | NM_001205259 | GCTTCGTTTACCAAGCTTGC | 4083 |
| TPD52L1 | NM_001003395 | GGCAGCAGGCATTATACCAA | 4084 |
| TPD52L1 | NM_003287 | CTCGCTTTATTGCGGGGGTC | 4085 |
| TPM1 | NM_001018008 | GGGGCGCGCGCCGTGGATCC | 4086 |
| TPPP3 | NM_016140 | GAGACCAGCGCTCTGCAGTT | 4087 |
| TPR | NM_003292 | GCGGTGCAGCATTGGGCTCC | 4088 |
| TPRA1 | NM_001136053 | TGTCTCTTTAAGAGGTCAGC | 4089 |
| TPSAB1 | NM_003294 | TGGCAGCTCCACCTGTCAGC | 4090 |
| TPSG1 | NM_012467 | CACCTCCATTTATCCCTGTG | 4091 |
| TPTE | NM_199259 | CGCCATCCGGCTTAACGTGG | 4092 |
| TRA2A | NM_001282759 | GGCGGCCTGCGCTCTCAACC | 4093 |
| TRA2B | NM_004593 | AATCCCTTCTAGAACTTTCC | 4094 |
| TRABD2A | NM_001080824 | GGGTGCCTCTTGATTGAAAG | 4095 |
| TRAF3IP2 | NM_001164281 | CGAGACCATCCTGGCTAACA | 4096 |
| TRAF3IP2 | NM_001164283 | AGCCGTGCAAAGACTTGGAA | 4097 |
| TRAF3IP2 | NM_001164283 | CCAACAAGGGAGGCTTTGTT | 4098 |
| TRAK2 | NM_015049 | GGTGCAGAGTTCCAAGCCCA | 4099 |
| TRAM2 | NM_012288 | AGGCGTACGGGGGCGGCGAG | 4100 |
| TRANK1 | NM_014831 | AGCACTCGTTTATTCAAAGG | 4101 |
| TRAPPC10 | NM_003274 | GGGACCGGGAGGTGGGAAGT | 4102 |
| TRAPPC13 | NM_001093756 | GGACAAAACGATTAAAGTTT | 4103 |
| TRAPPC9 | NM_031466 | GGCGCCAAGCTTGCTAAGTG | 4104 |
| TRDN | NM_006073 | TCTAAGATAATTACAGATCC | 4105 |
| TREH | NM_007180 | CAAAGTAGAAGCAAGGGAGG | 4106 |
| TREH | NM_007180 | CTGAGACTGTGAAATAGAAG | 4107 |
| TREM1 | NM_001242590 | CTTAACTGAGAAGTGAGTCT | 4108 |
| TREML1 | NM_001271808 | GCAGGCTTCTAGCTTTCTTC | 4109 |
| TREX2 | NM_080701 | AAAGCAGATAGCATCTCCCG | 4110 |
| TRIB2 | NM_021643 | CTTTGTTTACCTCCCCGGCC | 4111 |
| TRIB3 | NM_021158 | ACAGGCGCCCGCACCACGCC | 4112 |
| TRIM2 | NM_001130067 | TTCCCCGCCTGTCATCTTTG | 4113 |
| TRIM2 | NM_015271 | GAGCCAATGATCAGCCTCTT | 4114 |
| TRIM21 | NM_003141 | TTCAGAGGCTCTGCATGCCC | 4115 |
| TRIM22 | NM_001199573 | AGACTGCATTTCAAGAAGCT | 4116 |
| TRIM26 | NM_001242783 | ACTGAAATCAGGCGGGACCG | 4117 |
| TRIM3 | NM_006458 | ACCAAGGAGGCAGCGTCCGC | 4118 |
| TRIM34 | NM_001003827 | CTAGAGTAGTGGTGTGATCT | 4119 |
| TRIM34 | NM_001003827 | TCACTGCAACCTCTGTCTCC | 4120 |
| TRIM42 | NM_152616 | CAAATGACAACTAAACTTCC | 4121 |
| TRIM46 | NM_001256601 | CCCTCTCTTCGCAGCCATCC | 4122 |
| TRIM48 | NM_024114 | ATTTAGATCACACCTTTGCA | 4123 |
| TRIM49D1 | NM_001206627 | ACAGGCACTAGGAGTAGAAG | 4124 |
| TRIM50 | NM_001281451 | GGTGCTGGCCTTGGCCACTG | 4125 |
| TRIM54 | NM_187841 | ACTCCCTTGAGCAAGGGCAG | 4126 |
| TRIM59 | NM_173084 | GGCCAATGGGAACTATTGCT | 4127 |
| TRIM63 | NM_032588 | GAGGGCCAGTCTTTCAGGCC | 4128 |
| TRIM64 | NM_001136486 | TACTATGTCTCAGTTTGTGC | 4129 |
| TRIM66 | NM_014818 | CACACATTTACGATGCACAA | 4130 |
| TRIM73 | NM_198924 | GCACGGTGAGTTGCCAGGTG | 4131 |
| TRIML1 | NM_178556 | TGGTGAGGAGCCCAGTATAC | 4132 |
| TRMT2B | NM_001167972 | GAGAAAACTATTCCTTGAGT | 4133 |
| TRMT5 | NM_020810 | GTCGTCGGTCGCGCCAGAGG | 4134 |
| TRMT61A | NM_152307 | AAACAGAGCAGCTCACATGA | 4135 |
| TRMT61A | NM_152307 | TCGCCCAGGAAACGTCCTCT | 4136 |
| TRNAU1AP | NM_017846 | GGGTTTTTCCTGCAACCCAC | 4137 |
| TRNT1 | NM_182916 | ACCGGCTGAGGTTCGCCTCA | 4138 |
| TRPC7 | NM_001167576 | TACGTCGGGGAGAGGGGGTG | 4139 |
| TRPM6 | NM_001177311 | CCGGAGGGAGAGGAGTTCGG | 4140 |
| TRPM6 | NM_001177311 | GGCAGCTCTGATTCCGCTCC | 4141 |
| TRPM8 | NM_024080 | CTGCTATGCTTGGAGGCTTT | 4142 |
| TRPT1 | NM_001160393 | GAGCGCTGGGTGGGAGTATA | 4143 |
| TRPV1 | NM_018727 | GCTGCGGCTCTGATTCCCAG | 4144 |
| TRPV1 | NM_080704 | AAGCCTTCTTGTGATTGGTA | 4145 |
| TRPV1 | NM_080704 | GCAGAAACATCCATTTGAGT | 4146 |
| TRPV3 | NM_001258205 | ATGATAACATCTACTTTCCA | 4147 |
| TRUB1 | NM_139169 | TTAAATGTTGACTTTTCCTG | 4148 |
| TSC1 | NM_000368 | TCCACTCATAACTGACGATG | 4149 |
| TSC2 | NM_000548 | GCGGTCATGCCGGACTCCTG | 4150 |
| TSC22D1 | NM_001243798 | GTTTCTACTTAAAGGGGCAG | 4151 |
| TSC22D2 | NM_014779 | TCTCTGACTGAGGGAAGGAG | 4152 |
| TSEN15 | NM_052965 | CGCGCAGGTTCTAGCTACCT | 4153 |
| TSEN2 | NM_001145395 | TGCGCACTCGGCTGGCTTTG | 4154 |
| TSFM | NM_001172697 | TACCCCCCACCTCCCACCCC | 4155 |
| TSGA10 | NM_025244 | ACCCTTACTTAGCACTCCTG | 4156 |
| TSGA10 | NM_182911 | AGCCACCGCCGCGAAGCAGC | 4157 |
| TSLP | NM_033035 | AAAAGGAGTAGCTAAATCTA | 4158 |
| TSPAN10 | NM_001290212 | CGGAGCCGGGCGGGCGAAGC | 4159 |
| TSPAN19 | NM_001100917 | GAATCCCAGTCTTAAGACCC | 4160 |
| TSPO | NM_001256530 | AGTCTGGGCCTCCGCGGCCG | 4161 |
| TSPY4 | NM_001164471 | GCTTGGGCAGGGAAGGCGGG | 4162 |
| TSPYL1 | NM_003309 | AAACATTTGTTTTCAGACAC | 4163 |
| TSSK1B | NM_032028 | TCGTGTCTTGCTGGGACCTG | 4164 |
| TSSK3 | NM_052841 | GGAGGGCAGCATTGTGACCC | 4165 |
| TSSK6 | NM_032037 | CCAGGGCTCCACGTAGTCAC | 4166 |
| TST | NM_001270483 | AGAGCGGCAGAGCGAGTTGC | 4167 |
| TSTD2 | NM_139246 | CGCCTGGCCTCTCGGTTCCG | 4168 |
| TTBK2 | NM_173500 | GCGTTCCGAACTCGCAGCGT | 4169 |
| TTC21A | NM_145755 | CCAGTCCCGCTGCGCCTACC | 4170 |
| TTC36 | NM_001080441 | AAATGCTACAGCCATGGACA | 4171 |
| TTC39B | NM_001168342 | CATGATTTTTCACCTAATCC | 4172 |
| TTC7B | NM_001010854 | TCCGGCCCCGGTCAGTGCTG | 4173 |
| TTC9B | NM_152479 | GAGCATGGGGGAAGTCTCGA | 4174 |
| TTF1 | NM_007344 | GCTCCTGAAACGAAGAAAGT | 4175 |
| TTI2 | NM_025115 | TTTTGTTTCTACCTTAGCAA | 4176 |
| TTLL12 | NM_015140 | CTGGGAGGAGGACGGGGCGG | 4177 |
| TTYH2 | NM_052869 | GGGGGACATCCCTAAGGAAC | 4178 |
| TUBA3D | NM_080386 | CGCAGTAGCTGTTCCAACCC | 4179 |
| TUBB2A | NM_001069 | GGGACTGCGGCACCGCGAGG | 4180 |
| TULP3 | NM_003324 | GGGAGTTAAACGCGCCTGCG | 4181 |
| TULP4 | NM_020245 | CTGAAAAGTAACTCCTACTG | 4182 |
| TUSC5 | NM_172367 | GAGGCAAAATCCTGCCAGGG | 4183 |
| TVP23C | NM_145301 | AAGCTTCATGGTCTGTTTTA | 4184 |
| TXLNA | NM_175852 | AGGCGGGCGCCCCGGCAGGG | 4185 |
| TXNDC17 | NM_032731 | AGGATCCAGGTGTTGCAAGG | 4186 |
| TXNIP | NM_006472 | CAACAACCATTTTCCCAGCC | 4187 |
| TXNL1 | NM_004786 | GCAGACTGAGACTCAAAAGT | 4188 |
| TXNL4A | NM_006701 | GCGCCGCGCGAACGTGTAGT | 4189 |
| TXNRD1 | NM_001093771 | TGGAAAATGCAGAAATGGAA | 4190 |
| TXNRD3NB | NM_001039783 | TGTTTCTGTATTAAAGGATC | 4191 |
| TYMS | NM_001071 | TGTGGCACAGAACGGAGCCC | 4192 |
| TYR | NM_000372 | CATAGGCCTATCCCACTGGT | 4193 |
| TYSND1 | NM_173555 | GTCACGAGGAATCAGAGGAG | 4194 |
| TYW3 | NM_138467 | TGGGTGGAGCCTGCAAAAGT | 4195 |
| U2SURP | NM_001080415 | GTCCGGGAATTCAGAGAATC | 4196 |
| UACA | NM_001008224 | AGTTCTACTTTAGATTCCAT | 4197 |
| UACA | NM_001008224 | CATTCAGCTGTCAAGTCCTA | 4198 |
| UAP1 | NM_003115 | GCTCCAGAACTATTCCCATT | 4199 |
| UBA52 | NM_001033930 | CGCCCACCCGCTTCCGGTTG | 4200 |
| UBAC2 | NM_001144072 | GGGCCGACTGTCGTGGTCCC | 4201 |
| UBB | NM_001281718 | CCCCAAGGTCGTTACGGCTG | 4202 |
| UBE2C | NM_181801 | GAGAACACACCAGGAGCTCG | 4203 |
| UBE2D1 | NM_003338 | AGCTCTCACCTTAAGCTGCC | 4204 |
| UBE2I | NM_194260 | GACCGACGGGAGGAGAAGTG | 4205 |
| UBE2L3 | NM_003347 | CAGGCGTGAGCCCCCGCGCC | 4206 |
| UBE2Q2L | NM_001243531 | GTGTGTGTGTGTGTCTCCCA | 4207 |
| UBE2V2 | NM_003350 | AGCGAGGCCCCGCGACCCCT | 4208 |
| UBE2Z | NM_023079 | CGTGTGGGTCCTGCGCTGTG | 4209 |
| UBIAD1 | NM_013319 | GGCGGGCAGGGCCGAGTCAG | 4210 |
| UBP1 | NM_014517 | CGGGGAGTGGCCCTAAGCGC | 4211 |
| UBR5 | NM_015902 | GTTGAGCAGCCCAATCGAGG | 4212 |
| UBR7 | NM_175748 | GGGTGACGGCGACCCTTTCC | 4213 |
| UCHL5 | NM_015984 | ATCCGGGATCCTCGCCCCTC | 4214 |
| UCMA | NM_145314 | TGCTTCTGGAGACATTTGCC | 4215 |
| UEVLD | NM_001261385 | AGCATGCAAGTTTTGTAGTC | 4216 |
| UGT1A7 | NM_019077 | TAAGTACACGCCTTCTTTTG | 4217 |
| UGT2B11 | NM_001073 | TATAATAGTGTCAAGAACAG | 4218 |
| UGT2B7 | NM_001074 | AGATCCTTGATATTAGCTGA | 4219 |
| UHMK1 | NM_001184763 | TTCGAGTTTTCCCACCTTTC | 4220 |
| UHRF1 | NM_001290050 | ATCACTCAGCTCAGAGTTCC | 4221 |
| UHRF1BP1L | NM_015054 | GTCGCGAGGGCTAAGAACCC | 4222 |
| UIMC1 | NM_001199298 | AGACCGCGCAAGGTGCGAGC | 4223 |
| UIMC1 | NM_001199298 | GTATAGAACGGCCACTTTTG | 4224 |
| ULBP1 | NM_025218 | AGGGGAGAGTTGCGTCAGCC | 4225 |
| ULK1 | NM_003565 | GGGCGTGACGAACAGACGGG | 4226 |
| UNC13B | NM_006377 | GCAAGAAAGAAAGGAGGAAG | 4227 |
| UNC45A | NM_018671 | TGAGCTTTCTCCGGACTCCC | 4228 |
| UNC45A | NM_001039675 | GGCCATGGGGAGGGATTGCC | 4229 |
| UNC5B | NM_170744 | GCGCAGCGTTTTGAAAAACC | 4230 |
| UNC5CL | NM_173561 | AATGCCAGGCCACTCCTGCC | 4231 |
| UNC93A | NM_001143947 | AAACATATCACTTTACCATC | 4232 |
| UPF2 | NM_015542 | AGTCCTGATCGTCTTCCCTG | 4233 |
| UPK3A | NM_006953 | GGCCGCGGATTGGCCAGCCC | 4234 |
| UQCR10 | NM_013387 | CCACAGAGGTATTCCTATCC | 4235 |
| UQCRHL | NM_001089591 | ATAAAGAGAAGTTTCTGGCC | 4236 |
| UQCRQ | NM_014402 | AGGCTCCACCCCACCGGCCC | 4237 |
| URB2 | NM_014777 | TTGCGCGTTGGAGGCCCGAG | 4238 |
| UROD | NM_000374 | TGGGACTTGCGCCAAGCCTC | 4239 |
| USH1G | NM_001282489 | GCAGGGTGTTTAGGACCCAG | 4240 |
| USP10 | NM_001272075 | TGAGCCCCGCGACCCTCGGG | 4241 |
| USP16 | NM_006447 | TGCGCCGGATGTTCGGGTTT | 4242 |
| USP17L2 | NM_201402 | GGGGTGTTCGCGGTTGGTGG | 4243 |
| USP17L25 | NM_001242326 | ATTGAGTGCTGATATTTGAT | 4244 |
| USP17L25 | NM_001242326 | TCGCGCACCTGATGAGTGGG | 4245 |
| USP17L3 | NM_001256871 | GAGTTCTATAAGGGATGATG | 4246 |
| USP39 | NM_001256727 | TTCATGTCCAGCCGCCCCCC | 4247 |
| USP42 | NM_032172 | GGGTCGTCGCCCAAGAGCCG | 4248 |
| USP46 | NM_001286767 | CGGGGCCCGGGAACCCAGCC | 4249 |
| USP9Y | NM_004654 | TTCTGGGTTGTGTTTCATAC | 4250 |
| UTP11 | NM_016037 | AAGGCGAGATCTGGGTAGCG | 4251 |
| UTP14A | NM_006649 | CGCGCGGGTGTCTGTCCTCC | 4252 |
| UTP15 | NM_001284431 | GTGTAGTACTCCGGCAGGAT | 4253 |
| UTP20 | NM_014503 | GGTGTTCTTTTCACTCCCTT | 4254 |
| UTRN | NM_007124 | CATAACACCATTGCCTGGCT | 4255 |
| UTS2B | NM_198152 | TGCAAAGCCCTTGGAACTTA | 4256 |
| UVSSA | NM_020894 | CCCAAGACCTCTACCGCCAT | 4257 |
| UXS1 | NM_001253875 | AGTTGCCGCCTTTCTTGCCT | 4258 |
| UXT | NM_004182 | GCAGGGCTTCACGGAATCCG | 4259 |
| VAMP2 | NM_014232 | AGGGAGCTGCCGGGGCATGG | 4260 |
| VAMP8 | NM_003761 | CTGACAAGTTAGAAGACCTT | 4261 |
| VAPA | NM_003574 | GGAACGGGTGTGGAAGGAGG | 4262 |
| VCAN | NM_001126336 | CGCCAAGAGGTGGGAGTGCC | 4263 |
| VCL | NM_014000 | GGGTTTGGCGGCGCGGTGGC | 4264 |
| VCX3B | NM_001001888 | CAGGCTGGGTTCCTCAGAGA | 4265 |
| VGLL4 | NM_001128219 | GGGGAGAGACTCTAGAGACG | 4266 |
| VGLL4 | NM_001128221 | CAATGTCACTGCTTGGAATC | 4267 |
| VHL | NM_198156 | CACTGCAGCCTTGACCTCCC | 4268 |
| VILL | NM_015873 | ATGAGTGGGTTGGGCAGATT | 4269 |
| VIP | NM_194435 | CGTCACAGTATGACGGCCAT | 4270 |
| VMO1 | NM_182566 | CTCTGGGAGCCTCTGCCTCC | 4271 |
| VMP1 | NM_030938 | GGTACTGTAGGTAGGTTGGT | 4272 |
| VN1R4 | NM_173857 | AAGGGCAGAGCAATGGGAGG | 4273 |
| VN1R4 | NM_173857 | GGTGGAGAATGCTGGGTTGC | 4274 |
| VPS13D | NM_018156 | CGAGCGCCGAGTTATCGAGG | 4275 |
| VPS29 | NM_016226 | GCCTTCCGAGCCTGCTTTTT | 4276 |
| VPS37D | NM_001077621 | CCCGATCTCCCCGCCCCTCC | 4277 |
| VPS45 | NM_007259 | GAACAAAGGGAACGCCTTTT | 4278 |
| VPS4B | NM_004869 | TGCGCTCTCCTAGGTCTGCC | 4279 |
| VPS50 | NM_001257998 | TGTAAGACCGGCGATCGCAG | 4280 |
| VPS8 | NM_015303 | AATGGGTGATTCACATCTTG | 4281 |
| VPS8 | NM_015303 | ATACGCCGTCTTCCCCCCTA | 4282 |
| VRTN | NM_018228 | ACTTTTCTCTGGGCAGTTTG | 4283 |
| VSIG1 | NM_001170553 | TCTTACTAAAACGTTGTACT | 4284 |
| VSIG4 | NM_001100431 | TTGGAGCCAATGGGGCTTTC | 4285 |
| VTA1 | NM_001286372 | TTTGTTTGGTTTGTTGTTTG | 4286 |
| VTCN1 | NM_001253849 | CATACTTTGAACATCGAGTT | 4287 |
| VTI1A | NM_145206 | AGAGGTGCTCGGCTTGTAGC | 4288 |
| VTI1B | NM_006370 | ACGCAAACATACATCAAATC | 4289 |
| VWA1 | NM_199121 | ACCTCCCTGCTCGGCTCCCG | 4290 |
| VWA5A | NM_014622 | CAATCAGAGAACAGGCAAAG | 4291 |
| VWC2L | NM_001080500 | TTGCTTTGAATTCTGAAGAC | 4292 |
| WARS | NM_173701 | CGGTTCTCCCGGAGGCAGAC | 4293 |
| WBP2 | NM_012478 | ATGCATCCTTCCTCCAGCAT | 4294 |
| WBSCR27 | NM_152559 | GCTCTACCAAGGCTGGAGGA | 4295 |
| WDFY2 | NM_052950 | GCCTAACCCTTGGGTGTGTA | 4296 |
| WDFY2 | NM_052950 | GGAAAGCGCATGCGTCCTAG | 4297 |
| WDFY4 | NM_020945 | CCCAGGGTTCCCTTCATAGC | 4298 |
| WDR1 | NM_017491 | CCTTTCTGTTGCTAGCTTGT | 4299 |
| WDR11 | NM_018117 | GCCCTAAATTCACTTATCAA | 4300 |
| WDR13 | NM_017883 | TTGCACTTTTTGTGTATACA | 4301 |
| WDR4 | NM_001260475 | ATGAACATTAGGCAAGTACT | 4302 |
| WDR4 | NM_001260475 | GTTTGGCAGTTCACTCACCA | 4303 |
| WDR59 | NM_030581 | CCTCGCTCACTTCCGTCACT | 4304 |
| WDR60 | NM_018051 | AGCGGTCGTTGGTCTCCCCA | 4305 |
| WDR62 | NM_173636 | TAATCAGGCATCCAGTACAC | 4306 |
| WDR73 | NM_032856 | GGCCCGGCATGGGTGGGTTA | 4307 |
| WDR83OS | NM_016145 | GGCTGCAAGGAAGGAGTCCT | 4308 |
| WDSUB1 | NM_152528 | CCTCTGCTCTGGGTCTCCGC | 4309 |
| WDTC1 | NM_015023 | GGGAAAGCTGGGCTAAGCCC | 4310 |
| WEE1 | NM_003390 | AAGGACCAGCTACGCGATTT | 4311 |
| WEE1 | NM_003390 | GAACCCGCTGGCTCCACCCC | 4312 |
| WFDC11 | NM_147197 | TTTTCTGTTGTCTCTCTGCC | 4313 |
| WFDC9 | NM_147198 | TGCAGCATCTCCTGATGCTA | 4314 |
| WIPI1 | NM_017983 | CCCCTGCCTCCGGCCACCAT | 4315 |
| WIZ | NM_021241 | GTGGGGTGGGGGGGGCGCCC | 4316 |
| WLS | NM_024911 | CATCAACAGCAACCCCTAAA | 4317 |
| WNT10B | NM_003394 | AGATCAGGTGAGAGGAACTC | 4318 |
| WNT2B | NM_024494 | ACTGTAGGTTGGGGACAGGA | 4319 |
| WNT5B | NM_030775 | CACGGCTAGAGGGACTCTAA | 4320 |
| WRAP53 | NM_001143990 | GGAAAAAGATGACGTAAGTA | 4321 |
| WRAP53 | NM_001143990 | TGTAAATGCCACCTCGATTT | 4322 |
| WWOX | NM_016373 | ATGGGCGCCGCTTTTTAGTC | 4323 |
| WWOX | NM_016373 | GGTGGCGCCTGACCAAAAAG | 4324 |
| WWP1 | NM_007013 | GACCCCACACCTCCCTTCCT | 4325 |
| WWP1 | NM_007013 | GCGCCGCGTGGCCGCGTCGC | 4326 |
| WWP2 | NM_007014 | ATCGTCTCTGTAGTTGAAAG | 4327 |
| WWTR1 | NM_001168278 | TTTGTTGGCAAAACCCTTTT | 4328 |
| XAGE1B | NM_001097604 | ACTCACTCCATGACCGGGCG | 4329 |
| XAGE1B | NM_001097605 | GGATTCCAAAGTCGTTAATG | 4330 |
| XIAP | NM_001204401 | AGCTGGGGGCGGAGACTACG | 4331 |
| XK | NM_021083 | CGGAGCGCGTGGGCGTGTCC | 4332 |
| XPNPEP1 | NM_001167604 | TCCCCGCTCGCTGCAGGGAG | 4333 |
| XPNPEP2 | NM_003399 | GCCCCAGCCATTCCTTAATT | 4334 |
| XPO4 | NM_022459 | CTAGTCCCCTCCCAGCCACC | 4335 |
| YAF2 | NM_001190977 | CTGGCCGCGTTTGAAGTCTC | 4336 |
| YAP1 | NM_001195045 | ACTTCTATGCTGAATCAAGT | 4337 |
| YBX3 | NM_001145426 | CGGGTCGCGTTGCAGAACCA | 4338 |
| YDJC | NM_001017964 | CCTTTGTTCTCGCCACCTAG | 4339 |
| YEATS2 | NM_018023 | CGGCCCGCGAGGGCACTTCC | 4340 |
| YIPF1 | NM_018982 | GGTCGCTGAGTGTGACTACT | 4341 |
| YIPF6 | NM_173834 | AGAGGCAGGCTCTTTCCTAG | 4342 |
| YPEL3 | NM_001145524 | CGTCACACGGCGGCCGGCGC | 4343 |
| YY1AP1 | NM_018253 | TGGGACTCGGCCGGCCACCC | 4344 |
| YY2 | NM_206923 | TCACTGCAACCTCCGCCTCC | 4345 |
| ZAR1 | NM_175619 | GTAGGGAGAAGGACGAAGAG | 4346 |
| ZBED1 | NM_001171136 | GCTGGGGTCGGTTGTCCGCT | 4347 |
| ZBED1 | NM_001171136 | TGCGGGATCCCAGAGGGCCC | 4348 |
| ZBED2 | NM_024508 | TCTAGGGAAGCATTGTTTCC | 4349 |
| ZBTB1 | NM_014950 | AGCAGCCTCGCATCCTGCCC | 4350 |
| ZBTB21 | NM_001098403 | TCCATGAGGGGAGCCTGCGG | 4351 |
| ZBTB33 | NM_001184742 | CCCCTTGCGGAAAGAACCGA | 4352 |
| ZBTB38 | NM_001080412 | AGAAGCTAGTCTCCAAAGCT | 4353 |
| ZBTB43 | NM_001135776 | GGCGCCTGCGCAGTACACTC | 4354 |
| ZBTB45 | NM_032792 | CGCACGCTGAGAACGCGAGG | 4355 |
| ZBTB46 | NM_025224 | TGGGCAGCTCGCGGCAGCAG | 4356 |
| ZC2HC1C | NM_024643 | GTCCGGCCAACTCTGCAGCT | 4357 |
| ZC3H10 | NM_032786 | AGTGACACGCAAAGCGTGCT | 4358 |
| ZC3H12B | NM_001010888 | GGTATGTGTGTTTATTTGTA | 4359 |
| ZC3H12C | NM_033390 | AGTTGTGCAACCCAGGGAGG | 4360 |
| ZC3H12D | NM_207360 | GTGGTTGCTGAACTTTGATT | 4361 |
| ZC3H6 | NM_198581 | TCTCTGTGCAGCGGCGGAGG | 4362 |
| ZC3H8 | NM_032494 | AATTCTACTATCTGAGGTAA | 4363 |
| ZCCHC7 | NM_032226 | ACGAAGGAGATGCTATTTAC | 4364 |
| ZCCHC8 | NM_017612 | CACCTGTAATACCAACTACT | 4365 |
| ZCWPW2 | NM_001040432 | ATCTTCACAGAGTAAAAGTG | 4366 |
| ZDHHC12 | NM_032799 | GGCCGCAGATGCCATCCAAT | 4367 |
| ZDHHC12 | NM_032799 | TGTTGGCTTGAGGGTCCATT | 4368 |
| ZDHHC20 | NM_001286638 | ACAGGCTGGGCGGACGCGGG | 4369 |
| ZDHHC3 | NM_016598 | CGTCCAGGTAGCTACAGCAG | 4370 |
| ZDHHC8 | NM_013373 | TCGGAGGGGGCAGGACCCCG | 4371 |
| ZDHHC9 | NM_001008222 | TGGCTGCCGACGTGATTCCC | 4372 |
| ZEB1 | NM_001174094 | AAGGAATTACACGTACATTT | 4373 |
| ZEB1 | NM_001174096 | GCACTGCTGAATTTGAATTG | 4374 |
| ZFAND4 | NM_001282906 | CGAATGCCAAGAAGGCCCCA | 4375 |
| ZFAND5 | NM_006007 | GGCCTGGCAGTCGGCCCCTA | 4376 |
| ZFAND6 | NM_001242919 | GGCCACAGACTAGGTGAGTA | 4377 |
| ZFC3H1 | NM_144982 | AGTTGGGTGCATGCAGAAGT | 4378 |
| ZFHX2 | NM_033400 | ACTCCAGCCAGTGAATGAGG | 4379 |
| ZFP3 | NM_153018 | GGGTGCACTTTGCTGTTCCA | 4380 |
| ZFP30 | NM_014898 | CGGGTCTCGGCGGGGATAGT | 4381 |
| ZFP30 | NM_014898 | GGCAAGTCCCGCAGCTGCTC | 4382 |
| ZFR | NM_016107 | GGGGAAGCCCGCGGGGGAAG | 4383 |
| ZFR2 | NM_015174 | TGCGTAGGAGGCGGGGCCTC | 4384 |
| ZFX | NM_001178085 | AGGCCCCCTCCTCCGCCCGG | 4385 |
| ZFX | NM_001178086 | CACTGGGCTCCCCGGTCGCG | 4386 |
| ZFX | NM_003410 | GACAGGCCCCCTCCTCCGCC | 4387 |
| ZFYVE21 | NM_024071 | GCAGGGGCGGTGCCCTTACA | 4388 |
| ZKSCAN3 | NM_024493 | CAGCTATAACTAAGGGAGAA | 4389 |
| ZMIZ2 | NM_031449 | GGGGCTCTGCTGCTCTGGCC | 4390 |
| ZMYM2 | NM_001190965 | TCCTCACCAGCGCTAAAGCC | 4391 |
| ZMYM5 | NM_001142684 | TGGGCGTGCCCAAGGCGCCC | 4392 |
| ZMYND11 | NM_001202465 | AGCAGAGGACTCTGACTGAC | 4393 |
| ZMYND11 | NM_001202468 | AATGAGATGTGAAAGGTTGA | 4394 |
| ZNF132 | NM_003433 | CCATTGGCAGCCGAGGAGAC | 4395 |
| ZNF136 | NM_003437 | CACATCTGTCAAGATGCAGG | 4396 |
| ZNF136 | NM_003437 | TGAAGCATAGATGAGTGAAG | 4397 |
| ZNF140 | NM_003440 | AGACAAAGAACACGAGCTTC | 4398 |
| ZNF142 | NM_001105537 | GGGCTTCTCTGTGGGTGTGG | 4399 |
| ZNF160 | NM_033288 | GGGCTGAAGCAGGGGCCGCC | 4400 |
| ZNF169 | NM_194320 | ACAATTTCTCCTGGATGCTG | 4401 |
| ZNF177 | NM_003451 | CACAAGCCAATTAACTTGCT | 4402 |
| ZNF177 | NM_003451 | GCAGGTGCTCCTGCTCCCTT | 4403 |
| ZNF182 | NM_006962 | ATTGGCGGACGGGGTCTCAA | 4404 |
| ZNF189 | NM_001278232 | CTACATTTCCCAGCGTGCAA | 4405 |
| ZNF2 | NM_021088 | GAACGGCCCTGGCTGCAAGC | 4406 |
| ZNF205 | NM_001278158 | GCCTGGGTTGCACCTGCTCT | 4407 |
| ZNF213 | NM_004220 | TCTTCCTGTTCATTGGCCAT | 4408 |
| ZNF219 | NM_001102454 | CTGGAATGGAGAAAAGATCT | 4409 |
| ZNF226 | NM_001146220 | TGTTTCCCCTGCGGAATCCT | 4410 |
| ZNF226 | NM_001032372 | TAGGTAGTTGTAGGCACTTC | 4411 |
| ZNF234 | NM_006630 | GGATTACACTCAGAATGCTG | 4412 |
| ZNF236 | NM_007345 | TATAACCCACCGACTCCCAT | 4413 |
| ZNF254 | NM_203282 | AGAAGATGTGATCACACCCT | 4414 |
| ZNF260 | NM_001166037 | GATAGAGTAAACTAAGACTA | 4415 |
| ZNF268 | NM_001165886 | TAGTCCCTGCTTTACTGAAA | 4416 |
| ZNF284 | NM_001037813 | CGTTCTATAGTATCACCTTC | 4417 |
| ZNF296 | NM_145288 | ACGGCGGCCTAACTCAATCT | 4418 |
| ZNF3 | NM_032924 | CACTCGGGGATCTTTCGCTG | 4419 |
| ZNF30 | NM_194325 | GACCTGGTGTGTTAATGCCC | 4420 |
| ZNF316 | NM_001278559 | CGGGGCGAGGACGGGGCATG | 4421 |
| ZNF32 | NM_006973 | TCTCTGGCGCGCCCTGCGCT | 4422 |
| ZNF324B | NM_207395 | AGCTGCGCTACTCCATTTCC | 4423 |
| ZNF329 | NM_024620 | AGCATCGGGTTAAAAATCAG | 4424 |
| ZNF330 | NM_014487 | AATGCCCCATTCCTAAGCAG | 4425 |
| ZNF333 | NM_032433 | AGAGCCTAACCTCATCCCCC | 4426 |
| ZNF345 | NM_001242475 | GTGTGTTGTGTTTAGGTTTG | 4427 |
| ZNF354C | NM_014594 | CCAGGCTTGGCTAGGATTGC | 4428 |
| ZNF383 | NM_152604 | ATCAACATCCTCCACCAGAG | 4429 |
| ZNF395 | NM_018660 | CAGCGAGAGAAACTTTGGCT | 4430 |
| ZNF423 | NM_001271620 | CAAGGTGGCGCCACTCACCC | 4431 |
| ZNF428 | NM_182498 | ATCACTCCTTCCAGTGCGGG | 4432 |
| ZNF429 | NM_001001415 | AGCCTAGCTGCAGCCTTTTC | 4433 |
| ZNF444 | NM_018337 | ACGACGCTTTCGCGTATCTT | 4434 |
| ZNF473 | NM_015428 | GACTACAAACTGATGCCGCC | 4435 |
| ZNF474 | NM_207317 | TTAAATTTATCTGTCCCTGT | 4436 |
| ZNF479 | NM_033273 | ACTTTTGACCCTGCCCAAAG | 4437 |
| ZNF48 | NM_152652 | GGCGGTAGCTCTGTGGCCGG | 4438 |
| ZNF500 | NM_021646 | GGTAACGTAGTCCAGCACCT | 4439 |
| ZNF503 | NM_032772 | CCGAGGTGATTGGAGGGTCA | 4440 |
| ZNF510 | NM_014930 | AACAAAAAAACACTGACAGC | 4441 |
| ZNF513 | NM_001201459 | GGGGTCGGGCGGCCGCAGGC | 4442 |
| ZNF518A | NM_014803 | TTCGTTGACGTGGGCTACAA | 4443 |
| ZNF526 | NM_133444 | GGTCGCGTGCCCTGCGCTGC | 4444 |
| ZNF534 | NM_001291368 | CTCACTTGTTGATTTTCCTG | 4445 |
| ZNF536 | NM_014717 | TTTCTGAGTCCTGCCTCTGA | 4446 |
| ZNF556 | NM_024967 | CTTCTCTGCTCATCTCTGAT | 4447 |
| ZNF564 | NM_144976 | AATATCCTCCCCGGCACAGA | 4448 |
| ZNF569 | NM_152484 | AGCTCCAGCCGACTGTAAGA | 4449 |
| ZNF583 | NM_001159861 | AGTAACTACCCGCAACTGAG | 4450 |
| ZNF597 | NM_152457 | CAATTGGTCAACACAAAAGA | 4451 |
| ZNF598 | NM_178167 | GCGGTCGGCTCATGGTAGAG | 4452 |
| ZNF611 | NM_001161500 | AAACAGAGACGCTGGGAGCG | 4453 |
| ZNF611 | NM_001161500 | GGCAGAGGGCAGGGCCGGGG | 4454 |
| ZNF613 | NM_024840 | ATCTTTGAATCCTGCACGTA | 4455 |
| ZNF614 | NM_025040 | GTGCCCAGCCAAGGCCAACA | 4456 |
| ZNF616 | NM_178523 | TCGGAAAGAGGGGCCTGACT | 4457 |
| ZNF630 | NM_001037735 | TAGACCCGCAGCACTCAGCC | 4458 |
| ZNF641 | NM_001172682 | AGGAATTCCAGACTGTTGTC | 4459 |
| ZNF646 | NM_014699 | ACGGCTGACTCCGCCCACGT | 4460 |
| ZNF654 | NM_018293 | TGCACTCTCAATATTTTTTC | 4461 |
| ZNF669 | NM_024804 | CGCACCGCCTACAAACCGCT | 4462 |
| ZNF682 | NM_033196 | ATCTGAGAATGTGTTGAATA | 4463 |
| ZNF682 | NM_001077349 | GCTAAGACTCCACGACATCC | 4464 |
| ZNF687 | NM_020832 | GGGCTGAGCGACGGGGGCAA | 4465 |
| ZNF689 | NM_138447 | AGCTCTTGGCTTCGTTCAAA | 4466 |
| ZNF691 | NM_015911 | CTGAGTCTACGCGCTTCCTT | 4467 |
| ZNF692 | NM_001136036 | GCTGCTGTAGCCCGGAACTG | 4468 |
| ZNF697 | NM_001080470 | GGACAACGGTCCACTTTACG | 4469 |
| ZNF699 | NM_198535 | ATTGATGGGCTGCAACATCC | 4470 |
| ZNF7 | NM_003416 | GGCGGGGTACAGTCAGAGGC | 4471 |
| ZNF70 | NM_021916 | GGTGGGACCACCGAGACGCC | 4472 |
| ZNF700 | NM_144566 | TCTTCTATCAATAGCAAGTT | 4473 |
| ZNF703 | NM_025069 | CGGGCTGAGGCCGGCTCCAT | 4474 |
| ZNF704 | NM_001033723 | GCGTTCAAAGAGTGTGAGAT | 4475 |
| ZNF705A | NM_001278713 | AATTTTGACCACAGGAAAAG | 4476 |
| ZNF708 | NM_021269 | GCCTATGCTGCAGCCTTTTC | 4477 |
| ZNF718 | NM_001289931 | AAGCTTGAAGACTGCAATCC | 4478 |
| ZNF735 | NM_001159524 | GACGCCTCCGTAATTTTACC | 4479 |
| ZNF75D | NM_001185063 | ATTAACTCTTTCTTGCATCC | 4480 |
| ZNF75D | NM_001185063 | CTGGGATGGAAAGGACCCCC | 4481 |
| ZNF764 | NM_001172679 | ACCGCGGCCATTTTGGATGA | 4482 |
| ZNF764 | NM_001172679 | GCACGACTGCGTAGGGGCAA | 4483 |
| ZNF768 | NM_024671 | TGCAGCCCAGCCCGGGGCCG | 4484 |
| ZNF773 | NM_198542 | TCGGGTAGACCTCTTTTCAT | 4485 |
| ZNF780A | NM_001010880 | ATCACAGCTCAAGGCTTCTG | 4486 |
| ZNF790 | NM_001242800 | GGAGCTGACCCTATCCGAAC | 4487 |
| ZNF791 | NM_153358 | TGTTGAAGCAGAAATTGTTC | 4488 |
| ZNF799 | NM_001080821 | CTTAAGTGCAAATATCCCTC | 4489 |
| ZNF808 | NM_001039886 | AAGACGCGCAAGTCCCGCCC | 4490 |
| ZNF81 | NM_007137 | CTGTTAGCCAGGAGTCAACA | 4491 |
| ZNF821 | NM_001201552 | GGGCCTGAGGAGAGGGGCTC | 4492 |
| ZNF83 | NM_001277952 | AACGATGCTGAGAGACTCAC | 4493 |
| ZNF837 | NM_138466 | TTCGGTTATCATAGAAACAG | 4494 |
| ZNF85 | NM_001256172 | AGAAGAGCGAGTGACAGCCT | 4495 |
| ZNF85 | NM_001256172 | TCACTCAGGGCCTGAAAAGA | 4496 |
| ZNF850 | NM_001193552 | CTCTGCGATCCTCGTTGGAG | 4497 |
| ZNF862 | NM_001099220 | CCCGGAACGCAGGTCCTGAT | 4498 |
| ZNRF3 | NM_001206998 | GACGCCTCACAGCCCCATCA | 4499 |
| ZP1 | NM_207341 | TTTCTGCCTCCCGCTGCCTT | 4500 |
| ZP3 | NM_001110354 | GTGTTACTGATGCTTCTGGA | 4501 |
| ZRANB1 | NM_017580 | AGAAACATGTTGAGAAGTAA | 4502 |
| ZRANB1 | NM_017580 | TTTGAGGCTACAGATTATCA | 4503 |
| ZRANB3 | NM_032143 | ATTCATAGGTTGTACGTCCC | 4504 |
| ZSCAN2 | NM_017894 | GGCTGGGCCCAAGGCATTGT | 4505 |
| ZSCAN5B | NM_001080456 | ATATTACTGAGAAGAAACAG | 4506 |
| ZSWIM1 | NM_080603 | GAGGTAAAGATACTTGCATC | 4507 |
| ZSWIM3 | NM_080752 | AATCTAGGTTATGATTGGTC | 4508 |
| ZUFSP | NM_145062 | CAGGAGAATGGCGTGAACCC | 4509 |
| ZWILCH | NM_017975 | GATATTTTTTGTATCCGTGT | 4510 |
| ZYG11B | NM_024646 | GGCCTGGGAGGGGGAGAAGC | 4511 |
| ZZZ3 | NM_015534 | ATTTAAAACACTGAGACAGT | 4512 |

We claim:

1. A system for targeted genome engineering, the system comprising one or more vectors comprising:
(i) nucleic acids for integration in genomic DNA with no significant homology to the target sequence in genomic DNA;
(ii) a single guide RNA (sgRNA) that binds one or more vectors;
(iii) a sgRNA that binds a double-stranded nucleic sequence in genomic DNA where the vectors can be integrated; and
(iv) a nuclease that causes a double-stranded nucleic acid break of the targeted nucleic acid molecules.

2. The system of claim 1, wherein components (i), (ii), (iii), and (iv) are located on the same or different vectors of the system.

3. The system of claim 1, wherein the sgRNAs of components (ii) and (iii) are the same sgRNA.

4. The system of claim 1, wherein the sgRNAs of components (ii) and (iii) are different sgRNAs.

5. The system of claim 1, wherein the sgRNA of component (ii) is a universal sgRNA.

6. The system of claim 1, wherein the nuclease is expressed from an expression cassette.

7. The system of claim 1, wherein the one or more vectors further comprises a polynucleotide encoding for a marker protein.

8. The system of claim 7, wherein a sgRNA target site is cloned upstream of the marker protein.

9. The system of claim 7, wherein the marker protein is an antibiotic resistance protein or a florescent protein.

10. The system of claim 7, wherein the polynucleotide encoding for a marker protein is expressed on a vector separate from the one or more vectors comprising components (i)-(iv).

11. The system of claim 1, wherein the sgRNA of component (iii) is complementary to a portion of the nucleic acid sequence of a target DNA.

12. The system of claim 1, wherein the nucleic acids with no significant homology to the target nucleic acid molecule are about 0.1 kilobase to about 50 kilobases in size.

13. The system of claim 1, wherein the nuclease is Zinc finger nuclease (ZFN), RNA guided nucleases (RGN), or transcription activator-like effector nucleases (TALEN).

14. The system of claim 13, wherein the RGN is Caspase 9 (Cas9).

15. The system of claim 1, wherein the one or more vectors are plasmids or viral vectors.

16. The system of claim 15, wherein the viral vector is a lentivirus vector, an adenovirus vector, or an adeno-associated vector (AAV).

17. The system of claim 1, further comprising one or more additional sgRNA molecules that causes a double-stranded nucleic acid break of one or more additional target nucleic acid molecules.

18. The system of claim 1, wherein the system does not require the entire vector that can be integrated to have any homology with the target site.

19. A method of altering the expression of at least one gene product, the method comprising:
(i) introducing into a cell the system of claim 1; and
(ii) selecting for successfully transfected cells by applying selective pressure;
wherein the expression of at least one gene product is reduced or eliminated relative to a cell that has not been transfected with the system of claim 1.

20. The method of claim 19, wherein the method occurs in vivo or in vitro.

21. The method of claim 19, wherein the cell is a eukaryotic cell.

22. A system for targeted genome engineering, the system comprising one or more vectors comprising:
(i) at least one nucleic acid with no significant homology to the target genomic DNA site and that contains a promoter for controlling gene expression;
(ii) a primary sgRNA that binds the target nucleic acid molecule at or near the transcription start site of a gene in the target nucleic acid molecule;
(iii) a universal secondary sgRNA that binds one or more vectors; and
(iv) a nuclease that causes a double-stranded nucleic acid break of the targeted nucleic acid molecules.

23. The system of claim 22, wherein component (1) comprises:
(1) a nucleic acid promoter followed by a universal secondary sgRNA;
(2) two opposing, constitutive promoters separated by a universal secondary sgRNA; or
(3) two inducible promoters in opposite orientations separated by an universal secondary sgRNA.

24. The system of claim 22, wherein components (i), (ii), (iii), and (iv) are located on the same or different vectors of the system.

25. The system of claim 23, wherein each inducible promotor of component (3) contains multiple TetO repeats and a transferase gene operatively linked to a reverse tetracycline transactivator (rtTA) via a T2A peptide.

26. The system of claim 22, wherein the one or more vectors further comprise a polynucleotide encoding for a marker protein.

27. The system of claim 25, wherein the marker protein is an antibiotic resistance protein or a florescent protein.

28. The system of claim 22, wherein the nucleic acid promotor is heterologous to the promoter of the target nucleic acid molecule.

29. The system of claim 22, wherein the nuclease is a Zinc finger nuclease (ZFN), RNA guided nucleases (RGN), or transcription activator-like effector nucleases (TALEN).

30. The system of claim 29, wherein the RGN is Caspase 9 (Cas9).

31. The system of claim 22, wherein the one or more vectors are plasmid or viral vectors.

32. The system of claim 31, wherein the viral vector is a lentivirus vector, an adenovirus vector, or an adeno-associated vector (AAV).

33. A method of altering the expression of at least one gene product, the method comprising:
(i) introducing into a cell the system of claim 22;
(ii) selecting for successfully transfected cells by applying selective pressure; and
(iii) wherein the expression of at least one gene product is activated relative to a cell that is not transfected with the system of claim 22.

34. The method of claim 33, wherein the method occurs in vivo or in vitro.

35. The method of claim 33, wherein the cell is a eukaryotic cell.

36. A method of identifying the genetic basis of one or more medical symptoms exhibited by a subject, the method comprising:
(i) obtaining a biological sample from the subject and isolating a population of cells having a first phenotype from the biological sample;
(ii) transfecting a library of sgRNA into the cells;
(iii) introducing into the cells the system of claim 22;
(iv) selecting for successfully transfected cells by applying the selective pressure;
(v) selecting the cells that survive under the selective pressure,
(vi) determining the genomic loci of the DNA molecule that interacts with the first phenotype and identifying the genetic basis of the one or more medical symptoms exhibited by the subject.

37. The method of claim 36, wherein selective pressure is applied by contacting the cells with an antibiotic and selecting the cells that survive.

38. The method of claim 37, wherein the antibiotic is puromycin or hygromycin.


SEQUENCE LISTING (220 sequences)

SEQ ID NO: 1 - DNA, 19 bp, Artificial Sequence
  Synthetic oligonucleotide
agcaggagtatgacgagtc

SEQ ID NO: 2 - DNA, 19 bp, Artificial Sequence
  Synthetic oligonucleotide
cggtggacgatggaggggc

SEQ ID NO: 3 - DNA, 19 bp, Artificial Sequence
  Synthetic oligonucleotide
atggcccacatggcctcca

SEQ ID NO: 4 - DNA, 19 bp, Artificial Sequence
  Synthetic oligonucleotide
ggtgaggaggccgaagagg

SEQ ID NO: 5 - DNA, 20 bp, Artificial Sequence
  Synthetic oligonucleotide
cggtgaggaggccgaagagg

SEQ ID NO: 6 - DNA, 19 bp, Artificial Sequence
  Synthetic oligonucleotide
caggggcctgcccatgcca

SEQ ID NO: 7 - DNA, 19 bp, Artificial Sequence
  Synthetic oligonucleotide
aagtggataagagcgccgt

SEQ ID NO: 8 - DNA, 19 bp, Artificial Sequence
  Synthetic oligonucleotide
gcgctcttgtctactcggt

SEQ ID NO: 9 - DNA, 19 bp, Artificial Sequence
  Synthetic oligonucleotide
gctgtgctgatgagcgctc

SEQ ID NO: 10 - DNA, 20 bp, Artificial Sequence
  Synthetic oligonucleotide
aagcagaaactacccgttgc

SEQ ID NO: 11 - DNA, 19 bp, Artificial Sequence
  Synthetic oligonucleotide
tgtactctctgaggtgctc

SEQ ID NO: 12 - DNA, 20 bp, Artificial Sequence
  Synthetic oligonucleotide
accgggtcttcgagaagacc

SEQ ID NO: 13 - DNA, 20 bp, Artificial Sequence
  Synthetic oligonucleotide
tcgataagccagtaagcagt

SEQ ID NO: 14 - DNA, 19 bp, Artificial Sequence
  Synthetic oligonucleotide
cgtaatacgactcactata

SEQ ID NO: 15 - DNA, 20 bp, Artificial Sequence
  Synthetic oligonucleotide
tgagggccaagttttccgcg

SEQ ID NO: 16 - DNA, 20 bp, Artificial Sequence
  Synthetic oligonucleotide
ttacggggcggcgacctcgc

SEQ ID NO: 17 - DNA, 20 bp, Artificial Sequence
  Synthetic oligonucleotide
gtcacatccagggtcctcac

SEQ ID NO: 18 - DNA, 20 bp, Artificial Sequence
  Synthetic oligonucleotide
tctgcgcaagttaggttttg

SEQ ID NO: 19 - DNA, 20 bp, Artificial Sequence
  Synthetic oligonucleotide
agggccctgacaactctttt

SEQ ID NO: 20 - DNA, 20 bp, Artificial Sequence
  Synthetic oligonucleotide
aggggtctacatggcaactg

SEQ ID NO: 21 - DNA, 20 bp, Artificial Sequence
  Synthetic oligonucleotide
catggacgagatggagttca

SEQ ID NO: 22 - DNA, 20 bp, Artificial Sequence
  Synthetic oligonucleotide
gaatgggcaccagaaagaaa

SEQ ID NO: 23 - DNA, 20 bp, Artificial Sequence
  Synthetic oligonucleotide
gataaggggcagctgagtga

SEQ ID NO: 24 - DNA, 20 bp, Artificial Sequence
  Synthetic oligonucleotide
gtgcgatgaggtgcacatag

SEQ ID NO: 25 - DNA, 21 bp, Artificial Sequence
  Synthetic oligonucleotide
tgcccttgtcttgtagtttcc

SEQ ID NO: 26 - DNA, 20 bp, Artificial Sequence
  Synthetic oligonucleotide
atatctgcggggtgtttcac

SEQ ID NO: 27 - DNA, 20 bp, Artificial Sequence
  Synthetic oligonucleotide
gcctgactgtgggcttgtat

SEQ ID NO: 28 - DNA, 20 bp, Artificial Sequence
  Synthetic oligonucleotide
gcggtgagttcaggcttttt

SEQ ID NO: 29 - DNA, 19 bp, Artificial Sequence
  Synthetic oligonucleotide
ctcccttctcagcctcctg

SEQ ID NO: 30 - DNA, 20 bp, Artificial Sequence
  Synthetic oligonucleotide
gtttttccttttccggtgtg

SEQ ID NO: 31 - DNA, 20 bp, Artificial Sequence
  Synthetic oligonucleotide
gcgcttgatgtgtttgtgag

SEQ ID NO: 32 - DNA, 20 bp, Artificial Sequence
  Synthetic oligonucleotide
cctcatacgatggggaactg

SEQ ID NO: 33 - DNA, 19 bp, Artificial Sequence
  Synthetic oligonucleotide
cctccatcgtccaccgcaa

SEQ ID NO: 34 - DNA, 20 bp, Artificial Sequence
  Synthetic oligonucleotide
gtggatcagcaagcaggagt

SEQ ID NO: 35 - DNA, 20 bp, Artificial Sequence
  Synthetic oligonucleotide
tcccgagctctactgactcc

SEQ ID NO: 36 - DNA, 20 bp, Artificial Sequence
  Synthetic oligonucleotide
ttggcttgtagcaggacctt

SEQ ID NO: 37 - DNA, 21 bp, Artificial Sequence
  Synthetic oligonucleotide
tgcaaaatttgtggagaatga

SEQ ID NO: 38 - DNA, 20 bp, Artificial Sequence
  Synthetic oligonucleotide
atgcttttcagccacattca

SEQ ID NO: 39 - DNA, 20 bp, Artificial Sequence
  Synthetic oligonucleotide
caatgaccccttcattgacc

SEQ ID NO: 40 - DNA, 20 bp, Artificial Sequence
  Synthetic oligonucleotide
ttgattttggagggatctcg

SEQ ID NO: 41 - DNA, 20 bp, Artificial Sequence
  Synthetic oligonucleotide
ggaatccatggagggaagat

SEQ ID NO: 42 - DNA, 20 bp, Artificial Sequence
  Synthetic oligonucleotide
tgttctcgctcaggtcagtg

SEQ ID NO: 43 - DNA, 20 bp, Artificial Sequence
  Synthetic oligonucleotide
ttacagccagtagtgctcgc

SEQ ID NO: 44 - DNA, 20 bp, Artificial Sequence
  Synthetic oligonucleotide
cccaggcttgtccacatcat

SEQ ID NO: 45 - DNA, 20 bp, Artificial Sequence
  Synthetic oligonucleotide
gcacttatccccaggcttgt

SEQ ID NO: 46 - DNA, 20 bp, Artificial Sequence
  Synthetic oligonucleotide
ggttgttgttctgcgggttc

SEQ ID NO: 47 - DNA, 20 bp, Artificial Sequence
  Synthetic oligonucleotide
ccattttatgacggcggcag

SEQ ID NO: 48 - DNA, 20 bp, Artificial Sequence
  Synthetic oligonucleotide
gtgcgatgaggtgcacatag

SEQ ID NO: 49 - DNA, 20 bp, Artificial Sequence
  Synthetic oligonucleotide
gataaggggcagctgagtga

SEQ ID NO: 50 - DNA, 20 bp, Artificial Sequence
  Synthetic oligonucleotide
gagaaacactggaccccgta

SEQ ID NO: 51 - DNA, 18 bp, Artificial Sequence
  Synthetic oligonucleotide
tgtaaaacgacggccagt

SEQ ID NO: 52 - DNA, 17 bp, Artificial Sequence
  Synthetic oligonucleotide
caggaaacagctatgac

SEQ ID NO: 53 - DNA, 20 bp, Artificial Sequence
  Synthetic oligonucleotide
gataacactgcggccaactt

SEQ ID NO: 54 - DNA, 20 bp, Artificial Sequence
  Synthetic oligonucleotide
ggcacctatctcagcgatct

SEQ ID NO: 55 - DNA, 20 bp, Artificial Sequence
  Synthetic oligonucleotide
ccttctagttgccagccatc

SEQ ID NO: 56 - DNA, 19 bp, Artificial Sequence
  Synthetic oligonucleotide
gctgggtgtcccattgaaa

SEQ ID NO: 57 - DNA, 19 bp, Artificial Sequence
  Synthetic oligonucleotide
cagccgctcgctgcagcag

SEQ ID NO: 58 - DNA, 19 bp, Artificial Sequence
  Synthetic oligonucleotide
tggagagtttgcaaggagc

SEQ ID NO: 59 - DNA, 19 bp, Artificial Sequence
  Synthetic oligonucleotide
gtttattcagccgggagtc

SEQ ID NO: 60 - DNA, 25 bp, Artificial Sequence
  Synthetic oligonucleotide
  misc_feature (6)..(25): the 20 n residues represent the 20 bases of the genomic DNA at the 5′ end of the PAM
  misc_feature (6)..(25): n is a or g or c or t/u
caccgnnnnnnnnnnnnnnnnnnnn

SEQ ID NO: 61 - DNA, 24 bp, Artificial Sequence
  Synthetic oligonucleotide
  misc_feature (5)..(24): the n 20 residues represent the reverse compliment fo the target sequence
  misc_feature (5)..(24): n is a or g or c or t/u
aaacnnnnnnnnnnnnnnnnnnnn

SEQ ID NO: 62 - DNA, 24 bp, Artificial Sequence
  Synthetic oligonucleotide
caccggctgggtgtcccattgaaa

SEQ ID NO: 63 - DNA, 24 bp, Artificial Sequence
  Synthetic oligonucleotide
aaactttcaatgggacacccagcc

SEQ ID NO: 64 - DNA, 24 bp, Artificial Sequence
  Synthetic oligonucleotide
caccgcagccgctcgctgcagcag

SEQ ID NO: 65 - DNA, 24 bp, Artificial Sequence
  Synthetic oligonucleotide
aaacctgctgcagcgagcggctgc

SEQ ID NO: 66 - DNA, 24 bp, Artificial Sequence
  Synthetic oligonucleotide
caccgtggagagtttgcaaggagc

SEQ ID NO: 67 - DNA, 24 bp, Artificial Sequence
  Synthetic oligonucleotide
aaacgctccttgcaaactctccac

SEQ ID NO: 68 - DNA, 24 bp, Artificial Sequence
  Synthetic oligonucleotide
caccggtttattcagccgggagtc

SEQ ID NO: 69 - DNA, 24 bp, Artificial Sequence
  Synthetic oligonucleotide
aaacgactcccggctgaataaacc

SEQ ID NO: 70 - DNA, 18 bp, Artificial Sequence
  Synthetic oligonucleotide
tgtaaaacgacggccagt

SEQ ID NO: 71 - DNA, 20 bp, Artificial Sequence
  Synthetic oligonucleotide
ggagcttctcgacttcacca

SEQ ID NO: 72 - DNA, 20 bp, Artificial Sequence
  Synthetic oligonucleotide
aacgccactgacaagaaagc

SEQ ID NO: 73 - DNA, 24 bp, Artificial Sequence
  Synthetic oligonucleotide
caccgctctgattccgcgactcct

SEQ ID NO: 74 - DNA, 24 bp, Artificial Sequence
  Synthetic oligonucleotide
aaacaggagtcgcggaatcagagc

SEQ ID NO: 75 - DNA, 24 bp, Artificial Sequence
  Synthetic oligonucleotide
caccgccagaagtgagagagtgct

SEQ ID NO: 76 - DNA, 24 bp, Artificial Sequence
  Synthetic oligonucleotide
aaacagcactctctcacttctggc

SEQ ID NO: 77 - DNA, 25 bp, Artificial Sequence
  Synthetic oligonucleotide
caccgcgggagaaaggaacgggagg

SEQ ID NO: 78 - DNA, 25 bp, Artificial Sequence
  Synthetic oligonucleotide
aaaccctcccgttcctttctcccgc

SEQ ID NO: 79 - DNA, 25 bp, Artificial Sequence
  Synthetic oligonucleotide
caccgaagaacttgaagcaaagcgc

SEQ ID NO: 80 - DNA, 25 bp, Artificial Sequence
  Synthetic oligonucleotide
aaacgcgctttgcttcaagttcttc

SEQ ID NO: 81 - DNA, 25 bp, Artificial Sequence
  Synthetic oligonucleotide
cctcgaagaacttgaagcaaagcgc

SEQ ID NO: 82 - DNA, 25 bp, Artificial Sequence
  Synthetic oligonucleotide
cctcgaggccaataggaacactgcg

SEQ ID NO: 83 - DNA, 25 bp, Artificial Sequence
  Synthetic oligonucleotide
aaaccggtgaccctagaaattggac

SEQ ID NO: 84 - DNA, 25 bp, Artificial Sequence
  Synthetic oligonucleotide
caccgtccaatttctagggtcaccg

SEQ ID NO: 85 - DNA, 24 bp, Artificial Sequence
  Synthetic oligonucleotide
caccgttgtgagccgtcctgtagg

SEQ ID NO: 86 - DNA, 24 bp, Artificial Sequence
  Synthetic oligonucleotide
aaaccctacaggacggctcacaac

SEQ ID NO: 87 - DNA, 25 bp, Artificial Sequence
  Synthetic oligonucleotide
tcccagtattggtggaagcttctta

SEQ ID NO: 88 - DNA, 25 bp, Artificial Sequence
  Synthetic oligonucleotide
aaactaagaagcttccaccaatact

SEQ ID NO: 89 - DNA, 26 bp, Artificial Sequence
  Synthetic oligonucleotide
ttgtttgagagaatcccttgaagacg

SEQ ID NO: 90 - DNA, 26 bp, Artificial Sequence
  Synthetic oligonucleotide
aaaccgtcttcaagggattctctcaa

SEQ ID NO: 91 - DNA, 27 bp, Artificial Sequence
  Synthetic oligonucleotide
ttgtttgcttcccccgcacaatagcgg

SEQ ID NO: 92 - DNA, 27 bp, Artificial Sequence
  Synthetic oligonucleotide
aaacccgctattgtgcgggggaagcaa

SEQ ID NO: 93 - DNA, 25 bp, Artificial Sequence
  Synthetic oligonucleotide
caccgcgatttcctacattcaacaa

SEQ ID NO: 94 - DNA, 25 bp, Artificial Sequence
  Synthetic oligonucleotide
aaacttgttgaatgtaggaaatcgc

SEQ ID NO: 95 - DNA, 25 bp, Artificial Sequence
  Synthetic oligonucleotide
caccgaggggagcggttgtcggagg

SEQ ID NO: 96 - DNA, 25 bp, Artificial Sequence
  Synthetic oligonucleotide
aaaccctccgacaaccgctcccctc

SEQ ID NO: 97 - DNA, 25 bp, Artificial Sequence
  Synthetic oligonucleotide
caccgacctgcccatttgtatgccg

SEQ ID NO: 98 - DNA, 25 bp, Artificial Sequence
  Synthetic oligonucleotide
aaaccggcatacaaatgggcaggtc

SEQ ID NO: 99 - DNA, 24 bp, Artificial Sequence
  Synthetic oligonucleotide
tcccacctgcccatttgtatgccg

SEQ ID NO: 100 - DNA, 24 bp, Artificial Sequence
  Synthetic oligonucleotide
aaaccggcatacaaatgggcaggt

SEQ ID NO: 101 - DNA, 25 bp, Artificial Sequence
  Synthetic oligonucleotide
caccgaggtccgcggagtctctaac

SEQ ID NO: 102 - DNA, 25 bp, Artificial Sequence
  Synthetic oligonucleotide
aaacgttagagactccgcggacctc

SEQ ID NO: 103 - DNA, 24 bp, Artificial Sequence
  Synthetic oligonucleotide
caccgtcgccagttagagactccg

SEQ ID NO: 104 - DNA, 24 bp, Artificial Sequence
  Synthetic oligonucleotide
aaaccggagtctctaactggcgac

SEQ ID NO: 105 - DNA, 25 bp, Artificial Sequence
  Synthetic oligonucleotide
caccgtagaggggccgacggagatt

SEQ ID NO: 106 - DNA, 25 bp, Artificial Sequence
  Synthetic oligonucleotide
aaacaatctccgtcggcccctctac

SEQ ID NO: 107 - DNA, 25 bp, Artificial Sequence
  Synthetic oligonucleotide
caccggtgaaatgagggcttgcgaa

SEQ ID NO: 108 - DNA, 25 bp, Artificial Sequence
  Synthetic oligonucleotide
aaacttcgcaagccctcatttcacc

SEQ ID NO: 109 - DNA, 26 bp, Artificial Sequence
  Synthetic oligonucleotide
ttgtttgtgaaatgagggcttgcgaa

SEQ ID NO: 110 - DNA, 26 bp, Artificial Sequence
  Synthetic oligonucleotide
aaacttcgcaagccctcatttcacaa

SEQ ID NO: 111 - DNA, 25 bp, Artificial Sequence
  Synthetic oligonucleotide
caccgctctcctccacccatccagg

SEQ ID NO: 112 - DNA, 25 bp, Artificial Sequence
  Synthetic oligonucleotide
aaaccctggatgggtggaggagagc

SEQ ID NO: 113 - DNA, 25 bp, Artificial Sequence
  Synthetic oligonucleotide
caccgacctgcactgaggtcctgga

SEQ ID NO: 114 - DNA, 25 bp, Artificial Sequence
  Synthetic oligonucleotide
aaactccaggacctcagtgcaggtc

SEQ ID NO: 115 - DNA, 25 bp, Artificial Sequence
  Synthetic oligonucleotide
caccgcctttaatcatgacactggg

SEQ ID NO: 116 - DNA, 25 bp, Artificial Sequence
  Synthetic oligonucleotide
aaaccccagtgtcatgattaaaggc

SEQ ID NO: 117 - DNA, 25 bp, Artificial Sequence
  Synthetic oligonucleotide
caccgggaatgcctaggattctgga

SEQ ID NO: 118 - DNA, 25 bp, Artificial Sequence
  Synthetic oligonucleotide
aaactccagaatcctaggcattccc

SEQ ID NO: 119 - DNA, 25 bp, Artificial Sequence
  Synthetic oligonucleotide
caccgtcgataagccagtaagcagt

SEQ ID NO: 120 - DNA, 25 bp, Artificial Sequence
  Synthetic oligonucleotide
aaacactgcttactggcttatcgac

SEQ ID NO: 121 - DNA, 25 bp, Artificial Sequence
  Synthetic oligonucleotide
cctcgtcgataagccagtaagcagt

SEQ ID NO: 122 - DNA, 25 bp, Artificial Sequence
  Synthetic oligonucleotide
aaacactgcttactggcttatcgac

SEQ ID NO: 123 - DNA, 24 bp, Artificial Sequence
  Synthetic oligonucleotide
tcccattagaccgcgtcagtccgg

SEQ ID NO: 124 - DNA, 24 bp, Artificial Sequence
  Synthetic oligonucleotide
aaacccggactgacgcggtctaat

SEQ ID NO: 125 - DNA, 22 bp, Artificial Sequence
  Synthetic oligonucleotide
ttccttctttcactcgccctcc

SEQ ID NO: 126 - DNA, 20 bp, Artificial Sequence
  Synthetic oligonucleotide
ccagtttctccctcgctgtt

SEQ ID NO: 127 - DNA, 20 bp, Artificial Sequence
  Synthetic oligonucleotide
ggcccacactttgctttctg

SEQ ID NO: 128 - DNA, 20 bp, Artificial Sequence
  Synthetic oligonucleotide
ctttgggcagcctaggactc

SEQ ID NO: 129 - DNA, 22 bp, Artificial Sequence
  Synthetic oligonucleotide
tgaggggctagcaggtctatgc

SEQ ID NO: 130 - DNA, 21 bp, Artificial Sequence
  Synthetic oligonucleotide
ggaatcccccacacctcagag

SEQ ID NO: 131 - DNA, 22 bp, Artificial Sequence
  Synthetic oligonucleotide
tgctagctacgatgcacatcca

SEQ ID NO: 132 - DNA, 22 bp, Artificial Sequence
  Synthetic oligonucleotide
gccccgaattcgagctcggtac

SEQ ID NO: 133 - DNA, 20 bp, Artificial Sequence
  Synthetic oligonucleotide
tttccaatgccacctcctcc

SEQ ID NO: 134 - DNA, 20 bp, Artificial Sequence
  Synthetic oligonucleotide
atgacgatcaaaagcccaag

SEQ ID NO: 135 - DNA, 20 bp, Artificial Sequence
  Synthetic oligonucleotide
gaatagcaaggcaccacctt

SEQ ID NO: 136 - DNA, 20 bp, Artificial Sequence
  Synthetic oligonucleotide
agctgatggccctaaacaga

SEQ ID NO: 137 - DNA, 20 bp, Artificial Sequence
  Synthetic oligonucleotide
aagcccttgctgtagtggtg

SEQ ID NO: 138 - DNA, 20 bp, Artificial Sequence
  Synthetic oligonucleotide
caggaggactctggcaccta

SEQ ID NO: 139 - DNA, 20 bp, Artificial Sequence
  Synthetic oligonucleotide
cggcaggaaagcatctgtat

SEQ ID NO: 140 - DNA, 20 bp, Artificial Sequence
  Synthetic oligonucleotide
ctggagcctgtgtgaacaga

SEQ ID NO: 141 - DNA, 20 bp, Artificial Sequence
  Synthetic oligonucleotide
caaccacctccaggcagtag

SEQ ID NO: 142 - DNA, 20 bp, Artificial Sequence
  Synthetic oligonucleotide
ttcggcttcctgtccatgac

SEQ ID NO: 143 - DNA, 20 bp, Artificial Sequence
  Synthetic oligonucleotide
ctgcctcaccctccttcaag

SEQ ID NO: 144 - DNA, 20 bp, Artificial Sequence
  Synthetic oligonucleotide
gaaggagaagctggagcaaa

SEQ ID NO: 145 - DNA, 20 bp, Artificial Sequence
  Synthetic oligonucleotide
atcccagggtgatcctcttc

SEQ ID NO: 146 - DNA, 49 bp, Artificial Sequence
  Synthetic oligonucleotide
tgctcgcgctactctctcttgaatgaatgattctggcctggaggctatc

SEQ ID NO: 147 - DNA, 81 bp, Artificial Sequence
  Synthetic oligonucleotide
ctcgctccgtggccttagctgtgctcgcgctactctctttctggcctggaggctatccaggcgtgagtctctcctaccctc

SEQ ID NO: 148 - DNA, 81 bp, Artificial Sequence
  Synthetic oligonucleotide
gagggtaggagagactcacgcctggatagcctccaggccagaaagagagtagcgcgagcacagctaaggccacggagcgag

SEQ ID NO: 149 - DNA, 92 bp, Artificial Sequence
  Synthetic oligonucleotide
ctcgctccgtggccttagctgtgctcgcgctactctcttgaatgaatgattctggcctggaggctatccaggcgtgagtctctcctaccctc

SEQ ID NO: 150 - DNA, 92 bp, Artificial Sequence
  Synthetic oligonucleotide
gagggtaggagagactcacgcctggatagcctccaggccagaatcattcattcaagagagtagcgcgagcacagctaaggccacggagcgag

SEQ ID NO: 151 - DNA, 36 bp, Artificial Sequence
  Synthetic oligonucleotide
tgctcgcgctactctctctttctggtgaatgaatga

SEQ ID NO: 152 - DNA, 82 bp, Artificial Sequence
  Synthetic oligonucleotide
ctcgctccgtggccttagctgtgctcgcgctactctctctttctggcctggaggctatccagcgtgagtctctcctaccctc

SEQ ID NO: 153 - DNA, 82 bp, Artificial Sequence
  Synthetic oligonucleotide
gagggtaggagagactcacgctggatagcctccaggccagaaagagagagtagcgcgagcacagctaaggccacggagcgag

SEQ ID NO: 154 - DNA, 36 bp, Artificial Sequence; identical to SEQ ID NO: 151
  Synthetic oligonucleotide

SEQ ID NO: 155 - DNA, 36 bp, Artificial Sequence
  Synthetic oligonucleotide
tcattcattcaccagaaagagagagtagcgcgagca

SEQ ID NO: 156 - DNA, 77 bp, Artificial Sequence
  Synthetic oligonucleotide
gtgctcgcgctactctctttctggtgaatgaatgactcgcgctactctctctttctggcctggaggctatccagcgt

SEQ ID NO: 157 - DNA, 77 bp, Artificial Sequence
  Synthetic oligonucleotide
acgctggatagcctccaggccagaaagagagagtagcgcgagtcattcattcaccagaaagagagtagcgcgagcac

SEQ ID NO: 158 - DNA, 22 bp, Artificial Sequence
  Synthetic oligonucleotide
agcaggagtatgacgagtccgg

SEQ ID NO: 159 - DNA, 22 bp, Artificial Sequence
  Synthetic oligonucleotide
ccggactcgtcatactcctgct

SEQ ID NO: 160 - DNA, 22 bp, Artificial Sequence
  Synthetic oligonucleotide
atggcccacatggcctccaagg

SEQ ID NO: 161 - DNA, 22 bp, Artificial Sequence
  Synthetic oligonucleotide
ccttggaggccatgtgggccat

SEQ ID NO: 162 - DNA, 49 bp, Artificial Sequence
  Synthetic oligonucleotide
agcaagcaggagtatgacgagtccggcccctccatggtccaccgcaaat

SEQ ID NO: 163 - DNA, 49 bp, Artificial Sequence
  Synthetic oligonucleotide
atttgcggtggaccatggaggggccggactcgtcatactcctgcttgct

SEQ ID NO: 164 - DNA, 56 bp, Artificial Sequence
  Synthetic oligonucleotide
tgtggatcagcaagcaggagtatgacgagtccggcccctccatcgtccaccgcaaa

SEQ ID NO: 165 - DNA, 56 bp, Artificial Sequence
  Synthetic oligonucleotide
tttgcggtggacgatggaggggccggactcgtcatactcctgcttgctgatccaca

SEQ ID NO: 166 - DNA, 23 bp, Artificial Sequence
  Synthetic oligonucleotide
cggtgaggaggccgaagaggagg

SEQ ID NO: 167 - DNA, 23 bp, Artificial Sequence
  Synthetic oligonucleotide
cctcctcttcggcctcctcaccg

SEQ ID NO: 168 - DNA, 22 bp, Artificial Sequence
  Synthetic oligonucleotide
aagtggataagagcgccgttgg

SEQ ID NO: 169 - DNA, 22 bp, Artificial Sequence
  Synthetic oligonucleotide
ccaacggcgctcttatccactt

SEQ ID NO: 170 - DNA, 23 bp, Artificial Sequence
  Synthetic oligonucleotide
ccgaccgagtagacaagaagcgc

SEQ ID NO: 171 - DNA, 23 bp, Artificial Sequence
  Synthetic oligonucleotide
gcgcttcttgtctactcggtcgg

SEQ ID NO: 172 - DNA, 22 bp, Artificial Sequence
  Synthetic oligonucleotide
taagtggagtccctgtgctagg

SEQ ID NO: 173 - DNA, 22 bp, Artificial Sequence
  Synthetic oligonucleotide
cctagcacagggactccactta

SEQ ID NO: 174 - DNA, 19 bp, Artificial Sequence
  Synthetic oligonucleotide
ggtgaggaggccgaagagg

SEQ ID NO: 175 - DNA, 19 bp, Artificial Sequence
  Synthetic oligonucleotide
gatgaggaggacgaagagg

SEQ ID NO: 176 - DNA, 19 bp, Artificial Sequence
  Synthetic oligonucleotide
ctggaggaggccgaagagg

SEQ ID NO: 177 - DNA, 28 bp, Artificial Sequence
  Synthetic oligonucleotide
cctcatggcccacatggcctccaagggg

SEQ ID NO: 178 - DNA, 22 bp, Artificial Sequence
  Synthetic oligonucleotide
cctcatggcccactccaagggg

SEQ ID NO: 179 - DNA, 29 bp, Artificial Sequence
  Synthetic oligonucleotide
cctcatggcccacatggccttccaagggg

SEQ ID NO: 180 - DNA, 35 bp, Artificial Sequence
  Synthetic oligonucleotide
cctcatggcccacatggccatgtggatccaagggg

SEQ ID NO: 181 - DNA, 44 bp, Artificial Sequence
  Synthetic oligonucleotide
cgggtcttcgagaagacctatggcccacatggcctccaaggagt

SEQ ID NO: 182 - DNA, 28 bp, Artificial Sequence
  Synthetic oligonucleotide
cgatggcccacatggccttccaaggagt

SEQ ID NO: 183 - DNA, 20 bp, Artificial Sequence
  Synthetic oligonucleotide
cgatggcccacatggcctgt

SEQ ID NO: 184 - DNA, 11 bp, Artificial Sequence
  Synthetic oligonucleotide
cgatgacccct

SEQ ID NO: 185 - DNA, 48 bp, Artificial Sequence
  Synthetic oligonucleotide
cctcatggcccacatggcctccaagtcttctcgaagacccggtgggta

SEQ ID NO: 186 - DNA, 33 bp, Artificial Sequence
  Synthetic oligonucleotide
cctcatggcccacatgtgggccatcggtgggta

SEQ ID NO: 187 - DNA, 39 bp, Artificial Sequence
  Synthetic oligonucleotide
cctcatggcccacatggccatgtgggccatcggtgggta

SEQ ID NO: 188 - DNA, 28 bp, Artificial Sequence
  Synthetic oligonucleotide
cctcatggcatgtggccatcggtgggta

SEQ ID NO: 189 - DNA, 30 bp, Artificial Sequence
  Synthetic oligonucleotide
gatccatggcccacatggcctccaaggagt

SEQ ID NO: 190 - DNA, 19 bp, Artificial Sequence
  Synthetic oligonucleotide
gatcccctttccaaggagt

SEQ ID NO: 191 - DNA, 21 bp, Artificial Sequence
  Synthetic oligonucleotide
gatccccttggaccaaggagt

SEQ ID NO: 192 - DNA, 22 bp, Artificial Sequence
  Synthetic oligonucleotide
gatccccttggatccaaggagt

SEQ ID NO: 193 - DNA, 33 bp, Artificial Sequence
  Synthetic oligonucleotide
tcagcaagcaggagtatgacgagtccggggatc

SEQ ID NO: 194 - DNA, 34 bp, Artificial Sequence
  Synthetic oligonucleotide
tcagcaagcaggagtatgacgaagtccggggatc

SEQ ID NO: 195 - DNA, 29 bp, Artificial Sequence
  Synthetic oligonucleotide
tcagcaagcaggagtatgtcccggggatc

SEQ ID NO: 196 - DNA, 35 bp, Artificial Sequence
  Synthetic oligonucleotide
ccggctagcaggagtatgacgagtccggcaaatgc

SEQ ID NO: 197 - DNA, 16 bp, Artificial Sequence
  Synthetic oligonucleotide
ccgagcaggagtatgc

SEQ ID NO: 198 - DNA, 20 bp, Artificial Sequence
  Synthetic oligonucleotide
ccgagcaggagggcaaatgc

SEQ ID NO: 199 - DNA, 59 bp, Artificial Sequence
  Synthetic oligonucleotide
gcaagcaggagtatgacgaagcaggagtatgacgagtcaggtcttctcgaagacccggt

SEQ ID NO: 200 - DNA, 34 bp, Artificial Sequence
  Synthetic oligonucleotide
gcaagcaggagtatcgtcatactccctgctcggt

SEQ ID NO: 201 - DNA, 40 bp, Artificial Sequence
  Synthetic oligonucleotide
gcaagcaggagtatgacgatcgtcatactccctgctcggt

SEQ ID NO: 202 - DNA, 29 bp, Artificial Sequence
  Synthetic oligonucleotide
tccagcaggagtatgacgagtccggcccc

SEQ ID NO: 203 - DNA, 23 bp, Artificial Sequence
  Synthetic oligonucleotide
tccccggactgtcccgcggcccc

SEQ ID NO: 204 - DNA, 22 bp, Artificial Sequence
  Synthetic oligonucleotide
tccccggacgtcccgcggcccc

SEQ ID NO: 205 - DNA, 46 bp, Artificial Sequence
  Synthetic oligonucleotide
tctggctaactagagaacccactcatacaaatgggcaggtcacgtg

SEQ ID NO: 206 - DNA, 46 bp, Artificial Sequence
  Synthetic oligonucleotide
  misc_feature (24)..(37): n is a or g or c or t
tctggctaactagagaacccactnnnnnnnnnnnnnnggtcacgtg

SEQ ID NO: 207 - DNA, 71 bp, Artificial Sequence
  Synthetic oligonucleotide
  misc_feature (33)..(48): n is a or g or c or t
tctggctaactagagaaccgatatcccactttnnnnnnnnnnnnnnnncatacaaatgggcaggtcacgtg

SEQ ID NO: 208 - DNA, 71 bp, Artificial Sequence
  Synthetic oligonucleotide
tctggctaactagagaaccgatatcccactttcgataagccagtaagccatacaaatgggcaggtcacgtg

SEQ ID NO: 209 - DNA, 22 bp, Artificial Sequence
  Synthetic oligonucleotide
gggcctatttcccatgattcct

SEQ ID NO: 210 - DNA, 19 bp, Artificial Sequence
  Synthetic oligonucleotide
aacttctcggggactgtgg

SEQ ID NO: 211 - DNA, 100 bp, Artificial Sequence
  Synthetic oligonucleotide
aatgatacggcgaccaccgagatctacactctttccctacacgacgctcttccgatcttaagtagaggctttatatatcttgtggaaaggacgaaacacc

SEQ ID NO: 212 - DNA, 102 bp, Artificial Sequence
  Synthetic oligonucleotide
aatgatacggcgaccaccgagatctacactctttccctacacgacgctcttccgatctgatgcacatctgctttatatatcttgtggaaaggacgaaacacc

SEQ ID NO: 213 - DNA, 104 bp, Artificial Sequence
  Synthetic oligonucleotide
aatgatacggcgaccaccgagatctacactctttccctacacgacgctcttccgatcttcgatagcaattcgctttatatatcttgtggaaaggacgaaacacc

SEQ ID NO: 214 - DNA, 106 bp, Artificial Sequence
  Synthetic oligonucleotide
aatgatacggcgaccaccgagatctacactctttccctacacgacgctcttccgatctgatcgatccagttaggctttatatatcttgtggaaaggacgaaacacc

SEQ ID NO: 215 - DNA, 108 bp, Artificial Sequence
  Synthetic oligonucleotide
aatgatacggcgaccaccgagatctacactctttccctacacgacgctcttccgatctacgatcgatacacgatcgctttatatatcttgtggaaaggacgaaacacc

SEQ ID NO: 216 - DNA, 91 bp, Artificial Sequence
  Synthetic oligonucleotide
caagcagaagacggcatacgagattcgccttggtgactggagttcagacgtgtgctcttccgatctgccaagttgataacggactagcctt

SEQ ID NO: 217 - DNA, 91 bp, Artificial Sequence
  Synthetic oligonucleotide
caagcagaagacggcatacgagatatagcgtcgtgactggagttcagacgtgtgctcttccgatctgccaagttgataacggactagcctt

SEQ ID NO: 218 - DNA, 80 bp, Artificial Sequence
  Synthetic oligonucleotide
  misc_feature (1)..(20): n is a or g or c or t
nnnnnnnnnnnnnnnnnnnngttttagagctaggccaacatgaggatcacccatgtctgcagggcctagcaagttaaaat

SEQ ID NO: 219 - DNA, 25 bp, Artificial Sequence
  Synthetic oligonucleotide
caccgggtcttcgagaagacctgtt

SEQ ID NO: 220 - DNA, 25 bp, Artificial Sequence
  Synthetic oligonucleotide
aacaggtcttctcgaagacccggtg